(19) United States
(12) Patent Application Publication
(10) Pub. No.: US 2023/0301188 A1
(43) Pub. Date: Sep. 21, 2023

(54) ORGANIC ELECTROLUMINESCENT ELEMENT, ORGANIC ELECTROLUMINESCENT LIGHT-EMITTING DEVICE, AND ELECTRONIC EQUIPMENT

(71) Applicant: IDEMITSU KOSAN CO., LTD., Tokyo (JP)

(72) Inventors: Takushi SHIOMI, Tokyo (JP); Hisato MATSUMOTO, Tokyo (JP); Toshinari OGIWARA, Tokyo (JP)

(73) Assignee: IDEMITSU KOSAN CO., LTD., Tokyo (JP)

(21) Appl. No.: 18/018,325

(22) PCT Filed: Jul. 27, 2021
(86) PCT No.: PCT/JP2021/027628

§ 371 Date: Jan. 27, 2023

(30) Foreign Application Priority Data
Jul. 29, 2020 (JP) 2020-128150

(51) Int. Cl. H10K 85/60; C09K 11/02; C09K 11/06; C07D 405/14; C07F 5/02

(52) U.S. Cl. CPC H10K 85/6574; C09K 11/02; C09K 11/06; C07D 405/14; H10K 85/654; C07F 5/027; H10K 85/658; C09K 2211/1007; C09K 2211/1018; H10K 50/11

(57) ABSTRACT

An organic electroluminescence device includes: an anode; a cathode; an emitting layer provided between the anode and the cathode; and a first layer provided between the emitting layer and the cathode, in which the first layer contains a first compound having at least one deuterium atom, and the emitting layer contains a delayed fluorescent compound.

### TECHNICAL FIELD

The present invention relates to an organic electroluminescence device, an organic electroluminescence apparatus, and an electronic device.

### BACKGROUND ART

When voltage is applied to an organic electroluminescence device (hereinafter, occasionally referred to as an organic EL device), holes are injected from an anode and electrons are injected from a cathode into an emitting layer. The injected holes and electrons are recombined in the emitting layer to form excitons. Specifically, according to the electron spin statistics theory, singlet excitons and triplet excitons are generated at a ratio of 25%:75%.

A fluorescent organic EL device using light emission from singlet excitons has been applied to a full-color display such as a mobile phone and a television set, but an internal quantum efficiency is said to be at a limit of 25%. Studies have thus been made to improve performance of the organic EL device.

For instance, the organic EL device is expected to emit light more efficiently using triplet excitons in addition to singlet excitons. In view of the above, a highly-efficient fluorescent organic EL device using thermally activated delayed fluorescence (hereinafter simply referred to as “delayed fluorescence” in some cases) has been proposed and studied.

A thermally activated delayed fluorescence (TADF) mechanism uses such a phenomenon in which inverse intersystem crossing from triplet excitons to singlet excitons thermally occurs when a material having a small energy difference (ΔST) between singlet energy level and triplet energy level is used. Thermally activated delayed fluorescence is explained in “Yuki Hando-tai no Debaisu Bussei (Device Physics of Organic Semiconductors)” (edited by ADACHI Chihaya, published by Kodansha, issued on Apr. 1, 2012, on pages 261-268).

For instance, Patent Literatures 1 to 3 each disclose an organic EL device using the TADF mechanism.

Patent Literature 1 discloses, as a compound usable for a charge transporting material, a compound having a triazine skeleton.

Patent Literatures 2 and 3 each disclose, as a compound usable for an organic EL device, a compound having a deuterium atom.

### CITATION LIST

### Patent Literature(s)

Patent Literature 1: International Publication WO 2018/034340
Patent Literature 2: Korean Patent Publication No. 2019-072820 A
Patent Literature 3: Korean Patent Publication No. 2019-005805 A

### SUMMARY OF THE INVENTION

### Problems to be Solved by the Invention

A further improvement in performance of the organic EL device has been demanded for an improvement in performance of an electronic device such as a display.

An object of the invention is to provide an organic electroluminescence device excellent in performance (in particular, having a long lifetime), an organic electroluminescence apparatus including the organic electroluminescence device, and an electronic device including the organic electroluminescence device.

### Means for Solving the Problem(s)

According to an aspect of the invention, there is provided an organic electroluminescence device, including: an anode; a cathode; an emitting layer provided between the anode and the cathode; and a first layer provided between the emitting layer and the cathode, in which the first layer contains a first compound having at least one deuterium atom, and the emitting layer contains a delayed fluorescent compound.

According to another aspect of the invention, there is provided an organic electroluminescence apparatus, including: a first device that is the organic electroluminescence device according to the aspect of the invention; a second device that is an organic electroluminescence device different from the first device; and a substrate, in which the first device and the second device are arranged in parallel on the substrate, and the first layer of the first device is a common layer provided in common to the first device and the second device.

According to still another aspect of the invention, there is provided an electronic device including the organic electroluminescence device according to the aspect of the invention.

According to a further aspect of the invention, there is provided an electronic device including the organic electroluminescence apparatus according to the another aspect of the invention.

According to the aspects of the invention, there are provided an organic electroluminescence device excellent in performance (in particular, having a long lifetime), an organic electroluminescence apparatus including the organic electroluminescence device, and an electronic device including the organic electroluminescence device.

### BRIEF EXPLANATION OF DRAWING(S)

FIG. 1 schematically shows an exemplary arrangement of an organic electroluminescence device according to a first exemplary embodiment of the invention.

FIG. 2 schematically shows a device for measuring transient PL.

FIG. 3 shows an example of decay curves of the transient PL.

FIG. 4 schematically shows a relationship in energy level and energy transfer between a compound M1 and a compound M2 in an emitting layer of an exemplary organic electroluminescence device according to the first exemplary embodiment of the invention.

FIG. 5 schematically shows a relationship in energy level and energy transfer between the compound M1, the compound M2 and a compound M3 in an emitting layer of an exemplary organic electroluminescence device according to a second exemplary embodiment of the invention.

FIG. 6 schematically shows a relationship in energy level and energy transfer between the compound M2 and a compound M4 in an emitting layer of an exemplary organic electroluminescence device according to a third exemplary embodiment of the invention.

FIG. 7 schematically shows an exemplary arrangement of an organic electroluminescence device according to a fourth exemplary embodiment of the invention.

FIG. 8 schematically shows an exemplary arrangement of an organic electroluminescence apparatus according to a fifth exemplary embodiment of the invention.

FIG. 9 schematically shows an exemplary arrangement of an organic electroluminescence apparatus according to a sixth exemplary embodiment of the invention.

### DESCRIPTION OF EMBODIMENT(S)

### First Exemplary Embodiment

An arrangement of an organic EL device according to a first exemplary embodiment of the invention will be described below.

The organic EL device includes an anode, a cathode, and an organic layer between the anode and the cathode. The organic layer includes a plurality of layers formed from an organic compound(s). The organic layer may further contain an inorganic compound(s).

In the exemplary embodiment, at least two layers included in the organic layer are an emitting layer provided between the anode and the cathode and a first layer provided between the emitting layer and the cathode.

In the exemplary embodiment, the emitting layer contains a delayed fluorescent compound. The first layer contains a first compound having at least one deuterium atom.

Examples of the first layer, which are not particularly limited, include at least one selected from the group consisting of an electron injecting layer, an electron transporting layer, and a hole blocking layer. The first layer is preferably a hole blocking layer.

For instance, the organic layer may be provided in the form of the emitting layer and the first layer, or may further include a layer(s) usable in the organic EL device. Examples of the layer usable in the organic EL device, which are not particularly limited, include at least one selected from the group consisting of a hole injecting layer, a hole transporting layer, an electron injecting layer, an electron transporting layer, and a blocking layer.

The organic layer of the organic EL device in the exemplary embodiment preferably has a layer arrangement below.
electron blocking layer/emitting layer/hole blocking layer
hole injecting layer/electron blocking layer/emitting layer/hole blocking layer
hole transporting layer/electron blocking layer/emitting layer/hole blocking layer
hole injecting layer/hole transporting layer/electron blocking layer/emitting layer/hole blocking layer
electron blocking layer/emitting layer/hole blocking layer/electron injecting layer
electron blocking layer/emitting layer/hole blocking layer/electron transporting layer
electron blocking layer/emitting layer/hole blocking layer/electron transporting layer/electron injecting layer
hole injecting layer/electron blocking layer/emitting layer/hole blocking layer/electron injecting layer
hole injecting layer/electron blocking layer/emitting layer/hole blocking layer/electron transporting layer
hole injecting layer/electron blocking layer/emitting layer/hole blocking layer/electron transporting layer/electron injecting layer
hole transporting layer/electron blocking layer/emitting layer/hole blocking layer/electron injecting layer
hole transporting layer/electron blocking layer/emitting layer/hole blocking layer/electron transporting layer
hole transporting layer/electron blocking layer/emitting layer/hole blocking layer/electron transporting layer/electron injecting layer
hole injecting layer/hole transporting layer/electron blocking layer/emitting layer/hole blocking layer/electron injecting layer
hole injecting layer/hole transporting layer/electron blocking layer/emitting layer/hole blocking layer/electron transporting layer
hole injecting layer/hole transporting layer/electron blocking layer/emitting layer/hole blocking layer/electron transporting layer/electron injecting layer

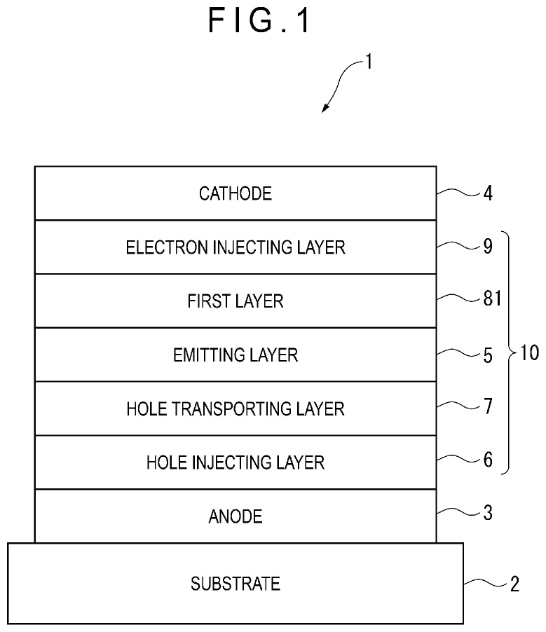

FIG. 1 schematically shows an exemplary arrangement of an organic EL device according to the exemplary embodiment.

An organic EL device 1 includes a light-transmissive substrate 2, an anode 3, a cathode 4, and an organic layer 10 provided between the anode 3 and the cathode 4. The organic layer 10 includes a hole injecting layer 6, a hole transporting layer 7, an emitting layer 5, a first layer 81, and an electron injecting layer 9 that are layered on the anode 3 in this order.

The first layer 81 is preferably in direct contact with the emitting layer 5.

The emitting layer 5 preferably contains no phosphorescent material (dopant material).

The emitting layer 5 preferably contains no phosphorescent metal complex.

The emitting layer 5 preferably contains no heavy metal complex. Examples of the heavy metal complex include an iridium complex, an osmium complex, and a platinum complex.

The emitting layer 5 preferably contains no phosphorescent rare-earth metal complex.

The emitting layer 5 may contain a metal complex, but preferably contains no metal complex.

Herein, a “deuterated compound” represents a compound in which at least part of protium atoms of the compound is substituted with a deuterium atom(s). Thus, the “first compound having at least one deuterium atom” in the exemplary embodiment is the “deuterated compound”.

Inventors of the invention have found out that an organic EL device using the TADF mechanism can be improved in performance (in particular, longer lifetime) by containing the “deuterated compound” in the first layer provided between the emitting layer and the cathode (in the exemplary embodiment, a layer with electron transportability, at least one of a hole blocking layer, an electron transporting layer, or an electron injecting layer).

Presumably, the layer with electron transportability through which electrons easily flow is likely to deteriorate due to holes that form pairs with electrons. For instance, “carbon-deuterium bond” is stronger than “carbon-protium bond”. Thus, it is assumed that deterioration in the layer with electron transportability due to holes can be inhibited by containing the “deuterated compound” in the layer with electron transportability, resulting in a long lifetime of the organic EL device.

Especially, in an organic EL device using the TADF mechanism, recombination positions of holes and electrons in the emitting layer are likely to be close to an electron transporting zone. Thus, when the “deuterated compound” is used in a layer with electron transportability, the effect of providing a long lifetime may be large.

Presumably, the effect of providing a long lifetime is further enhanced by deuteration in the vicinity of an electron-withdrawing group that is considered to be vulnerable to holes (group with electron injectability or electron transportability) such as azine.

The organic EL device according to the exemplary embodiment achieves a long lifetime, because the emitting layer contains a delayed fluorescent compound and the first layer contains the “deuterated compound”.

Further, the organic EL device according to the exemplary embodiment is expect to have high performance.

High performance means that the device has at least one of improved device lifetime, luminous efficiency, drive voltage, or luminance.

The organic EL device according to the exemplary embodiment is thus expected to be improved in at least one of luminous efficiency, drive voltage, or luminance, in addition to the device lifetime.

The wording “deuteration in the vicinity of an electron-withdrawing group” is exemplified by deuteration of an electron-withdrawing group itself, deuteration of a substituent E1 when an electron-withdrawing group has the substituent E1, deuteration of a substituent E2 when the substituent E1 further has the substituent E2, and deuteration of a protium atom bonded to at least one of the first to the eleventh atoms counting from an electron-withdrawing group.

An example of the first exemplary embodiment in which the emitting layer 5 contains a compound M2 as a delayed fluorescent compound and a fluorescent compound M1 is explained below.

In this example, the compound M2 is preferably a host material (occasionally also referred to as a matrix material). The compound M1 is preferably a dopant material (occasionally also referred to as a guest material, an emitter, or a luminescent material).

The first layer 81 is explained first, and the emitting layer 5 is explained next.

In the following explanation, the “first compound having at least one deuterium atom” is occasionally referred to as a “deuterated compound D1”. Further, a compound in which all the hydrogen atoms in the first compound are protium atoms is occasionally referred to as a “protium compound d1”.

### First Layer

### First Compound

The first layer 81 contains the first compound having at least one deuterium atom.

In the exemplary embodiment, the content ratio of the protium compound d1 to the total of the deuterated compound D1 and the protium compound d1 contained in the first layer 81 is 99 mol % or less. The content ratio of the protium compound d1 is determined by mass spectrometry.

In the exemplary embodiment, the content ratio of the deuterated compound D1 to the total of the deuterated compound D1 and the protium compound d1 contained in the first layer 81 is preferably 30 mol % or more, 50 mol % or more, 70 mol % or more, 90 mol % or more, 95 mol % or more, 99 mol % or more, or 100 mol %.

In the exemplary embodiment, also preferably, the ratio of the number of the deuterium atoms to the total number of the hydrogen atoms in the first compound is 10% or more, 20% or more, 30% or more, 40% or more, 50% or more, 60% or more, 70% or more, or 80% or more.

### Determination Method for the Presence of Deuterium Atom in First Compound and Specifying Method of Bonding Position of Deuterium Atom in First Compound

Whether the first compound has a deuterium atom is determined by mass spectrometry or ¹H-NMR spectrometry. A bonding position of a deuterium atom in the first compound is specified by the ¹H-NMR spectrometry.

Specifically, mass spectrometry is performed on a target compound. When a molecular weight of the target compound is increased by, for example, one as compared with a related compound in which all the hydrogen atoms in the target compound are replaced by protium atoms, it can be determined that the first compound has one deuterium atom. Further, since a signal of a deuterium atom does not appear in ¹H-NMR spectrometry, the number of deuterium atoms in a molecule can be determined by an integral value obtained by performing ¹H-NMR spectrometry on the target compound. Furthermore, a bonding position of a deuterium atom is specified by conducting ¹H-NMR spectrometry on the target compound to perform signal assignment.

In the exemplary embodiment, the first compound preferably includes at least one of partial structures represented by formulae (11) to (28) below in one molecule.

When the first compound includes a plurality of partial structures represented by any of the formulae (11) to (14), the partial structures represented by the formula (11) are mutually the same or different, the partial structures represented by the formula (12) are mutually the same or different, the partial structures represented by the formula (13) are mutually the same or different, and the partial structures represented by the formula (14) are mutually the same or different.

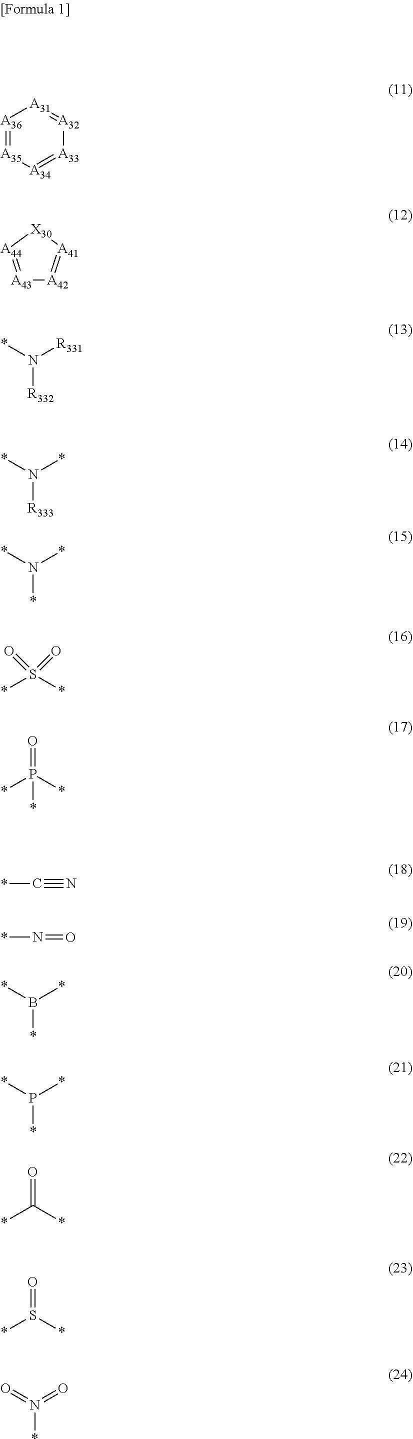

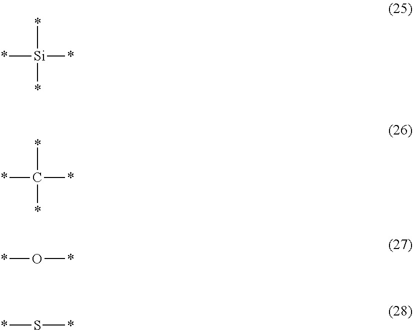

In the formula (11):
A₃₁ to A₃₆ are each independently a nitrogen atom, CR₃₁, or a carbon atom bonded to another atom or another structure in the molecule of the first compound;
at least one of A₃₁ to A₃₆ is a carbon atom bonded to another atom or another structure in the molecule of the first compound; and
each R₃₁ is independently a hydrogen atom or a substituent, or at least one combination of combinations of adjacent ones of R₃₁ are mutually bonded to form a ring,
in the formula (12):
A₄₁ to A₄₄ are each independently a nitrogen atom, CR₃₂, or a carbon atom bonded to another atom or another structure in the molecule of the first compound;
each R₃₂ is independently a hydrogen atom or a substituent, or at least one combination of combinations of adjacent ones of R₃₂ are mutually bonded to form a ring;
X₃₀ is NR₃₃, CR₃₄R₃₅, SiR₃₆R₃₇, an oxygen atom, a sulfur atom, a nitrogen atom bonded to another atom or another structure in the molecule of the first compound, a carbon atom bonded to R₃₈ and to another atom or another structure in the molecule of the first compound, or a silicon atom bonded to R₃₉ and to another atom or another structure in the molecule of the first compound;
at least one of carbon atoms for A₄₁ to A₄₄, a nitrogen atom for X₃₀, a carbon atom for X₃₀, or a silicon atom for X₃₀ is bonded to another atom or another structure in the molecule of the first compound; and
R₃₃ to R₃₉ are each independently a hydrogen atom or a substituent, or at least one combination of a combination of adjacent R₃₄ and R₃₅ or a combination of R₃₆ and R₃₇ are mutually bonded to form a ring,
in the formulae (13) and (14):
R₃₃₁ to R₃₃₃ are each independently a hydrogen atom or a substituent, or a combination of adjacent R₃₃₁ and R₃₃₂ are mutually bonded to form a ring;
R₃₁ to R₃₉ and R₃₃₁ to R₃₃₃ as the substituents are each independently a halogen atom, a cyano group, a substituted or unsubstituted aryl group having 6 to ring carbon atoms, a substituted or unsubstituted heterocyclic group having 5 to ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms;
a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted germanium group, a substituted phosphine oxide group, a nitro group, a substituted or unsubstituted carbonyl group, or a substituted boryl group;
a plurality of R₃₁ are mutually the same or different;
a plurality of R₃₂ are mutually the same or different; and
* is a bonding portion to another atom or another structure in the molecule of the first compound.

In the formula (12), when X₃₀ is “a nitrogen atom bonded to another atom or another structure in the molecule of the first compound”, the formula (12) is represented by a formula (12-1) below.

In the formula (12), when X₃₀ is “a carbon atom bonded to R₃₅ and to another atom or another structure in the molecule of the first compound”, the formula (12) is represented by a formula (12-2) below.

In the formula (12), when X₃₀ is “a silicon atom bonded to R₃₉ and to another atom or another structure in the molecule of the first compound, the formula (12) is represented by a formula (12-3) below.

In the formulae (12-1) to (12-3), A₄₁ to A₄₄, R₃₅ and R₃₉ each independently represent the same as A₄₁ to A₄₄, R₃₅ and R₃₉ in the formula (12), and * is a bonding portion to another atom or another structure in the molecule of the first compound.

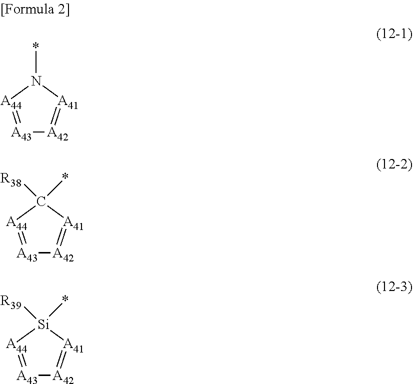

In the formulae (11) to (14) of the first compound, at least one of R₃₁ for CR₃₁, R₃₂ for CR₃₂, R₃₃ to R₃₉ for X₃₀, or R₃₃₁ to R₃₃₃ is preferably a deuterium atom.

In the formulae (11) and (12) of the first compound, at least one of R₃₁ for CR₃₁, R₃₂ for CR₃₂, or R₃₃ to R₃₉ for X₃₀ is more preferably a deuterium atom.

In the first compound, the partial structure represented by each of the formulae (11) to (28) is preferably a partial structure represented by any of formulae (111) to (138) below.

When the first compound includes a plurality of partial structures represented by any of the formulae (111) to (138), the plurality of partial structures represented by any of the formulae (111) to (138) are mutually the same or different. For instance, when the first compound includes a plurality of partial structures represented by the formula (111), the plurality of partial structures represented by the formula (111) are the same or different. The same applies to a case where the first compound includes a plurality of partial structures represented by any of the formulae (112) to (138).

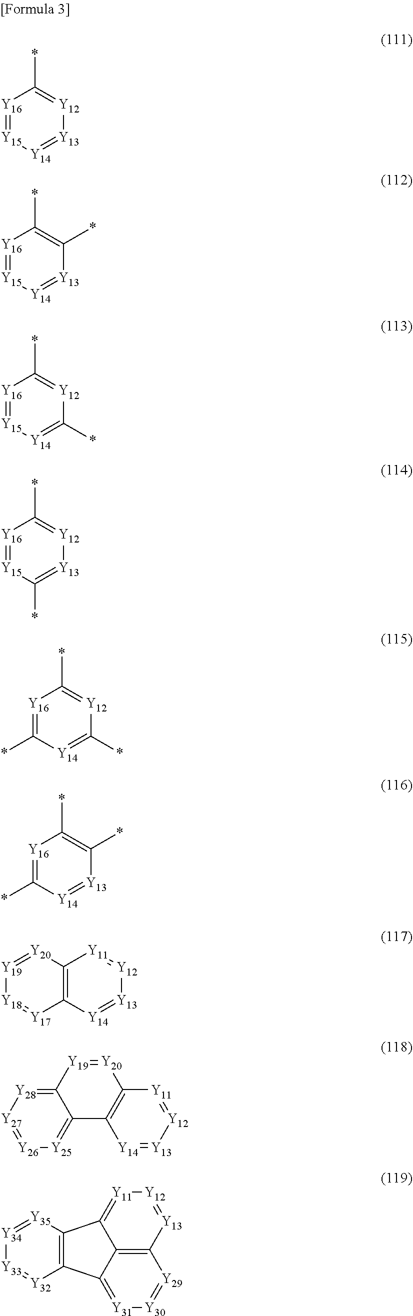

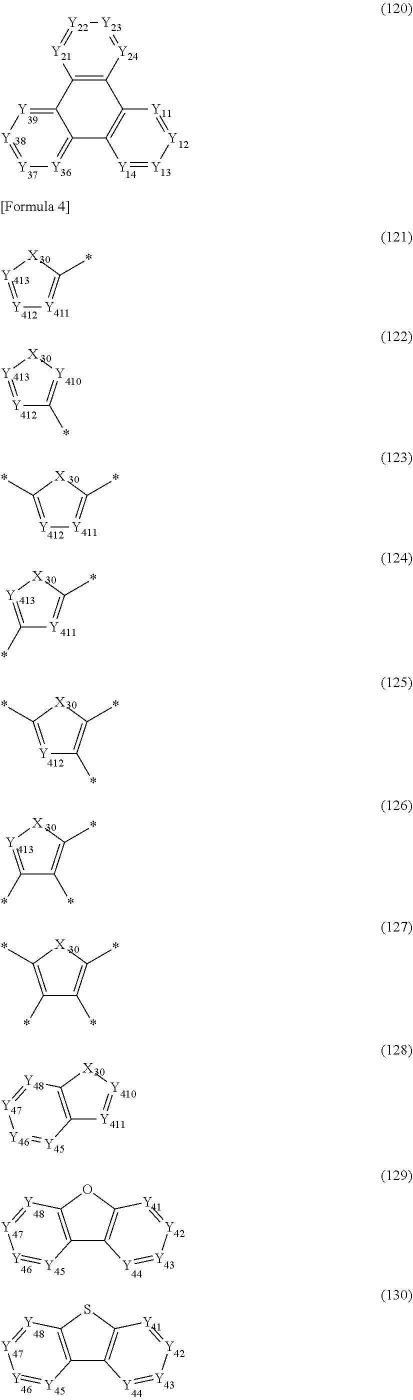

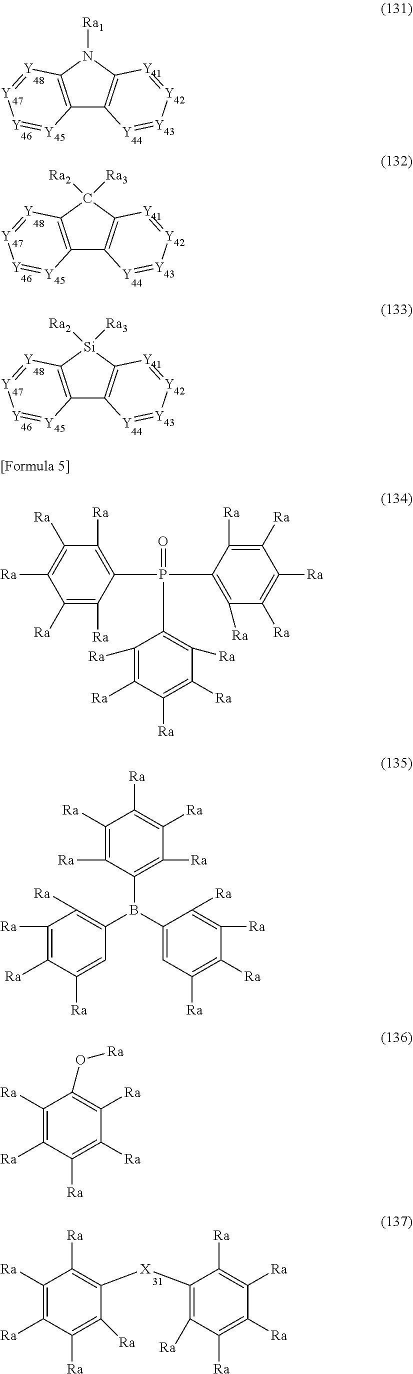

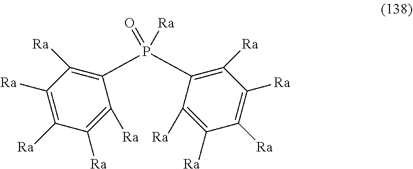

In the formulae (111) to (116), Y₁₂ to Y₁₆ are each independently a nitrogen atom or CR₃₁, each R₃₁ independently represents the same as R₃₁ in the formula (11), and * is a bonding portion to another atom or another structure in the molecule of the first compound;
in the formulae (117) to (120), Y₁₁ to Y₁₄ and Y₁₇ to Y₃₉ are each independently a nitrogen atom or CR₃₁, or a carbon atom bonded to another atom or another structure in the molecule of the first compound, each R₃₁ independently represents the same as R₃₁ in the formula (11), and at least one of Y₁₁ to Y₁₄ or Y₁₇ to Y₃₉ is a carbon atom bonded to another atom or another structure in the molecule of the first compound;
in the formulae (121) to (127), Y₄₁₀ to Y₄₁₃ are each independently a nitrogen atom or CR₃₂, each R₃₂ independently represents the same as R₃₂ in the formula (12), X₃₀ represents the same as X₃₀ in the formula (12), and * is a bonding portion to another atom or another structure in the molecule of the first compound;
in the formula (128), Y₄₁₀ to Y₄₁₁ and Y₄₅ to Y₄₈ are each independently a nitrogen atom or CR₃₂, or a carbon atom bonded to another atom or another structure in the molecule of the first compound, each R₃₂ independently represents the same as R₃₂ in the formula (12), X₃₀ represents the same as X₃₀ in the formula (12), at least one of carbon atoms for Y₄₁₀ to Y₄₁₁ and Y₄₅ to Y₄₈, a nitrogen atom for X₃₀, a carbon atom for X₃₀, or a silicon atom for X₃₀ is bonded to another atom or another structure in the molecule of the first compound;
in the formulae (129) to (133), Y₄₁ to Y₄₈ are each independently a nitrogen atom or CR₃₂, or a carbon atom bonded to another atom or another structure in the molecule of the first compound, Rₐ₁ to Rₐ₃ are each independently a hydrogen atom or a substituent, or a combination of Rₐ₂ and Rₐ₃ are mutually bonded to form a ring, R₃₂ represents the same as R₃₂ in the formula (12), Rₐ₁ to Rₐ₃ as the substituents each independently represent the same as R₃₂ as the substituent in the formula (12), when a plurality of Rₐ₂ are present, the plurality of Rₐ₂ are mutually the same or different, when a plurality of Rₐ₃ are present, the plurality of Rₐ₃ are mutually the same or different, and at least one of carbon atoms for Y₄₁ to Y₄₈, a nitrogen atom bonded to Rₐ₁, a carbon atom bonded to Rₐ₂, a carbon atom bonded to Rₐ₃, a silicon atom bonded to Rₐ₂, or a silicon atom bonded to Rₐ₃ is bonded to another atom or another structure in the molecule of the first compound, and
in the formulae (134) to (138), each Ra is independently a hydrogen atom or a substituent, at least one combination of combinations of adjacent ones of Ra are mutually bonded to form a ring, or Ra is a single bond bonded to another atom or another structure in the molecule of the first compound, each Ra as the substituent independently represents the same as R₃₁ as the substituent in the formula (11), a plurality of Ra are mutually the same or different, X₃₁ represents the same as X₃₀ in the formula (12), and at least one Ra is a single bond bonded to another atom or another structure in the molecule of the first compound.

In the first compound, R₃₁ to R₃₉, R₃₃₁ to R₃₃₃, Rₐ₁ to Rₐ₃ and Ra are preferably each independently a hydrogen atom, a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, or a substituted or unsubstituted aryloxy group having 6 to 30 ring carbon atoms.

In the formulae (117) to (120), it is preferable that a combination of adjacent ones of R₃₁ are not mutually bonded.

In the formulae (128) to (133), it is preferable that a combination of adjacent ones of R₃₂ are not mutually bonded.

In the formulae (132) and (133), it is preferable that a combination of adjacent Rₐ₂ and Rₐ₃ are not mutually bonded.

In the formulae (134) to (138), it is preferable that a combination of adjacent ones of Ra are not mutually bonded.

In the first compound, it is also preferable that at least one combination of a combination of adjacent ones of R₃₁, a combination of adjacent ones of R₃₂, a combination of adjacent R₃₄ and R₃₅, a combination of adjacent R₃₆ and R₃₇, a combination of adjacent R₃₃₁ and R₃₃₂, a combination of adjacent Rₐ₂ and Rₐ₃, or a combination of adjacent ones of Ra are mutually bonded to form a ring.

The partial structures represented by the formulae (111) to (117) and (128) are preferably each independently bonded to a metallic atom. Examples of the metallic atom include aluminium, zinc, and lithium.

The first compound preferably includes a partial structure represented by the formula (18).

In the exemplary embodiment, the first compound preferably includes, as the partial structure, a cyano group, or at least one monovalent or higher-valent residue derived from any of a substituted or unsubstituted benzene, a substituted or unsubstituted naphthalene, a substituted or unsubstituted indole, a substituted or unsubstituted carbazole, a substituted or unsubstituted dibenzofuran, a substituted or unsubstituted dibenzothiophene, a substituted or unsubstituted fluorene, a substituted or unsubstituted triazine, a substituted or unsubstituted pyrimidine, a substituted or unsubstituted pyridine, a substituted or unsubstituted pyridazine, a substituted or unsubstituted pyrazine, a substituted or unsubstituted imidazole, a substituted or unsubstituted benzimidazole, a substituted or unsubstituted benzothiazole, a substituted or unsubstituted benzisothiazol, a substituted or unsubstituted phenanthrene, a substituted or unsubstituted phenanthroline, a substituted or unsubstituted quinolone, a substituted or unsubstituted isoquinoline, and a substituted or unsubstituted silole.

In the formulae (111) to (138) of the exemplary embodiment, at least one of R₃₁ for CR₃₁, R₃₂ for CR₃₂, R₃₃ to R₃₉ for X₃₀, R₃₃ to R₃₉ for X₃₁, Rₐ₁ to Rₐ₃, or Ra is preferably a deuterium atom.

In the exemplary embodiment, Rₐ₁ in the formula (131) preferably has no deuterium atom.

In the exemplary embodiment, the first compound preferably includes no partial structure represented by the formula (131).

In the exemplary embodiment, the first compound preferably includes no partial structure represented by the formula (132) in which a combination of Rₐ₂ and Rₐ₃ are mutually bonded.

In the exemplary embodiment, the first compound preferably has no spirofluorene structure.

In the exemplary embodiment, the first compound is preferably not a compound represented by a formula (133A) below.

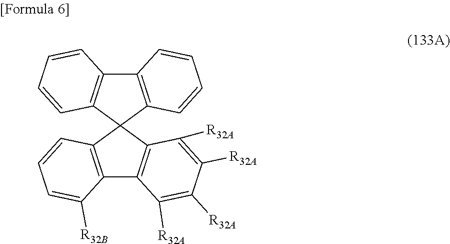

In the formula (133A), each R_{32A} is independently a hydrogen atom or a substituent, or at least one combination of combinations of adjacent ones of R_{32A} are mutually bonded to form a ring, each R_{32A} as the substituent is independently a substituted or unsubstituted aryl group having 6 to 30 ring carbon atoms or a substituted or unsubstituted heteroaryl group having 5 to 30 ring atoms, and a plurality of R_{32A} are mutually the same or different, and
R_{32B} is a substituted or unsubstituted aryl group having 6 to 30 ring carbon atoms or a substituted or unsubstituted heteroaryl group having 5 to 30 ring atoms.

In the exemplary embodiment, the first compound preferably has a substituted or unsubstituted electron-withdrawing group.

In the first compound, the electron-withdrawing group preferably has at least one deuterium atom.

In the first compound, when the electron-withdrawing group has a substituent E1, the substituent E1 preferably has at least one deuterium atom.

In the first compound, when the substituent E1 further has a substituent E2, the substituent E2 preferably has at least one deuterium atom.

In the first compound, when the electron-withdrawing group has a substituent E1, the substituent E1 preferably has at least one deuterium atom, or when the substituent E1 further has a substituent E2, the substituent E2 preferably has at least one deuterium atom.

Preferably, the substituent E1 and the substituent E2 are each independently a halogen atom, a cyano group, a substituted or unsubstituted aryl group having 6 to ring carbon atoms, a substituted or unsubstituted heterocyclic group having 5 to ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted germanium group, a substituted phosphine oxide group, a nitro group, a substituted or unsubstituted carbonyl group, or a substituted boryl group.

In the substituent E1 and the substituent E2, the substituent for the substituted or unsubstituted group is preferably an unsubstituted group.

In the first compound, the substituent E1 and the substituent E2 are preferably each independently a substituted or unsubstituted alkyl group having 1 to 6 carbon atoms, a substituted or unsubstituted aryl group having 6 to 22 ring carbon atoms, or a substituted or unsubstituted heterocyclic group having 5 to 20 ring atoms.

In the first compound, the substituent E1 and the substituent E2 are more preferably each independently a substituted or unsubstituted aryl group having 6 to 22 ring carbon atoms, or a substituted or unsubstituted heterocyclic group having 5 to 20 ring atoms.

In the exemplary embodiment, when the first compound has a substituted or unsubstituted electron-withdrawing group, a deuterium atom is preferably bonded to at least one of the first to the eleventh atoms counting from the electron-withdrawing group. Further preferably, deuterium atoms are bonded at at least 11 positions of the first to the eleventh atoms counting from the electron-withdrawing group.

A deuterium atom is preferably bonded to at least one of the first to the eighth atoms counting from the electron-withdrawing group. Further preferably, deuterium atoms are bonded at at least eight positions of the first to the eighth atoms counting from the electron-withdrawing group.

A deuterium atom is preferably bonded to at least one of the first to the fourth atoms counting from the electron-withdrawing group. Further preferably, deuterium atoms are bonded at at least four positions of the first to the fourth atoms counting from the electron-withdrawing group.

First to Eleventh Atoms Counting from Electron-Withdrawing Group

How to count atoms from an electron-withdrawing group is as follows: an atom bonded and nearest to the electron-withdrawing group is regarded as the first atom, an atom bonded and nearest to the first atom is the second atom, . . . and an atom bonded and nearest to the tenth atom is regarded as the eleventh atom. Thus, each of the first to the eleventh atoms occasionally include a plurality of atoms. The electron-withdrawing group in the wording of “counting from the electron-withdrawing group” is an electron-withdrawing group assuming that the electron-withdrawing group is an unsubstituted group.

Further, the first compound may have a plurality of electron-withdrawing groups in one molecule. In this case, if n=1 to 11 is satisfied when counting the n-th atom (n is an integer of one or more) from any one of the plurality of electron-withdrawing groups, the n-th atom corresponds to one of “the first to the eleventh atoms counting from the electron-withdrawing group”. For example, when n=1 to 8 is satisfied, the n-th atom corresponds to one of “the first to the eighth atoms counting from the electron-withdrawing group”. When n=1 to 4 is satisfied, the n-th atom corresponds to one of “the first to the fourth atoms counting from the electron-withdrawing group”.

A plurality of electron-withdrawing groups are mutually the same or different.

Explanation is made in detail using formulae (E-1) and (E-2) below.

A compound represented by a formula (E-1) has, in one molecule, triazine as an electron-withdrawing group. A compound represented by a formula (E-2) has, in one molecule, dibenzofuran as an electron-withdrawing group. These compounds are the same compound.

In the formula (E-1), numbers 1 to 13 are attached to positions corresponding to the first to the thirteenth atoms counting from triazine.

In the formula (E-2), numbers 1 to 14 are attached to positions corresponding to the first to the fourteenth atoms counting from each of two dibenzofurans.

In the compounds represented by the formulae (E-1) and (E-2), “the first to the eleventh atoms counting from the electron-withdrawing group” mean atoms at positions attached with the numbers 1 to 11 in the formulae (E-1) and (E-2). Thus, the wording of “a deuterium atom is bonded to at least one of the first to the eleventh atoms counting from the electron-withdrawing group” means that at least one of hydrogen atoms bonded to the atoms at positions attached with the numbers 1 to 11 in the formulae (E-1) and (E-2) is a deuterium atom.

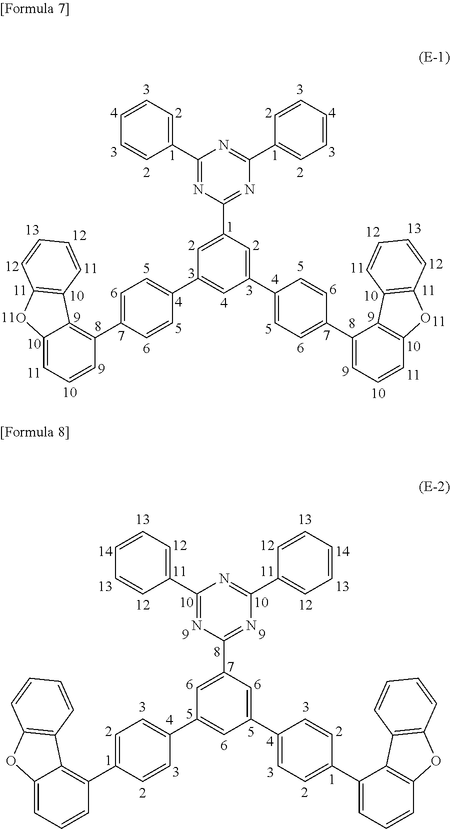 [SMILES: c1ccc(-c2nc(-c3ccccc3)nc(-c3cc(-c4ccc(-c5cccc6oc7ccccc7c56)cc4)cc(-c4ccc(-c5cccc6oc7ccccc7c56)cc4)c3)n2)cc1 ; c1ccc(-c2nc(-c3ccccc3)nc(-c3cc(-c4ccc(-c5cccc6oc7ccccc7c56)cc4)cc(-c4ccc(-c5cccc6oc7ccccc7c56)cc4)c3)n2)cc1]

In the exemplary embodiment, it is preferable that each electron-withdrawing group is independently a halogen atom, a cyano group, a carbonyl group, a nitro group, or a substituted or unsubstituted alkyl halide group; a monovalent or higher-valent group obtained by removing at least one hydrogen atom from a compound selected from the group consisting of a substituted or unsubstituted phosphine oxide, a substituted or unsubstituted sulfone, a substituted or unsubstituted sulfoxide, a substituted or unsubstituted nitroso, a substituted or unsubstituted pyridine, a substituted or unsubstituted pyrimidine, a substituted or unsubstituted pyridazine, a substituted or unsubstituted pyrazine, a substituted or unsubstituted triazine, a substituted or unsubstituted imidazole, a substituted or unsubstituted oxazole, a substituted or unsubstituted thiazole, a substituted or unsubstituted triazole, a substituted or unsubstituted benzimidazole, a substituted or unsubstituted benzoxazole, a substituted or unsubstituted benzothiazole, a substituted or unsubstituted benzotriazole, a substituted or unsubstituted boryl, a substituted or unsubstituted dibenzofuran, a substituted or unsubstituted dibenzothiophene, a substituted or unsubstituted fluoranthene, a substituted or unsubstituted phenanthrene, a substituted or unsubstituted chrysene, a substituted or unsubstituted triphenylene and, a substituted or unsubstituted naphthalene; a monovalent or higher-valent group formed by further fusing the above monovalent or higher-valent group itself; or a monovalent or higher-valent group obtained by removing at least one hydrogen atom from a compound selected from the group consisting of a substituted or unsubstituted azadibenzofuran and a substituted or unsubstituted azadibenzothiophene.

In the exemplary embodiment, it is more preferable that each electron-withdrawing group is independently a halogen atom, a cyano group, a carbonyl group, a nitro group, or a substituted or unsubstituted alkyl halide group; a monovalent or higher-valent group obtained by removing at least one hydrogen atom from a compound selected from the group consisting of a substituted or unsubstituted phosphine oxide, a substituted or unsubstituted sulfone, a substituted or unsubstituted sulfoxide, a substituted or unsubstituted nitroso, a substituted or unsubstituted pyridine, a substituted or unsubstituted pyrimidine, a substituted or unsubstituted pyridazine, a substituted or unsubstituted pyrazine, a substituted or unsubstituted triazine, a substituted or unsubstituted imidazole, a substituted or unsubstituted oxazole, a substituted or unsubstituted thiazole, a substituted or unsubstituted triazole, a substituted or unsubstituted benzimidazole, a substituted or unsubstituted benzoxazole, a substituted or unsubstituted benzothiazole, a substituted or unsubstituted benzotriazole, a substituted or unsubstituted boryl, a substituted or unsubstituted dibenzofuran, a substituted or unsubstituted dibenzothiophene, and a substituted or unsubstituted fluoranthene; a monovalent or higher-valent group formed by further fusing the above monovalent or higher-valent group itself; or a monovalent or higher-valent group obtained by removing at least one hydrogen atom from a compound selected from the group consisting of a substituted or unsubstituted azadibenzofuran and a substituted or unsubstituted azadibenzothiophene.

Herein, azadibenzofuran means a compound in which at least one of eight C—H groups in a dibenzofuran ring is substituted with a nitrogen atom.

Herein, azadibenzothiophene means a compound in which at least one of eight C—H groups in a dibenzofuran ring is substituted with a nitrogen atom.

In the exemplary embodiment, the first compound is preferably a compound represented by a formula (1) below.

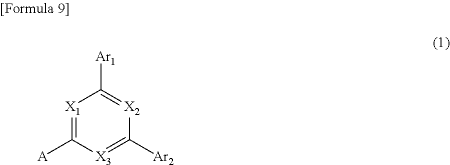

In the formula (1):
X₁ to X₃ are each independently a nitrogen atom or CR₁;
R₁ is a hydrogen atom or a substituent, or at least one combination of combinations of adjacent two or more of a plurality of R₁ are bonded to each other to form a ring;
at least one of X₁ to X₃ is a nitrogen atom;
each R₁ as the substituent is independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted germanium group, a substituted phosphine oxide group, a nitro group, a substituted or unsubstituted carbonyl group, or a substituted boryl group;
a plurality of R₁ are mutually the same or different;
Ar₁ and Ar₂ are each independently represented by a formula (11) below, or are each independently a substituted or unsubstituted aryl group having 6 to 30 ring carbon atoms, or a substituted or unsubstituted heteroaryl group having 5 to 30 ring atoms; and
A is represented by the formula (11), or is a substituted or unsubstituted aryl group having 6 to 30 ring carbon atoms, or a substituted or unsubstituted heteroaryl group having 5 to 30 ring atoms.

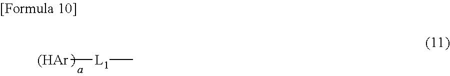

In the formula (11):
HAr is represented by a formula (12) below;
a is 1, 2, 3, 4 or 5;
when a is 1, L₁ is a single bond or a divalent linking group;
when a is 2, 3, 4 or 5, L₁ is a trivalent to hexavalent linking group;
a plurality of HAr are mutually the same or different;
L₁ as the linking group is a substituted or unsubstituted arylene group having 6 to 30 ring carbon atoms, or a trivalent, tetravalent, pentavalent, or hexavalent group derived from the arylene group; a substituted or unsubstituted divalent heterocyclic group having 5 to 30 ring atoms, or a trivalent, tetravalent, pentavalent, or hexavalent group derived from the heterocyclic group; or a divalent group formed by bonding two groups selected from the group consisting of a substituted or unsubstituted arylene group having 6 to 30 ring carbon atoms and a substituted or unsubstituted divalent heterocyclic group having 5 to 30 ring atoms, or a trivalent, tetravalent, pentavalent, or hexavalent group derived from the divalent group; and
the mutually bonded groups are mutually the same or different.

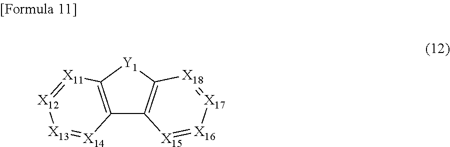

In the formula (12):
X₁₁ to X₁₈ are each independently a nitrogen atom, CR₁₃, or a carbon atom bonded to L₁;
a plurality of R₁₃ are mutually the same or different;
Y₁ is an oxygen atom, a sulfur atom, NR₁₈, SiR₁₁R₁₂, CR₁₄R₁₅, a nitrogen atom bonded to L₁, a silicon atom bonded to each of R₁₆ and L₁, or a carbon atom bonded to each of R₁₇ and L₁;
among carbon atoms for X₁₁ to X₁₈, a nitrogen atom for Y₁, a silicon atom for Y₁, and a carbon atom for Y₁, any one atom is bonded to L₁;
R₁₁ to R₁₈ are each independently a hydrogen atom or a substituent, or at least one combination of a combination of adjacent ones of R₁₃, a combination of R₁₁ and R₁₂, or a combination of R₁₄ and R₁₅ are bonded to each other to form a ring; and
R₁₁ to R₁₈ as the substituents are each independently a halogen atom, a cyano group, a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms; a substituted or unsubstituted silyl group, a substituted or unsubstituted alkoxy group having 1 to 30 carbon atoms, a substituted or unsubstituted aralkyl group having 7 to 30 carbon atoms, or a substituted or unsubstituted aryloxy group having 6 to 30 ring carbon atoms.

In the formula (11), when a is 1, L₁ is a divalent linking group, and the formula (11) is represented by a formula (111) below.

In the formula (11), when a is 2, 3, 4 or 5 or less, L₁ is a trivalent to hexavalent linking group. For instance, when a is 2, L₁ is a trivalent linking group, and the formula (11) is represented by a formula (112) below.

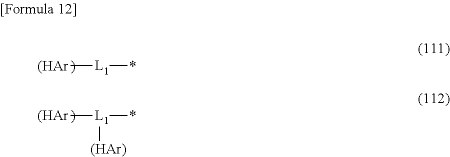

In the formulae (111) and (112), L₁ and HAr each independently represent the same as L₁ and HAr in the formula (11), and * represents a bonding position to a six-membered ring in the formula (1). A plurality of HAr are mutually the same or different.

In the formula (1), A is preferably a group represented by the formula (11).

In the formula (11), a is preferably 1, 2, or 3, more preferably 1 or 2.

In the formula (12), X₁₁ to X₁₈ are preferably each independently CR₁₃.

In the formula (12), Y₁ is preferably an oxygen atom, a sulfur atom, NR₁₈, CR₁₄R₁₅, a nitrogen atom bonded to L₁, or a carbon atom bonded to each of R₁₇ and L₁.

In the formula (12), X₁₃ or X₁₆ is preferably a carbon atom bonded to L₁ by a single bond.

In the formula (12), X₁₁ or X₁₈ is also preferably a carbon atom bonded to L₁ by a single bond.

In the formula (12), X₁₂ or X₁₇ is also preferably a carbon atom bonded to L₁ by a single bond.

In the formula (12), X₁₄ or X₁₅ is also preferably a carbon atom bonded to L₁ by a single bond.

In the exemplary embodiment, the first compound is preferably a compound represented by a formula (1A) below.

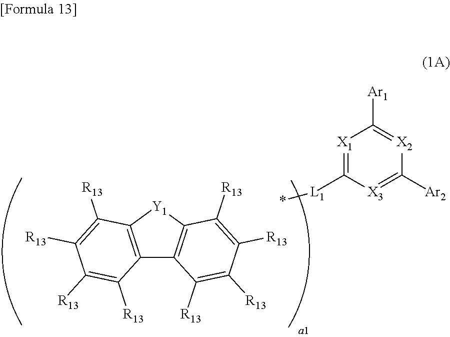

In the formula (1A):
X₁ to X₃, Ar₁ and Ar₂ each independently represent the same as X₁ to X₃, Ar₁ and Ar₂ in the formula (1);
L₁ represents the same as L₁ in the formula (11);
a1 is 1, 2, or 3;
Y₁ represents the same as Y₁ in the formula (12);
each R₁₃ independently represents the same as R₁₃ in the formula (12);
when a1 is 1, L₁ is a single bond or a divalent linking group;
when a1 is 2, L₁ is a trivalent linking group;
when a1 is 3, L₁ is a tetravalent linking group; and
among a carbon atom bonded to R₁₃, a nitrogen atom for Y₁, a silicon atom for Y₁, and a carbon atom for Y₁, any one atom is a bonding position * to L₁.

In the first compound, it is preferable that Ar₁ and Ar₂ are each independently represented by the formula (11), or are each independently a substituted or unsubstituted aryl group having 6 to 30 ring carbon atoms.

In the first compound, each R₁ for CR₁ is preferably independently a hydrogen atom, a substituted or unsubstituted aryl group having 6 to 30 ring carbon atoms, or a substituted or unsubstituted heteroaryl group having 5 to 30 ring atoms.

In the first compound, each R₁₃ is preferably independently a hydrogen atom, a substituted or unsubstituted alkyl group having 1 to 30 ring carbon atoms, a substituted or unsubstituted aryl group having 6 to 30 ring carbon atoms, or a substituted or unsubstituted heteroaryl group having 5 to 30 ring atoms.

In the first compound, L₁ is preferably a single bond; a substituted or unsubstituted arylene group having 6 to 30 ring carbon atoms, or a trivalent, tetravalent, pentavalent, or hexavalent group derived from the arylene group; or a substituted or unsubstituted divalent heterocyclic group having 5 to 30 ring atoms, or a trivalent, tetravalent, pentavalent, or hexavalent group derived from the heterocyclic group.

In the first compound, L₁ is more preferably a single bond; a substituted or unsubstituted arylene group having 6 to 30 ring carbon atoms, or a trivalent group derived from the arylene group; or a substituted or unsubstituted divalent heterocyclic group having 5 to 30 ring atoms, or a trivalent group derived from the heterocyclic group.

In the first compound, one or two of X₁, X₂, and X₃ are preferably nitrogen atoms.

In the first compound, X₁, X₂, and X₃ are each preferably a nitrogen atom.

In the first compound, at least one R₁₃ for CR₁₃ is preferably a deuterium atom.

In the first compound, at least one of Ar₁ or Ar₂ preferably has at least one deuterium atom.

In the first compound, it is also preferable that all of R₁₃ for CR₁₃ are deuterium atoms.

In the first compound, when Ar₁ includes one or more hydrogen atoms, it is also preferable that all the one or more hydrogen atoms are deuterium atoms.

In the first compound, when Ar₂ includes one or more hydrogen atoms, it is also preferable that all the one or more hydrogen atoms are deuterium atoms.

In the formula (1A), a1 is preferably 1 or 2.

The compound represented by the formula (1) is also preferably a compound represented by a formula (1-1) or (1-2) below.

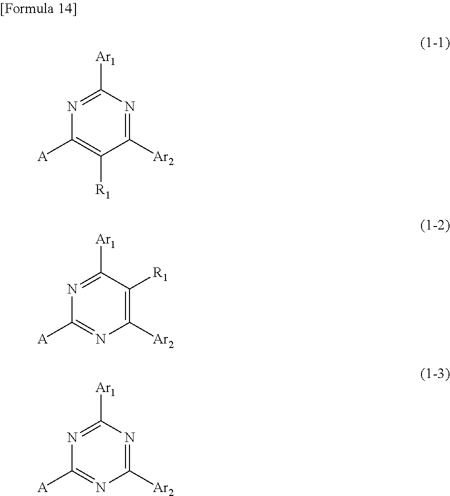

In the formulae (1-1) to (1-3), Ar₁, Ar₂, A and R₁ each independently represent the same as Ar₁, Ar₂, A and R₁ in the formula (1).

### Method for Producing First Compound (Deuterated Compound D1)

The first compound can be produced by a known method.

The first compound can be produced by, for instance, a method described later in Examples.

Further, the first compound can also be produced by reactions described in later-described Examples and using known alternative reactions or raw materials suitable for the desired substances.

### Specific Examples of First Compound

Specific examples of the first compound according to the exemplary embodiment include the following compounds. It should however be noted that the invention is not limited to the specific examples of the compound.

Description of hydrogen atoms is omitted in some of the specific examples of the first compound.

A specific example of the first compound in which description of hydrogen atoms is omitted is explained below.

For instance, a specific example of the first compound in which description of hydrogen atoms is omitted is represented by a formula (D-10) below. When hydrogen atoms are not omitted but described, the specific example of the first compound is represented by a formula (D-11) below.

In the formula (D-11), “H_{D}” represents a protium atom or a deuterium atom. At least one of a plurality of “H_{D}” is a deuterium atom.

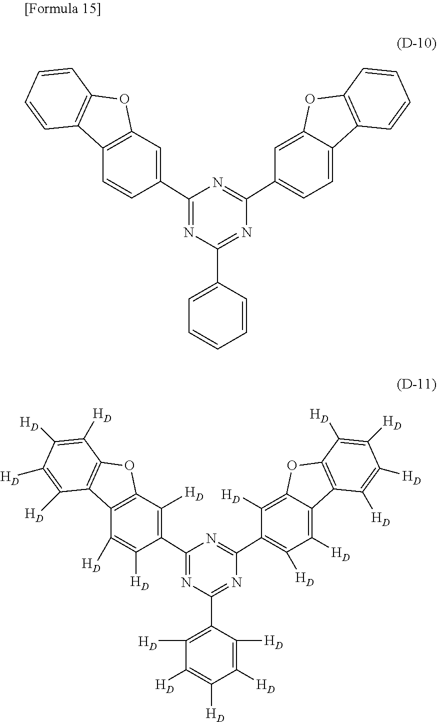 [SMILES: c1ccc(-c2nc(-c3ccc4c(c3)oc3ccccc34)nc(-c3ccc4c(c3)oc3ccccc34)n2)cc1 ; c1ccc(-c2nc(-c3ccc4c(c3)oc3ccccc34)nc(-c3ccc4c(c3)oc3ccccc34)n2)cc1]

Similarly, for instance, a specific example of the first compound in which description of hydrogen atoms is omitted is represented by a formula (D-20) below. When hydrogen atoms are not omitted but described, the specific example of the first compound is represented by a formula (D-21) below.

In the formula (D-21), “H_{D}” represents a protium atom or a deuterium atom. At least one of a plurality of “H_{D}” is a deuterium atom.

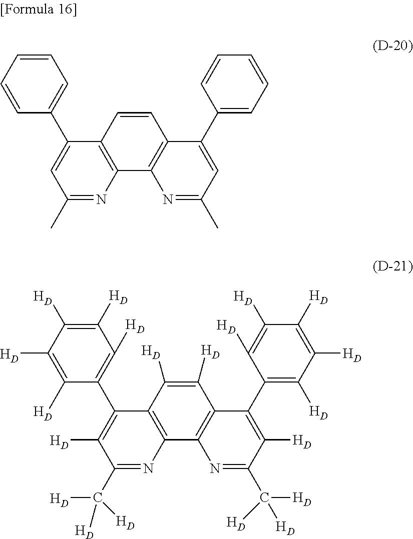 [SMILES: Cc1cc(-c2ccccc2)c2ccc3c(-c4ccccc4)cc(C)nc3c2n1 ; Cc1cc(-c2ccccc2)c2ccc3c(-c4ccccc4)cc(C)nc3c2n1]

Specific examples of the first compound shown below are specific examples in which description of hydrogen atoms is omitted.

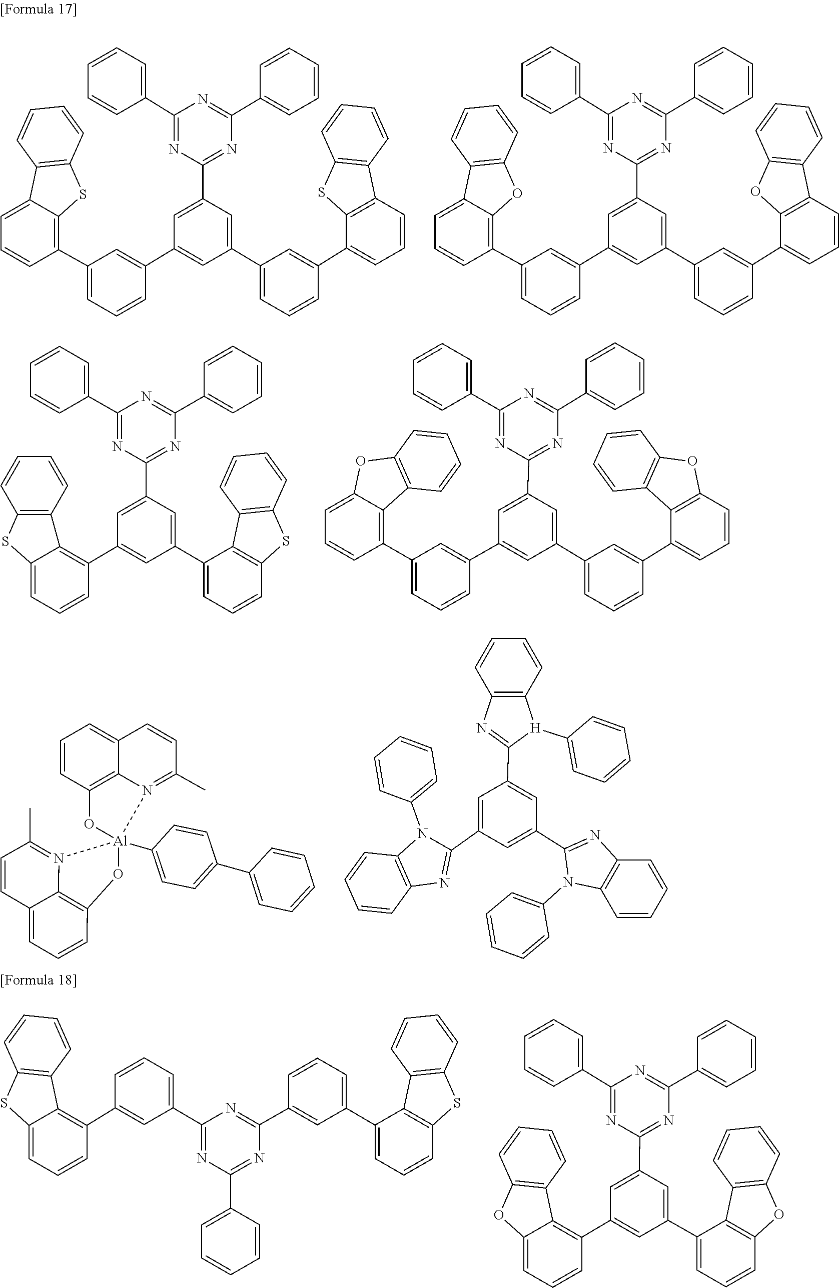

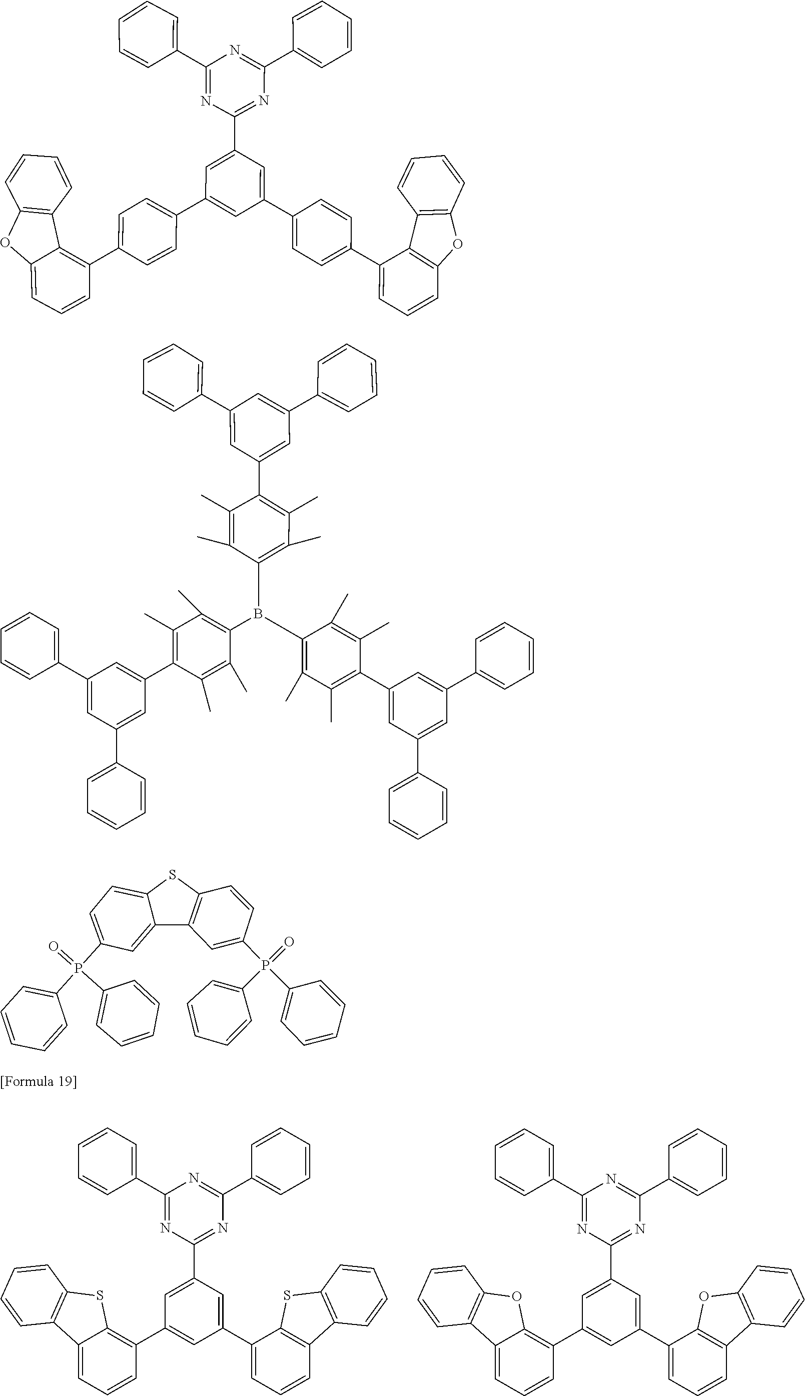

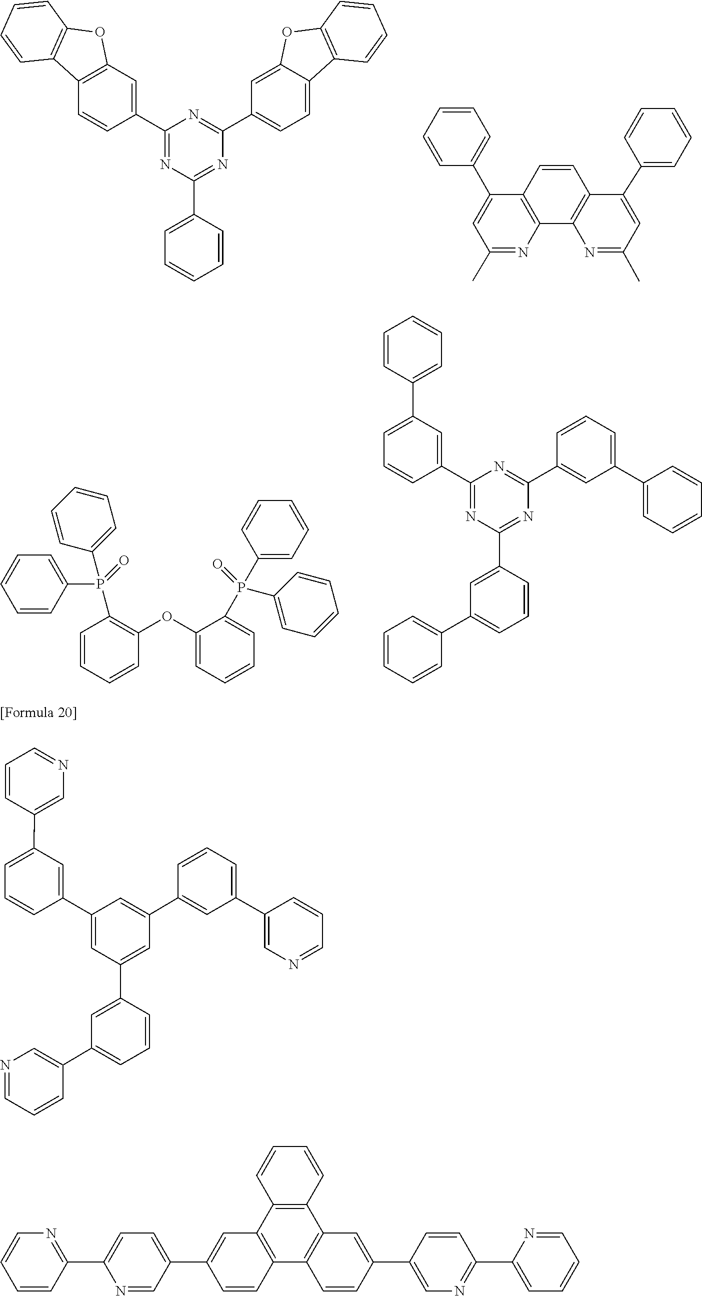 [SMILES: c1ccc(-c2nc(-c3ccc4c(c3)oc3ccccc34)nc(-c3ccc4c(c3)oc3ccccc34)n2)cc1 ; Cc1cc(-c2ccccc2)c2ccc3c(-c4ccccc4)cc(C)nc3c2n1 ; O=P(c1ccccc1)(c1ccccc1)c1ccccc1Oc1ccccc1P(=O)(c1ccccc1)c1ccccc1 ; c1ccc(-c2cccc(-c3nc(-c4cccc(-c5ccccc5)c4)nc(-c4cccc(-c5ccccc5)c4)n3)c2)cc1 ; c1cncc(-c2cccc(-c3cc(-c4cccc(-c5cccnc5)c4)cc(-c4cccc(-c5cccnc5)c4)c3)c2)c1 ; c1ccc(-c2ccc(-c3ccc4c5ccc(-c6ccc(-c7ccccn7)nc6)cc5c5ccccc5c4c3)cn2)nc1]

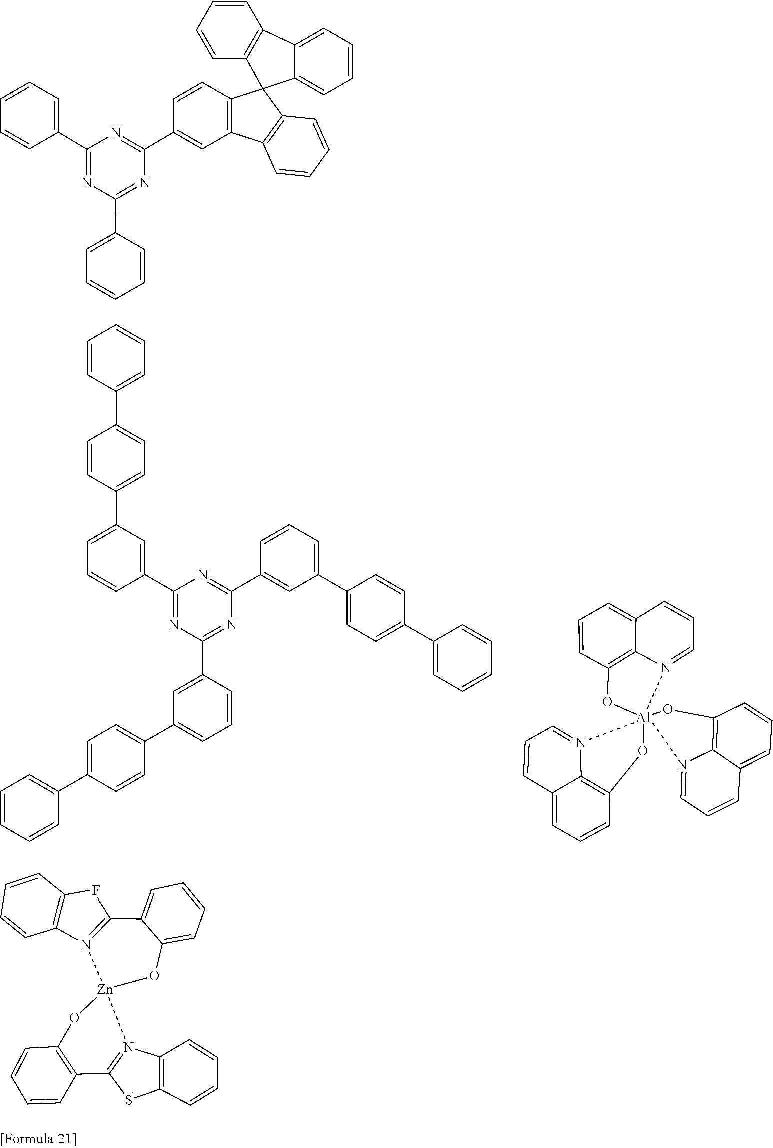

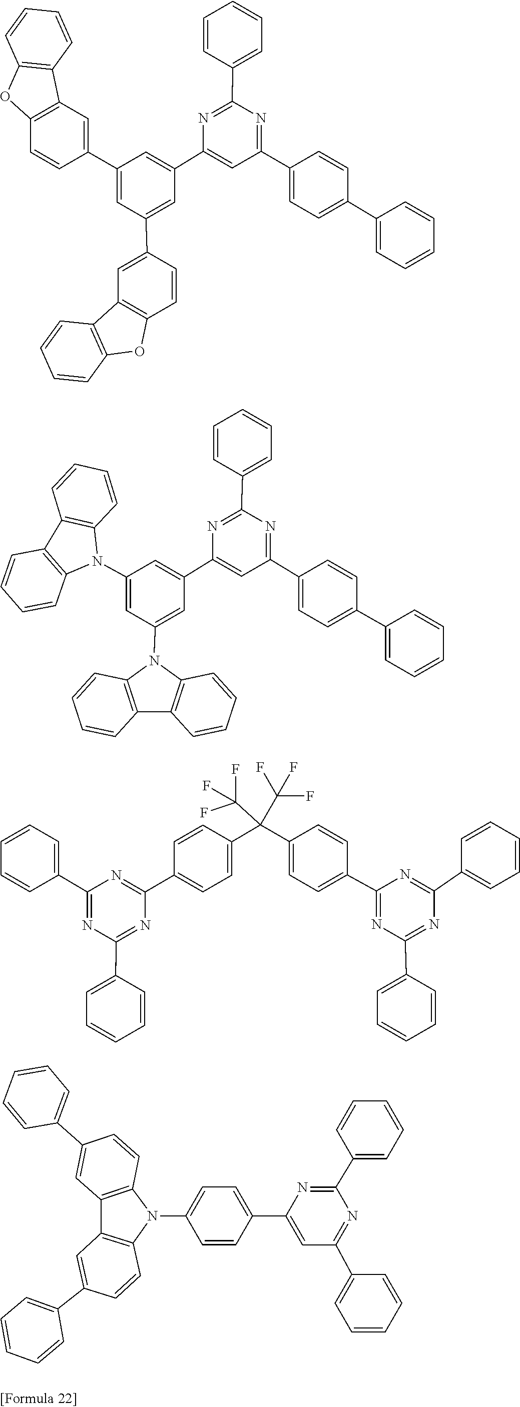

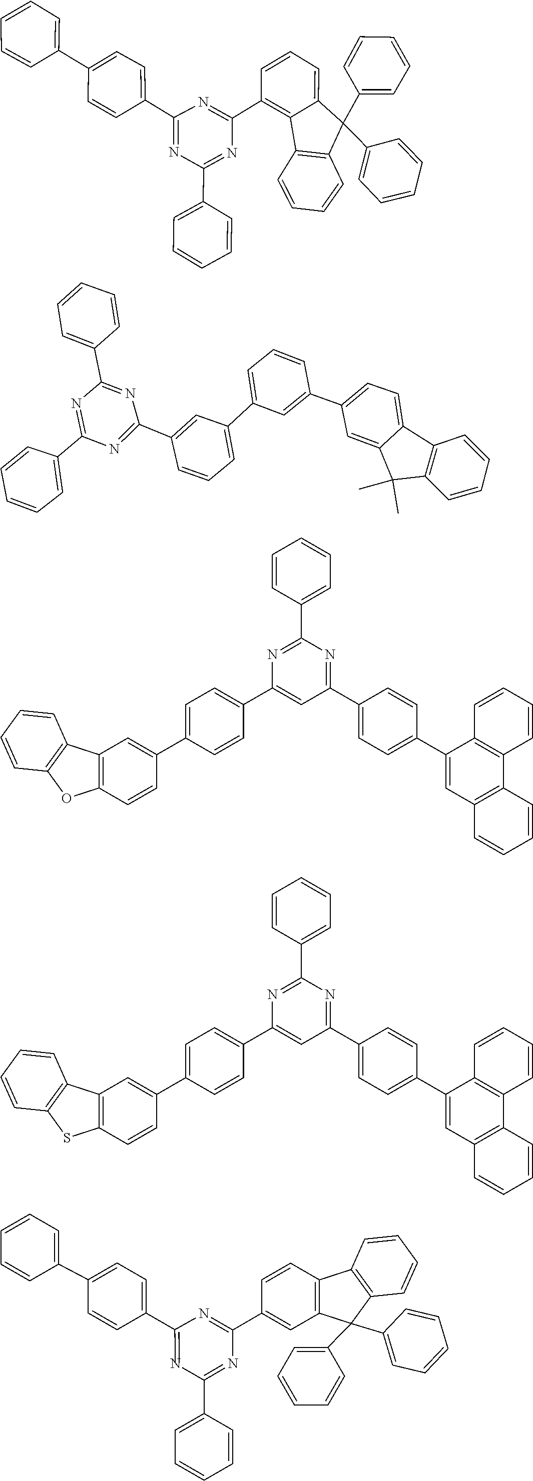 [SMILES: c1ccc(C2=NC(c3cccc4c3-c3ccccc3C4(c3ccccc3)c3ccccc3)=NC(c3ccc(-c4ccccc4)cc3)=NC2)cc1 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccc(-c4ccc5oc6ccccc6c5c4)cc3)cc(-c3ccc(-c4cc5ccccc5c5ccccc45)cc3)n2)cc1 ; c1ccc(-c2nc(-c3ccc(-c4ccc5sc6ccccc6c5c4)cc3)cc(-c3ccc(-c4cc5ccccc5c5ccccc45)cc3)n2)cc1 ; C=C(/N=C(\N=C(/N)c1ccccc1)c1ccc2c(c1)C(c1ccccc1)(c1ccccc1)c1ccccc1-2)c1ccc(-c2ccccc2)cc1]

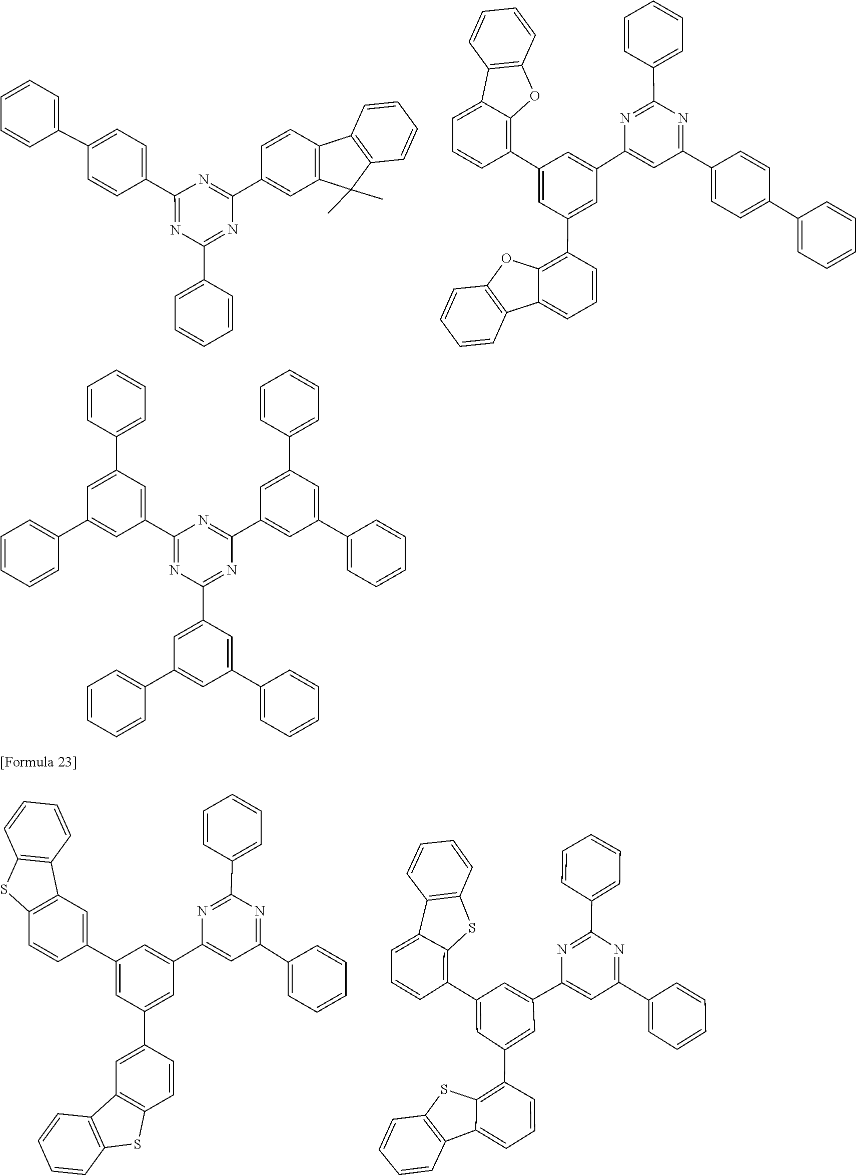

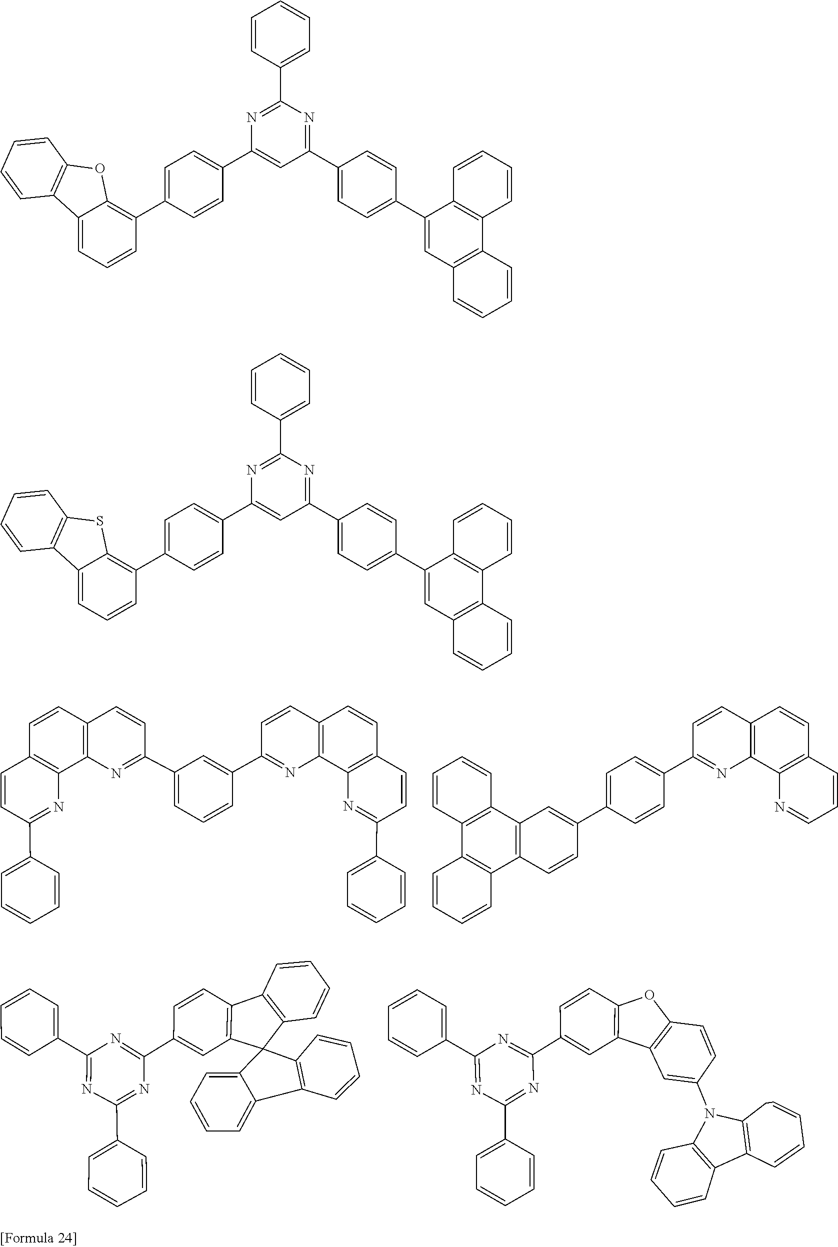

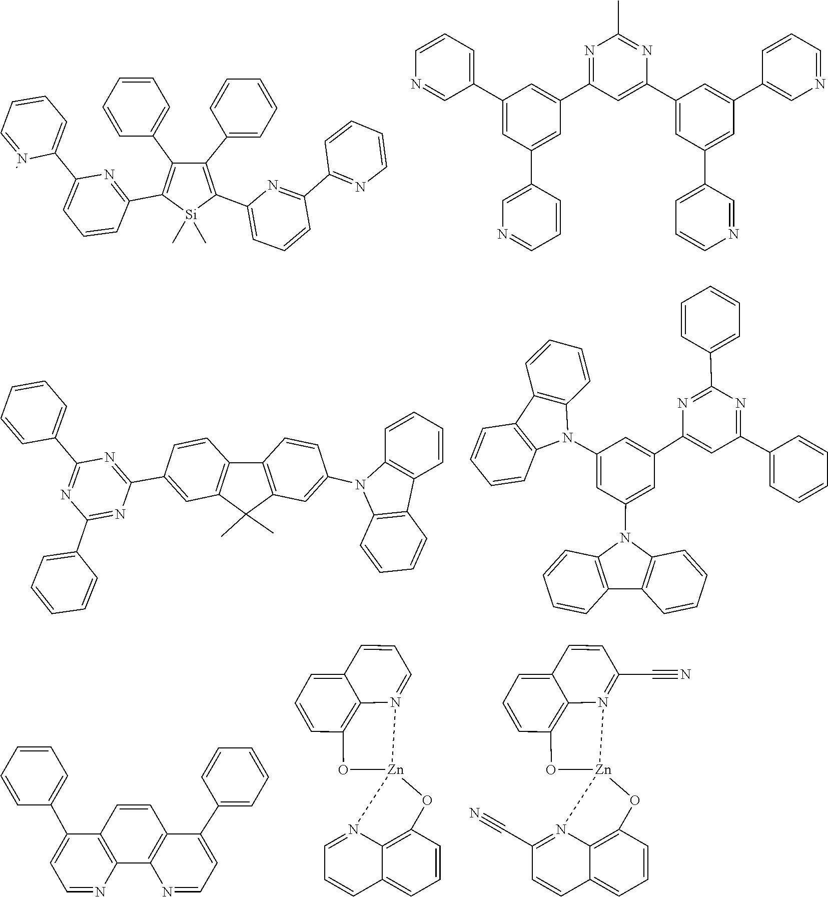

Specific examples of the first compound shown below are specific examples in which description of hydrogen atoms is not omitted.

In the following specific examples, “D” represents a deuterium atom.

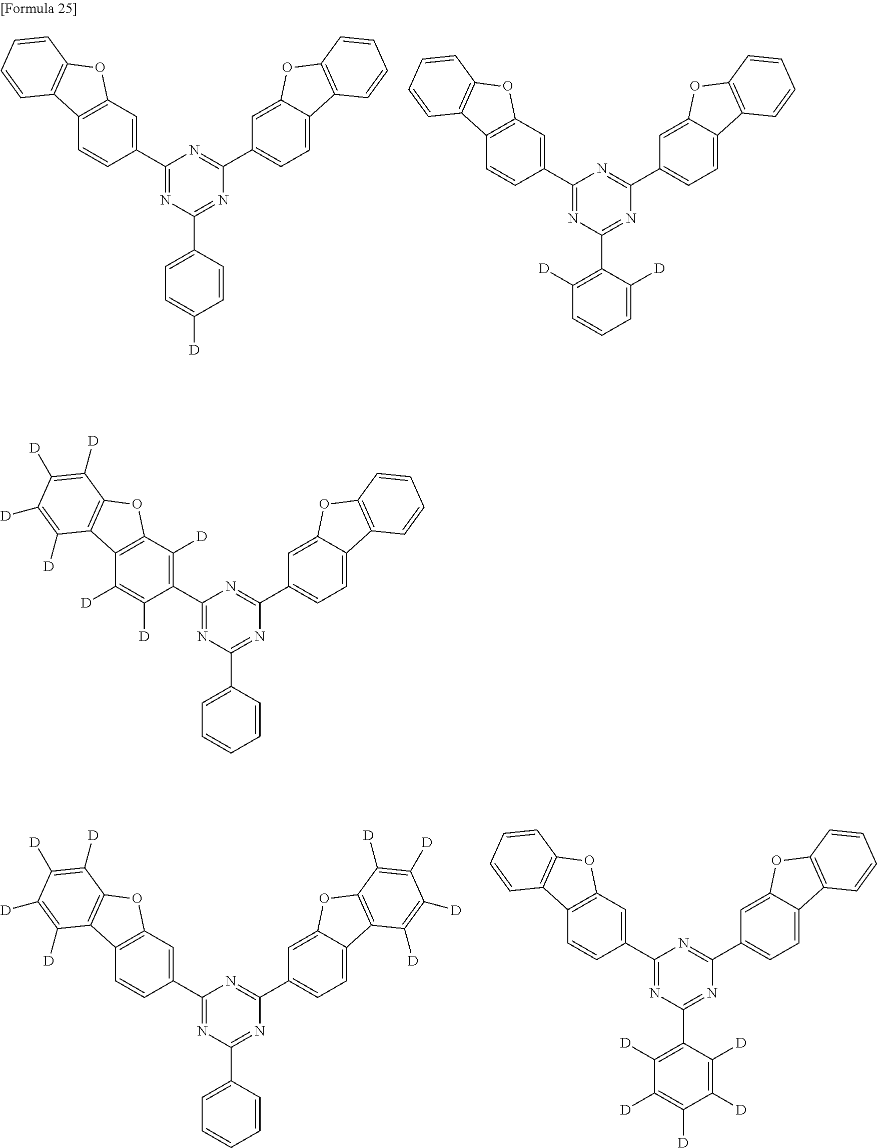

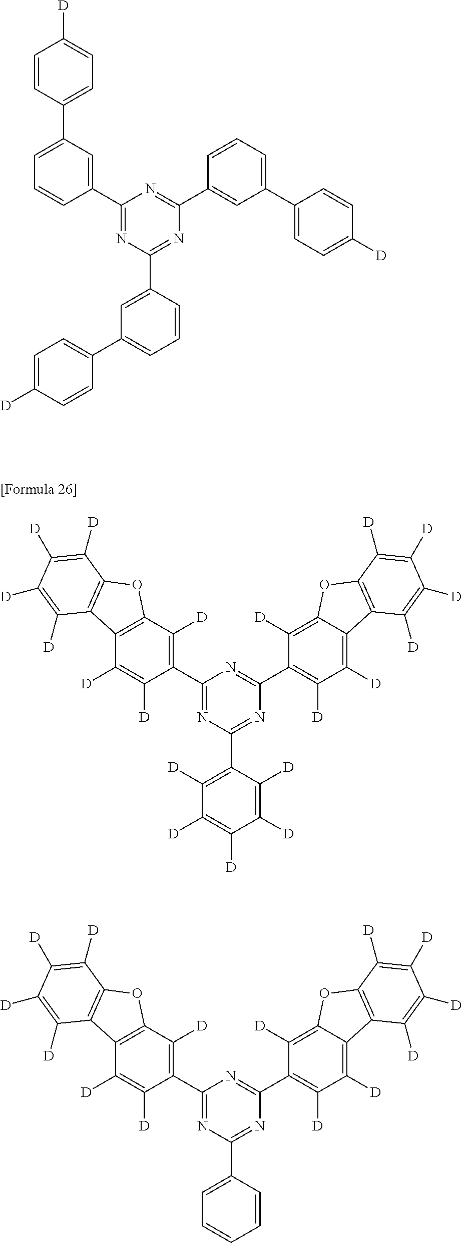 [SMILES: [2H]c1ccc(-c2cccc(-c3nc(-c4cccc(-c5ccc([2H])cc5)c4)nc(-c4cccc(-c5ccc([2H])cc5)c4)n3)c2)cc1 ; [2H]c1c([2H])c([2H])c(-c2nc(-c3c([2H])c([2H])c4c(oc5c([2H])c([2H])c([2H])c([2H])c54)c3[2H])nc(-c3c([2H])c([2H])c4c(oc5c([2H])c([2H])c([2H])c([2H])c54)c3[2H])n2)c([2H])c1[2H] ; [2H]c1c([2H])c([2H])c2c(oc3c([2H])c(-c4nc(-c5ccccc5)nc(-c5c([2H])c([2H])c6c(oc7c([2H])c([2H])c([2H])c([2H])c76)c5[2H])n4)c([2H])c([2H])c32)c1[2H]]

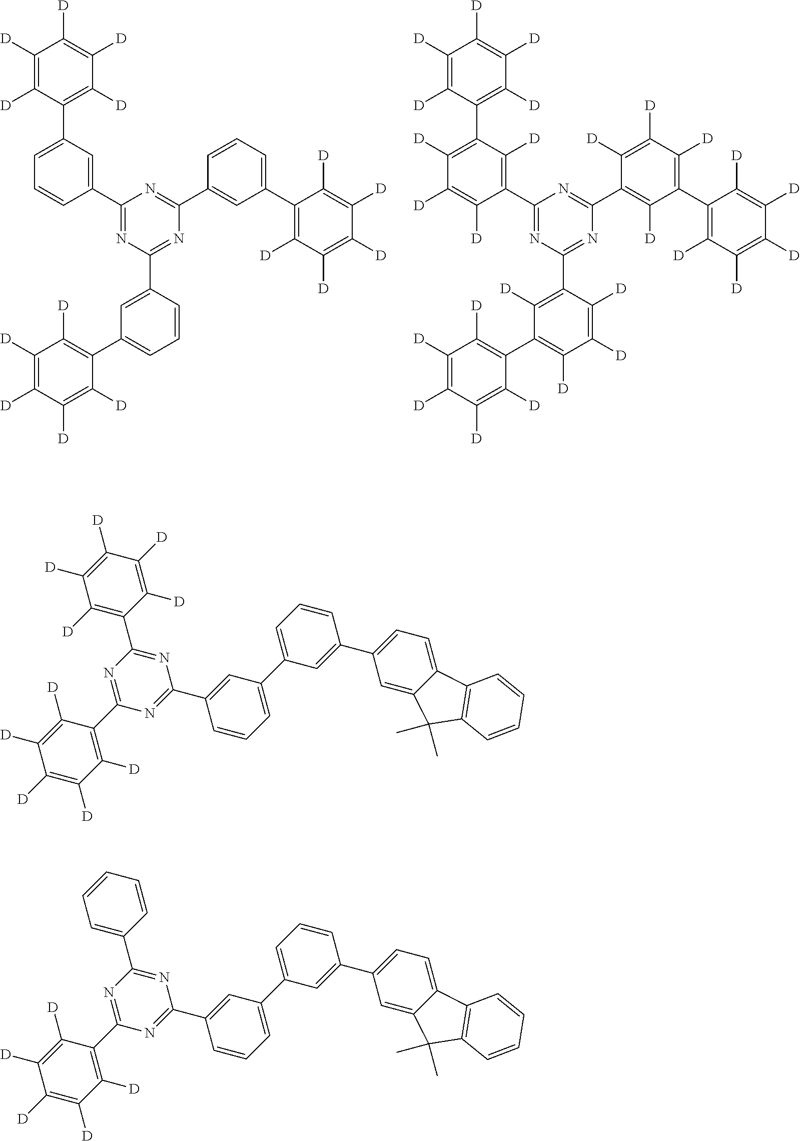

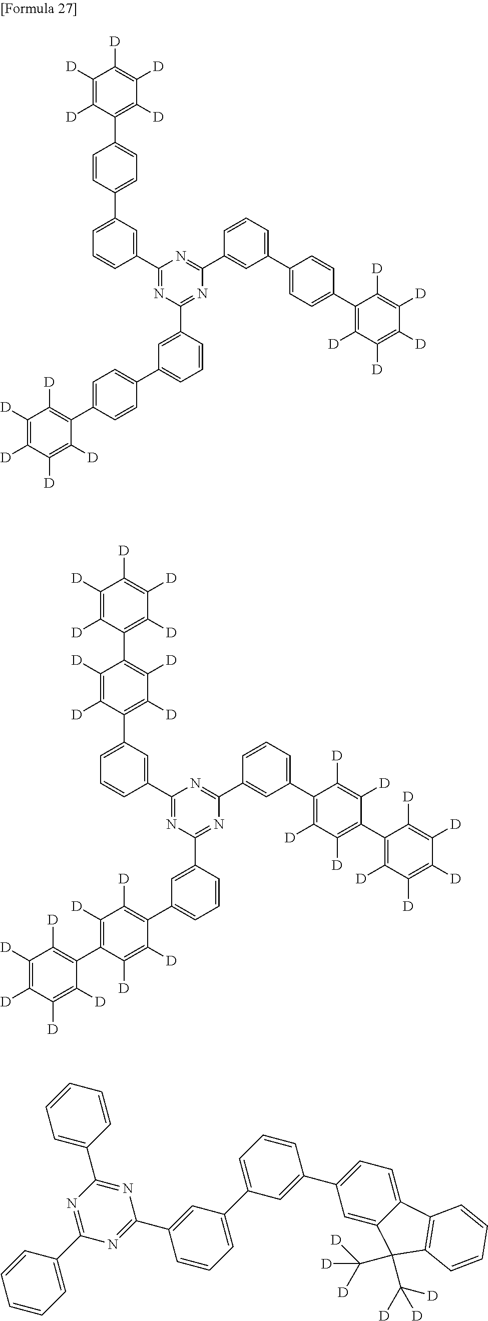

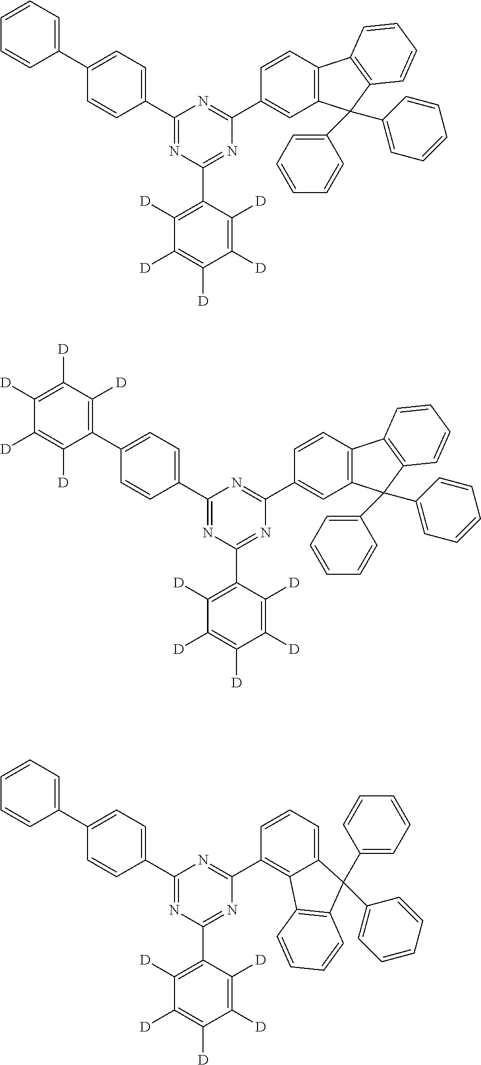 [SMILES: [2H]c1c([2H])c([2H])c(-c2nc(-c3ccc(-c4ccccc4)cc3)nc(-c3ccc4c(c3)C(c3ccccc3)(c3ccccc3)c3ccccc3-4)n2)c([2H])c1[2H] ; [2H]c1c([2H])c([2H])c(-c2ccc(-c3nc(-c4ccc5c(c4)C(c4ccccc4)(c4ccccc4)c4ccccc4-5)nc(-c4c([2H])c([2H])c([2H])c([2H])c4[2H])n3)cc2)c([2H])c1[2H] ; [2H]c1c([2H])c([2H])c(-c2nc(-c3ccc(-c4ccccc4)cc3)nc(-c3cccc4c3-c3ccccc3C4(c3ccccc3)c3ccccc3)n2)c([2H])c1[2H]]

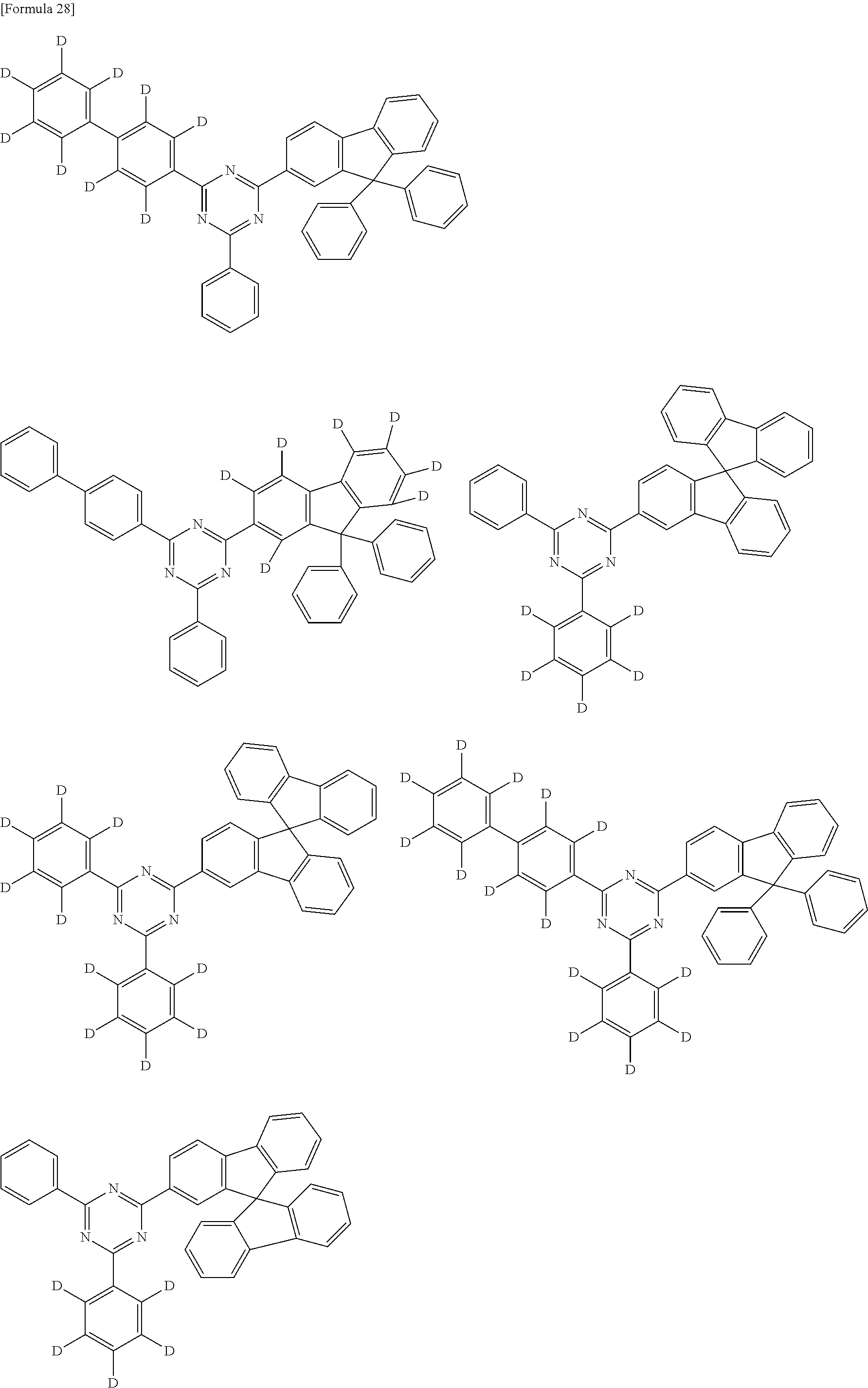

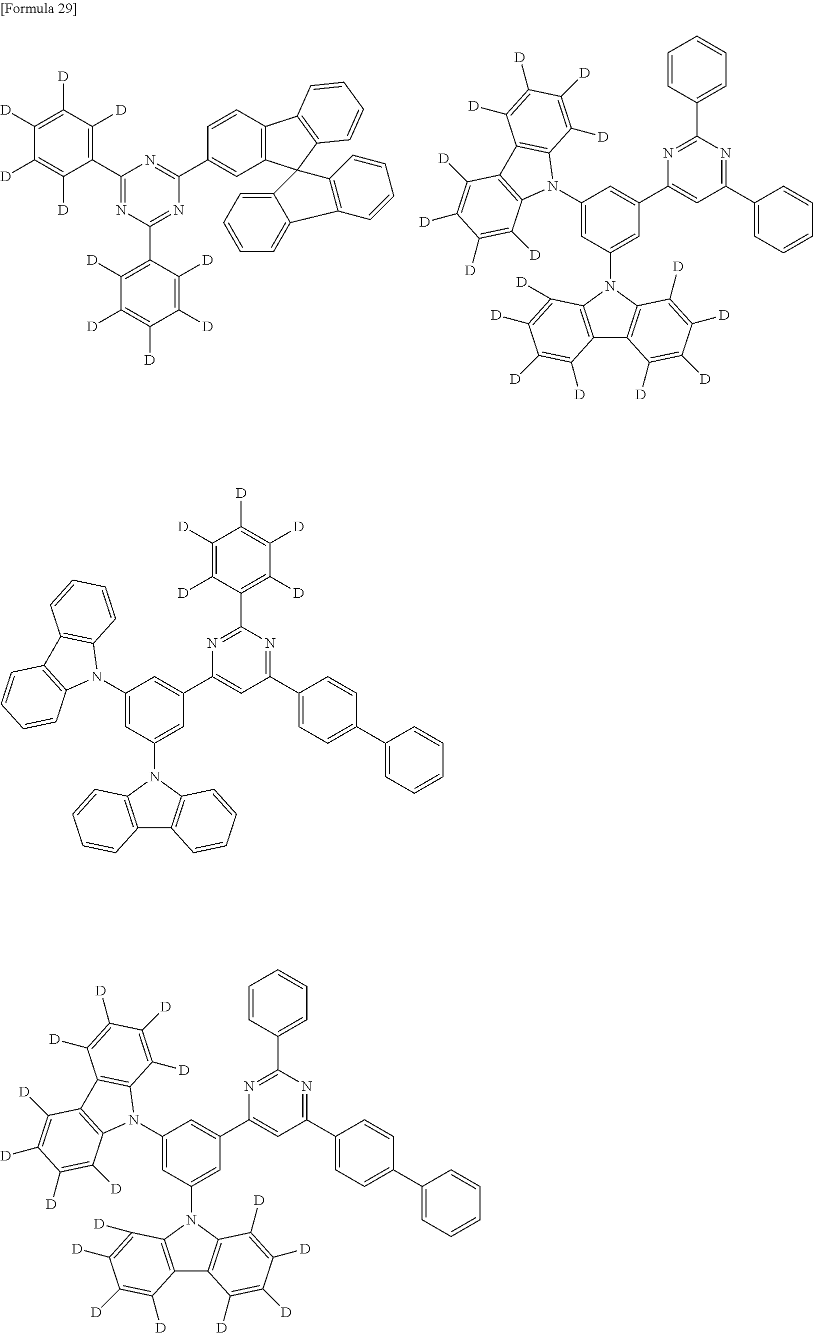

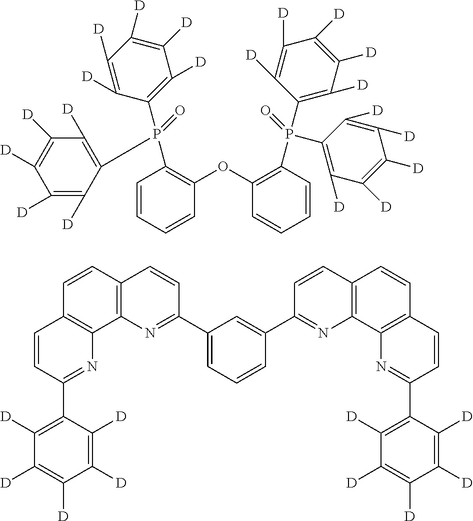

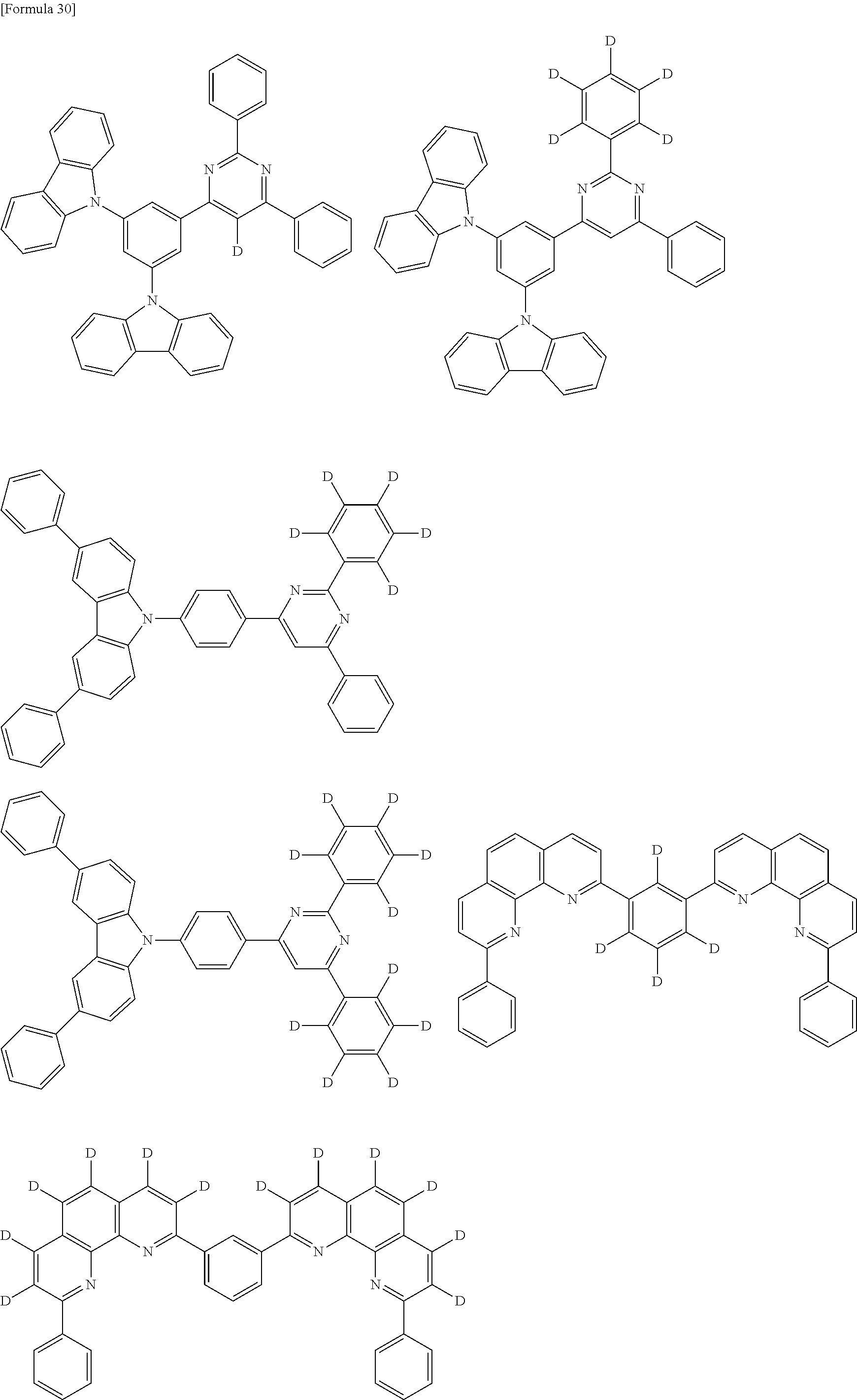

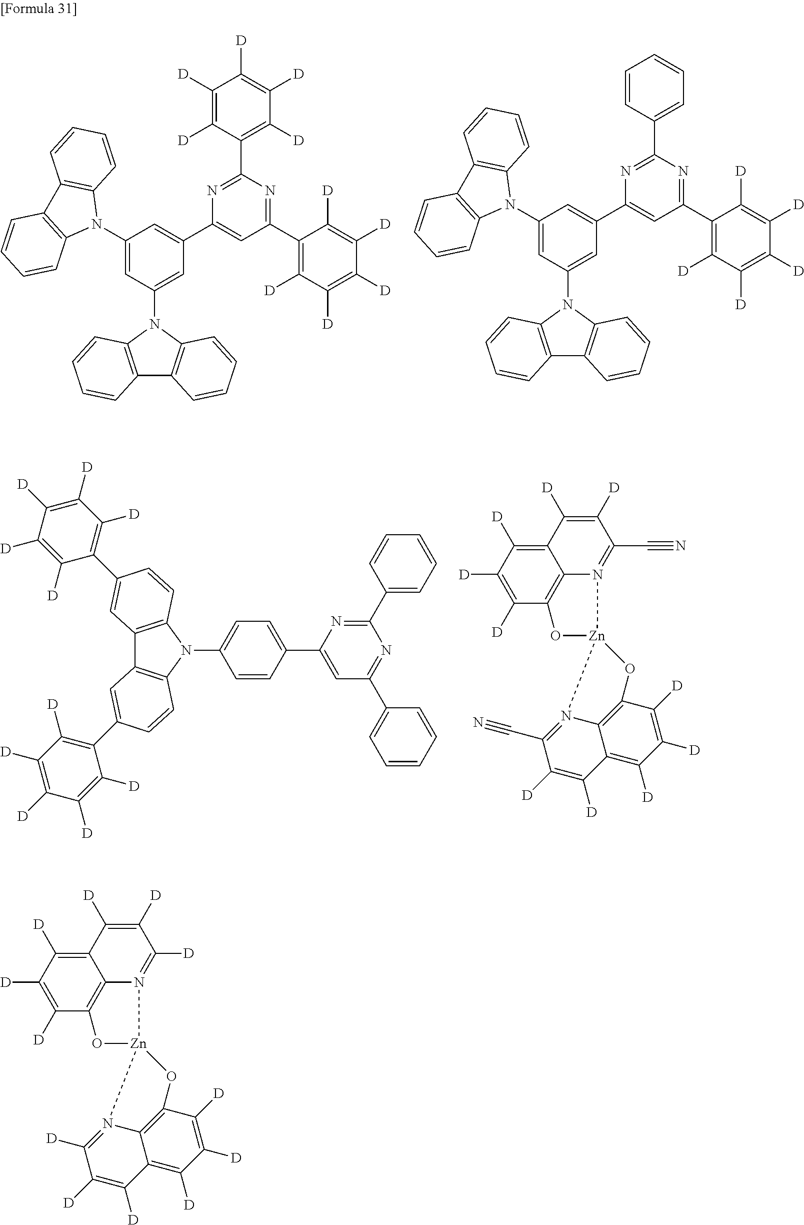

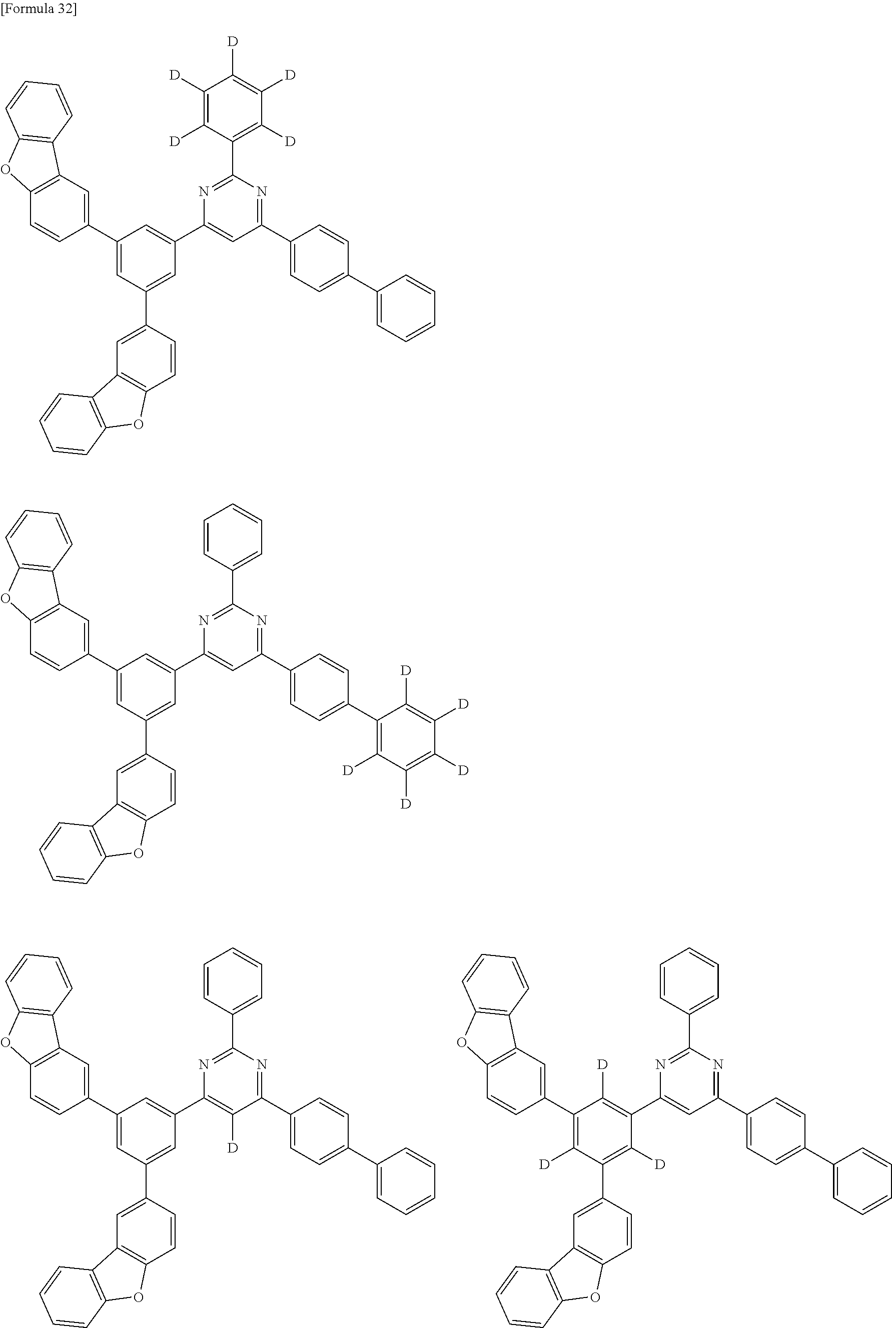 [SMILES: [2H]c1c([2H])c([2H])c(-c2nc(-c3ccc(-c4ccccc4)cc3)cc(-c3cc(-c4ccc5oc6ccccc6c5c4)cc(-c4ccc5oc6ccccc6c5c4)c3)n2)c([2H])c1[2H] ; [2H]c1c([2H])c([2H])c(-c2ccc(-c3cc(-c4cc(-c5ccc6oc7ccccc7c6c5)cc(-c5ccc6oc7ccccc7c6c5)c4)nc(-c4ccccc4)n3)cc2)c([2H])c1[2H] ; [2H]c1c(-c2ccc(-c3ccccc3)cc2)nc(-c2ccccc2)nc1-c1cc(-c2ccc3oc4ccccc4c3c2)cc(-c2ccc3oc4ccccc4c3c2)c1 ; [2H]c1c(-c2ccc3oc4ccccc4c3c2)c([2H])c(-c2cc(-c3ccc(-c4ccccc4)cc3)nc(-c3ccccc3)n2)c([2H])c1-c1ccc2oc3ccccc3c2c1]

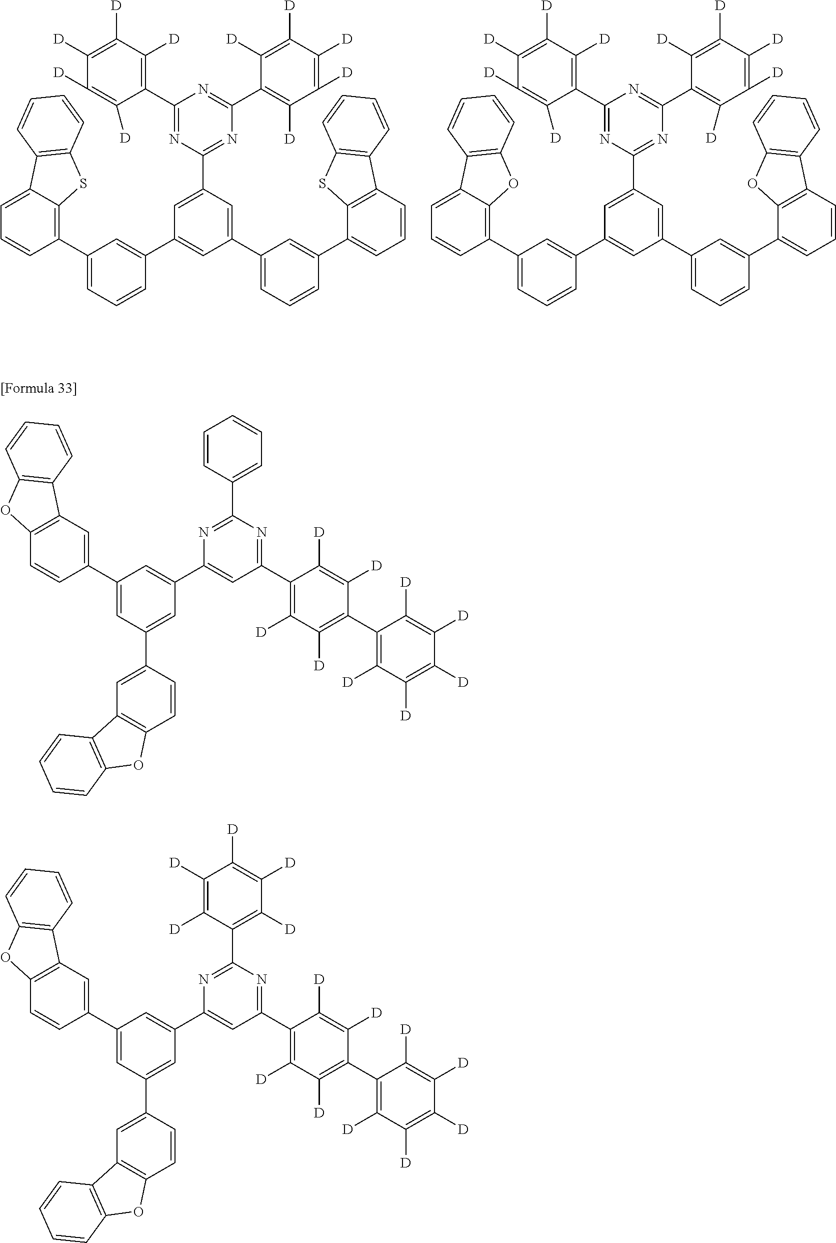

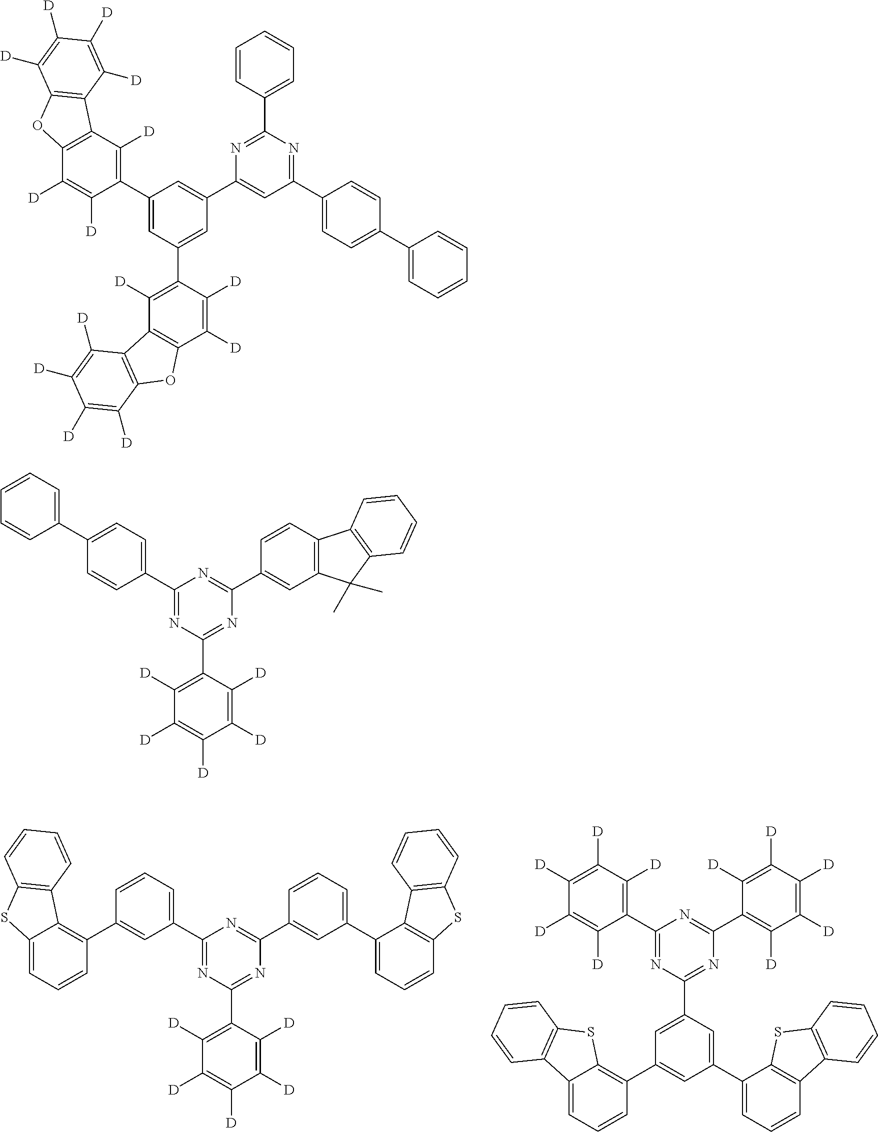

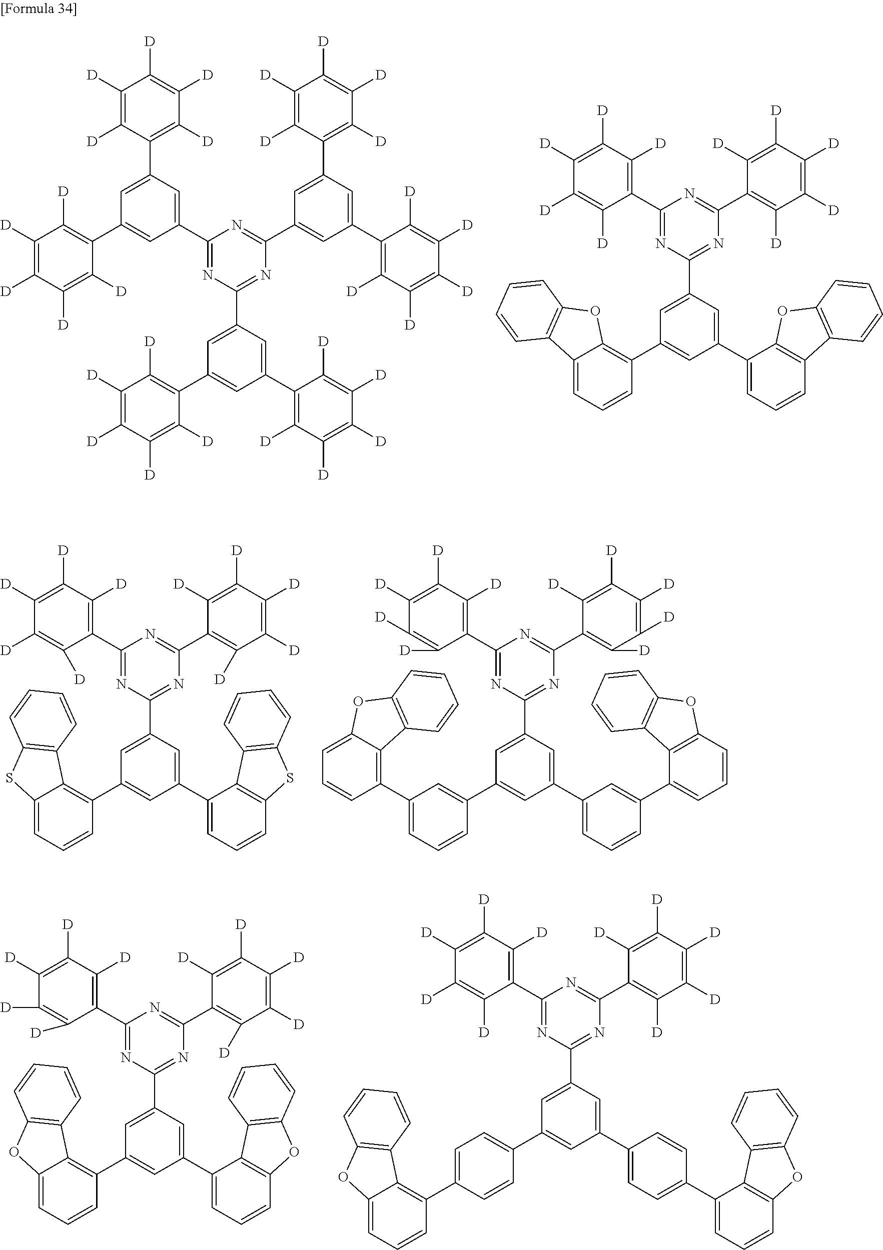

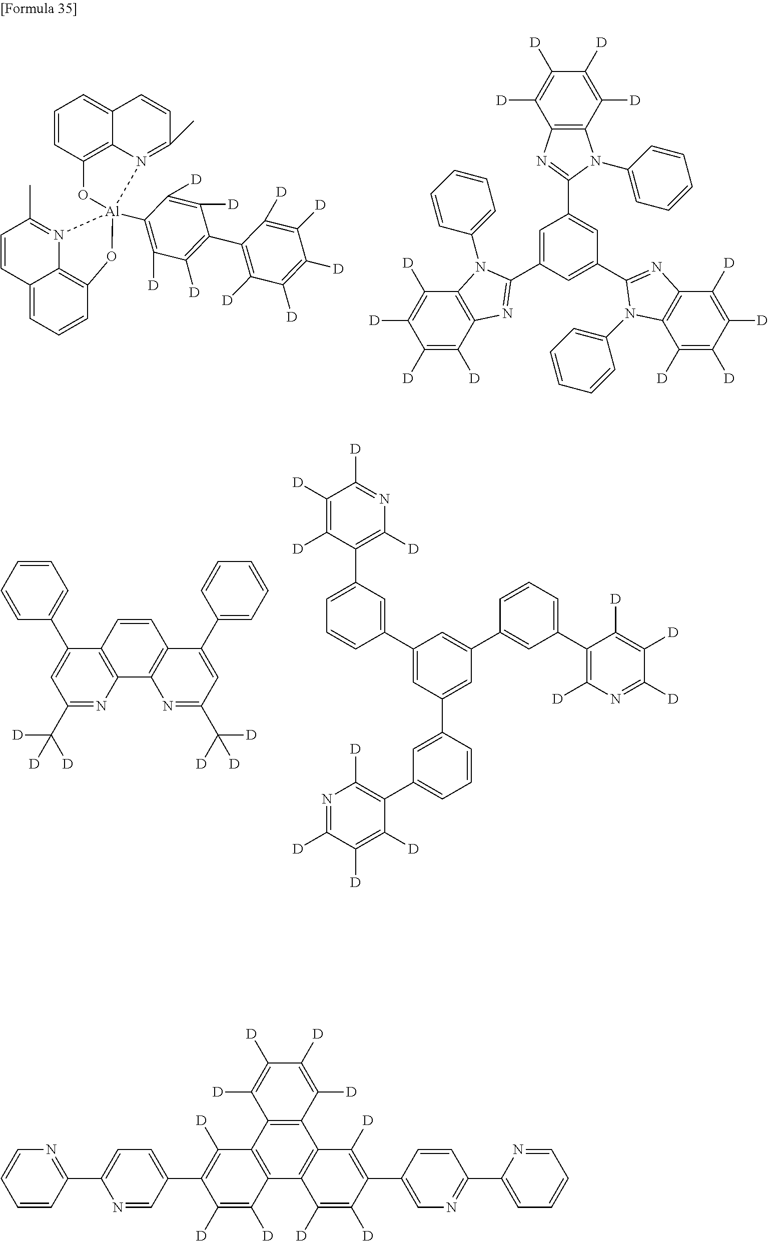

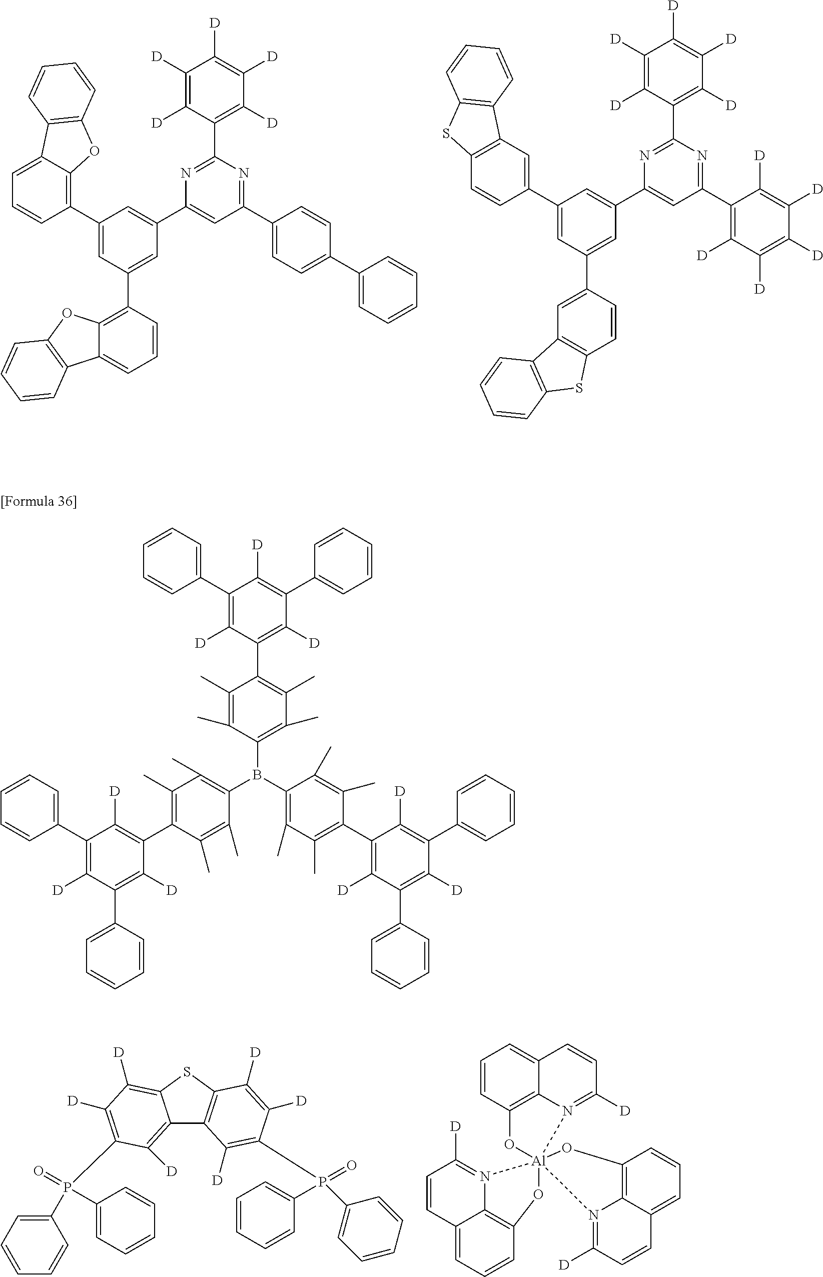

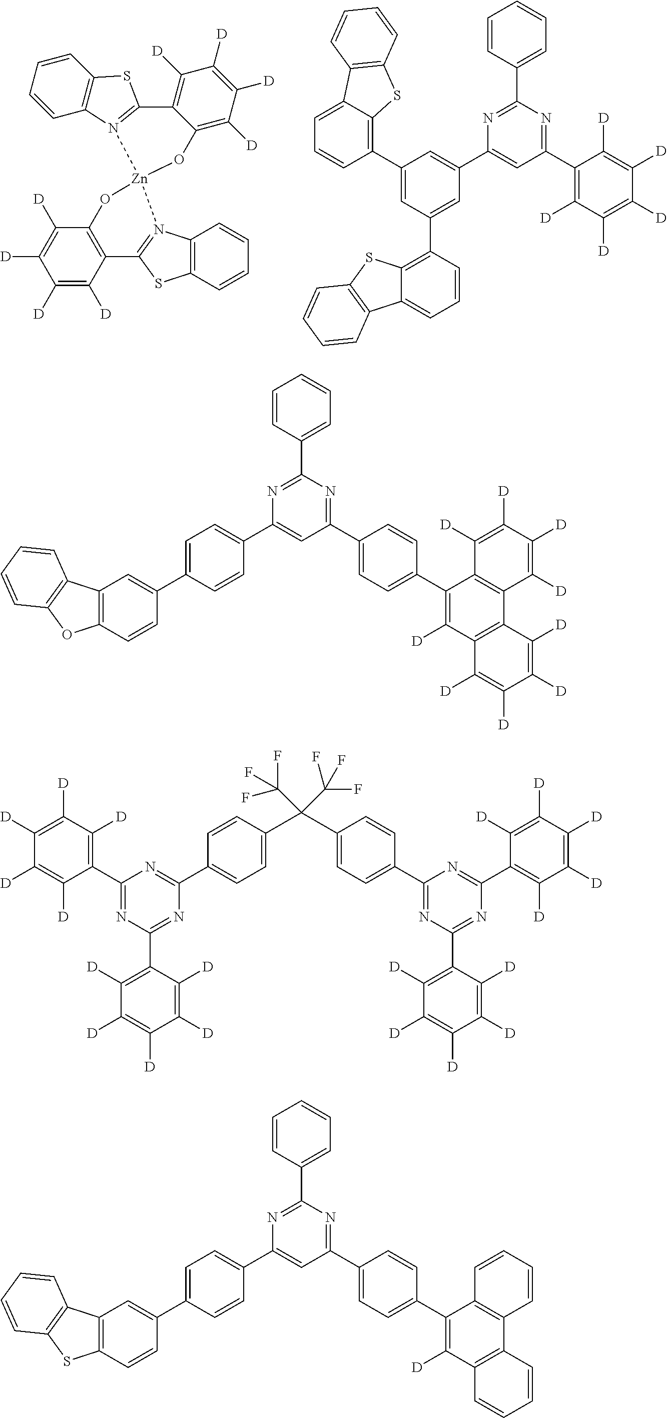

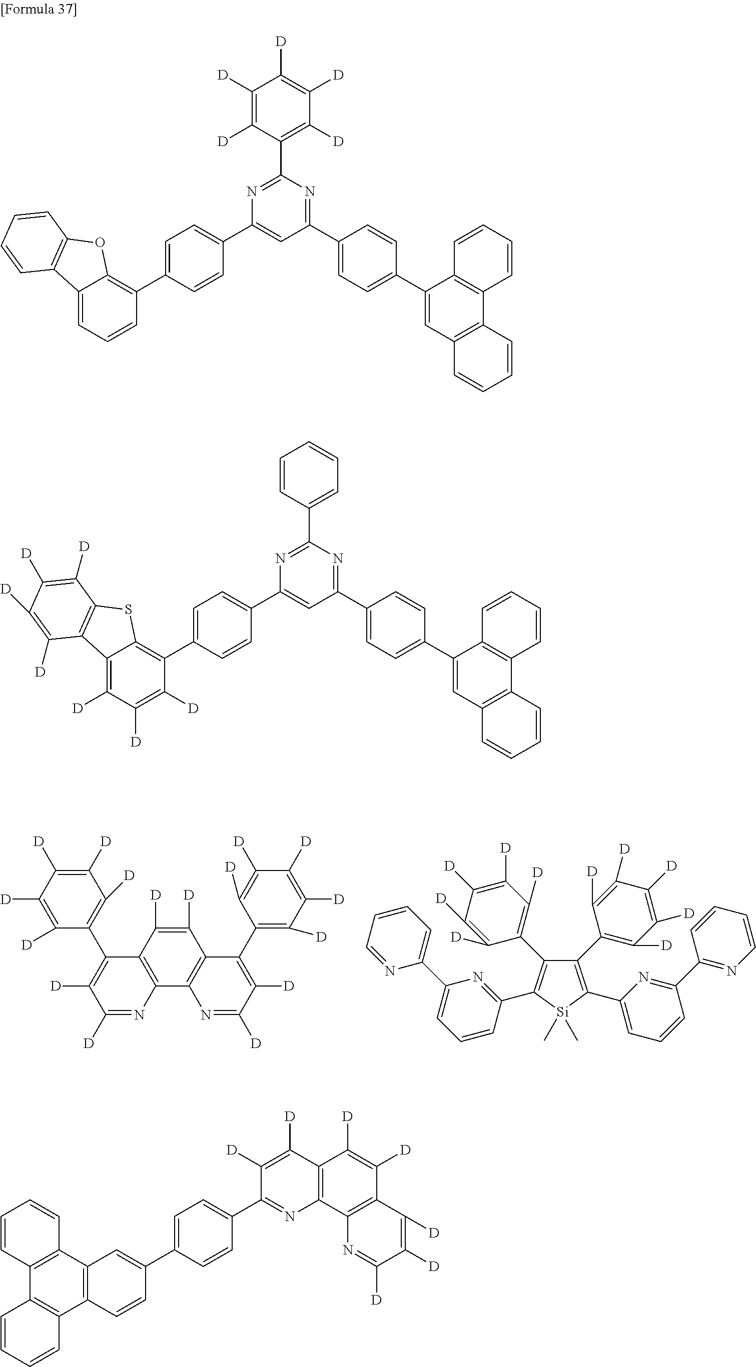

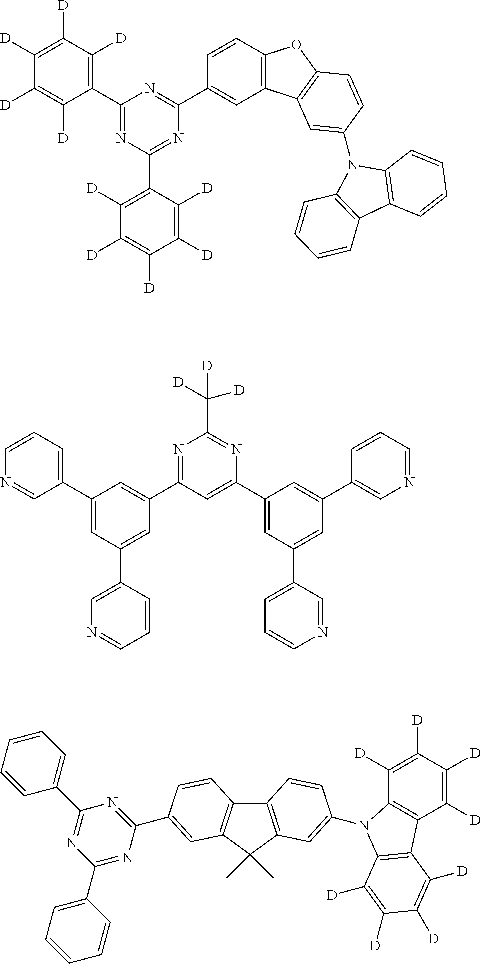 [SMILES: [2H]c1c([2H])c([2H])c(-c2nc(-c3ccc4oc5ccc(-n6c7ccccc7c7ccccc76)cc5c4c3)nc(-c3c([2H])c([2H])c([2H])c([2H])c3[2H])n2)c([2H])c1[2H] ; [2H]C([2H])([2H])c1nc(-c2cc(-c3cccnc3)cc(-c3cccnc3)c2)cc(-c2cc(-c3cccnc3)cc(-c3cccnc3)c2)n1 ; [2H]c1c([2H])c([2H])c2c(c1[2H])c1c([2H])c([2H])c([2H])c([2H])c1n2-c1ccc2c(c1)C(C)(C)c1cc(-c3nc(-c4ccccc4)nc(-c4ccccc4)n3)ccc1-2]

### Emitting Layer

In an exemplary arrangement of the first exemplary embodiment, the emitting layer 5 contains the delayed fluorescent compound M2 and the fluorescent compound M1.

### Compound M2

### Delayed Fluorescence

Delayed fluorescence is explained in “Yuki Hando-tai no Debaisu Bussei (Device Physics of Organic Semiconductors)” (edited by ADACHI, Chihaya, published by Kodansha, on pages 261-268). This document describes that, if an energy difference ΔE₁₃ of a fluorescent material between a singlet state and a triplet state is reducible, a reverse energy transfer from the triplet state to the singlet state, which usually occurs at a low transition probability, would occur at a high efficiency to express thermally activated delayed fluorescence (TADF). Further, a generation mechanism of delayed fluorescence is explained in FIG. 10.38 in the document. The compound M2 of the exemplary embodiment is preferably a compound exhibiting thermally activated delayed fluorescence generated by such a mechanism.

In general, emission of delayed fluorescence can be confirmed by measuring the transient PL (Photo Luminescence).

The behavior of delayed fluorescence can also be analyzed based on the decay curve obtained from the transient PL measurement. The transient PL measurement is a method of irradiating a sample with a pulse laser to excite the sample, and measuring the decay behavior (transient characteristics) of PL emission after the irradiation is stopped. PL emission in TADF materials is classified into a light emission component from a singlet exciton generated by the first PL excitation and a light emission component from a singlet exciton generated via a triplet exciton. The lifetime of the singlet exciton generated by the first PL excitation is on the order of nanoseconds and is very short. Therefore, light emission from the singlet exciton rapidly attenuates after irradiation with the pulse laser.

On the other hand, the delayed fluorescence is gradually attenuated due to light emission from a singlet exciton generated via a triplet exciton having a long lifetime. As described above, there is a large temporal difference between the light emission from the singlet exciton generated by the first PL excitation and the light emission from the singlet exciton generated via the triplet exciton. Therefore, the luminous intensity derived from delayed fluorescence can be determined.

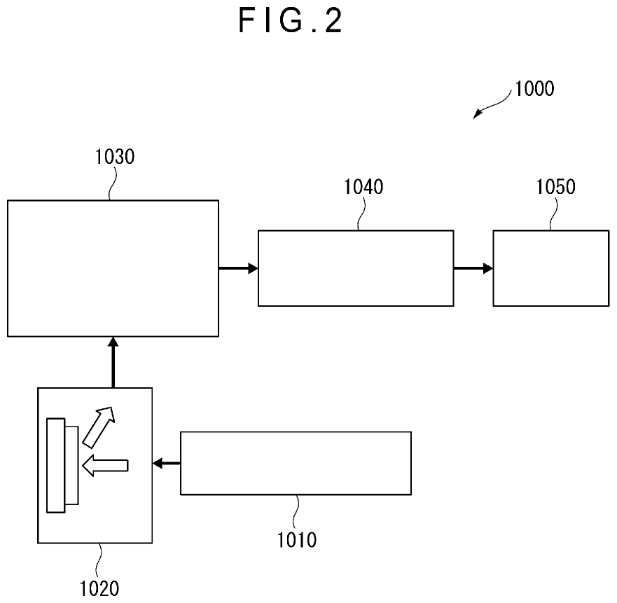

FIG. 2 is a schematic diagram of an exemplary device for measuring the transient PL. An example of a method of measuring a transient PL using FIG. 2 and an example of behavior analysis of delayed fluorescence will be described.

A transient PL measuring device 1000 in FIG. 2 includes: a pulse laser 1010 capable of radiating a light having a predetermined wavelength; a sample chamber 1020 configured to house a measurement sample; a spectrometer 1030 configured to divide a light radiated from the measurement sample; a streak camera 1040 configured to provide a two-dimensional image; and a personal computer 1050 configured to import and analyze the two-dimensional image. A device for measuring the transient PL is not limited to the device shown in FIG. 2.

The sample housed in the sample chamber 1020 is obtained by forming a thin film, in which a matrix material is doped with a doping material at a concentration of 12 mass %, on the quartz substrate.

The thin film sample housed in the sample chamber 1020 is irradiated with the pulse laser from the pulse laser 1010 to excite the doping material. Emission is extracted in a direction of 90 degrees with respect to a radiation direction of the excited light. The extracted emission is divided by the spectrometer 1030 to form a two-dimensional image in the streak camera 1040. As a result, the two-dimensional image is obtainable in which the ordinate axis represents a time, the abscissa axis represents a wavelength, and a bright spot represents a luminous intensity. When this two-dimensional image is taken out at a predetermined time axis, an emission spectrum in which the ordinate axis represents the luminous intensity and the abscissa axis represents the wavelength is obtainable. Moreover, when this two-dimensional image is taken out at the wavelength axis, a decay curve (transient PL) in which the ordinate axis represents a logarithm of the luminous intensity and the abscissa axis represents the time is obtainable.

For instance, a thin film sample A was prepared as described above from a reference compound H1 as the matrix material and a reference compound D1 as the doping material and was measured in terms of the transient PL.

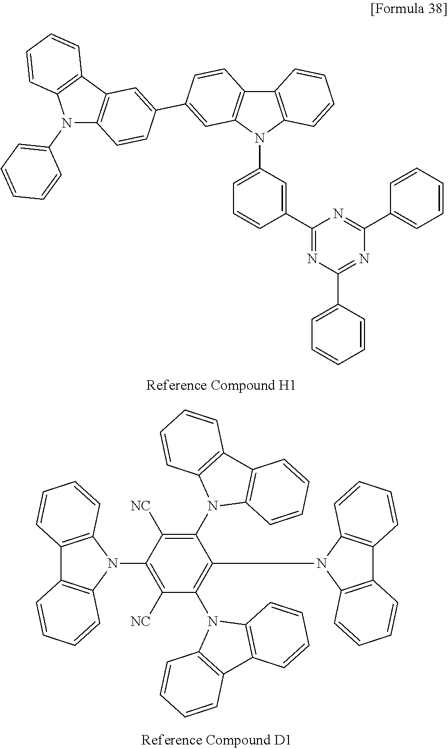 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21]

The decay curve was analyzed with respect to the above thin film sample A and a thin film sample B. The thin film sample B was produced in the same manner as described above from a reference compound H2 as the matrix material and the reference compound D1 as the doping material.

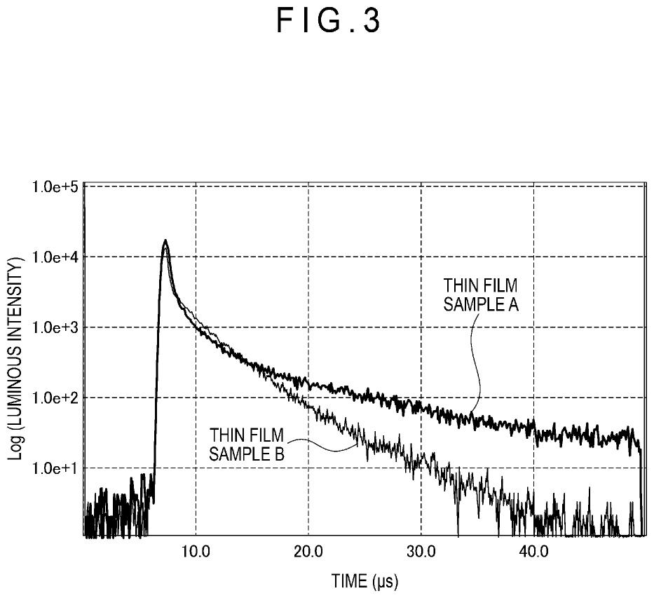

FIG. 3 shows decay curves obtained from transient PL obtained by measuring the thin film samples A and B.

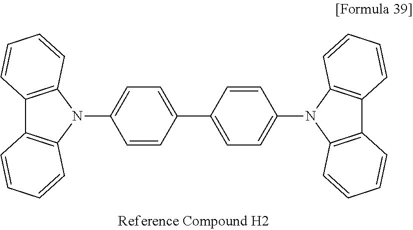 [SMILES: c1ccc2c(c1)c1ccccc1n2-c1ccc(-c2ccc(-n3c4ccccc4c4ccccc43)cc2)cc1]

As described above, an emission decay curve in which the ordinate axis represents the luminous intensity and the abscissa axis represents the time can be obtained by the transient PL measurement. Based on the emission decay curve, a fluorescence intensity ratio between fluorescence emitted from a singlet state generated by photo-excitation and delayed fluorescence emitted from a singlet state generated by reverse energy transfer via a triplet state can be estimated. In a delayed fluorescent material, a ratio of the intensity of the slowly decaying delayed fluorescence to the intensity of the promptly decaying fluorescence is relatively large.

Specifically, Prompt emission and Delay emission are present as emission from the delayed fluorescent material. Prompt emission is observed promptly when the excited state is achieved by exciting the compound of the exemplary embodiment with a pulse beam (i.e., a beam emitted from a pulse laser) having a wavelength absorbable by the delayed fluorescent material. Delay emission is observed not promptly when the excited state is achieved but after the excited state is achieved.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

Herein, a sample produced by the following method is used for measuring delayed fluorescence of the compound M2. For instance, the compound M2 is dissolved in toluene to prepare a dilute solution with an absorbance of 0.05 or less at the excitation wavelength to eliminate the contribution of self-absorption. In order to prevent quenching due to oxygen, the sample solution is frozen and degassed and then sealed in a cell with a lid under an argon atmosphere to obtain an oxygen-free sample solution saturated with argon.

The fluorescence spectrum of the sample solution is measured with a spectrofluorometer FP-8600 (produced by JASCO Corporation), and the fluorescence spectrum of a 9,10-diphenylanthracene ethanol solution is measured under the same conditions. Using the fluorescence area intensities of both spectra, the total fluorescence quantum yield is calculated by an equation (1) in Morris et al. J. Phys. Chem. 80 (1976) 969.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

In the exemplary embodiment, provided that an amount of Prompt emission of a measurement target compound (compound M2) is denoted by XP and an amount of Delay emission is denoted by XD, a value of XD/XP is preferably 0.05 or more.

The amounts of Prompt emission and Delay emission and a ratio of the amounts thereof in compounds other than the compound M2 herein are measured in the same manner as those of the compound M2.

In the exemplary embodiment, the delayed fluorescent compound M2 is preferably a compound represented by a formula (2) or a formula (22) below.

### Compound Represented by Formula (2)

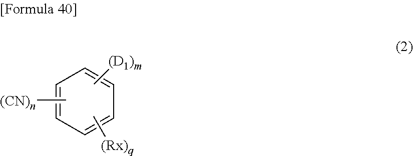

In the formula (2):
n is 1, 2, 3 or 4;
m is 1, 2, 3 or 4;
q is 0, 1, 2, 3 or 4;
m+n+q=6 is satisfied;
CN is a cyano group;
D₁ is a group represented by a formula (2a), (2b) or (2c) below, and when a plurality of D₁ are present, the plurality of D₁ are mutually the same or different;
Rx is a hydrogen atom or a substituent, or at least one combination of combinations of adjacent ones of Rx are mutually bonded to form a ring, and when a plurality of Rx are present, the plurality of Rx are mutually the same or different;
each Rx as the substituent is independently a halogen atom, a substituted or unsubstituted aryl group having 6 to 30 ring carbon atoms, a substituted or unsubstituted heterocyclic group having 5 to 30 ring atoms, a substituted or unsubstituted amino group, a substituted or unsubstituted carbonyl group, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkylsilyl group having 3 to 30 carbon atoms, or a substituted or unsubstituted arylsilyl group having 6 to 60 ring carbon atoms; and
CN, D₁ and Rx are bonded to respective carbon atoms of a six-membered ring.

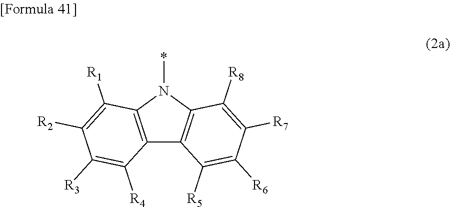

In the formula (2a):
R₁ to R₈ are each independently a hydrogen atom or a substituent, or at least one combination of a combination of R₁ and R₂, a combination of R₂ and R₃, a combination of R₃ and R₄, a combination of R₅ and R₆, a combination of R₆ and R₇, or a combination of R₇ and R₈ are mutually bonded to form a ring;
R₁ to R₈ as the substituents are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkylsilyl group having 3 to 30 carbon atoms, a substituted or unsubstituted arylsilyl group having 6 to 60 ring carbon atoms, a hydroxy group, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 ring carbon atoms, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and
* represents a bonding position to a carbon atom in a six-membered ring in the formula (2).

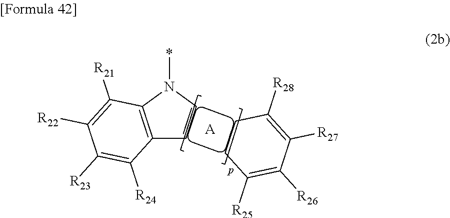

In the formula (2b):
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
R₂₁ to R₂₈ as the substituents each independently represent the same as R₁ to R₈ in the formula (2a);
A represents a cyclic structure represented by a formula (211) or (212) below, and the cyclic structure A is fused with adjacent cyclic structure(s) at any position(s);
p is 1, 2, 3 or 4;
when p is 2, 3 or 4, a plurality of cyclic structures A are mutually the same or different; and
* represents a bonding position to a carbon atom in a six-membered ring in the formula (2).

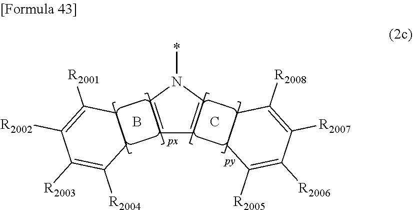

In the formula (2c):
R₂₀₀₁ to R₂₀₀₈ are each independently a hydrogen atom or a substituent, or at least one combination of a combination of R₂₀₀₁ and R₂₀₀₂, a combination of R₂₀₀₂ and R₂₀₀₃, a combination of R₂₀₀₃ and R₂₀₀₄, a combination of R₂₀₀₅ and R₂₀₀₆, a combination of R₂₀₀₆ and R₂₀₀₇, or a combination of R₂₀₀₇ and R₂₀₀ are mutually bonded to form a ring;
R₂₀₀₁ to R₂₀₀₈ as the substituents each independently represent the same as R₁ to R₈ as the substituents in the formula (2a);
B represents a cyclic structure represented by the formula (211) or (212), and the cyclic structure B is fused with adjacent cyclic structure(s) at any position(s);
px is 1, 2, 3 or 4;
when px is 2, 3 or 4, a plurality of cyclic structures B are mutually the same or different;
C represents a cyclic structure represented by the formula (211) or (212), and the cyclic structure C is fused with adjacent cyclic structure(s) at any position(s);
py is 1, 2, 3 or 4;
when py is 2, 3 or 4, a plurality of cyclic structures C are mutually the same or different; and
* represents a bonding position to a carbon atom in a six-membered ring in the formula (2).

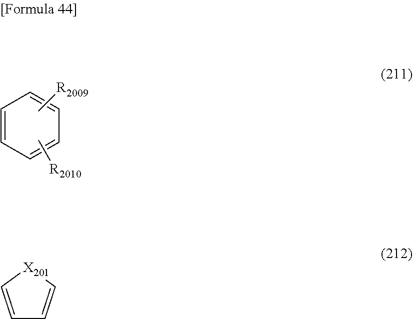

In the formula (211), R₂₀₀₉ and R₂₀₁₀ are each independently a hydrogen atom or a substituent, or bonded to a part of an adjacent cyclic structure to form a ring, or a combination of R₂₀₀₉ and R₂₀₁₀ are mutually bonded to form a ring;
in the formula (212), X₂₀₁ is CR₂₀₁₁R₂₀₁₂, NR₂₀₁₃, a sulfur atom, or an oxygen atom, and R₂₀₁₁, R₂₀₁₂ and R₂₀₁₃ are each independently a hydrogen atom or a substituent, or R₂₀₁₁ and R₂₀₁₂ are mutually bonded to form a ring; and
R₂₀₀₉, R₂₀₁₀, R₂₀₁₁, R₂₀₁₂ and R₂₀₁₃ as the substituents each independently represent the same as R₁ to R₈ as the substituents in the formula (2a).

In the formula (211), R₂₀₀₉ and R₂₀₁₀ are each independently bonded to a part of an adjacent cyclic structure to form a ring, which specifically means any of (I) to (IV) below.

In the formula (211), a combination of R₂₀₀₉ and R₂₀₁₀ are mutually bonded to form a ring, which specifically means (V) below.

(I) When the cyclic structures represented by the formula (211) are adjacent to each other, between the two adjacent rings, at least one combination of the following are mutually bonded to form a ring: R₂₀₀₉ of one of the rings and R₂₀₀₉ of the other of the rings; R₂₀₀₉ of one of the rings and R₂₀₁₀ of the other of the rings; or R₂₀₁₀ of one of the rings and R₂₀₁₀ of the other of the rings.

(II) When the cyclic structure represented by the formula (211) and the benzene ring having R₂₅ to R₂₈ in the formula (2b) are adjacent to each other, between the two adjacent rings, at least one combination of the following are mutually bonded to form a ring: R₂₀₉ of one of the rings and R₂₅ of the other of the rings; R₂₀₉ of one of the rings and R₂₈ of the other of the rings; R₂₁₀ of one of the rings and R₂₅ of the other of the rings; or R₂₁₀ of one of the rings and R₂₈ of the other of the rings.

(III) When the cyclic structure represented by the formula (211) and the benzene ring having R₂₀₀₁ to R₂₀₀₄ in the formula (2c) are adjacent to each other, between the two adjacent rings, at least one combination of the following are mutually bonded to form a ring: R₂₀₀₉ of one of the rings and R₂₀₀₁ of the other of the rings; R₂₀₀₉ of one of the rings and R₂₀₀₄ of the other of the rings; R₂₀₁₀ of one of the rings and R₂₀₀₁ of the other of the rings; or R₂₀₁₀ of one of the rings and R₂₀₀₄ of the other of the rings.

(IV) When the cyclic structure represented by the formula (211) and the benzene ring having R₂₀₀₅ to R₂₀₀₈ in the formula (2c) are adjacent to each other, between the two adjacent rings, at least one combination of the following are mutually bonded to form a ring: R₂₀₀₉ of one of the rings and R₂₀₀₅ of the other of the rings; R₂₀₀₉ of one of the rings and R₂₀₀₈ of the other of the rings; R₂₀₁₀ of one of the rings and R₂₀₀₅ of the other of the rings; or R₂₀₁₀ of one of the rings and R₂₀₀₈ of the other of the rings.

(V) The combination of R₂₀₀₉ and R₂₀₁₀ of the cyclic structure represented by the formula (211) are mutually bonded to form a ring. In other words, (V) means that the combination of R₂₀₀₉ and R₂₀₁₀, which are bonded to the same ring, are mutually bonded to form a ring.

In the compound M2, it is preferable that each Rx is independently a hydrogen atom, an unsubstituted aryl group having 6 to 30 ring carbon atoms, an unsubstituted heterocyclic group having 5 to 30 ring atoms, or an unsubstituted alkyl group having 1 to 30 carbon atoms.

When Rx is an unsubstituted heterocyclic group having 5 to 30 ring atoms, Rx as an unsubstituted heterocyclic group having 5 to 30 ring atoms is a pyridyl group, pyrimidinyl group, triazinyl group, dibenzofuranyl group, or dibenzothienyl group.

Herein, the triazinyl group refers to a group obtained by excluding one hydrogen atom from 1,3,5-triazine, 1,2,4-triazine, or 1,2,3-triazine.

The triazinyl group is preferably a group obtained by excluding one hydrogen atom from 1,3,5-triazine.

In the compound M2, it is more preferable that each Rx is independently a hydrogen atom, an unsubstituted aryl group having 6 to 30 ring carbon atoms, an unsubstituted dibenzofuranyl group, or an unsubstituted dibenzothienyl group.

In the compound M2, Rx is further preferably a hydrogen atom.

In the compound M2, it is preferable that R₁ to R₈, R₂₁ to R₂₈, R₂₀₀₁ to R₂₀₀₈, R₂₀₀₉ to R₂₀₁₀ and R₂₀₁₁ to R₂₀₁₃ as the substituents are each independently an unsubstituted aryl group having 6 to 30 ring carbon atoms, an unsubstituted heterocyclic group having 5 to 30 ring atoms, or an unsubstituted alkyl group having 1 to 30 carbon atoms.

In the compound M2, D₁ is preferably a group represented by one of formulae (D-21) to (D-37) below.

### Groups Represented by Formulae (D-21) to (D-25)

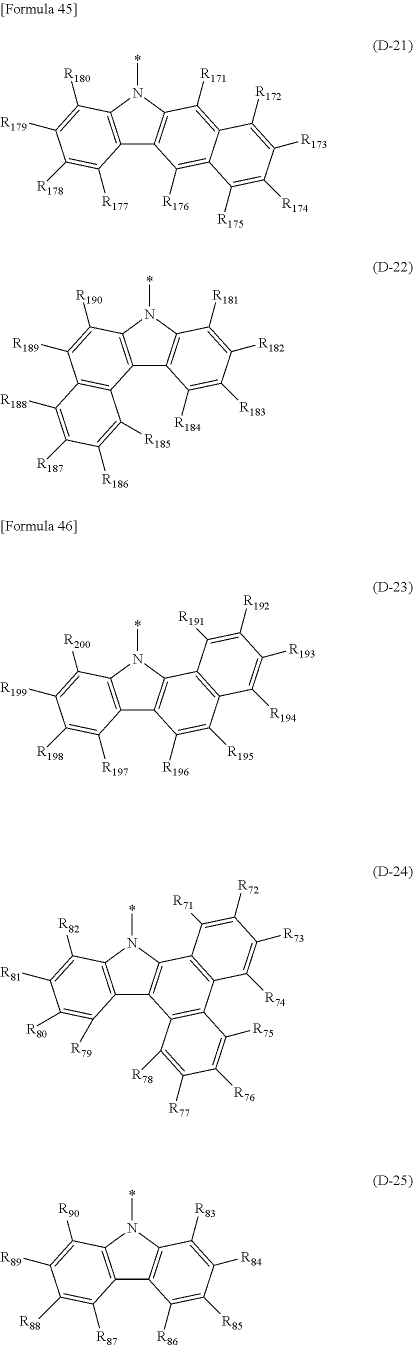

In the formulae (D-21) to (D-25), R₁₇₁ to R₂₀₀ and R₇₁ to R₉₀ are each independently a hydrogen atom or a substituent, or at least one combination of a combination of R₁₇₁ and R₁₇₂, a combination of R₁₇₂ and R₁₇₃, a combination of R₁₇₃ and R₁₇₄, a combination of R₁₇₄ and R₁₇₅, a combination of R₁₇₅ and R₁₇₆, a combination of R₁₇₇ and R₁₇₈, a combination of R₁₇₈ and R₁₇₉, a combination of R₁₇₉ and R₁₈₀, a combination of R₁₈₁ and R₁₈₂, a combination of R₁₈₂ and R₁₈₃, a combination of R₁₈₃ and R₁₈₄, a combination of R₁₈₅ and R₁₈₆, a combination of R₁₈₆ and R₁₈₇, a combination of R₁₈₇ and R₁₈₈, a combination of R₁₈₈ and R₁₈₉, a combination of R₁₈₉ and R₁₉₀, a combination of R₁₉₁ and R₁₉₂, a combination of R₁₉₂ and R₁₉₃, a combination of R₁₉₃ and R₁₉₄, a combination of R₁₉₄ and R₁₉₅, a combination of R₁₉₅ and R₁₉₆, a combination of R₁₉₇ and R₁₉₈, a combination of R₁₉₈ and R₁₉₉, a combination of R₁₉₉ and R₂₀₀, a combination of R₇₁ and R₇₂, a combination of R₇₂ and R₇₃, a combination of R₇₃ and R₇₄, a combination of R₇₅ and R₇₆, a combination of R₇₆ and R₇₇, a combination of R₇₇ and R₇₈, a combination of R₇₉ and R₈₀, a combination of R₈₀ and R₈₁, or a combination of R₈₁ and R₈₂ are bonded to each other to form a ring;
R₁₇₁ to R₂₀₀ and R₇₁ to R₉₀ as the substituents are each independently a halogen atom, a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkylsilyl group having 3 to 6 carbon atoms, a hydroxy group, a substituted or unsubstituted alkoxy group having 1 to 6 carbon atoms, a substituted or unsubstituted alkoxy halide group having 1 to 6 carbon atoms, a substituted or unsubstituted aryloxy group having 6 to 14 ring carbon atoms, a substituted or unsubstituted alkylamino group having 2 to 12 carbon atoms, a thiol group, a substituted or unsubstituted alkylthio group having 1 to 6 carbon atoms, or a substituted or unsubstituted arylthio group having 6 to 14 ring carbon atoms; and
* represents a bonding position to a carbon atom in a six-membered ring in the formula (2).

In the compound M2, it is preferable that R₁₇₁ to R₂₀₀ and R₇₁ to R₉₀ as the substituents are each independently an unsubstituted aryl group having 6 to 14 ring carbon atoms, an unsubstituted heterocyclic group having 5 to 14 ring atoms, or an unsubstituted alkyl group having 1 to 6 carbon atoms.

In the compound M2, R₁₇₁ to R₂₀₀ and R₇₁ to R₉₀ are also preferably hydrogen atoms.

Each of the groups represented by one of the formulae (D-21) to (D-25) is preferably a group represented by any of formulae (2-5) to (2-14) below.

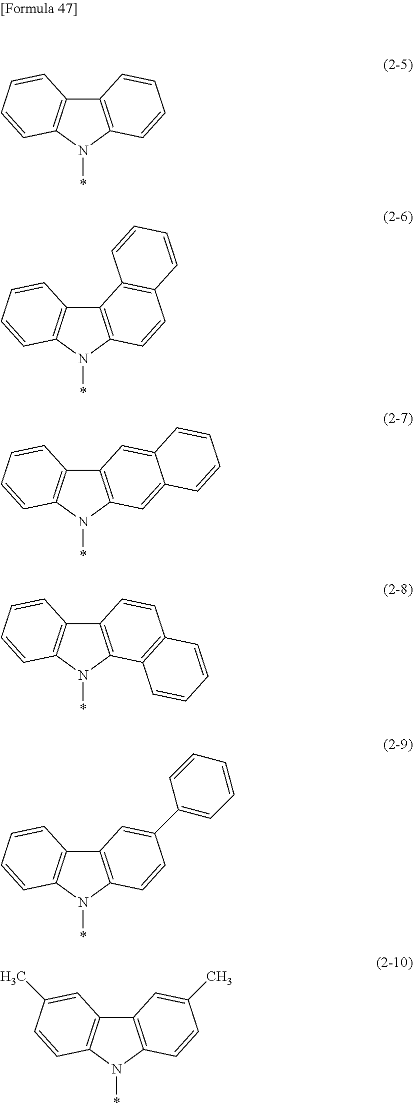

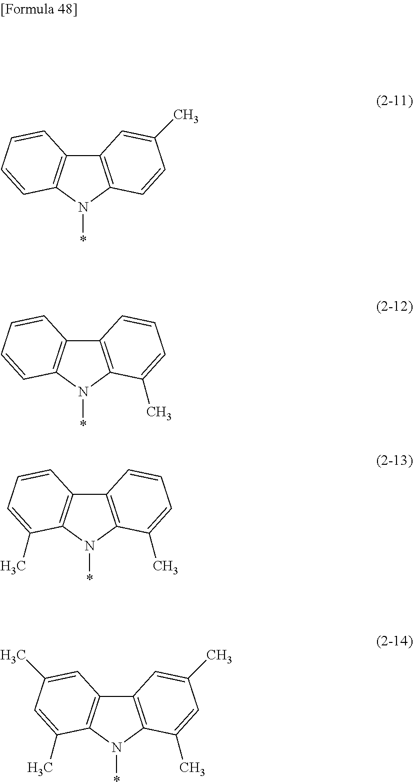

In the formula (2-5) to (2-14), * represents a bonding position to a carbon atom in a six-membered ring in the formula (2).

### Groups Represented by Formulae (D-26) to (D-31)

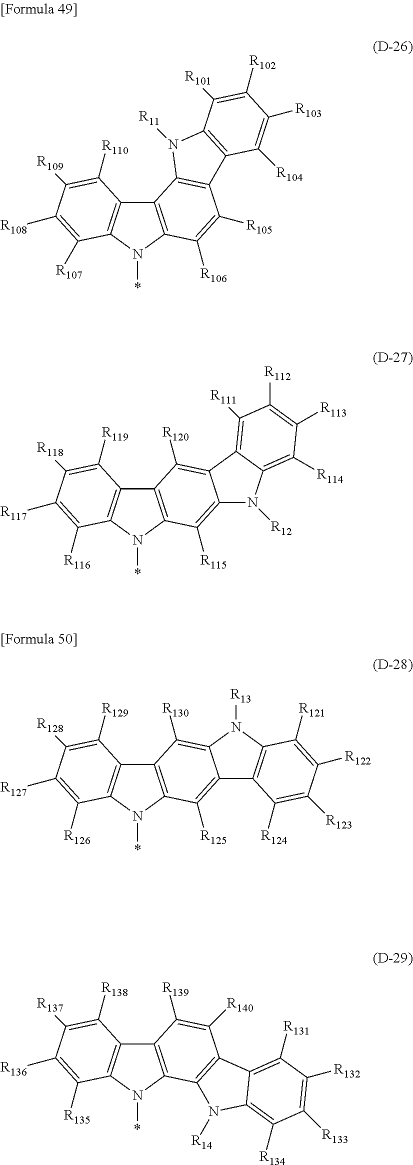

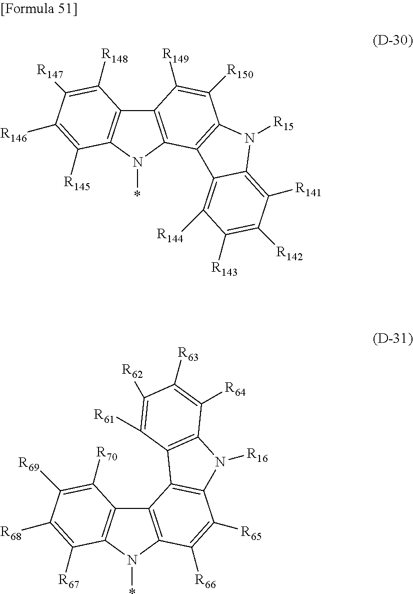

In the formulae (D-26) to (D-31), R₁₁ to R₁₆ are each a substituent, and R₁₀₁ to R₁₅₀ and R₆₁ to R₇₀ are each independently a hydrogen atom or a substituent, or at least one combination of a combination of R₁₁ and R₁₀₂, a combination of R₁₀₂ and R₁₀₃, a combination of R₁₀₃ and R₁₀₄, a combination of R₁₀₅ and R₁₀₆, a combination of R₁₀₇ and R₁₀₈, a combination of R₁₀₈ and R₁₀₉, a combination of R₁₀₉ and R₁₁₀, a combination of R₁₁₁ and R₁₁₂, a combination of R₁₁₂ and R₁₁₃, a combination of R₁₁₃ and R₁₁₄, a combination of R₁₁₆ and R₁₁₇, a combination of R₁₁₇ and R₁₁₈, a combination of R₁₁₈ and R₁₁₉, a combination of R₁₂₁ and R₁₂₂, a combination of R₁₂₂ and R₁₂₃, a combination of R₁₂₃ and R₁₂₄, a combination of R₁₂₆ and R₁₂₇, a combination of R₁₂₇ and R₁₂₈, a combination of R₁₂₈ and R₁₂₉, a combination of R₁₃₁ and R₁₃₂, a combination of R₁₃₂ and R₁₃₃, a combination of R₁₃₃ and R₁₃₄, a combination of R₁₃₅ and R₁₃₆, a combination of R₁₃₆ and R₁₃₇, a combination of R₁₃₇ and R₁₃₈, a combination of R₁₃₉ and R₁₄₀, a combination of R₁₄₁ and R₁₄₂, a combination of R₁₄₂ and R₁₄₃, a combination of R₁₄₃ and R₁₄₄, a combination of R₁₄₅ and R₁₄₆, a combination of R₁₄₆ and R₁₄₇, a combination of R₁₄₇ and R₁₄₈, a combination of R₁₄₉ and R₁₅₀, a combination of R₆₁ and R₆₂, a combination of R₆₂ and R₆₃, a combination of R₆₃ and R₆₄, a combination of R₆₅ and R₆₆, a combination of R₆₇ and R₆₈, a combination of R₆₈ and R₆₉, or a combination of R₆₉ and R₇₀ are bonded to each other to form a ring;
R₁₀₁ to R₁₅₀ and R₆₁ to R₇₀ as the substituents are each independently a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkylsilyl group having 3 to 6 carbon atoms, a hydroxy group, a substituted or unsubstituted alkoxy group having 1 to 6 carbon atoms, a substituted or unsubstituted aryloxy group having 6 to 14 ring carbon atoms, a substituted or unsubstituted arylamino group having 6 to 28 ring carbon atoms, a substituted or unsubstituted alkylamino group having 2 to 12 carbon atoms, a thiol group, a substituted or unsubstituted alkylthio group having 1 to 6 carbon atoms, or a substituted or unsubstituted arylthio group having 6 to 14 ring carbon atoms;
R₁₁ to R₁₆ as the substituents are each independently a substituted or unsubstituted alkyl group having 1 to 6 carbon atoms, a substituted or unsubstituted aryl group having 6 to 14 ring carbon atoms, a substituted or unsubstituted heterocyclic group having 5 to 14 ring atoms, a substituted or unsubstituted alkylsilyl group having 3 to 6 carbon atoms, a substituted or unsubstituted aryloxy group having 6 to 14 ring carbon atoms, a substituted or unsubstituted alkylamino group having 2 to 12 carbon atoms, a substituted or unsubstituted alkylthio group having 1 to 6 carbon atoms, or a substituted or unsubstituted arylthio group having 6 to 14 ring carbon atoms; and
* represents a bonding position to a carbon atom in a six-membered ring in the formula (2).

In the compound M2, it is preferable that R₁₀₁ to R₁₅₀ and R₆₁ to R₇₀ as the substituents are each independently an unsubstituted aryl group having 6 to 14 ring carbon atoms, an unsubstituted heterocyclic group having 5 to 14 ring atoms, or an unsubstituted alkyl group having 1 to 6 carbon atoms, and
R₁₁ to R₁₆ as the substituents are each independently an unsubstituted aryl group having 6 to 14 ring carbon atoms or an unsubstituted heterocyclic group having to 14 ring atoms.

In the compound M2, it is also preferable that R₁₀₁ to R₁₅₀ and R₆₁ to R₇₀ are each a hydrogen atom, and R₁₁ to R₁₆ as the substituents are each independently an unsubstituted aryl group having 6 to 14 ring carbon atoms, or an unsubstituted heterocyclic group having 5 to 14 ring atoms.

### Groups Represented by Formulae (D-32) to (D-37)

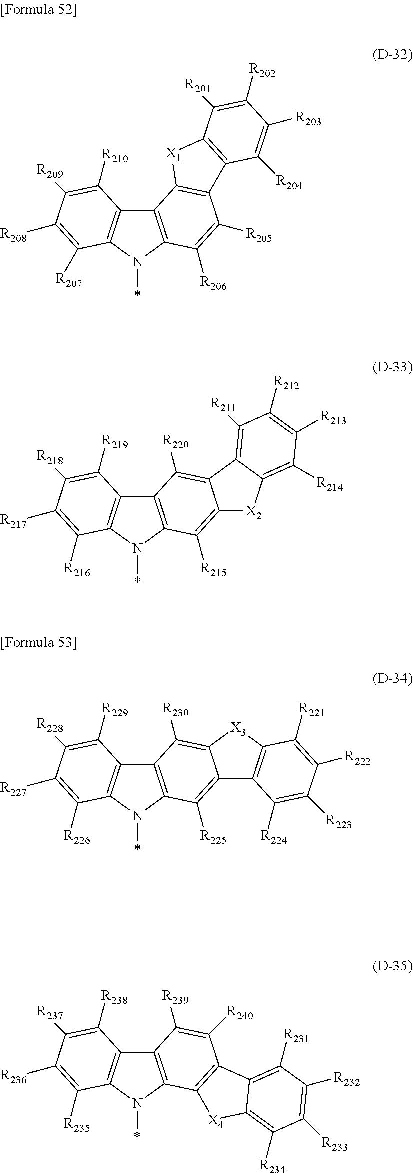

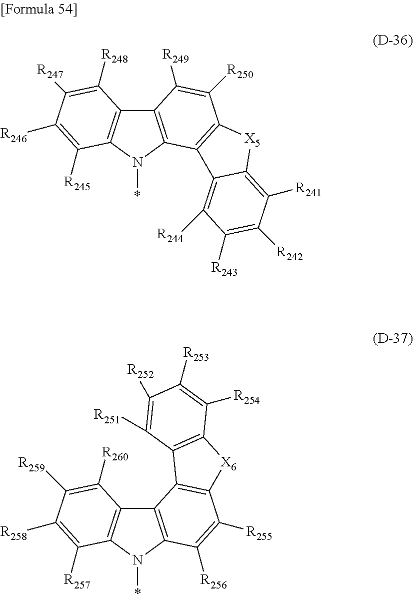

In the formulae (D-32) to (D-37), X₁ to X₆ are each independently an oxygen atom, a sulfur atom, or CR₁₅₁R₁₅₂;
R₁₅₁ and R₁₅₂ are each independently a hydrogen atom or a substituent, or R₁₅₁ and R₁₅₂ are bonded to each other to form a ring;
R₂₀₁ to R₂₆₀ are each independently a hydrogen atom or a substituent, or at least one combination of a combination of R₂₀₁ and R₂₀₂, a combination of R₂₀₂ and R₂₀₃, a combination of R₂₀₃ and R₂₀₄, a combination of R₂₀₅ and R₂₀₆, a combination of R₂₀₇ and R₂₀₈, a combination of R₂₀₈ and R₂₀₉, a combination of R₂₀₉ and R₂₁₀, a combination of R₂₁₁ and R₂₁₂, a combination of R₂₁₂ and R₂₁₃, a combination of R₂₁₃ and R₂₁₄, a combination of R₂₁₆ and R₂₁₇, a combination of R₂₁₇ and R₂₁₈, a combination of R₂₁₈ and R₂₁₉, a combination of R₂₂₁ and R₂₂₂, a combination of R₂₂₂ and R₂₂₃, a combination of R₂₂₃ and R₂₂₄, a combination of R₂₂₆ and R₂₂₇, a combination of R₂₂₇ and R₂₂₈, a combination of R₂₂₈ and R₂₂₉, a combination of R₂₃₁ and R₂₃₂, a combination of R₂₃₂ and R₂₃₃, a combination of R₂₃₃ and R₂₃₄, a combination of R₂₃₅ and R₂₃₆, a combination of R₂₃₆ and R₂₃₇, a combination of R₂₃₇ and R₂₃₈, a combination of R₂₃₉ and R₂₄₀, a combination of R₂₄₁ and R₂₄₂, a combination of R₂₄₂ and R₂₄₃, a combination of R₂₄₃ and R₂₄₄, a combination of R₂₄₅ and R₂₄₆, a combination of R₂₄₆ and R₂₄₇, a combination of R₂₄₇ and R₂₄₈, a combination of R₂₄₉ and R₂₅₀, a combination of R₂₅₁ and R₂₅₂, a combination of R₂₅₂ and R₂₅₃, a combination of R₂₅₃ and R₂₅₄, a combination of R₂₅₅ and R₂₅₆, a combination of R₂₅₇ and R₂₅₈, a combination of R₂₅₈ and R₂₅₉, or a combination of R₂₅₉ and R₂₆₀ are bonded to each other to form a ring;
R₁₅₁, R₁₅₂ and R₂₀₁ to R₂₆₀ as the substituents are each independently a halogen atom, a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkyl halide group having 1 to 6 carbon atoms, a substituted or unsubstituted cycloalkyl group having 3 to 30 ring carbon atoms, a substituted or unsubstituted alkylsilyl group having 3 to 6 carbon atoms, a hydroxy group, a substituted or unsubstituted alkoxy group having 1 to 6 carbon atoms, a substituted or unsubstituted alkoxy halide group having 1 to 6 carbon atoms, a substituted or unsubstituted aryloxy group having 6 to 14 ring carbon atoms, a substituted or unsubstituted arylamino group having 6 to 28 ring carbon atoms, a substituted or unsubstituted alkylamino group having 2 to 12 carbon atoms, a thiol group, a substituted or unsubstituted alkylthio group having 1 to 6 carbon atoms, or a substituted or unsubstituted arylthio group having 6 to 14 ring carbon atoms; and
* represents a bonding position to a carbon atom in a six-membered ring in the formula (2).

In the compound M2, it is preferable that R₂₀₁ to R₂₆₀ as the substituents are each independently a halogen atom, an unsubstituted aryl group having 6 to 14 ring carbon atoms, an unsubstituted heterocyclic group having 5 to 14 ring atoms, or an unsubstituted alkyl group having 1 to 6 carbon atoms, and
R₁₅₁ and R₁₅₂ as the substituents are each independently an unsubstituted aryl group having 6 to 14 ring carbon atoms or an unsubstituted alkyl group having 1 to 6 carbon atoms.

In the compound M2, it is more preferable that R₂₀₁ to R₂₆₀ as the substituents are each independently an unsubstituted aryl group having 6 to 14 ring carbon atoms, an unsubstituted heterocyclic group having 5 to 14 ring atoms, or an unsubstituted alkyl group having 1 to 6 carbon atoms, and
R₁₅₁ and R₁₅₂ as the substituents are each independently an unsubstituted aryl group having 6 to 14 ring carbon atoms or an unsubstituted alkyl group having 1 to 6 carbon atoms.

In the compound M2, it is also preferable that R₂₀₁ to R₂₆₀ are each a hydrogen atom; and
R₁₅₁ and R₁₅₂ as the substituents are each independently an unsubstituted aryl group having 6 to 14 ring carbon atoms or an unsubstituted alkyl group having 1 to 6 carbon atoms.

### Compound Represented by Formula (22)

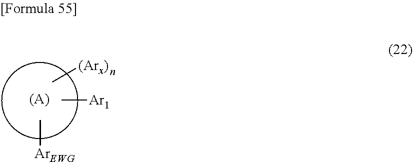

In the formula (22):
Ar₁ is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by formulae (1a) to (1j) below;
Ar_{EWG} is a substituted or unsubstituted heteroaryl group having 5 to 30 ring atoms that includes at least one nitrogen atom in a ring, or an aryl group having 6 to ring carbon atoms that is substituted by at least one cyano group;
each Ar_{X} is independently a hydrogen atom or a substituent, and Ar_{X} as the substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by the formulae (1a) to (1j);
n is 0, 1, 2, 3, 4 or 5, and when n is 2, 3, 4 or 5, a plurality of Ar_{X} are mutually the same or different;
a ring (A) is a substituted or unsubstituted aromatic hydrocarbon ring or a substituted or unsubstituted heterocycle, the ring (A) is a five-membered ring, a six-membered ring, or a seven-membered ring, and Ar_{EWG}, Ar₁ and Ar_{X} are bonded to respective ones of elements forming the ring (A); and
at least one of Ar₁ or Ar_{X} is a group selected from the group consisting of groups represented by the formulae (1a) to (1j).

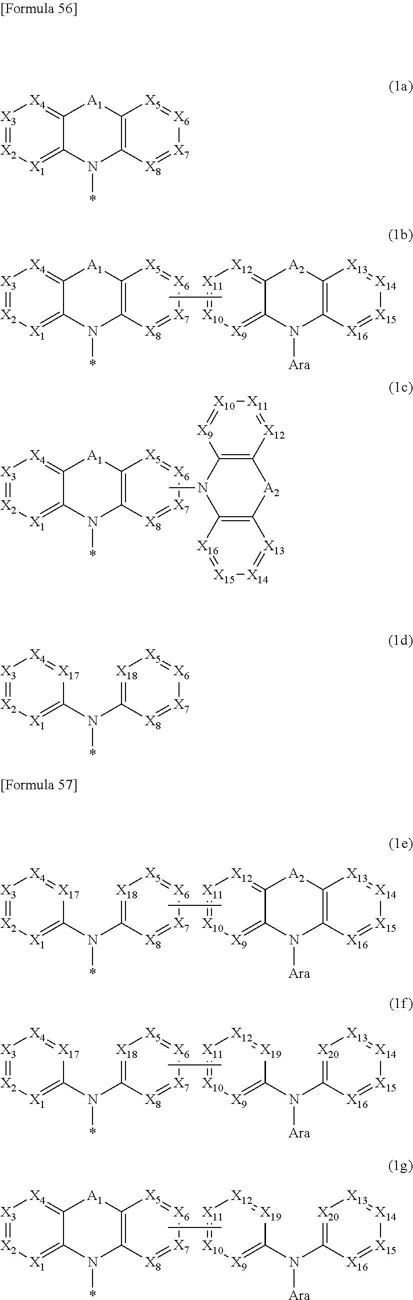

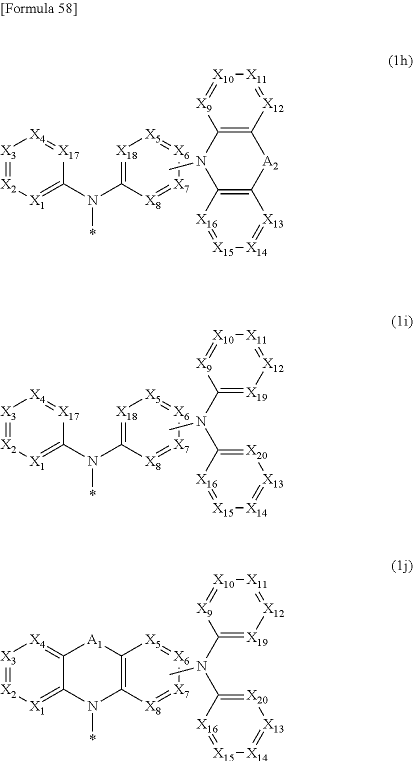

In the formulae (1a) to (1j), X₁ to X₂₀ are each independently a nitrogen atom (N) or a carbon atom bonded with R_{A1} (C—R_{A1}).

In the formula (1b), one of X₅ to X₈ is a carbon atom bonded to one of X₉ to X₁₂, and one of X₉ to X₁₂ is a carbon atom bonded to one of X₅ to X₈.

In the formula (1c), one of X₅ to X₈ is a carbon atom bonded to a nitrogen atom in a ring including A₂.

In the formula (1e), one of X₅ to X₈ and X₁₈ is a carbon atom bonded to one of X₉ to X₁₂, and one of X₉ to X₁₂ is a carbon atom bonded to one of X₅ to X₈ and X₁₈.

In the formula (1f), one of X₅ to X8 and X₁₈ is a carbon atom bonded to one of X₉ to X₁₂ and X₁₉, and one of X₉ to X₁₂ and X₁₉ is a carbon atom bonded to one of X₅ to X8 and X₁₈.

In the formula (1g), one of X₅ to X8 is a carbon atom bonded to one of X9 to X₁₂ and X₁₉, and one of X₉ to X₁₂ and X₁₉ is a carbon atom bonded to one of X₅ to X8.

In the formula (1h), one of X₅ to X8 and X₁₈ is a carbon atom bonded to a nitrogen atom in a ring including A₂.

In the formula (1i), one of X₅ to X8 and X₁₈ is a carbon atom bonded to a nitrogen atom that links a ring including X₉ to X₁₂ and X₁₉ with a ring including X₁₃ to X₁₆ and X₂₀.

In the formula (1j), one of X₅ to X8 is a carbon atom bonded to a nitrogen atom that links a ring including X9 to X₁₂ and X₁₉ with a ring including X₁₃ to X₁₆ and X₂O.

Each R_{A1} is independently a hydrogen atom or a substituent, or at least one combination of combinations of a plurality of R_{A1} are mutually directly bonded to form a ring or bonded via a hetero atom to form a ring.

R_{A1} as the substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group.

When a plurality of R_{A1} as the substituents are present, the plurality of R_{A1} are mutually the same or different.

In the formulae (1a) to (1j), * represents a bonding position to the ring (A).

In the formulae (1a) to (1j), A₁ and A₂ are each independently a single bond, an oxygen atom (O), a sulfur atom (S), C(R₂₀₂₁)(R₂₀₂₂), Si(R₂₀₂₃)(R₂₀₂₄), C(═O), S(═O), SO₂ or N(R₂₀₂₅). R₂₀₂₁ to R₂₀₂₅ are each independently a hydrogen atom or a substituent, and R₂₀₂₁ to R₂₀₂₅ as the substituents are each independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group.

In the formulae (1a) to (1j), Ara is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, and a substituted silyl group.

In the formula (1a), when X₁ to X₈ are each a carbon atom bonded with R_{A1} (C—R_{A1}), a plurality of R_{A1} preferably form no ring.

Ara is preferably a substituted or unsubstituted aryl group having 6 to 30 ring carbon atoms or a substituted or unsubstituted heteroaryl group having 5 to 30 ring atoms.

The formula (1a) is represented by a formula (1aa) below when A₁ is a single bond, represented by a formula (1ab) below when A₁ is O, represented by a formula (1ac) below when A₁ is S, represented by a formula (1ad) below when A₁ is C(R₂₀₂₁)(R₂₀₂₂), represented by a formula (1ae) below when A₁ is Si(R₂₀₂₃)(R₂₀₂₄), represented by a formula (1af) below when A₁ is C(═O), represented by a formula (1ag) below when A₁ is S(═O), represented by a formula (1ah) below when A₁ is SO₂, and represented by a formula (1ai) below when A₁ is N(R₂₀₂₅). In the formulae (1aa) to (1ai), X₁ to X₈ and R₂₀₂₁ to R₂₀₂₅ represent the same as described above. Linkages between rings via A₁ and A₂ in the formulae (1b), (1c), (1e) and (1g) to (1j) are the same as those in the formulae (1aa) to (1ai). In the formula (1aa), when X₁ to X8 are each a carbon atom bonded with R_{A1} (C—R_{A1}), a plurality of R_{A1} as substituents preferably form no ring.

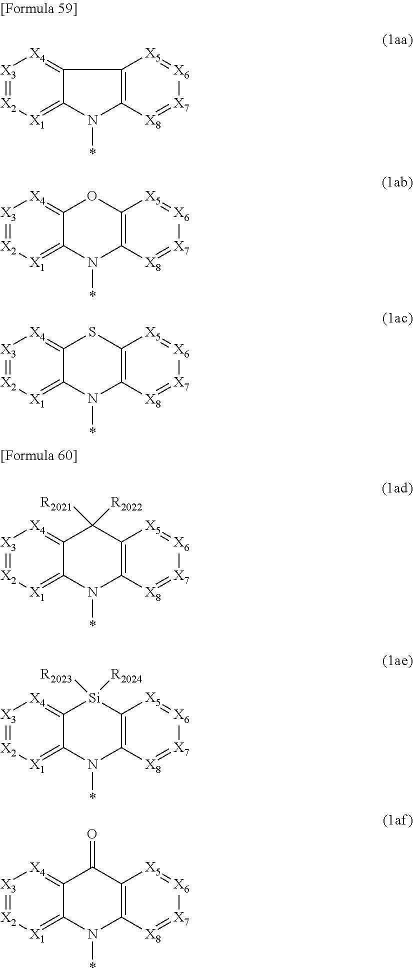

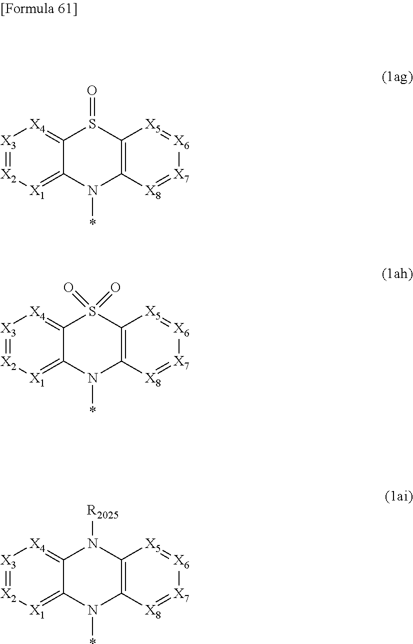

The compound M2 is also preferably represented by a formula (221) below.

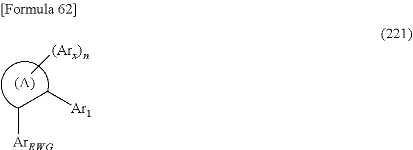

Ar₁, Ar_{EWG}, Ar_{X}, n and a ring (A) in the formula (221) respectively represent the same as Ar₁, Ar_{EWG}, Ar_{X}, n and the ring (A) in the formula (22).

The compound M2 is also preferably represented by a formula (222) below.

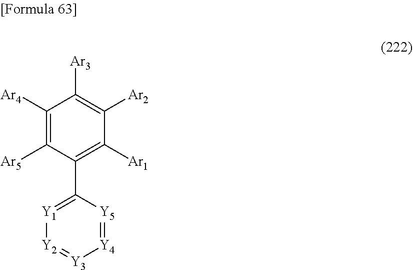

In the formula (222), Y₁ to Y₅ are each independently a nitrogen atom (N), a carbon atom bonded with a cyano group (C—CN), or a carbon atom bonded with R_{A2} (C—R_{A2}), and at least one of Y₁ to Y₅ is N or C—CN. A plurality of R_{A2} are mutually the same or different. R_{A2} are each independently a hydrogen atom or a substituent, R_{A2} as the substituent being a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group; and
a plurality of R_{A2} are mutually the same or different.

In the formula (222), Ar₁ represents the same as Ar₁ in the formula (22).

In the formula (222), Ar₂ to Ar₅ are each independently a hydrogen atom or a substituent, and Ar₂ to Ar₅ as the substituents are each independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by the formulae (1a) to (1c).

It is preferable that, when one or more of Ar₂ to Ar₅ are hydrogen atoms in the formula (222), all of the hydrogen atom(s) are protium atoms, at least one of the hydrogen atom(s) is a deuterium atom, or all of the hydrogen atom(s) are deuterium atoms.

It is preferable that, when one or more of Ar₂ to Ar₅ are substituents and the substituents include one or more hydrogen atoms in the formula (222), all of the hydrogen atom(s) are protium atoms, at least one of the hydrogen atom(s) is a deuterium atom, or all of the hydrogen atom(s) are deuterium atoms.

In the formula (222), at least one of Ar₁ to Ar₅ is a group selected from the group consisting of groups represented by the formulae (1a) to (1c).

The compound M2 is also preferably a compound represented by a formula (11aa), (11 bb) or (11cc) below.

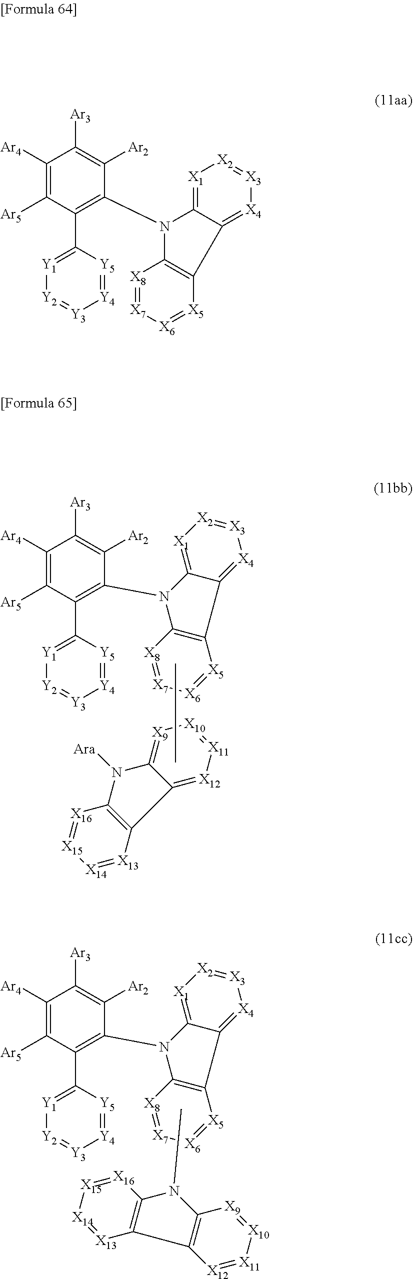

In the formulae (11a), (11b) and (11c), Y₁ to Y₅, R_{A2}, Ar₂ to Ar₅, X₁ to X₁₆, R_{A1} and Ara respectively represent the same as the above-described Y₁ to Y₅, R_{A2}, Ar₂ to Ar₅, X₁ to X₁₆, R_{A1} and Ara.

The compound M2 is exemplified by a compound represented by a formula (23) below.

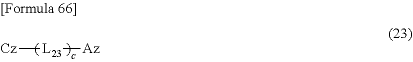

In the formula (23):
Az is a cyclic structure selected from the group consisting of a substituted or unsubstituted pyridine ring, a substituted or unsubstituted pyrimidine ring, a substituted or unsubstituted triazine ring, and a substituted or unsubstituted pyrazine ring;
c is 0, 1, 2, 3, 4 or 5;
when c is 0, Cz and Az are bonded by a single bond;
when c is 1, 2, 3, 4 or 5, L₂₃ is a linking group selected from the group consisting of a substituted or unsubstituted arylene group having 6 to 30 ring carbon atoms, and a substituted or unsubstituted heteroarylene group having 5 to 30 ring atoms;
when c is 2, 3, 4 or 5, a plurality of L₂₃ are mutually the same or different;
the plurality of L₂₃ are mutually bonded to form a ring or not bonded to form no ring; and
Cz is represented by a formula (23a) below.

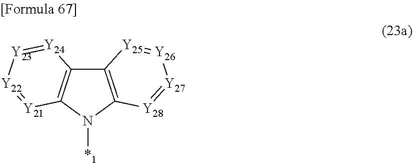

In the formula (23a):
Y₂₁ to Y₂₈ are each independently a nitrogen atom or CR_{A3};
each R_{A3} is independently a hydrogen atom or a substituent, or at least one combination of combinations among a plurality of R_{A3} are mutually bonded to form a ring;
each R_{A3} as the substituent is independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;
a plurality of R_{A3} are mutually the same or different; and
*1 represents a bonding position to a carbon atom in a structure of a linking group represented by L₂₃, or a bonding position to a carbon atom in a cyclic structure represented by Az.

Y₂₁ to Y₂₈ are also preferably CR_{A3}.

c in the formula (23) is preferably 0 or 1.

Cz is also preferably represented by a formula (23b), (23c) or (23d) below.

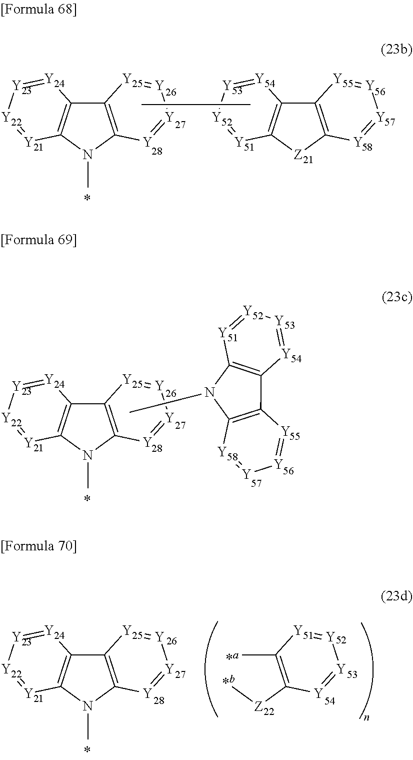

In the formulae (23b), (23c) and (23d), Y₂₁ to Y₂₈ and Y₅₁ to Y₅₈ are each independently a nitrogen atom or CR_{A4};
in the formula (23b), at least one of Y₂₅ to Y₂₈ is a carbon atom bonded to one of Y₅₁ to Y₅₄, and at least one of Y₅₁ to Y₅₄ is a carbon atom bonded to one of Y₂₅ to Y₂₈;
in the formula (23c), at least one of Y₂₅ to Y₂₈ is a carbon atom bonded to a nitrogen atom in a five-membered ring of a nitrogen-containing fused ring including Y₅₁ to Y₅₈;
in the formula (23d), *a and *b each represent a bonding position to one of Y₂₁ to Y₂₈, at least one of Y₂₅ to Y₂₈ is the bonding position represented by *a, and at least one of Y₂₅ to Y₂₈ is the bonding position represented by *b;
n is 1, 2, 3 or 4;
each R_{A4} is independently a hydrogen atom or a substituent, or at least one combination of combinations among a plurality of R_{A4} are mutually bonded to form a ring;
each R_{A4} as the substituent is independently a substituent selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;
a plurality of R_{A4} are mutually the same or different;
Z₂₁ and Z₂₂ are each independently any one selected from the group consisting of an oxygen atom, a sulfur atom, NR₄₅ and CR₄₆R₄₇;
R₄₅ is a hydrogen atom or a substituent;
R₄₆ and R₄₇ are each independently a hydrogen atom or a substituent, or a combination of R₄₆ and R₄₇ are mutually bonded to form a ring;
R₄₅, R₄₆ and R₄₇ as the substituents are each independently a substituent selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;
a plurality of R₄₅ are mutually the same or different;
a plurality of R₄₆ are mutually the same or different;
a plurality of R₄₇ are mutually the same or different; and
* represents a bonding position to a carbon atom in a cyclic structure represented by Az.

Z₂₁ is preferably NR₄₅.

When Z₂₁ is NR₄₅, R₄₅ is preferably a substituted or unsubstituted aryl group having 6 to 30 ring carbon atoms.

Z₂₂ is preferably NR₄₅.

When Z₂₂ is NR₄₅, R₄₅ is preferably a substituted or unsubstituted aryl group having 6 to 30 ring carbon atoms.

Y₅₁ to Y₅₈ are preferably CR_{A4}, provided that at least one of Y₅₁ to Y₅₈ is a carbon atom bonded to a cyclic structure represented by the formula (23a).

It is also preferable that Cz is represented by the formula (23d) and n is 1.

Az is preferably a cyclic structure selected from the group consisting of a substituted or unsubstituted pyrimidine group and a substituted or unsubstituted triazine group.

Az is a cyclic structure selected from the group consisting of a substituted pyrimidine ring and a substituted triazine ring, in which a substituent of each of the substituted pyrimidine ring and the substituted triazine ring is more preferably a group selected from the group consisting of a substituted or unsubstituted aryl group having 6 to 30 ring carbon atoms and a substituted or unsubstituted heteroaryl group having to 30 ring atoms, and still more preferably a substituted or unsubstituted aryl group having 6 to 30 ring carbon atoms.

When the pyrimidine ring and the triazine ring as Az have a substituted or unsubstituted aryl group as a substituent, the aryl group preferably has 6 to 20 ring carbon atoms, more preferably 6 to 14 ring carbon atoms, and still more preferably 6 to 12 ring carbon atoms.

When Az has a substituted or unsubstituted aryl group as a substituent, the substituent is preferably a group selected from the group consisting of a substituted or unsubstituted phenyl group, a substituted or unsubstituted biphenyl group, a substituted or unsubstituted naphthyl group, a substituted or unsubstituted phenanthryl group, a substituted or unsubstituted terphenyl group, and a substituted or unsubstituted fluorenyl group, more preferably a group selected from the group consisting of a substituted or unsubstituted phenyl group, a substituted or unsubstituted biphenyl group, and a substituted or unsubstituted naphthyl group.

When Az has a substituted or unsubstituted heteroaryl group as a substituent, the substituent is preferably a substituent selected from the group consisting of a substituted or unsubstituted carbazolyl group, a substituted or unsubstituted dibenzofuranyl group, and a substituted or unsubstituted dibenzothiophenyl group.

It is preferable that each R_{A4} is independently a hydrogen atom or a substituent, and R_{A4} as the substituent is a substituent selected from the group consisting of a substituted or unsubstituted aryl group having 6 to 30 ring carbon atoms and a substituted or unsubstituted heteroaryl group having 5 to 30 ring atoms.

When R_{A4} as the substituent is a substituted or unsubstituted aryl group having 6 to 30 ring carbon atoms, R_{A4} as the substituent is preferably a substituent selected from the group consisting of a substituted or unsubstituted phenyl group, a substituted or unsubstituted biphenyl group, a substituted or unsubstituted naphthyl group, a substituted or unsubstituted phenanthryl group, a substituted or unsubstituted terphenyl group, and a substituted or unsubstituted fluorenyl group, more preferably a substituent selected from the group consisting of a substituted or unsubstituted phenyl group, a substituted or unsubstituted biphenyl group, and a substituted or unsubstituted naphthyl group.

When R_{A4} as the substituent is a substituted or unsubstituted heteroaryl group having 5 to 30 ring atoms, R_{A4} as the substituent is preferably a substituent selected from the group consisting of a substituted or unsubstituted carbazolyl group, a substituted or unsubstituted dibenzofuranyl group, and a substituted or unsubstituted dibenzothiophenyl group.

R₄₅, R₄₆ and R₄₇ as the substituents are preferably each independently a substituent selected from the group consisting of a substituted or unsubstituted aryl group having 6 to 30 ring carbon atoms, a substituted or unsubstituted heteroaryl group having 5 to 30 ring atoms, and a substituted or unsubstituted alkyl group having 1 to 30 carbon atoms.

### Method for Producing Compound M2

The compound M2 can be produced by a known method.

Specific examples of the compound M2 include the following compounds. It should however be noted that the invention is not limited to the specific examples of the compound.

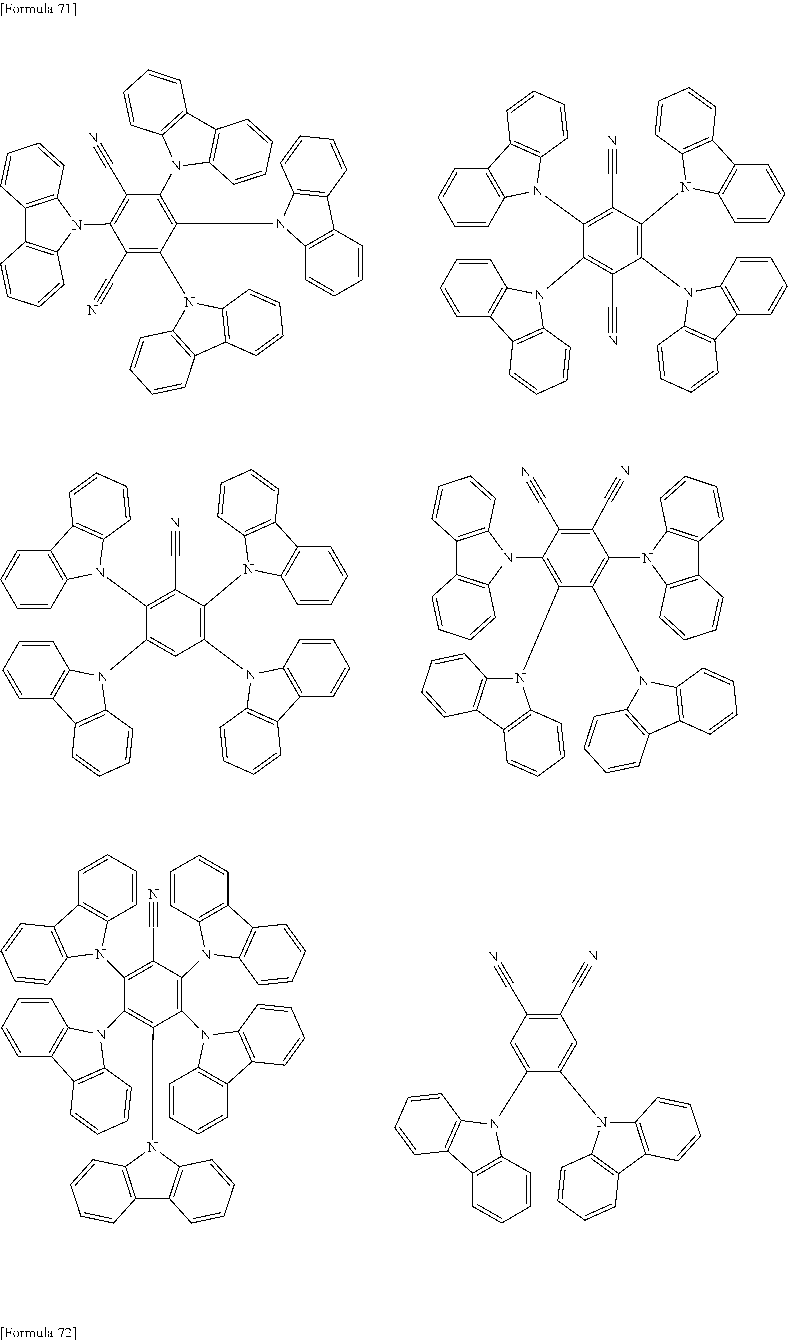

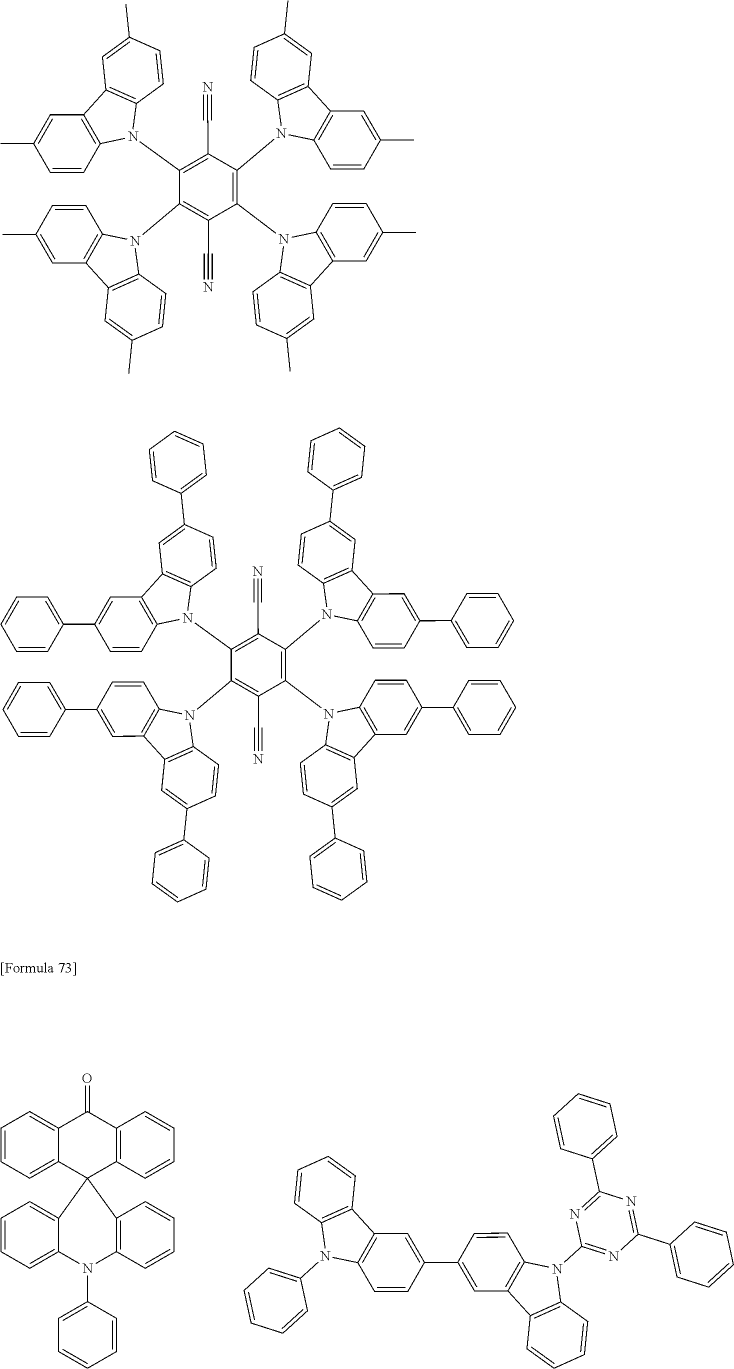

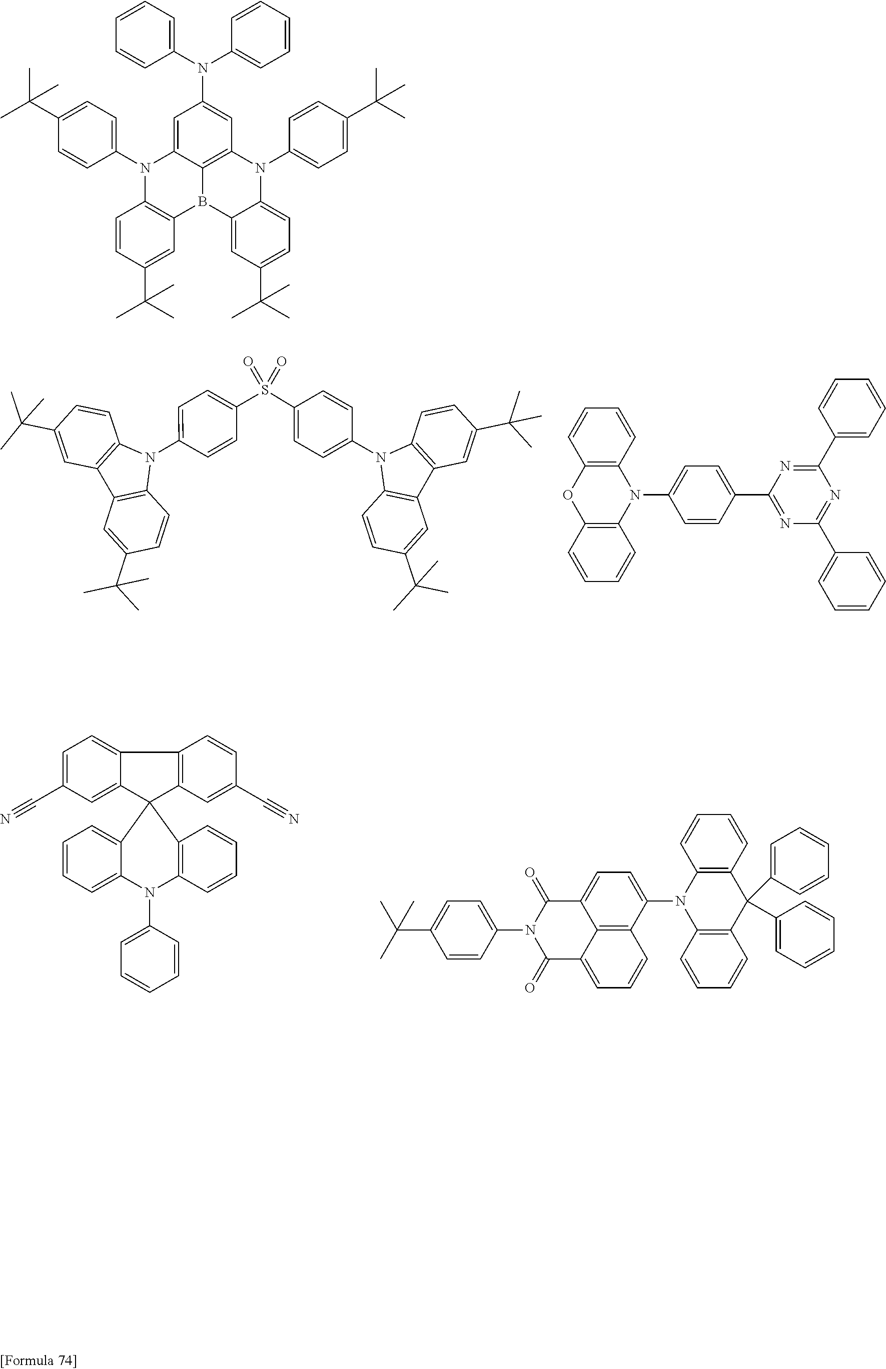

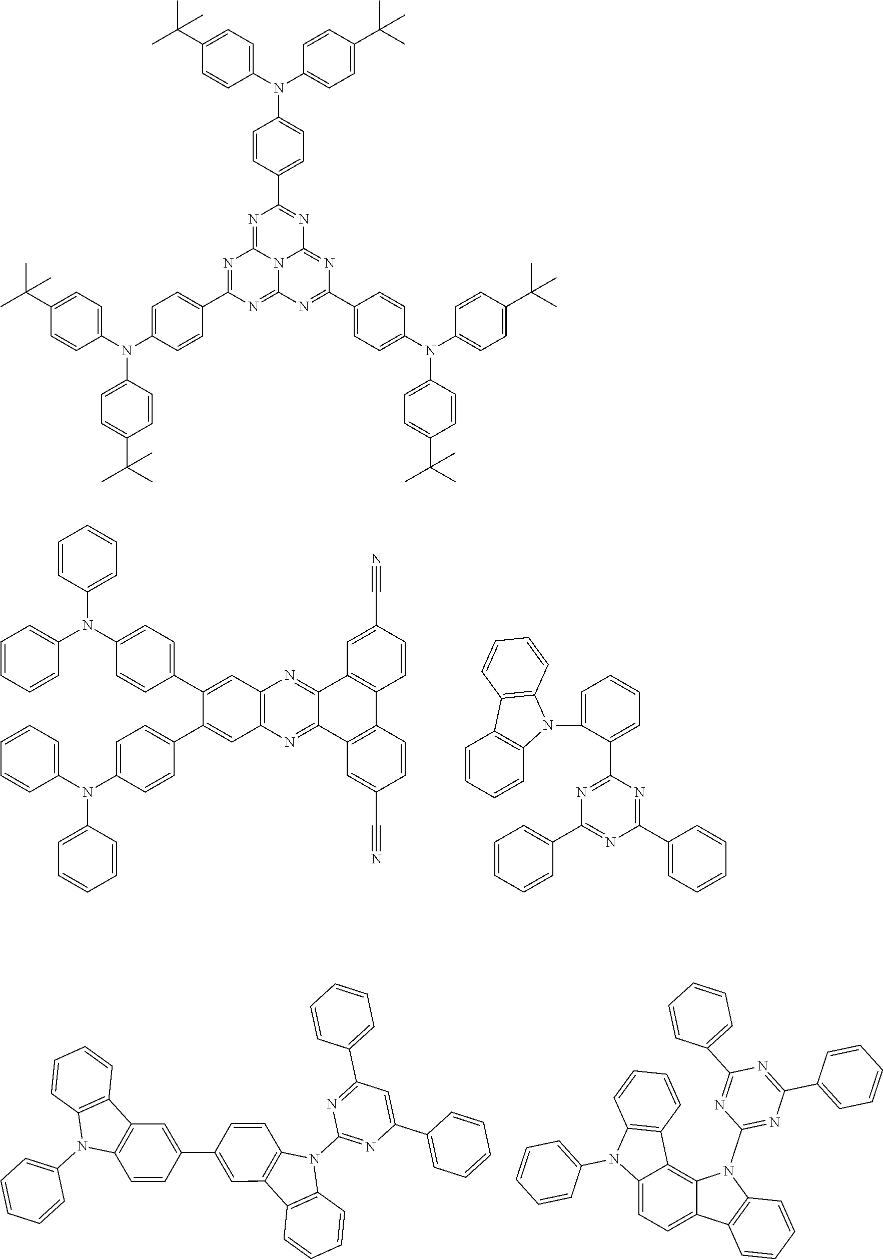

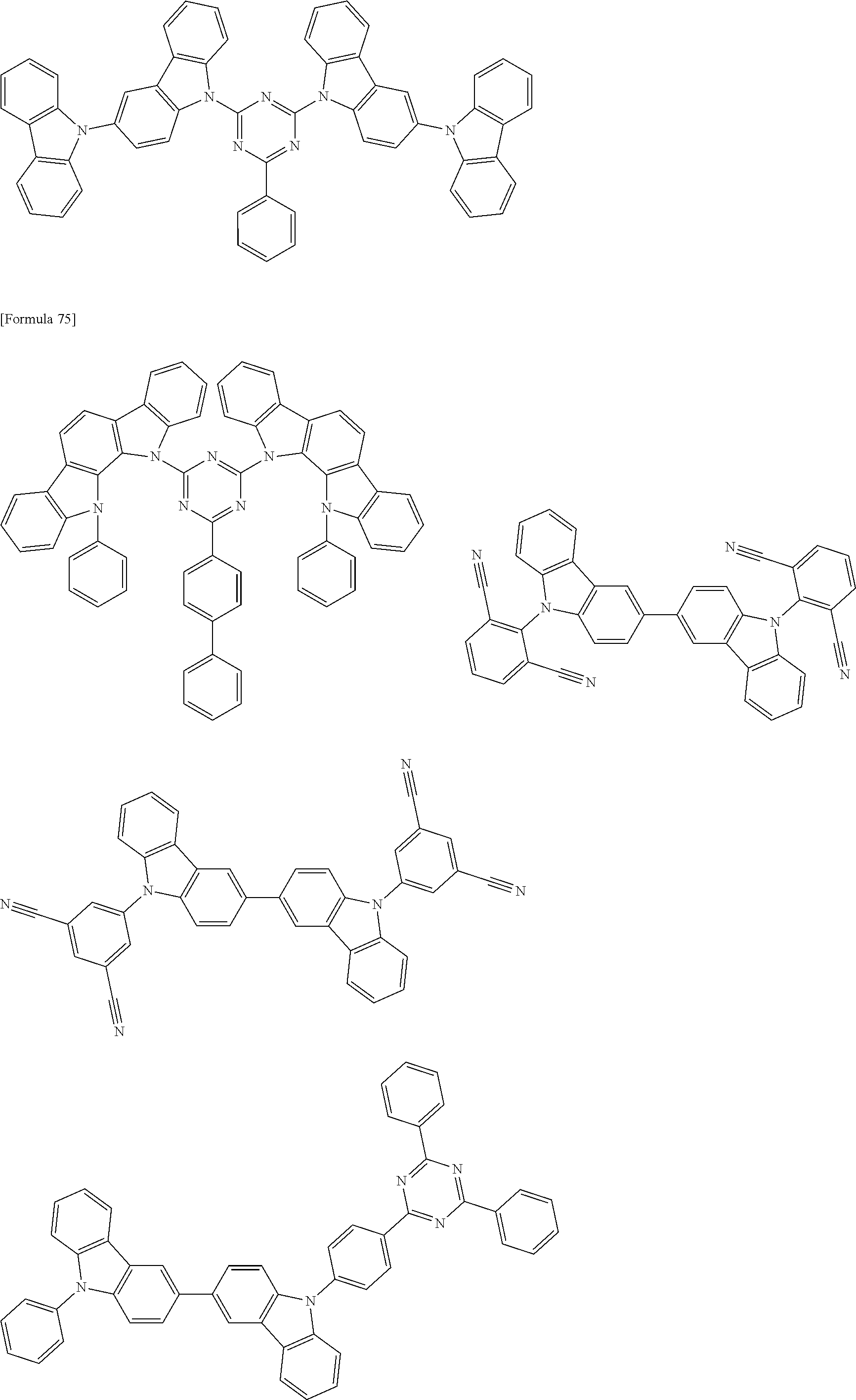 [SMILES: c1ccc(-c2nc(-n3c4ccccc4c4cc(-n5c6ccccc6c6ccccc65)ccc43)nc(-n3c4ccccc4c4cc(-n5c6ccccc6c6ccccc65)ccc43)n2)cc1 ; c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; N#Cc1cccc(C#N)c1-n1c2ccccc2c2cc(-c3ccc4c(c3)c3ccccc3n4-c3c(C#N)cccc3C#N)ccc21 ; N#Cc1cc(C#N)cc(-n2c3ccccc3c3cc(-c4ccc5c(c4)c4ccccc4n5-c4cc(C#N)cc(C#N)c4)ccc32)c1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

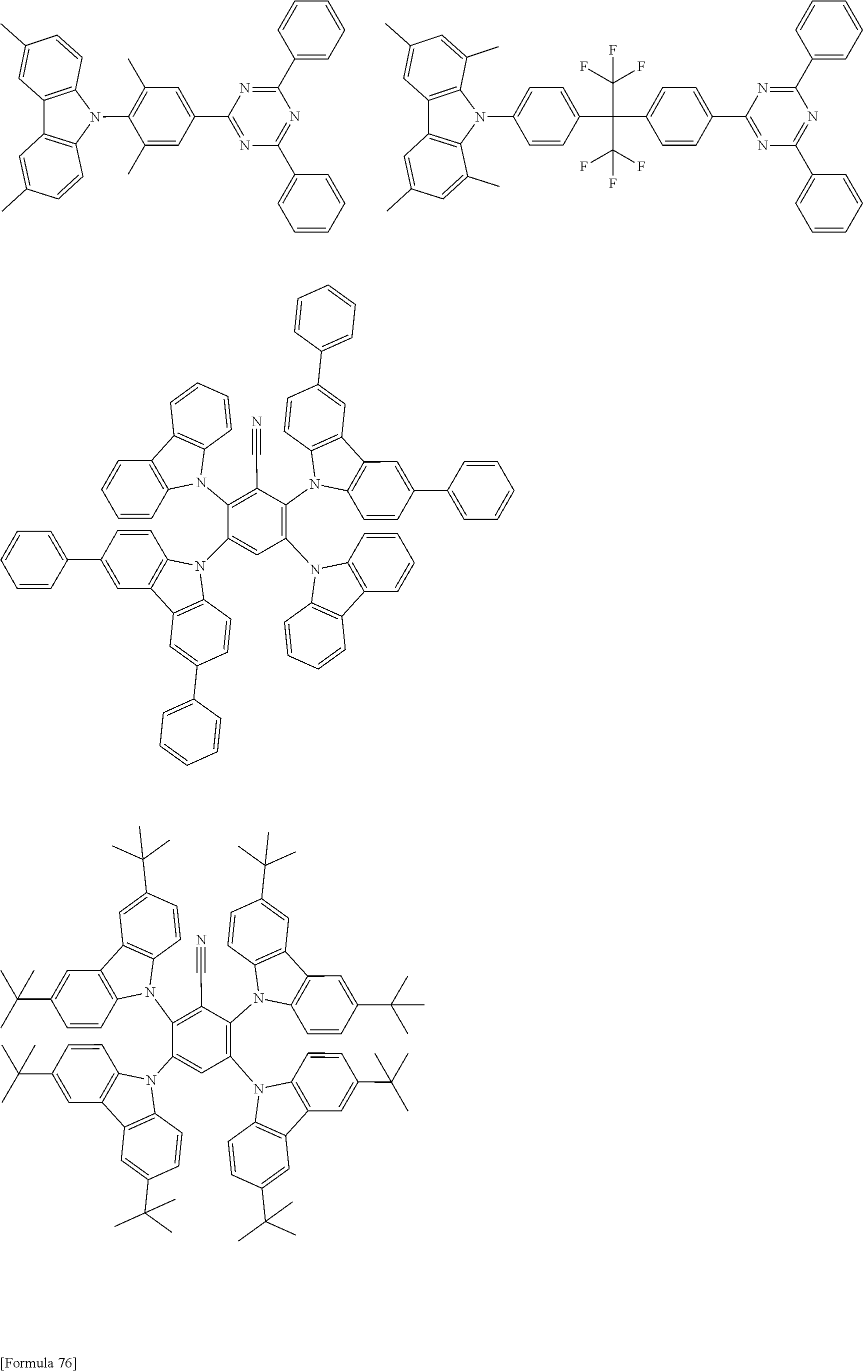

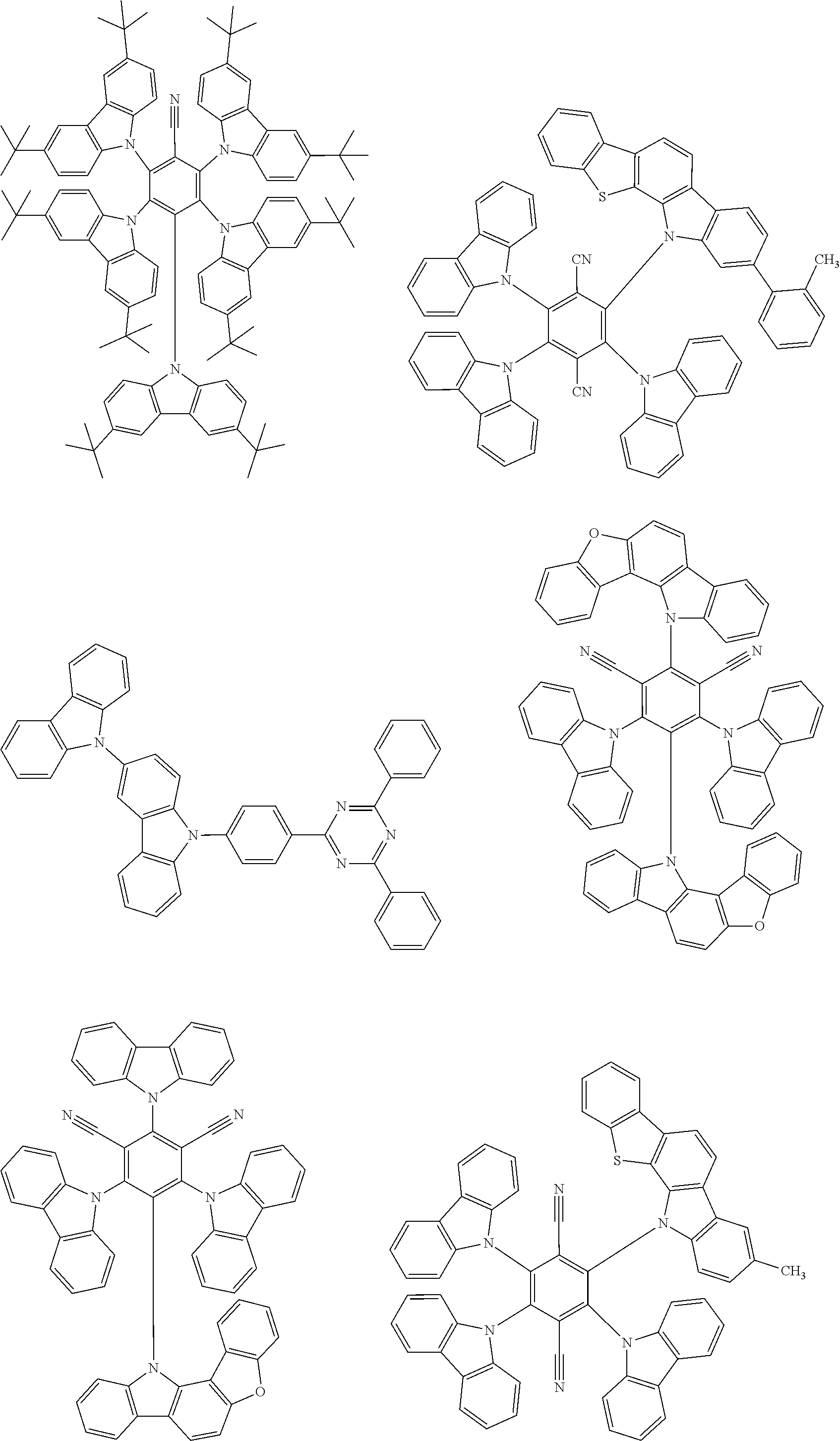

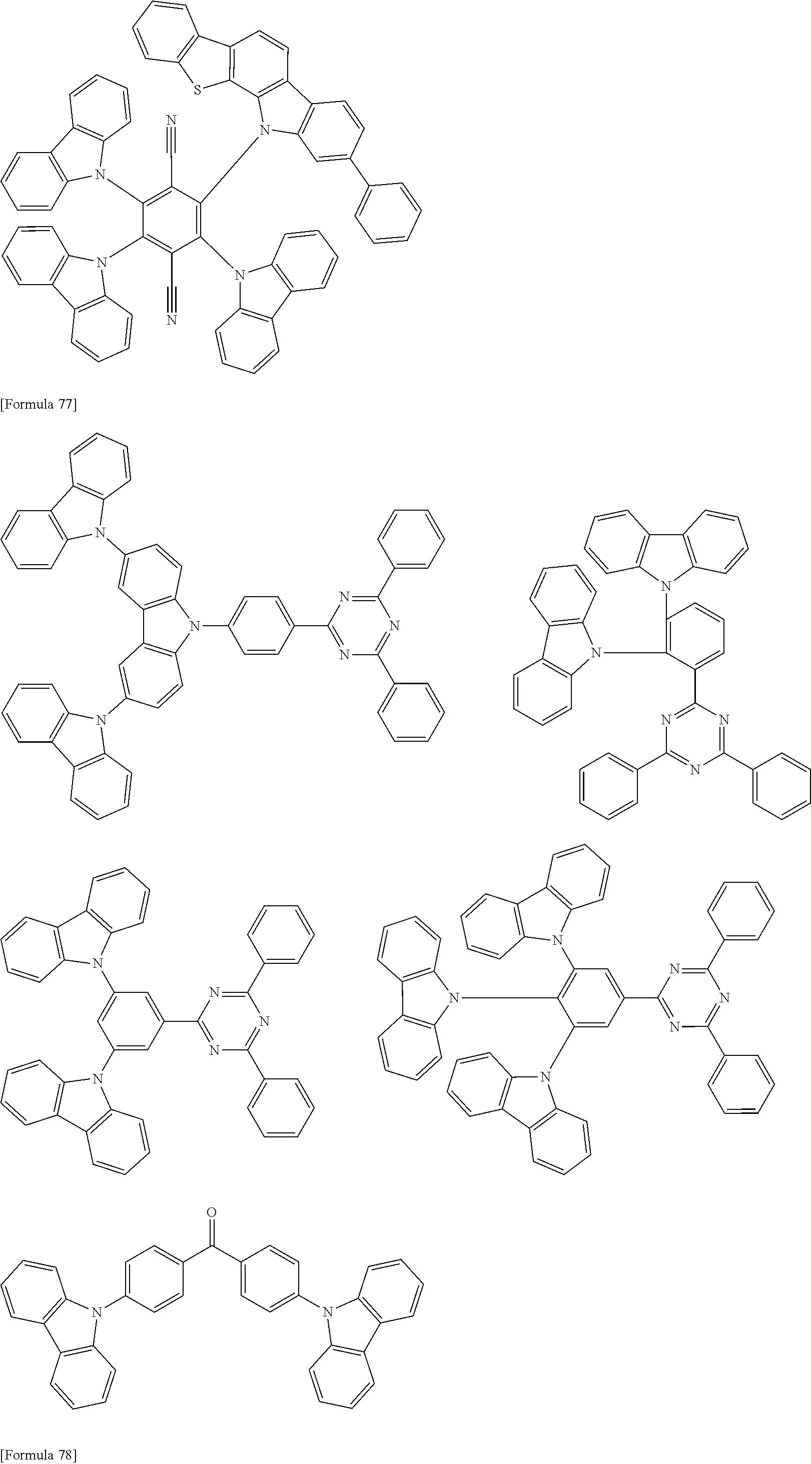

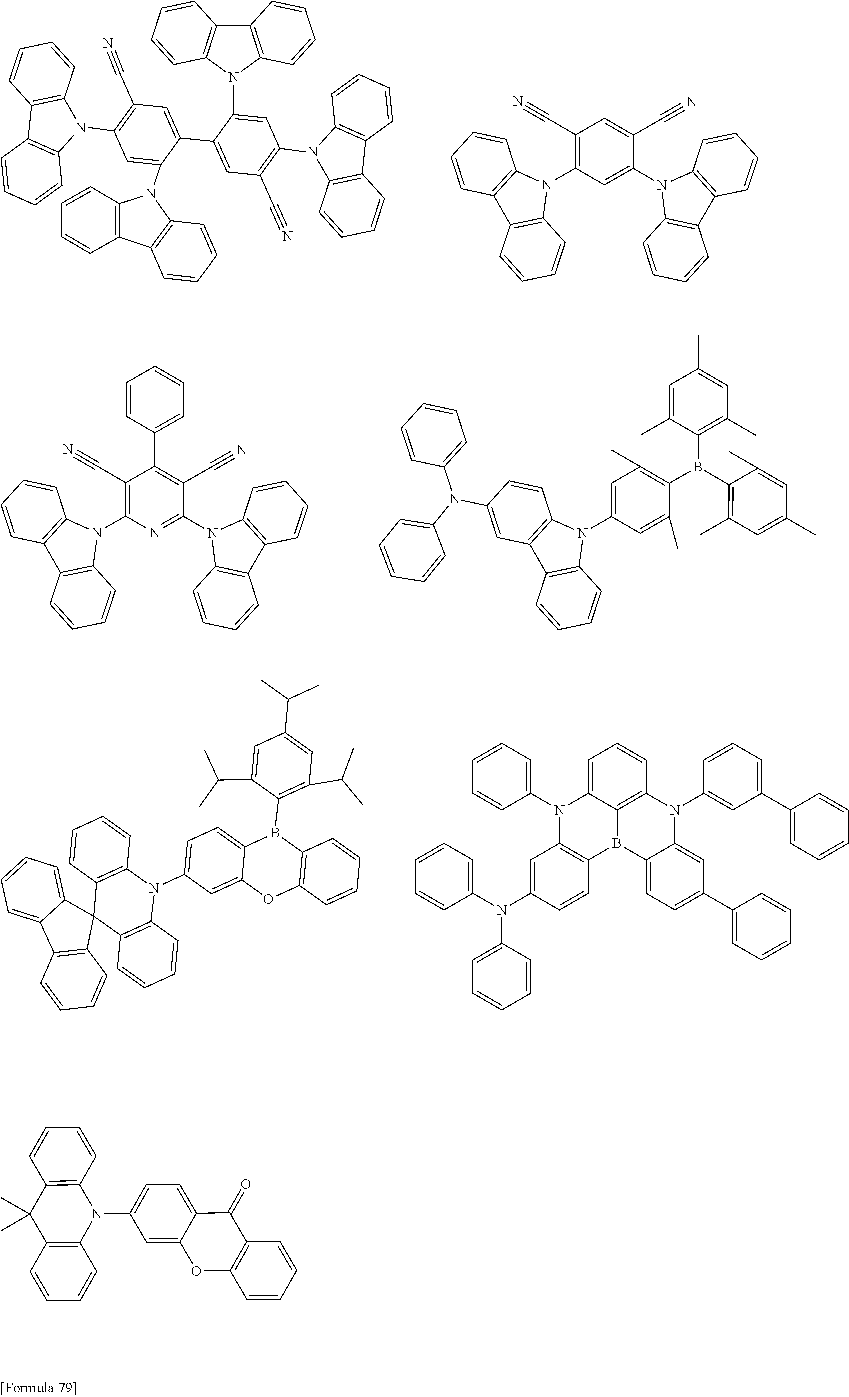

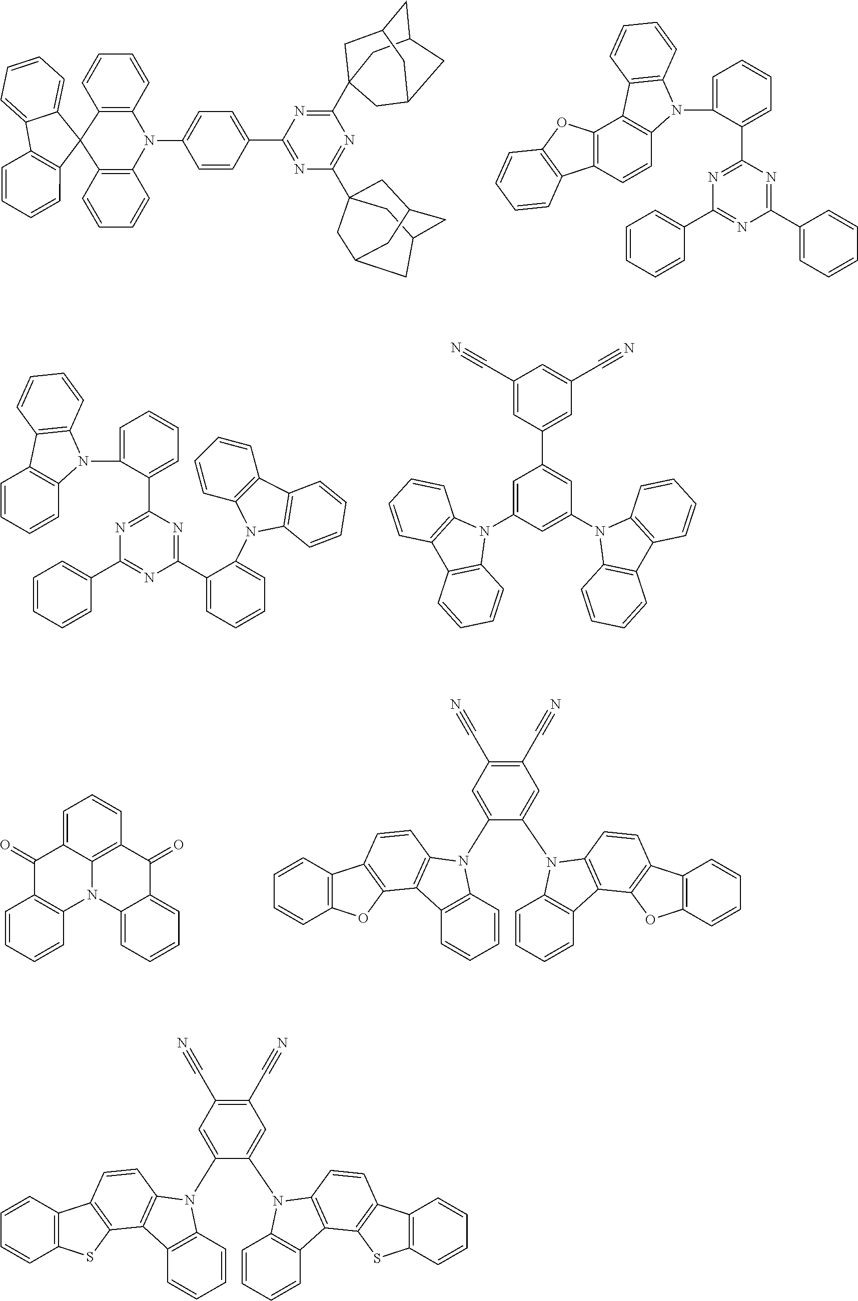

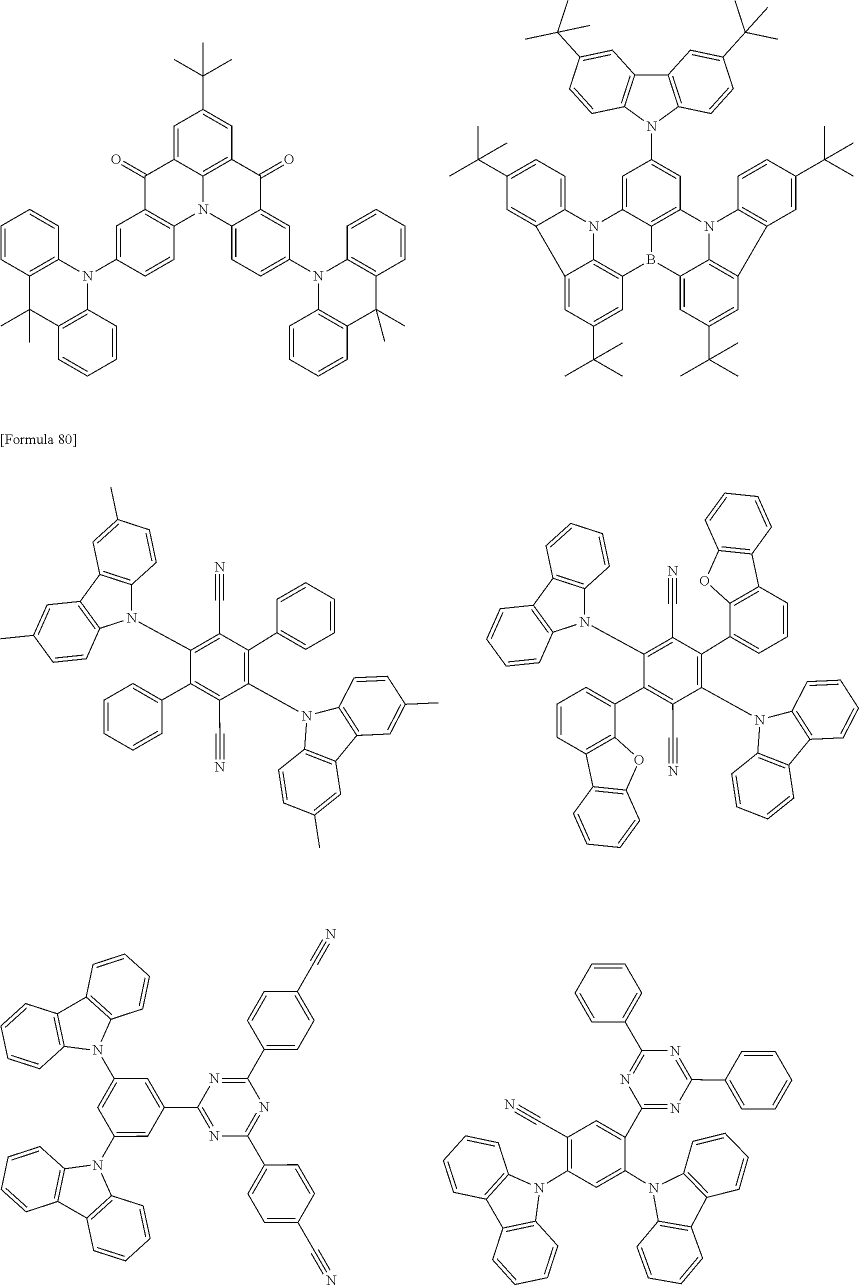

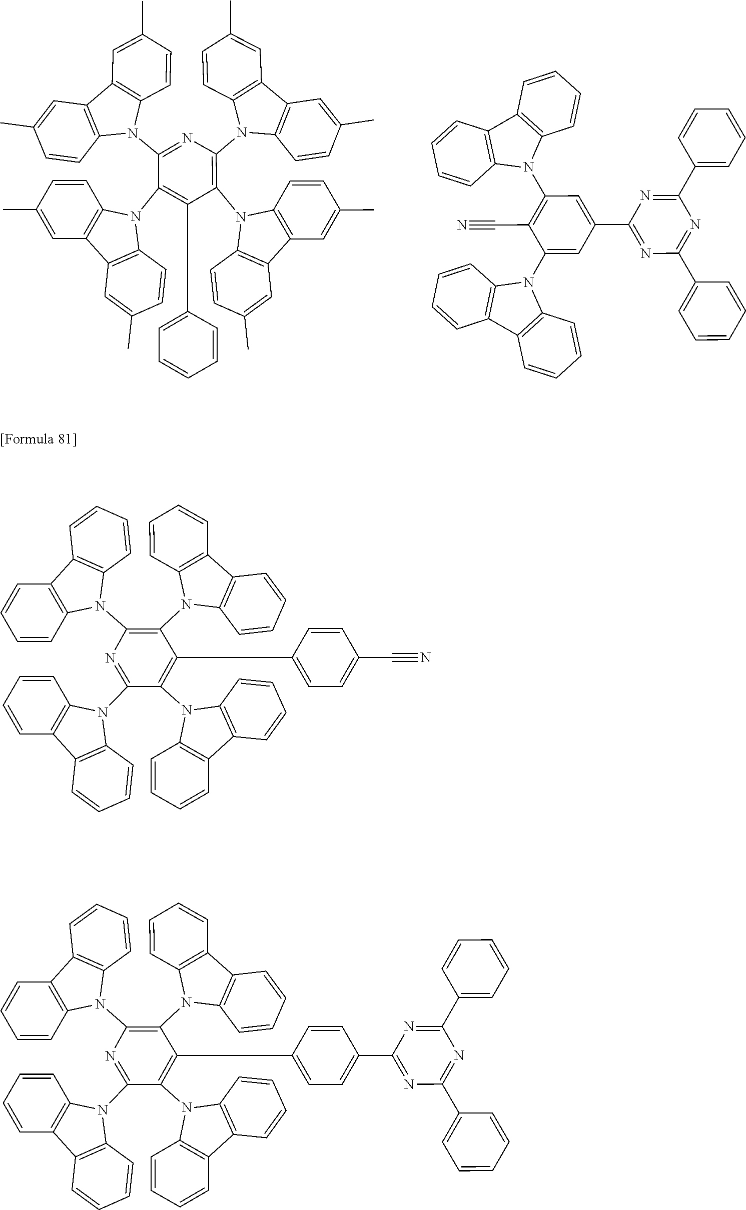

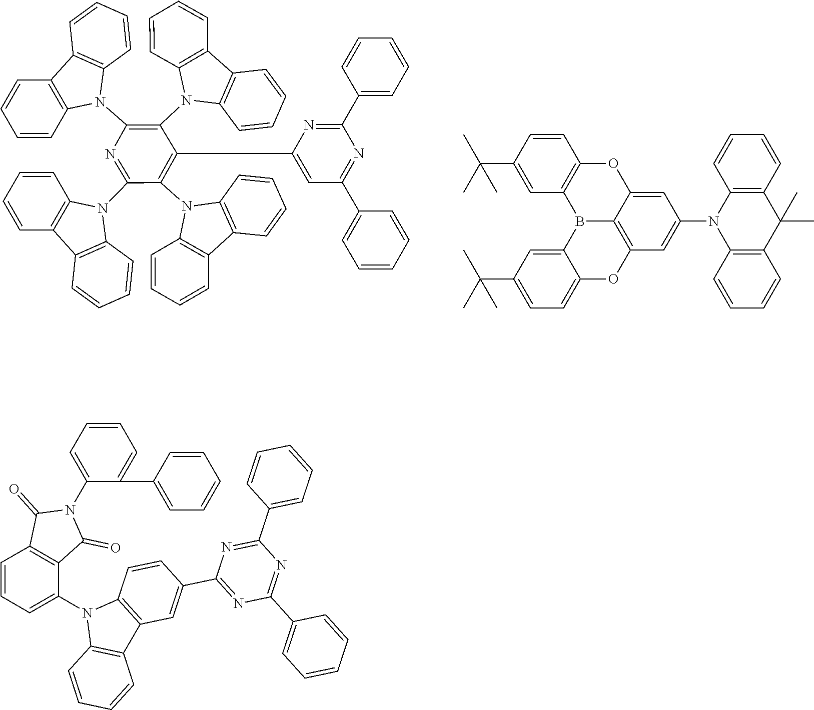 [SMILES: c1ccc(-c2cc(-c3c(-n4c5ccccc5c5ccccc54)c(-n4c5ccccc5c5ccccc54)nc(-n4c5ccccc5c5ccccc54)c3-n3c4ccccc4c4ccccc43)nc(-c3ccccc3)n2)cc1 ; CC(C)(C)c1ccc2c(c1)B1c3cc(C(C)(C)C)ccc3Oc3cc(N4c5ccccc5C(C)(C)c5ccccc54)cc(c31)O2 ; O=C1c2cccc(-n3c4ccccc4c4cc(-c5nc(-c6ccccc6)nc(-c6ccccc6)n5)ccc43)c2C(=O)N1c1ccccc1-c1ccccc1]

### Compound M1

In an exemplary arrangement of the first exemplary embodiment, the compound M1 is not a phosphorescent metal complex. The compound M1 is preferably not a heavy metal complex. Further, the compound M1 is preferably not a metal complex.

Further, the compound M1 is preferably a compound exhibiting no thermally activated delayed fluorescence.

A fluorescent material is usable as the compound A. Specific examples of the fluorescent material include a bisarylaminonaphthalene derivative, aryl-substituted naphthalene derivative, bisarylaminoanthracene derivative, aryl-substituted anthracene derivative, bisarylaminopyrene derivative, aryl-substituted pyrene derivative, bisarylamino chrysene derivative, aryl-substituted chrysene derivative, bisarylaminofluoranthene derivative, aryl-substituted fluoranthene derivative, indenoperylene derivative, acenaphthofluoranthene derivative, compound including a boron atom, pyromethene boron complex compound, compound having a pyromethene skeleton, metal complex of the compound having a pyrromethene skeleton, diketopyrrolopyrrole derivative, perylene derivative, and naphthacene derivative.

### Compound Represented by Formula (20)

In the organic EL device 1 of the exemplary embodiment, the compound M1 is preferably a compound represented by a formula (20) below.

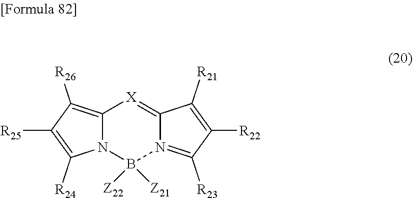

In the formula (20): X is a nitrogen atom, or a carbon atom bonded to Y;
Y is a hydrogen atom or a substituent;
R₂₁ to R₂₆ are each independently a hydrogen atom or a substituent, or at least one of a combination of R₂₁ and R₂₂, a combination of R₂₂ and R₂₃, a combination of R₂₄ and R₂₅, or a combination of R₂₅ and R₂₆ are mutually bonded to form a ring;
Y and R₂₁ to R₂₆ as the substituents are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted aralkyl group having 7 to 30 carbon atoms, a substituted or unsubstituted heteroaryl group having 5 to 30 ring atoms, a halogen atom, a carboxy group;
a substituted or unsubstituted ester group, a substituted or unsubstituted carbamoyl group, a substituted or unsubstituted amino group, a nitro group, a cyano group, a substituted or unsubstituted silyl group, and a substituted or unsubstituted siloxanyl group;
Z₂₁ and Z₂₂ are each independently a substituent, or Z₂₁ and Z₂₂ are mutually bonded to form a ring; and
Z₂₁ and Z₂₂ as the substituents are each independently selected from the group consisting of a halogen atom, a substituted or unsubstituted alkyl group having 1 to 30 carbon atoms, a substituted or unsubstituted alkyl halide group having 1 to carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

When the compound M1 is a fluorescent compound, the compound M1 preferably emits light having a main peak wavelength in a range from 400 nm to 700 nm.

Herein, the main peak wavelength means a peak wavelength of a fluorescence spectrum exhibiting a maximum luminous intensity among fluorescence spectra measured in a toluene solution in which a measurement target compound is dissolved at a concentration ranging from 10⁻⁶ mol/l to 10⁻⁵ mol/l. A spectrophotofluorometer (F-7000 produced by Hitachi High-Tech Science Corporation) is used as a measurement device.

The compound M1 preferably exhibits red or green light emission.

Herein, the red light emission refers to light emission whose main peak wavelength of fluorescence spectrum is in a range from 600 nm to 660 nm.

When the compound M1 is a red fluorescent compound, the main peak wavelength of the compound M1 is preferably in a range from 600 nm to 660 nm, more preferably in a range from 600 nm to 640 nm, and still more preferably in a range from 610 nm to 630 nm.

Herein, the green light emission refers to light emission whose main peak wavelength of fluorescence spectrum is in a range from 500 nm to 560 nm.

When the compound M1 is a green fluorescent compound, the main peak wavelength of the compound M1 is preferably in a range from 500 nm to 560 nm, more preferably in a range from 500 nm to 540 nm, and still more preferably in a range from 510 nm to 540 nm.

Herein, the blue light emission refers to a light emission whose main peak wavelength of fluorescence spectrum is in a range from 430 nm to 480 nm.

When the compound M1 is a blue fluorescent compound, the main peak wavelength of the compound M1 is preferably in a range from 430 nm to 480 nm, more preferably in a range from 440 nm to 480 nm.

The main peak wavelength of the light emitted from the organic EL device 1 is measured as follows.

Voltage is applied to the organic EL device such that a current density becomes 10 mA/cm², where spectral radiance spectrum is measured by a spectroradiometer CS-2000 (produced by Konica Minolta, Inc.).

A peak wavelength of an emission spectrum, at which the luminous intensity of the resultant spectral radiance spectrum is at the maximum, is measured and defined as the main peak wavelength (unit: nm).

### Method for Producing Compound M1

The compound M1 can be produced by a known method.

Specific examples of the compound represented by the formula (20) are shown below. Note that the compound represented by the formula (20) of the invention is not limited to the specific examples.

A coordinate bond between a boron atom and a nitrogen atom in a pyrromethene skeleton is shown by various means such as a solid line, a broken line, an arrow, and omission. Herein, the coordinate bond is shown by a solid line or a broken line, or the description of the coordinate bond is omitted.

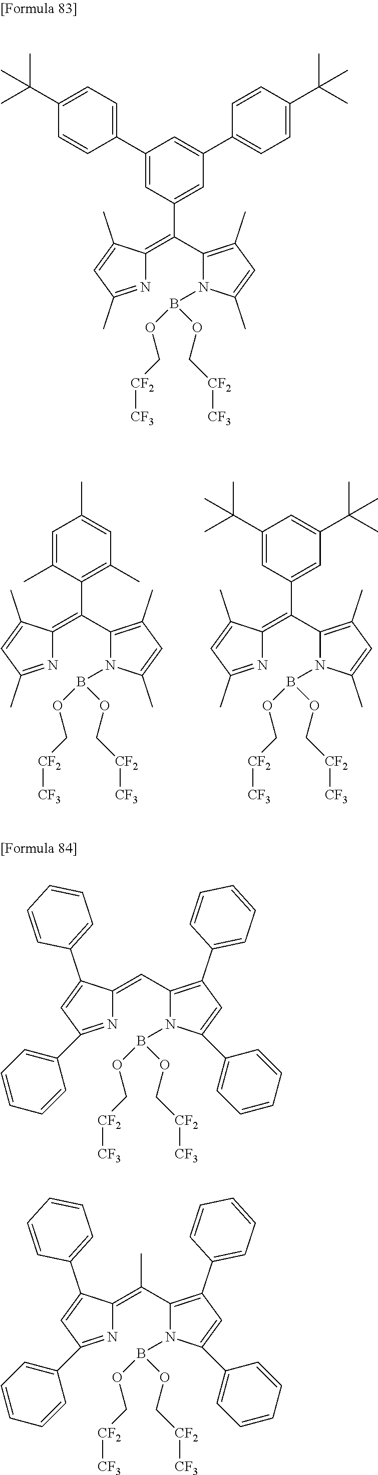

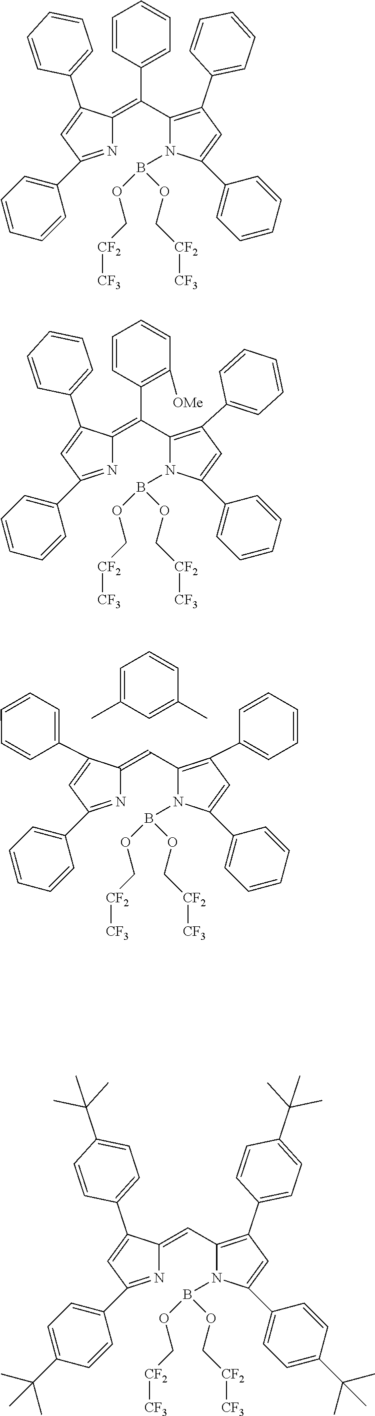

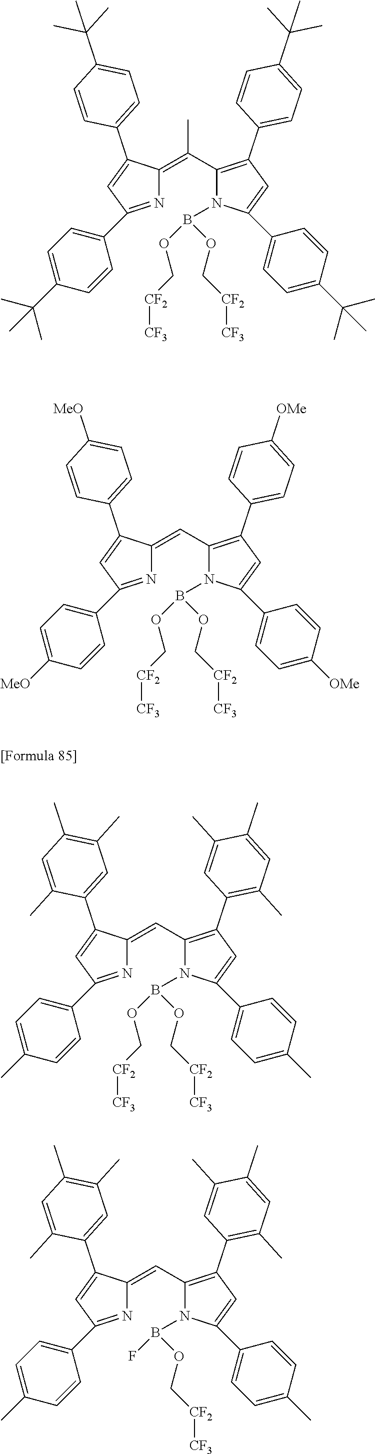

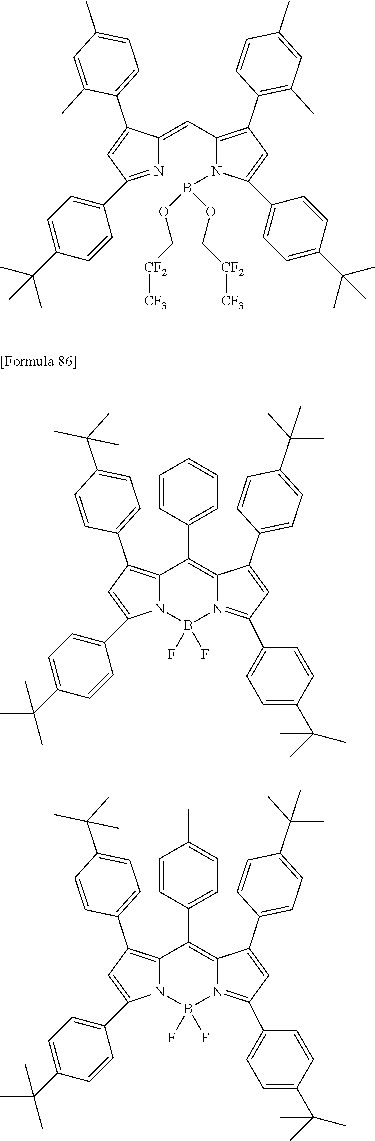

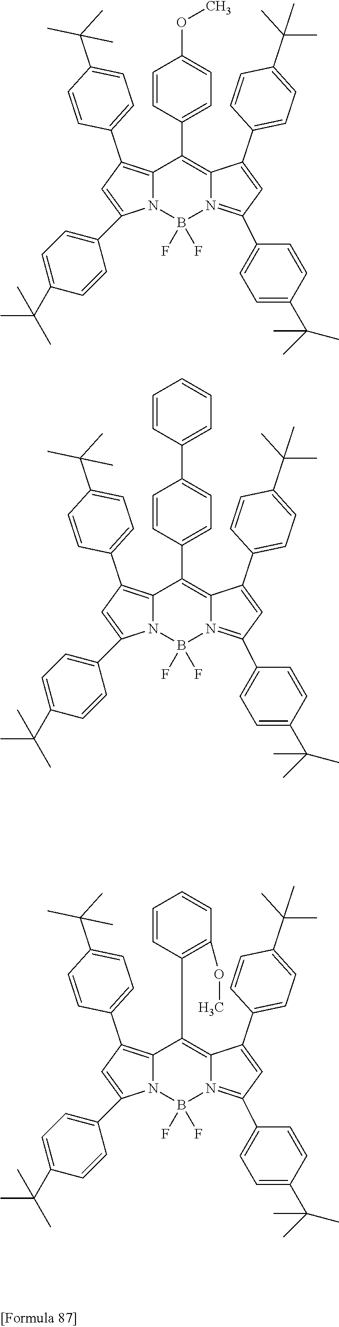

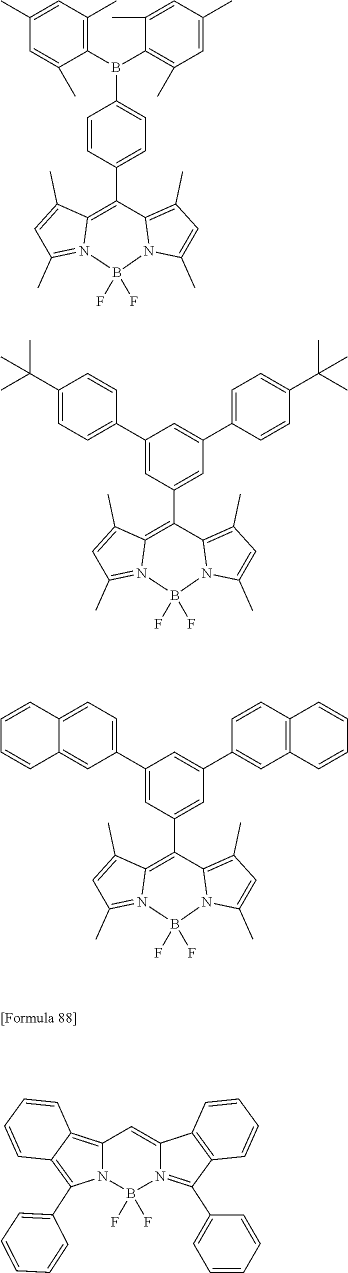

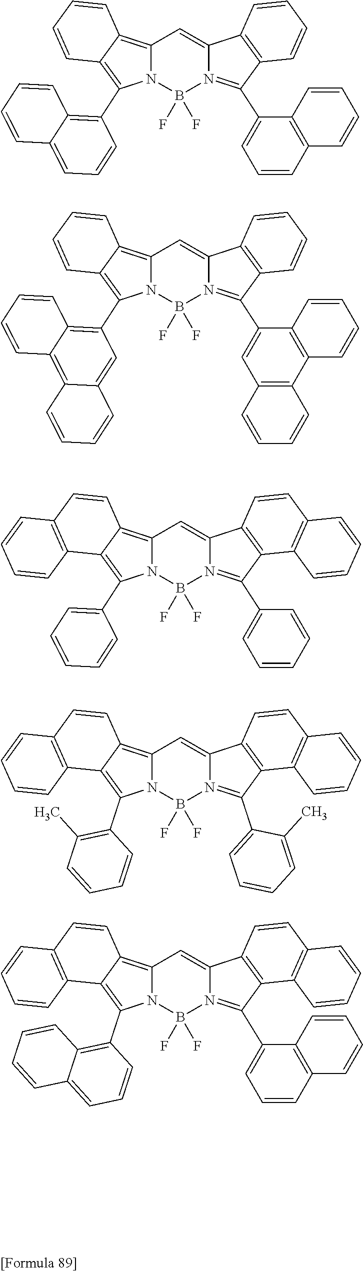

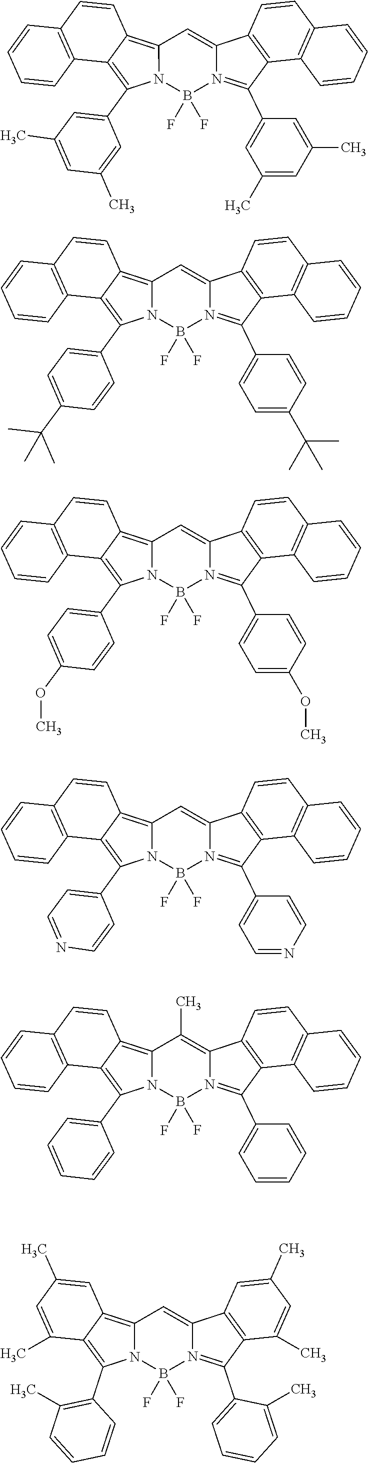

### Relationship Between Compound M1 and Compound M2 in Emitting Layer

In the organic EL device 1 of the exemplary embodiment, a singlet energy S₁(Mat2) of the compound M2 as a delayed fluorescent compound and a singlet energy S₁(Mat1) of the fluorescent compound M1 preferably satisfy a relationship of a numerical formula (Numerical Formula 1) below.

S₁(Mat2)>S₁(Mat1) (Numerical Formula 1)

An energy gap T_{77K}(Mat2) at 77K of the compound M2 and an energy gap T_{77K}(Mat1) at 77K of the compound M1 preferably satisfy a relationship of a numerical formula (Numerical Formula 4) below.

T_{77K}(Mat2)>T_{77K}(Mat1) (Numerical Formula 4)

It is preferable that, when the organic EL device 1 of the exemplary embodiment emits light, the fluorescent compound M1 mainly emits light in the emitting layer 5.

### Relationship Between Triplet Energy and Energy Gap at 77K

Here, a relationship between a triplet energy and an energy gap at 77K will be described. In the exemplary embodiment, the energy gap at 77K is different from a typical triplet energy in some aspects.

The triplet energy is measured as follows. First, a solution in which a compound (measurement target) is dissolved in an appropriate solvent is encapsulated in a quartz glass tube to prepare a sample. A phosphorescent spectrum (ordinate axis: phosphorescent luminous intensity, abscissa axis: wavelength) of the sample is measured at a low temperature (77K). A tangent is drawn to the rise of the phosphorescent spectrum close to the short-wavelength region. The triplet energy is calculated by a predetermined conversion equation based on a wavelength value at an intersection of the tangent and the abscissa axis.

Here, the thermally activated delayed fluorescent compound among the compounds of the exemplary embodiment is preferably a compound having a small ΔST. When ΔST is small, intersystem crossing and inverse intersystem crossing are likely to occur even at a low temperature (77K), so that the singlet state and the triplet state coexist. As a result, the spectrum to be measured in the same manner as the above includes emission from both the singlet state and the triplet state. Although it is difficult to distinguish the emission from the singlet state from the emission from the triplet state, the value of the triplet energy is basically considered dominant.

Accordingly, in the exemplary embodiment, the triplet energy is measured by the same method as a typical triplet energy T, but a value measured in the following manner is referred to as an energy gap T_{77K} in order to differentiate the measured energy from the typical triplet energy in a strict meaning. The measurement target compound is dissolved in EPA (diethylether:isopentane:ethanol=5:5:2 in volume ratio) at a concentration of 10 μmol/L, and the obtained solution is encapsulated in a quartz cell to provide a measurement sample. A phosphorescent spectrum (ordinate axis: phosphorescent luminous intensity, abscissa axis: wavelength) of the measurement sample is measured at a low temperature (77K). A tangent is drawn to the rise of the phosphorescent spectrum close to the short-wavelength region. An energy amount is calculated by a conversion equation (F1) below based on a wavelength value λ_{edge} [nm] at an intersection of the tangent and the abscissa axis and is defined as an energy gap T_{77K} at 77K.

T_{77K} [eV]=1239.85/λ_{edge} Conversion Equation (F1):

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (produced by Hitachi High-Technologies Corporation) is usable. Any device for phosphorescence measurement is usable. A combination of a cooling unit, a low temperature container, an excitation light source and a light-receiving unit may be used for phosphorescence measurement.

### Singlet Energy S₁

A method of measuring the singlet energy S₁ with use of a solution (occasionally referred to as a solution method) is exemplified by a method below.

A toluene solution in which a measurement target compound is dissolved at a concentration of 10 μmol/L is prepared and is encapsulated in a quartz cell to provide a measurement sample. Absorption spectrum (ordinate axis: absorption intensity, abscissa axis: wavelength) of the sample is measured at normal temperature (300K). A tangent was drawn to the fall of the absorption spectrum close to the long-wavelength region, and a wavelength value λedge (nm) at an intersection of the tangent and the abscissa axis was assigned to a conversion equation (F2) below to calculate the singlet energy.

S₁ [eV]=1239.85/λedge Conversion Equation (F2):

Any device for measuring absorption spectrum is usable. For instance, a spectrophotometer (U3310 produced by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

In the exemplary embodiment, a difference (S₁−T_{77K}) between the singlet energy S₁ and the energy gap T_{77K} at 77K is defined as ΔST.

In the exemplary embodiment, a difference ΔST(Mat2) between the singlet energy S₁(Mat2) of the compound M2 and the energy gap T_{77K}(Mat2) at 77K of the compound M2 is preferably less than 0.3 eV, more preferably less than 0.2 eV, still more preferably less than 0.1 eV, and still further more preferably less than 0.01 eV. That is, ΔST(Mat2) preferably satisfies a relationship of one of numerical formulae (Numerical Formula 1A) to (Numerical Formula 1 D) below.

ΔST(Mat2)=S₁(Mat2)−T_{77K}(Mat2)<0.3 eV (Numerical Formula 1A)

ΔST(Mat2)=S₁(Mat2)−T_{77K}(Mat2)<0.2 eV (Numerical Formula 1B)

ΔST(Mat2)=S₁(Mat2)−T_{77K}(Mat2)<0.1 eV (Numerical Formula 1C)

ΔST(Mat2)=S₁(Mat2)−T_{77K}(Mat2)<0.01 eV (Numerical Formula 1D)

The organic EL device 1 of the exemplary embodiment preferably emits red light or green light.

When the organic EL device 1 of the exemplary embodiment emits green light, the main peak wavelength of the light emitted from the organic EL device 1 is preferably in a range from 500 nm to 560 nm.

When the organic EL device 1 of the exemplary embodiment emits red light, the main peak wavelength of the light emitted from the organic EL device 1 is preferably in a range from 600 nm to 660 nm.

When the organic EL device 1 of the exemplary embodiment emits blue light, the main peak wavelength of the light emitted from the organic EL device 1 is preferably in a range from 430 nm to 480 nm.

The main peak wavelength of the light emitted from the organic EL device is measured as follows.

Voltage is applied to the organic EL device such that a current density becomes 10 mA/cm², where spectral radiance spectrum is measured by a spectroradiometer CS-2000 (produced by Konica Minolta, Inc.).

A peak wavelength of an emission spectrum, at which the luminous intensity of the resultant spectral radiance spectrum is at the maximum, is measured and defined as the main peak wavelength (unit: nm).

### Film Thickness of Emitting Layer

The film thickness of the emitting layer 5 of the organic EL device 1 in the exemplary embodiment is preferably in a range from 5 nm to 50 nm, more preferably in a range from 7 nm to 50 nm, most preferably in a range from 10 nm to 50 nm. When the film thickness of the emitting layer is 5 nm or more, the formation of the emitting layer and the adjustment of the chromaticity are easy. When the film thickness of the emitting layer is 50 nm or less, an increase in the drive voltage is likely to be reducible.

### Content Ratios of Compounds in Emitting Layer

For instance, content ratios of the compound M2 and the compound M1 in the emitting layer 5 preferably fall within ranges shown below.

The content ratio of the compound M2 is preferably in a range from 10 mass % to 80 mass %, more preferably in a range from 10 mass % to 60 mass %, and still more preferably in a range from 20 mass % to 60 mass %.

The content ratio of the compound M1 is preferably in a range from 0.01 mass % to 10 mass %, more preferably in a range from 0.01 mass % to 5 mass %, and still more preferably in a range from 0.01 mass % to 1 mass %.

It should be noted that the emitting layer 5 according to the exemplary embodiment may contain a material other than the compound M2 and the compound M1.

The emitting layer 5 may contain a single type of the compound M2 or may contain two or more types of the compound M2. The emitting layer 5 may contain a single type of the compound M1 or may contain two or more types of the compound M1.

### TADF Mechanism

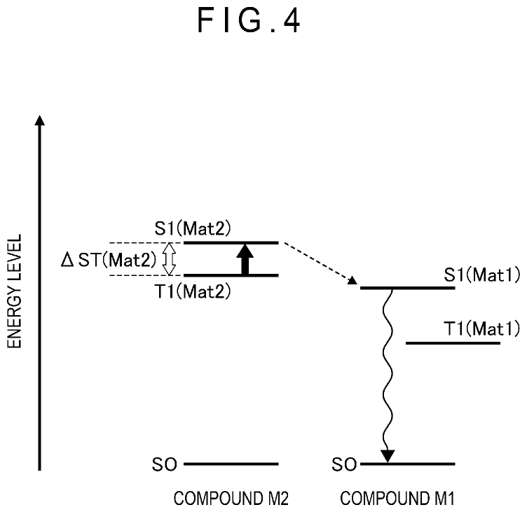

FIG. 4 shows an example of a relationship between energy levels of the compound M2 and the compound M1 in the emitting layer. In FIG. 4, S0 represents a ground state. S1(Mat2) represents a lowest singlet state of the compound M2. T1(Mat2) represents a lowest triplet state of the compound M2. S1(Mat1) represents a lowest singlet state of the compound M1. T1(Mat1) represents a lowest triplet state of the compound M1.

A dashed arrow directed from S1(Mat2) to S1(Mat1) in FIG. 4 represents Förster energy transfer from the lowest singlet state of the compound M2 to the lowest singlet state of the compound M1.

As shown in FIG. 4, when a compound having a small ΔST(Mat2) is used as the compound M2, inverse intersystem crossing from the lowest triplet state T1(Mat2) to the lowest singlet state S1(Mat2) can be caused by heat energy. Subsequently, Förster energy transfer from the lowest singlet state S1(Mat2) of the compound M2 to the compound M1 occurs to generate the lowest singlet state S1(Mat1). Consequently, fluorescence from the lowest singlet state S1(Mat1) of the compound M1 can be observed. It is inferred that the internal quantum efficiency can be theoretically raised up to 100% also by using delayed fluorescence by the TADF mechanism.

In the organic EL device 1 of the first exemplary embodiment, the first layer 81 contains the first compound (compound having at least one deuterium atom), and the emitting layer 5 contains the delayed fluorescent compound M2 and the fluorescent compound M1 in the exemplary embodiment.

In the first exemplary embodiment, an organic EL device excellent in performance (in particular, having a long lifetime) can be provided.

The organic EL device 1 according to the first exemplary embodiment is usable in an organic electroluminescence apparatus (hereinafter, occasionally referred to as an organic EL apparatus).

Further, the organic EL device 1 according to the first exemplary embodiment is usable in an electronic device such as a display device and a light-emitting unit.

An arrangement of the organic EL device 1 is further described below. It should be noted that the reference numerals are occasionally omitted below.

### Substrate

The substrate is used as a support for the organic EL device. For instance, glass, quartz, plastics and the like are usable for the substrate. A flexible substrate is also usable. The flexible substrate is a bendable substrate, which is exemplified by a plastic substrate. Examples of the material for the plastic substrate include polycarbonate, polyarylate, polyethersulfone, polypropylene, polyester, polyvinyl fluoride, polyvinyl chloride, polyimide, and polyethylene naphthalate. Moreover, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include ITO (Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

The elements belonging to the group 1 or 2 of the periodic table, which are a material having a small work function, specifically, an alkali metal such as lithium (Li) and cesium (Cs), an alkaline earth metal such as magnesium (Mg), calcium (Ca) and strontium (Sr), an alloy containing the alkali metal and the alkaline earth metal (e.g., MgAg, AlLi), a rare earth metal such as europium (Eu) and ytterbium (Yb), and an alloy containing the rare earth metal are usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode. Examples of materials for the cathode include elements belonging to the group 1 or 2 of the periodic table, specifically, an alkali metal such as lithium (Li) and cesium (Cs), an alkaline earth metal such as magnesium (Mg), calcium (Ca) and strontium (Sr), an alloy containing the alkali metal and the alkaline earth metal (e.g., MgAg, AlLi), a rare earth metal such as europium (Eu) and ytterbium (Yb), and an alloy containing the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance include: an aromatic amine compound, which is a low-molecule organic compound, such that 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl(abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA₃B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,3-f:20,30-h]quinoxaline-2,3,6,7,10,11-hexacarbonitrile (HAT-CN).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

### Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V·s) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

When the hole transporting layer includes two or more layers, one of the layers with a larger energy gap is preferably provided closer to the emitting layer.

### Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the exemplary embodiment, a benzimidazole compound is preferably usable. The above-described substances mostly have an electron mobility of 10⁻⁶ cm²/(V·s) or more. It should be noted that any substance other than the above substance may be used for the electron transporting layer as long as the substance exhibits a higher electron transportability than the hole transportability. The electron transporting layer may be provided in the form of a single layer or a laminate of two or more layers of the above substance(s).

Further, a high polymer compound is usable for the electron transporting layer. For instance, poly[(9,9-dihexylfluorene-2,7-diyl)-co-(pyridine-3,5-diyl)](abbreviation: PF-Py), poly[(9,9-dioctylfluorene-2,7-diyl)-co-(2,2′-bipyridine-6,6′-diyl)](abbreviation: PF-BPy) and the like are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

In the organic EL device 1 of the exemplary embodiment, an electron transporting zone including one or more organic layers is provided between the cathode 4 and the emitting layer 5. In a case of FIG. 1, the electron transporting zone is formed by the first layer 81 and the electron injecting layer 9.

The electron transporting zone preferably includes a plurality of organic layers. An arrangement in which the electron transporting zone includes the first layer 81 and a second layer provided between the first layer 81 and the cathode 4 is explained in a fourth exemplary embodiment.

### Layer Formation Method

A method for forming each layer of the organic EL device in the exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

A thickness of each of the organic layers in the organic EL device according to the exemplary embodiment is not limited except for the above particular description. In general, the thickness preferably ranges from several nanometers to 1 μm because excessively small film thickness is likely to cause defects (e.g. pin holes) and excessively large thickness leads to the necessity of applying high voltage and consequent reduction in efficiency.

### Second Exemplary Embodiment

An arrangement of an organic EL device according to a second exemplary embodiment of the invention is described below. In the description of the second exemplary embodiment, the same components as those in the first exemplary embodiment are denoted by the same reference signs and names to simplify or omit an explanation of the components. In the second exemplary embodiment, the same materials and compounds as described in the first exemplary embodiment are usable unless otherwise specified.

In the organic EL device of the second exemplary embodiment, the first layer contains the first compound having at least one deuterium atom, and the emitting layer contains the delayed fluorescent compound M2, the fluorescent compound M1, and a compound M3.

In this arrangement, the compound M2 is preferably a host material, the compound M1 is preferably a dopant material, and the compound M3 is preferably a host material. One of the compound M2 and the compound M3 is occasionally referred to as a first host material, and the other is occasionally referred to as a second host material.

The first compound, the compound M2, and the compound M1 explained in the first exemplary embodiment are each independently usable as the first compound, the compound M2, and the compound M1 in the second exemplary embodiment.

### Compound M3

The compound M3 may be a delayed fluorescent compound or a compound exhibiting no delayed fluorescence.

The compound M3 preferably includes at least one of partial structures represented by formulae (311) to (336) below in one molecule.

When the compound M3 includes a plurality of partial structures represented by any of the formulae (311) to (336), the plurality of partial structures represented by any of the formulae (311) to (336) are the same or different. For instance, when the compound M3 includes a plurality of partial structures represented by the formula (311), the plurality of partial structures represented by the formula (311) are the same or different. The same applies to a case where the compound M3 includes a plurality of partial structures represented by any of the formulae (311) to (317) and (319) to (331).

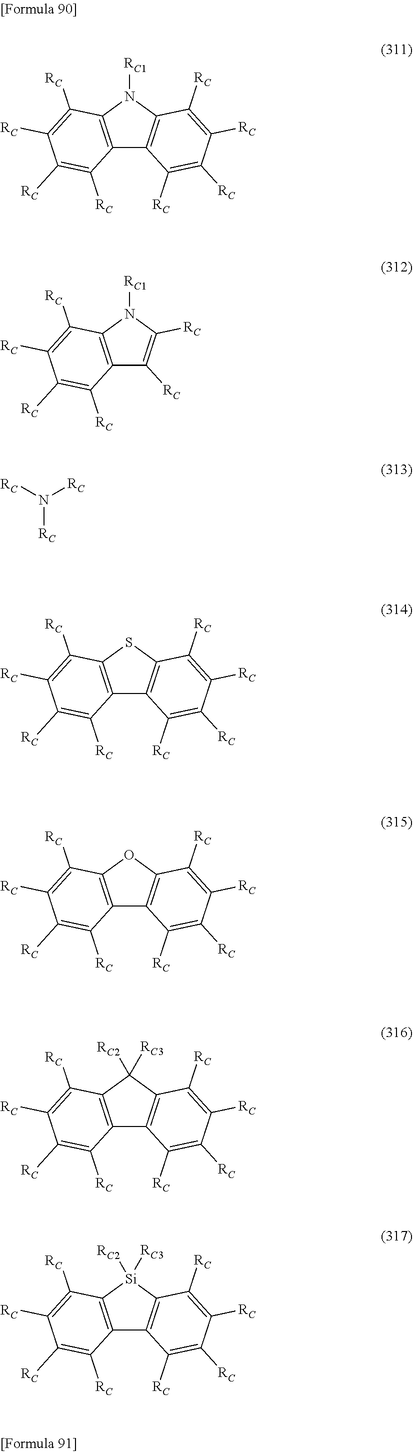

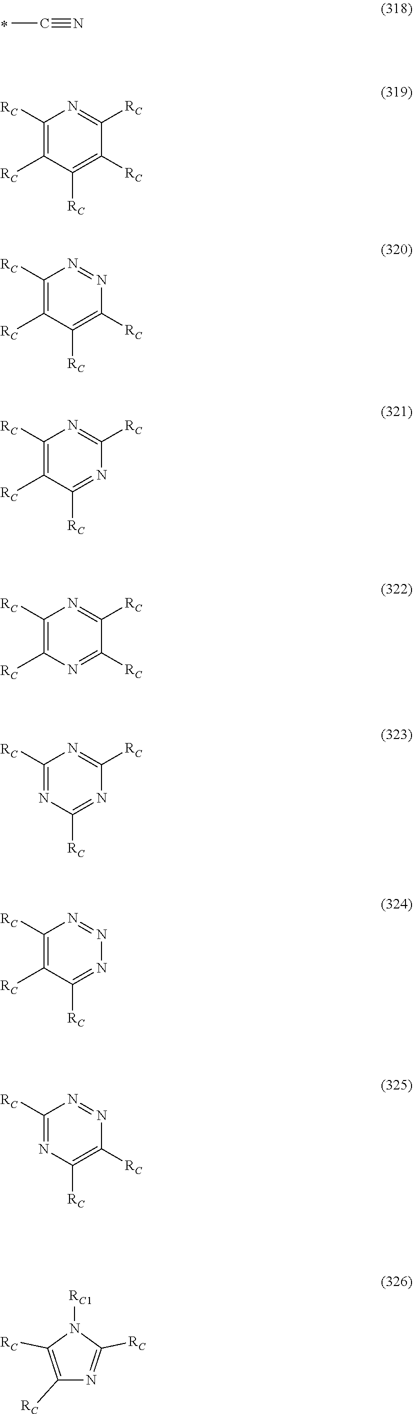

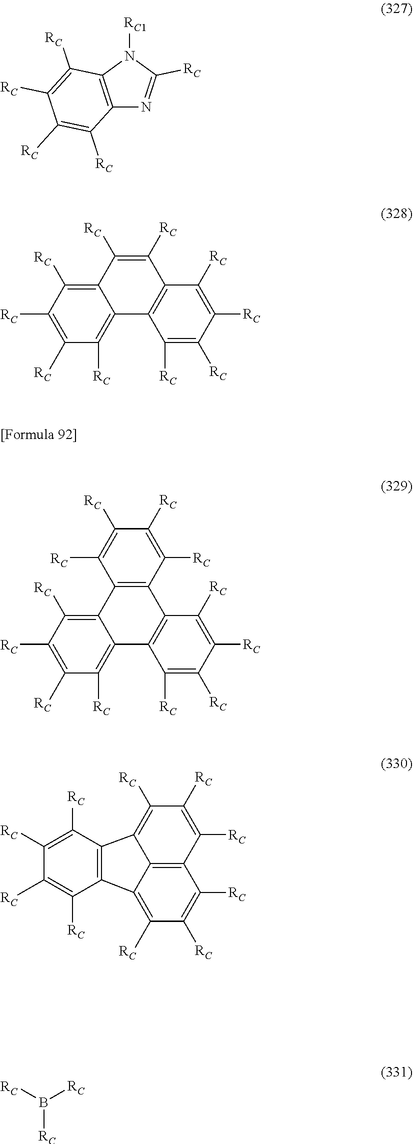

In the formulae (311) to (317) and (319) to (331):
R_{C} and R_{C1} to R_{C3} are each independently a hydrogen atom or a substituent; at least one combination of combinations of adjacent ones of R_{C} or a combination of R_{C2} and R_{C3} are mutually bonded to form a ring; or R_{C} and R_{C1} to R_{C3} are each independently a single bond bonded to another atom or another structure in the molecule of the compound M3; and
at least one of R_{C} or R_{C1} to R_{C3} is a single bond bonded to another atom or another structure in the molecule of the compound M3;
* in the formula (318) represents a bonding position to another atom or another structure in the molecule of the compound M3;
R_{C} and R_{C1} to R_{C3} as the substituents are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted germanium group, a substituted phosphine oxide group, a nitro group, a substituted or unsubstituted carbonyl group, or a substituted boryl group;
a plurality of R_{C} are mutually the same or different;
when a plurality of R_{C1} are present, the plurality of R_{C1} are mutually the same or different;
when a plurality of R_{C2} are present, the plurality of R_{C2} are mutually the same or different; and
when a plurality of R_{C3} are present, the plurality of R_{C3} are mutually the same or different.

In the exemplary embodiment, the compound M3 preferably includes at least one partial structure represented by the formula (311), the formulae (314) to (319), the formula (321), the formula (323), or the formula (330) in one molecule.

In the exemplary embodiment, the compound M3 more preferably includes at least one partial structure represented by the formula (311), the formulae (314) to (315), the formula (321), or the formula (323) in one molecule.

In the exemplary embodiment, the compound M3 still more preferably includes a partial structure represented by the formula (311) in one molecule.

The partial structures represented by the formulae (311) and (314) to (317) are each also preferably a partial structure represented by any of formulae (301) to (306) below.

When the compound M3 includes a plurality of partial structures represented by any of the formulae (301) to (306), the plurality of partial structures represented by any of the formulae (301) to (306) are the same or different.

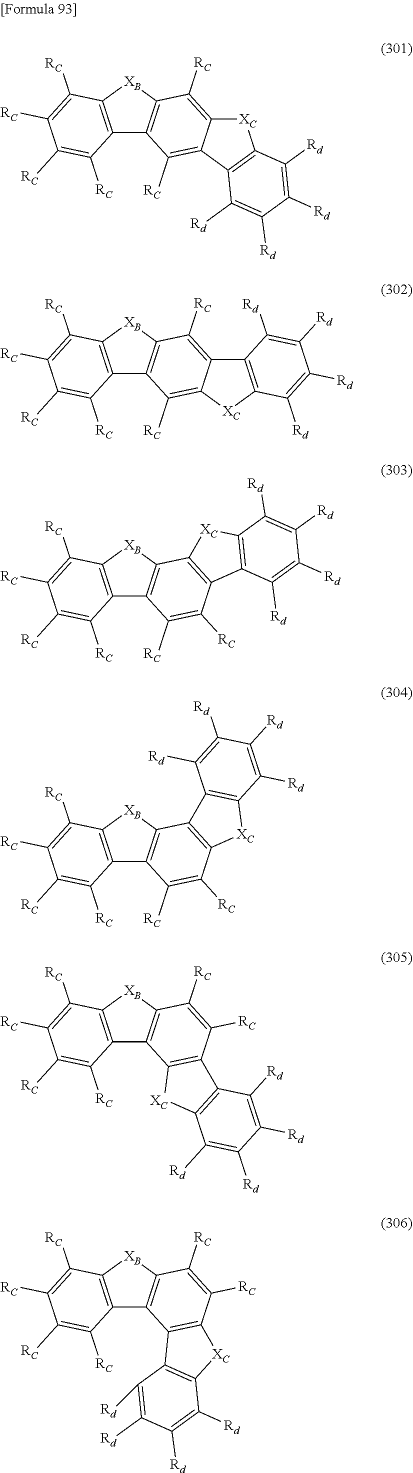

In the formulae (301) to (306):
X_{B} and X_{C} are each independently NR_{C1}, CR_{C2}R_{C3}, SiR_{C2}R_{C3}, an oxygen atom, or a sulfur atom;
X_{B} and X_{C} are mutually the same or different;
R_{C1} represents the same as R_{C1} in the formula (311);
R_{C2} and R_{C3} each independently represent the same as R_{C2} and R_{C3} in the formulae (316) and (317);
each R_{C} independently represents the same as R_{C} in the formula (311);
each R_{d} is independently a hydrogen atom or a substituent; at least one combination of combinations of adjacent ones of R_{d} are mutually bonded to form a ring; or a single bond bonded to another atom or another structure in the molecule of the compound M3;
each R_{d} as the substituent independently represents the same as R_{C} as the substituent in the formula (311);
a plurality of Rd are mutually the same or different; and
at least one of R_{C}, R_{C1} to R_{C3} or R_{d} is a single bond bonded to another atom or another structure in the molecule of the compound M3.

In the compound M3, R_{C}, R_{C1} to R_{C3}, and R_{d} are preferably each independently a hydrogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, or a substituted or unsubstituted alkyl halide group having 1 to 30 carbon atoms.

### Method for Producing Compound M3

The compound M3 can be produced by a known method.

### Specific Examples of Compound M3

Specific examples of the compound M3 include the following compounds. It should however be noted that the invention is not limited to the specific examples of the compound.

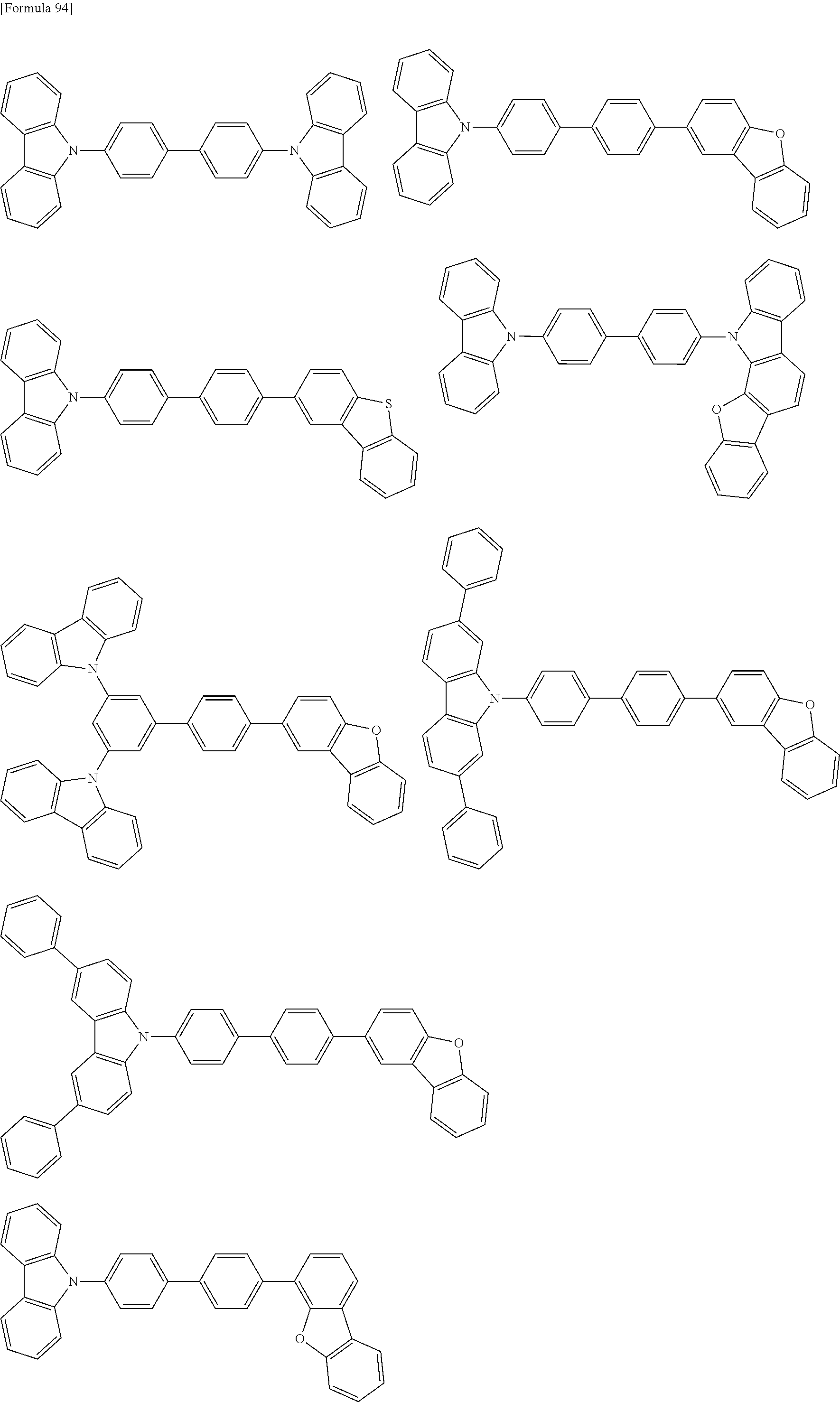

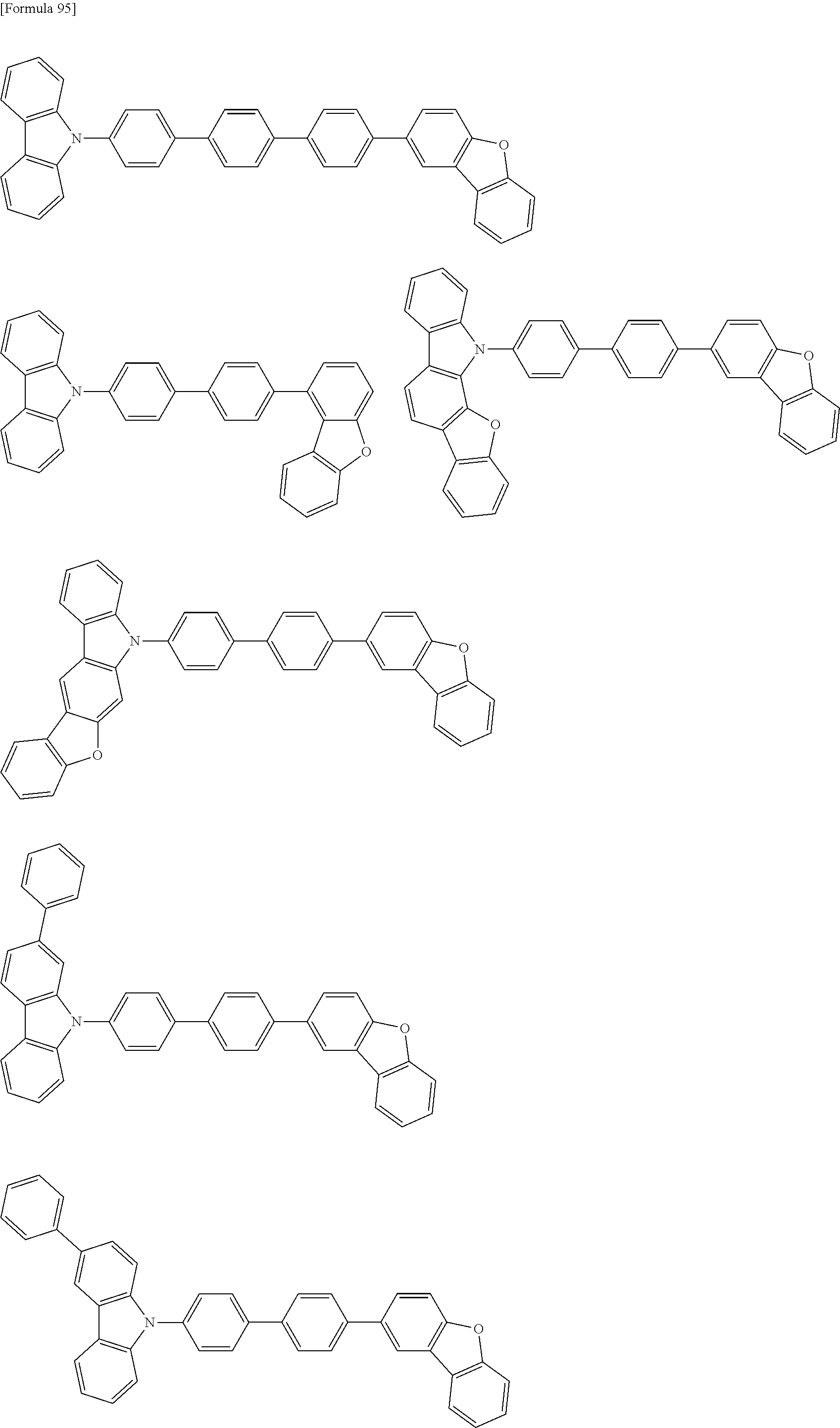

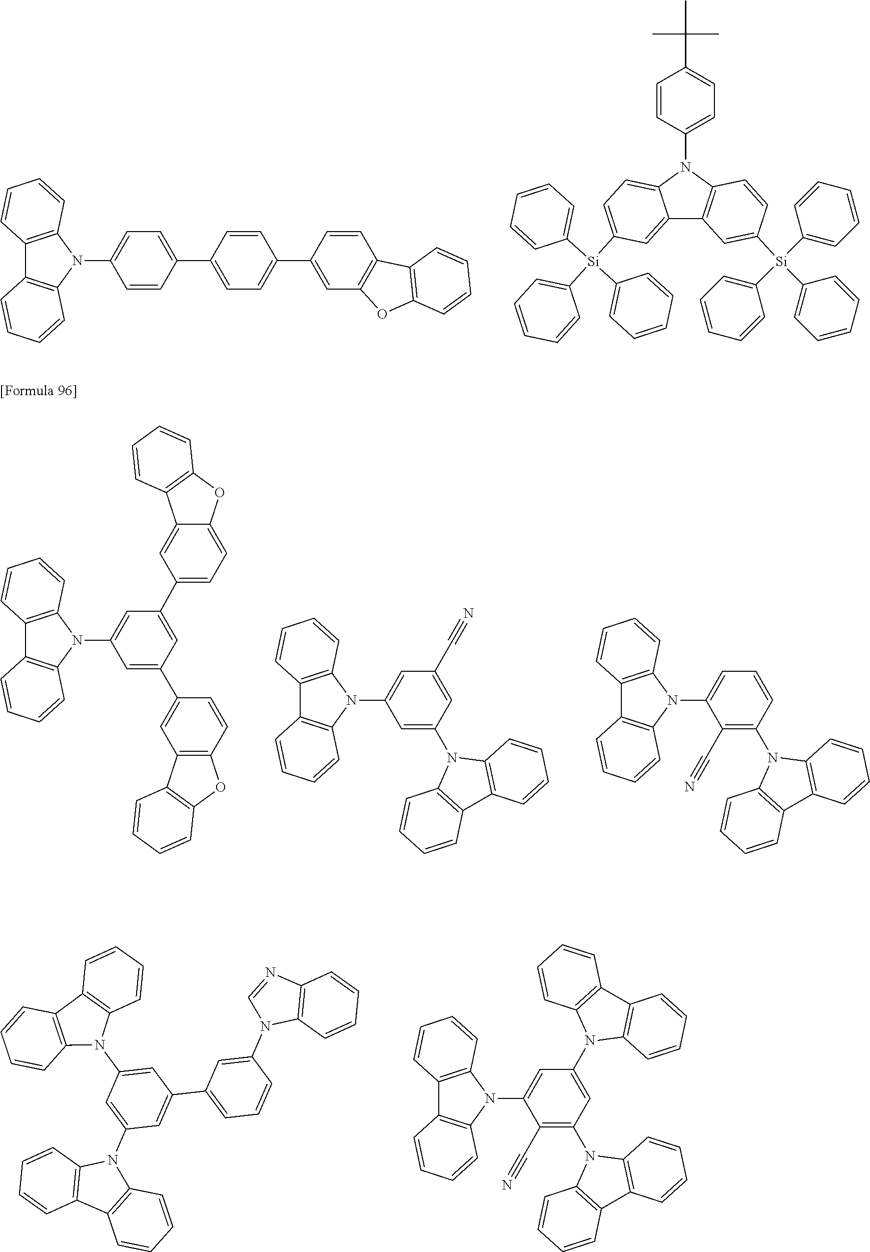

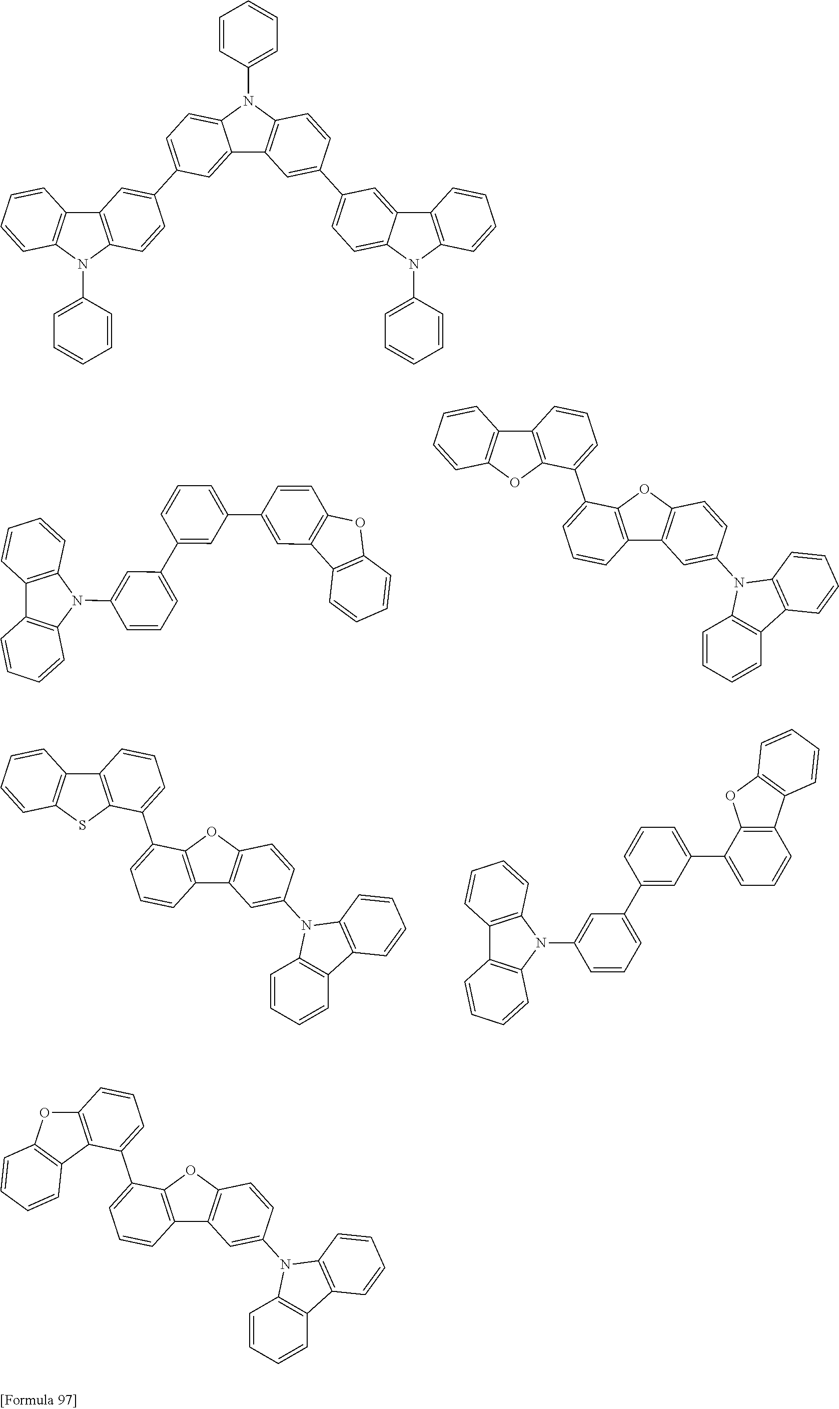 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1cc(-c2cccc(-n3c4ccccc4c4ccccc43)c2)cc(-c2ccc3oc4ccccc4c3c2)c1 ; c1ccc2c(c1)oc1c(-c3cccc4c3oc3ccc(-n5c6ccccc6c6ccccc65)cc34)cccc12 ; c1ccc2c(c1)sc1c(-c3cccc4c3oc3ccc(-n5c6ccccc6c6ccccc65)cc34)cccc12 ; c1cc(-c2cccc(-n3c4ccccc4c4ccccc43)c2)cc(-c2cccc3c2oc2ccccc23)c1 ; c1ccc2c(c1)oc1cccc(-c3cccc4c3oc3ccc(-n5c6ccccc6c6ccccc65)cc34)c12]

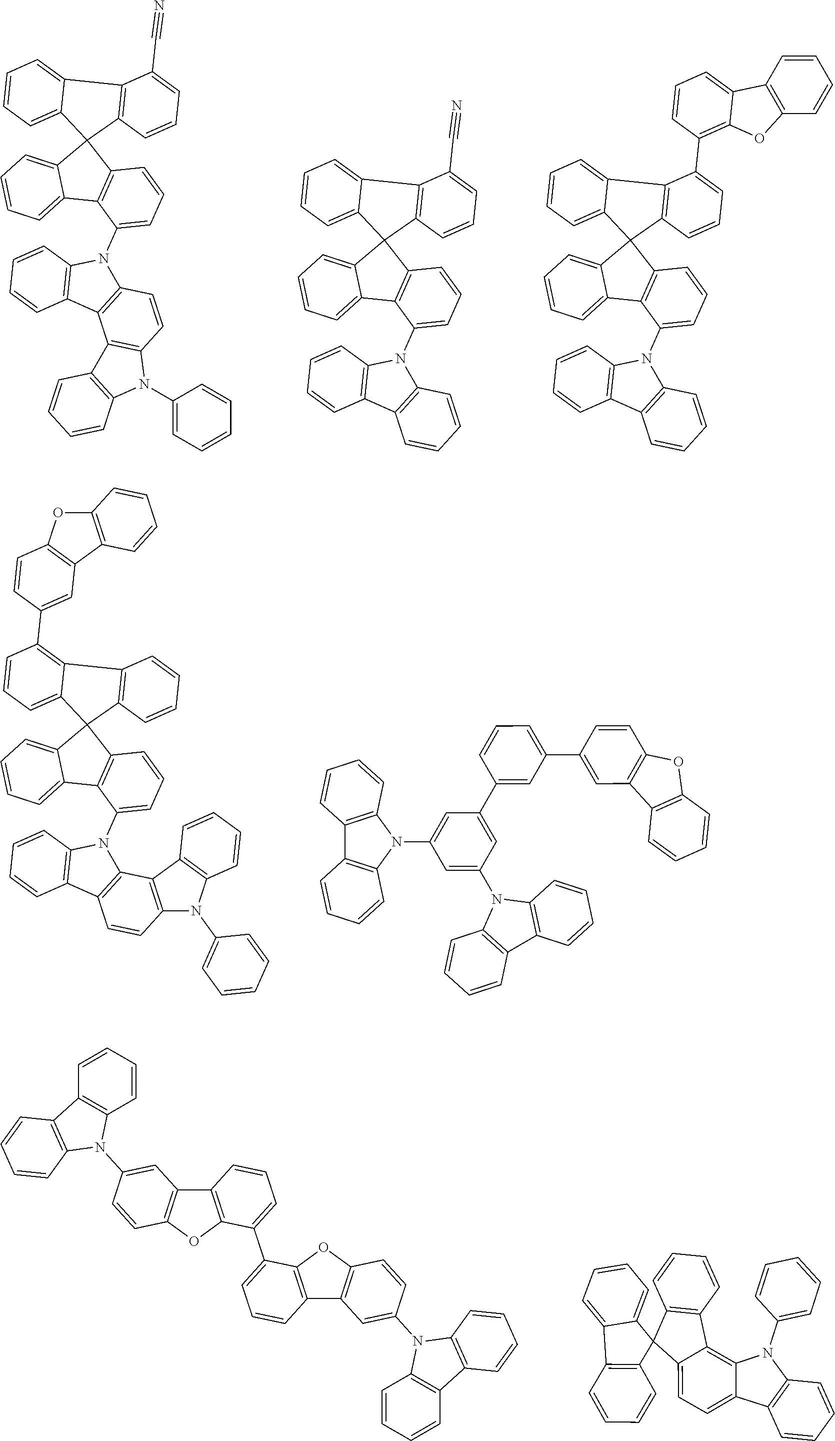

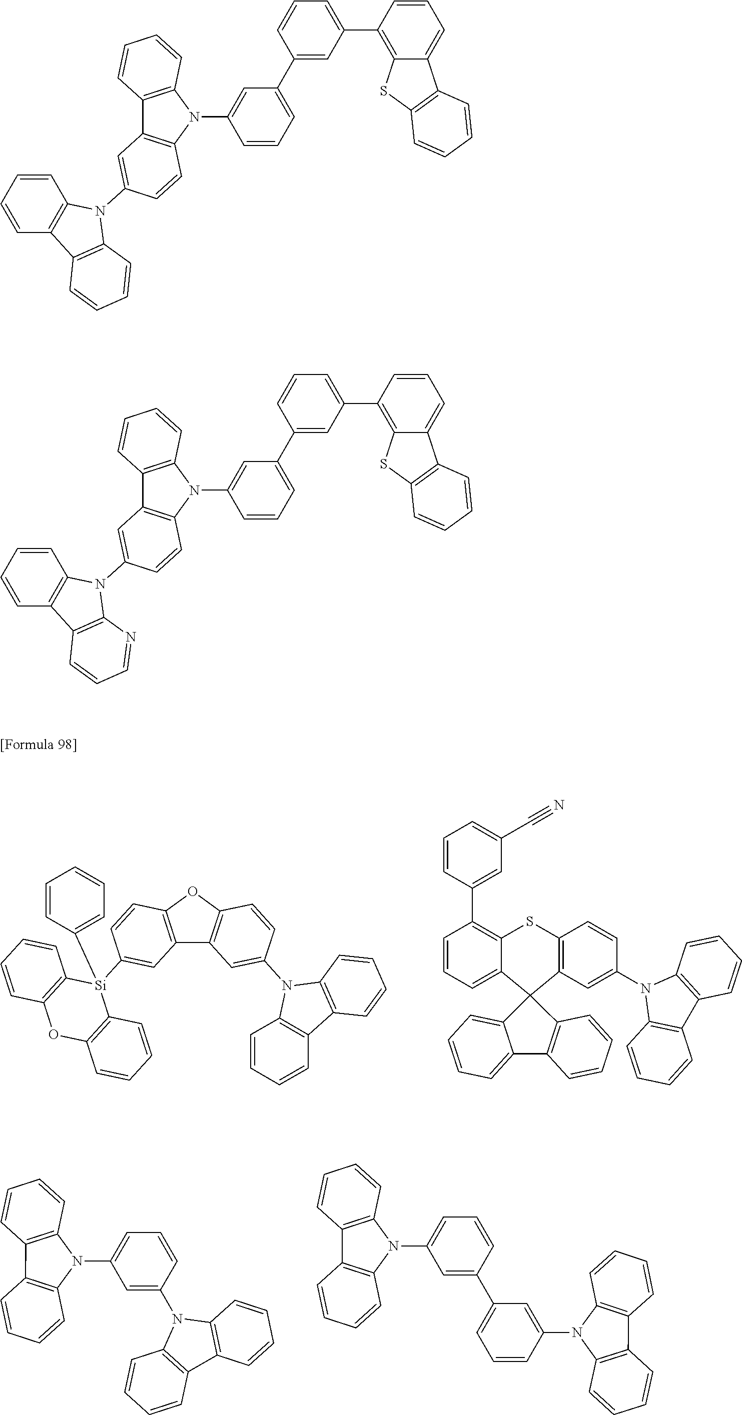 [SMILES: c1cc(-c2cccc(-n3c4ccccc4c4cc(-n5c6ccccc6c6ccccc65)ccc43)c2)cc(-c2cccc3c2sc2ccccc23)c1 ; c1cc(-c2cccc(-n3c4ccccc4c4cc(-n5c6ccccc6c6cccnc65)ccc43)c2)cc(-c2cccc3c2sc2ccccc23)c1 ; c1ccc([Si]2(c3ccc4oc5ccc(-n6c7ccccc7c7ccccc76)cc5c4c3)c3ccccc3Oc3ccccc32)cc1 ; N#Cc1cccc(-c2cccc3c2Sc2ccc(-n4c5ccccc5c5ccccc54)cc2C32c3ccccc3-c3ccccc32)c1 ; c1cc(-n2c3ccccc3c3ccccc32)cc(-n2c3ccccc3c3ccccc32)c1 ; c1cc(-c2cccc(-n3c4ccccc4c4ccccc43)c2)cc(-n2c3ccccc3c3ccccc32)c1]

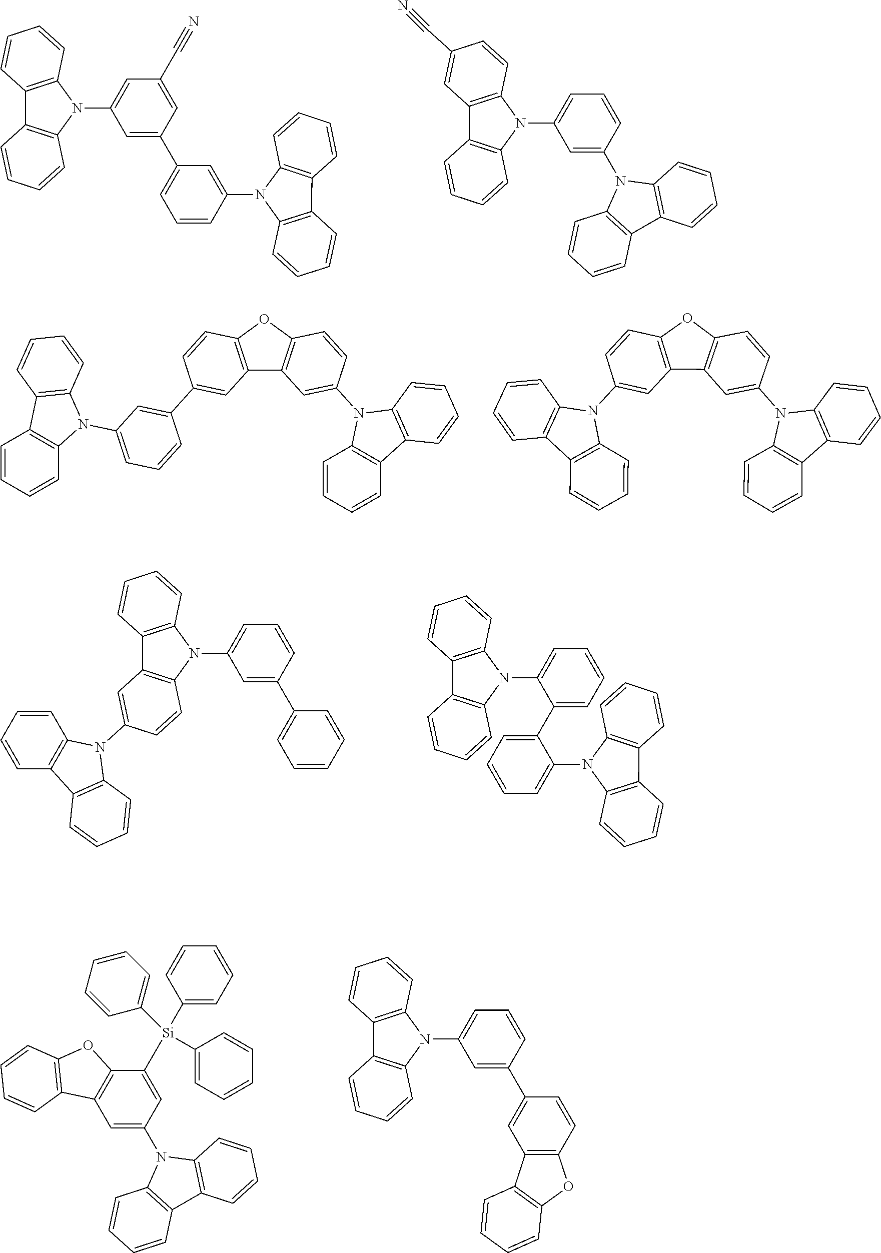

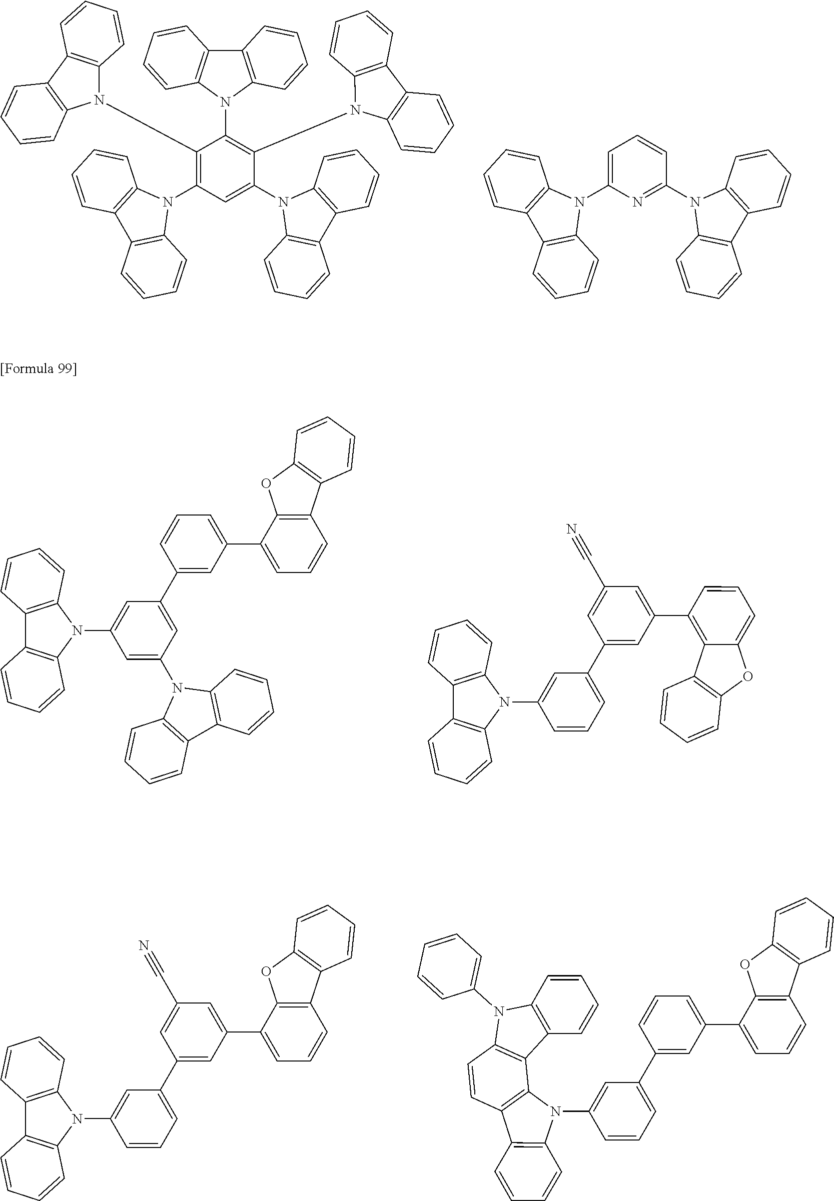 [SMILES: c1ccc2c(c1)c1ccccc1n2-c1cc(-n2c3ccccc3c3ccccc32)c(-n2c3ccccc3c3ccccc32)c(-n2c3ccccc3c3ccccc32)c1-n1c2ccccc2c2ccccc21 ; c1cc(-n2c3ccccc3c3ccccc32)nc(-n2c3ccccc3c3ccccc32)c1 ; c1cc(-c2cc(-n3c4ccccc4c4ccccc43)cc(-n3c4ccccc4c4ccccc43)c2)cc(-c2cccc3c2oc2ccccc23)c1 ; N#Cc1cc(-c2cccc(-n3c4ccccc4c4ccccc43)c2)cc(-c2cccc3oc4ccccc4c23)c1 ; N#Cc1cc(-c2cccc(-n3c4ccccc4c4ccccc43)c2)cc(-c2cccc3c2oc2ccccc23)c1 ; c1ccc(-n2c3ccccc3c3c2ccc2c4ccccc4n(-c4cccc(-c5cccc(-c6cccc7c6oc6ccccc67)c5)c4)c23)cc1]

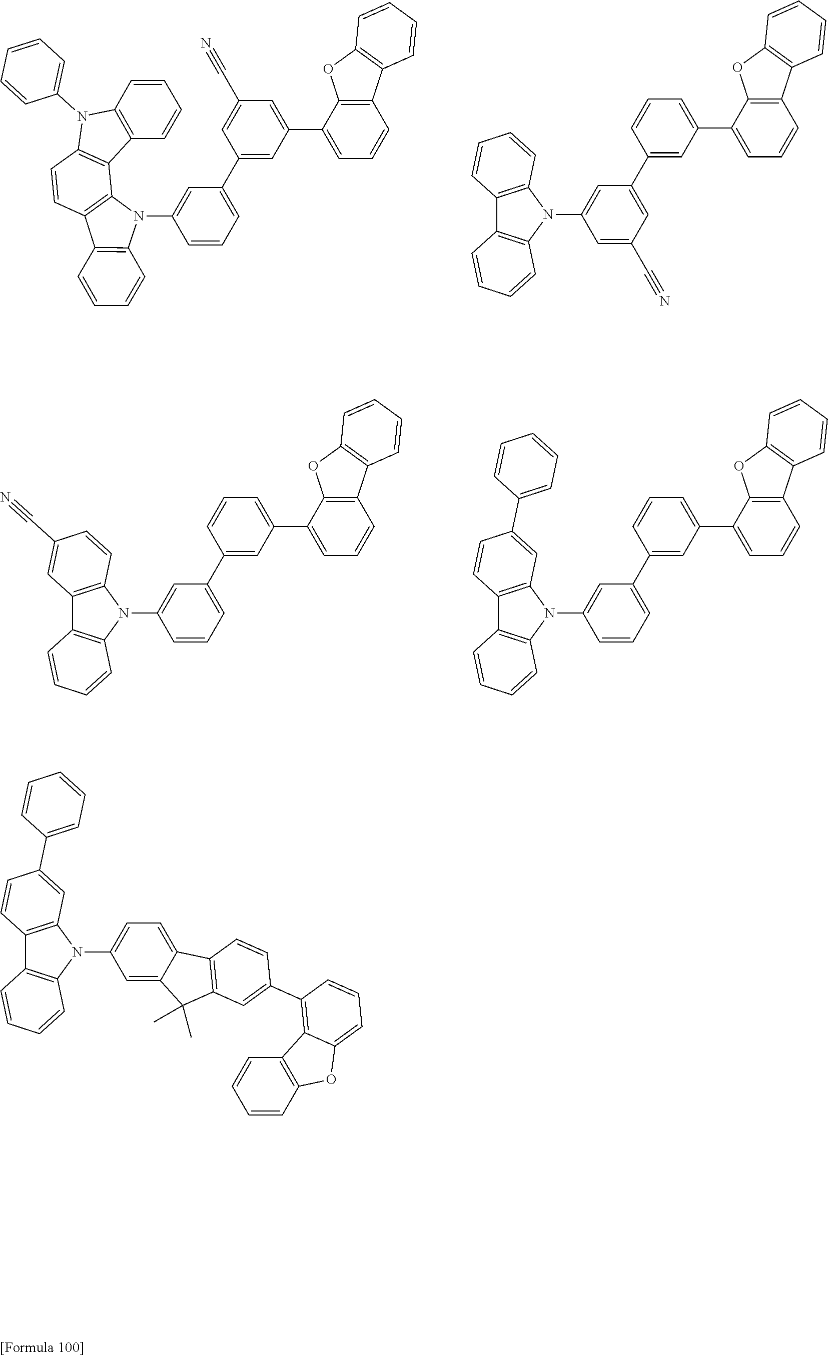 [SMILES: N#Cc1cc(-c2cccc(-n3c4ccccc4c4ccc5c(c6ccccc6n5-c5ccccc5)c43)c2)cc(-c2cccc3c2oc2ccccc23)c1 ; N#Cc1cc(-c2cccc(-c3cccc4c3oc3ccccc34)c2)cc(-n2c3ccccc3c3ccccc32)c1 ; N#Cc1ccc2c(c1)c1ccccc1n2-c1cccc(-c2cccc(-c3cccc4c3oc3ccccc34)c2)c1 ; c1ccc(-c2ccc3c4ccccc4n(-c4cccc(-c5cccc(-c6cccc7c6oc6ccccc67)c5)c4)c3c2)cc1 ; CC1(C)c2cc(-c3cccc4oc5ccccc5c34)ccc2-c2ccc(-n3c4ccccc4c4ccc(-c5ccccc5)cc43)cc21]

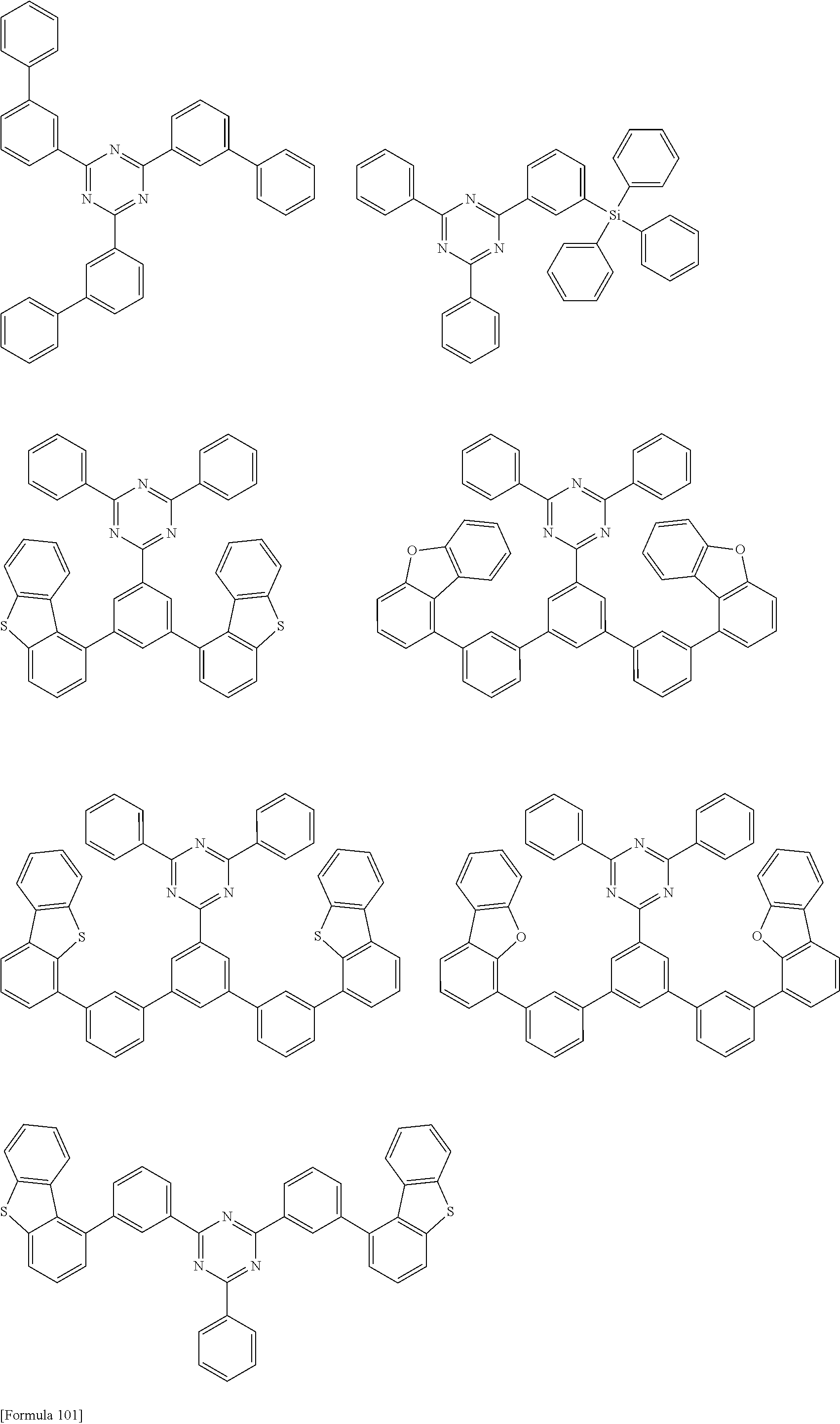

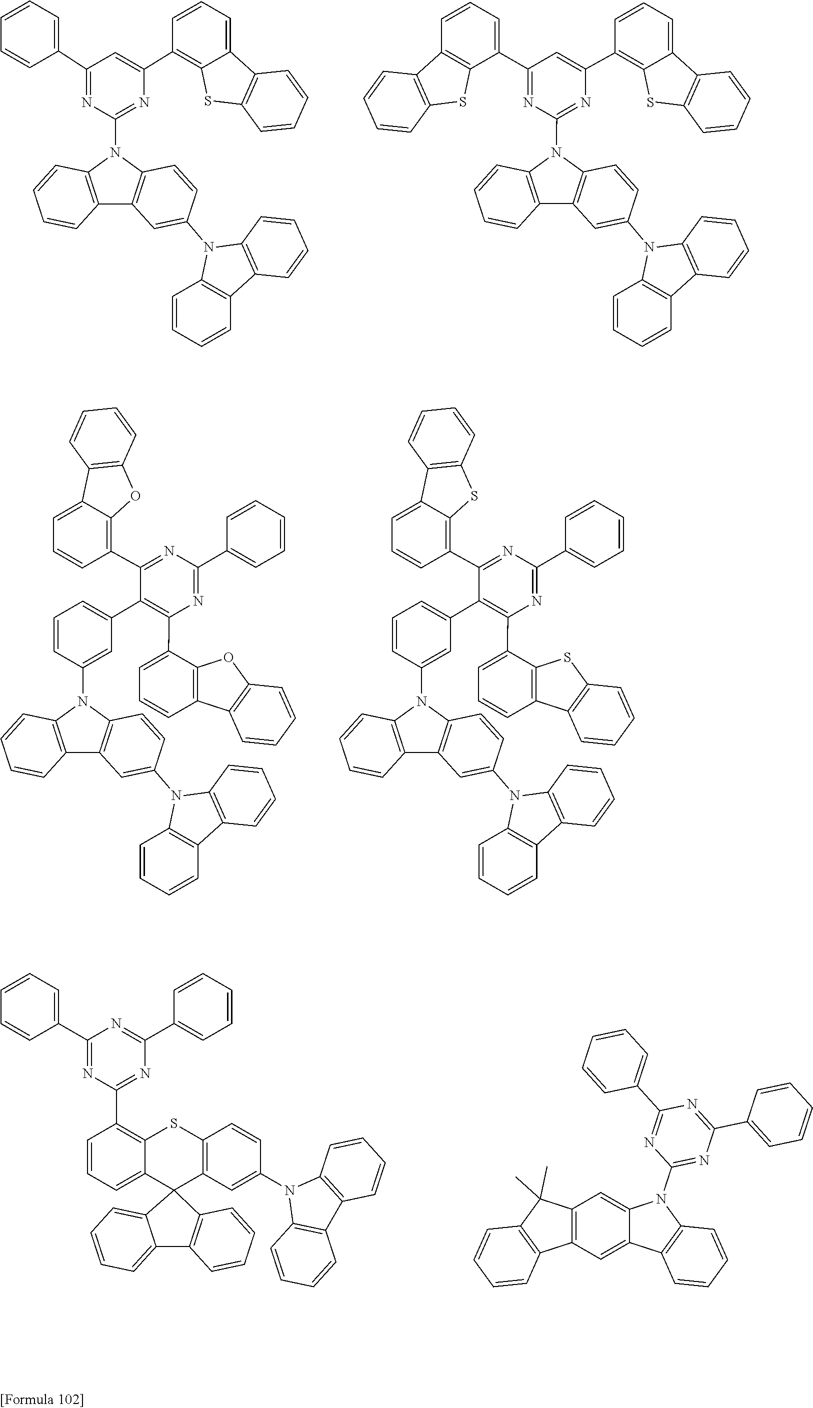

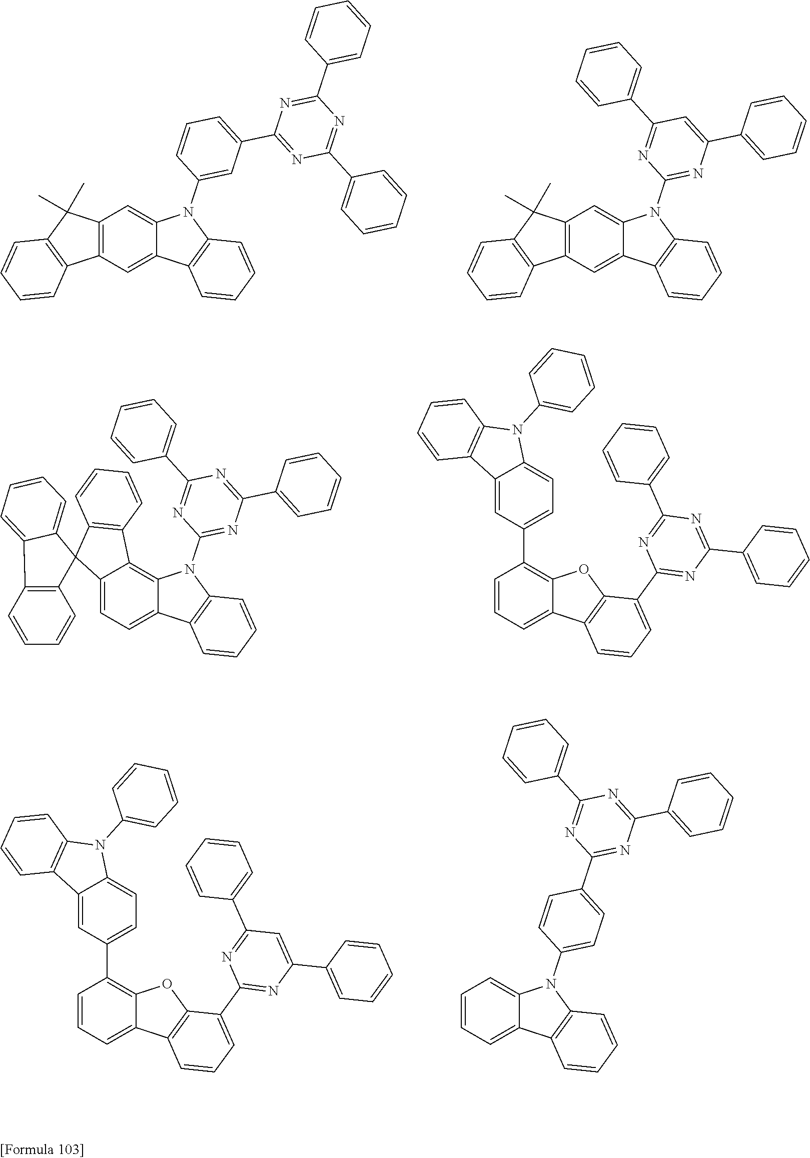 [SMILES: CC1(C)c2ccccc2-c2cc3c4ccccc4n(-c4cccc(-c5nc(-c6ccccc6)nc(-c6ccccc6)n5)c4)c3cc21 ; CC1(C)c2ccccc2-c2cc3c4ccccc4n(-c4nc(-c5ccccc5)cc(-c5ccccc5)n4)c3cc21 ; c1ccc(-c2nc(-c3ccccc3)nc(-n3c4ccccc4c4ccc5c(c43)-c3ccccc3C53c4ccccc4-c4ccccc43)n2)cc1 ; c1ccc(-c2nc(-c3ccccc3)nc(-c3cccc4c3oc3c(-c5ccc6c(c5)c5ccccc5n6-c5ccccc5)cccc34)n2)cc1 ; c1ccc(-c2cc(-c3ccccc3)nc(-c3cccc4c3oc3c(-c5ccc6c(c5)c5ccccc5n6-c5ccccc5)cccc34)n2)cc1 ; c1ccc(-c2nc(-c3ccccc3)nc(-c3ccc(-n4c5ccccc5c5ccccc54)cc3)n2)cc1]

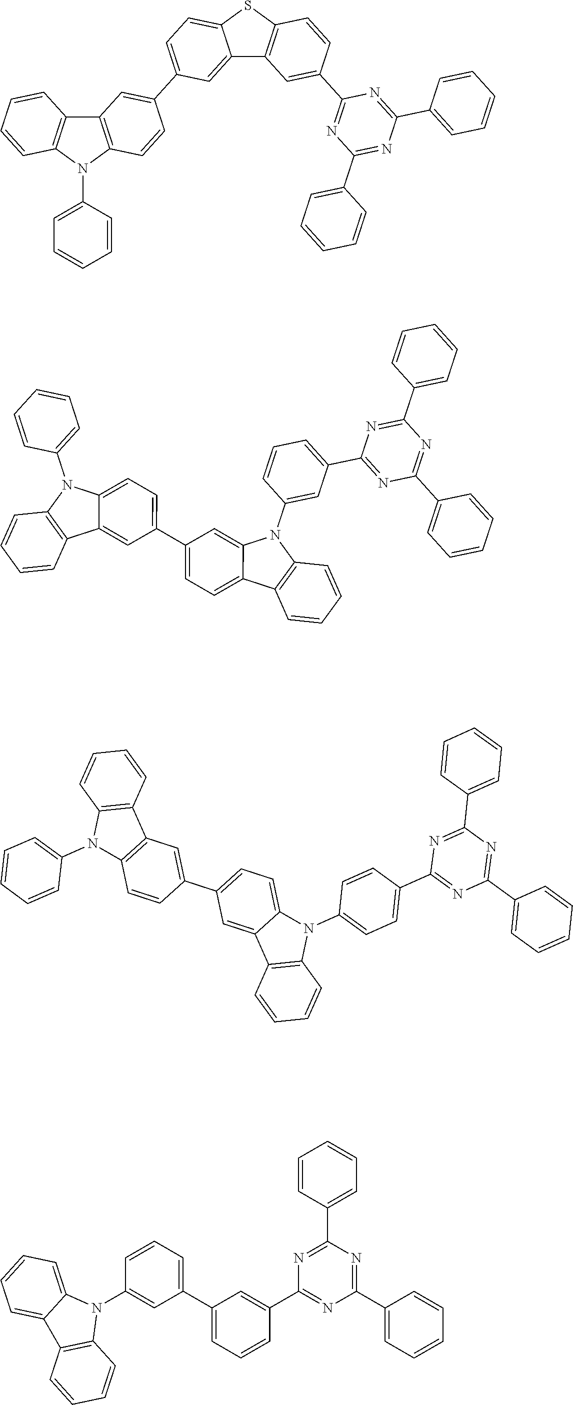 [SMILES: c1ccc(-c2nc(-c3ccccc3)nc(-c3ccc4sc5ccc(-c6ccc7c(c6)c6ccccc6n7-c6ccccc6)cc5c4c3)n2)cc1 ; c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-c3cccc(-c4cccc(-n5c6ccccc6c6ccccc65)c4)c3)n2)cc1]

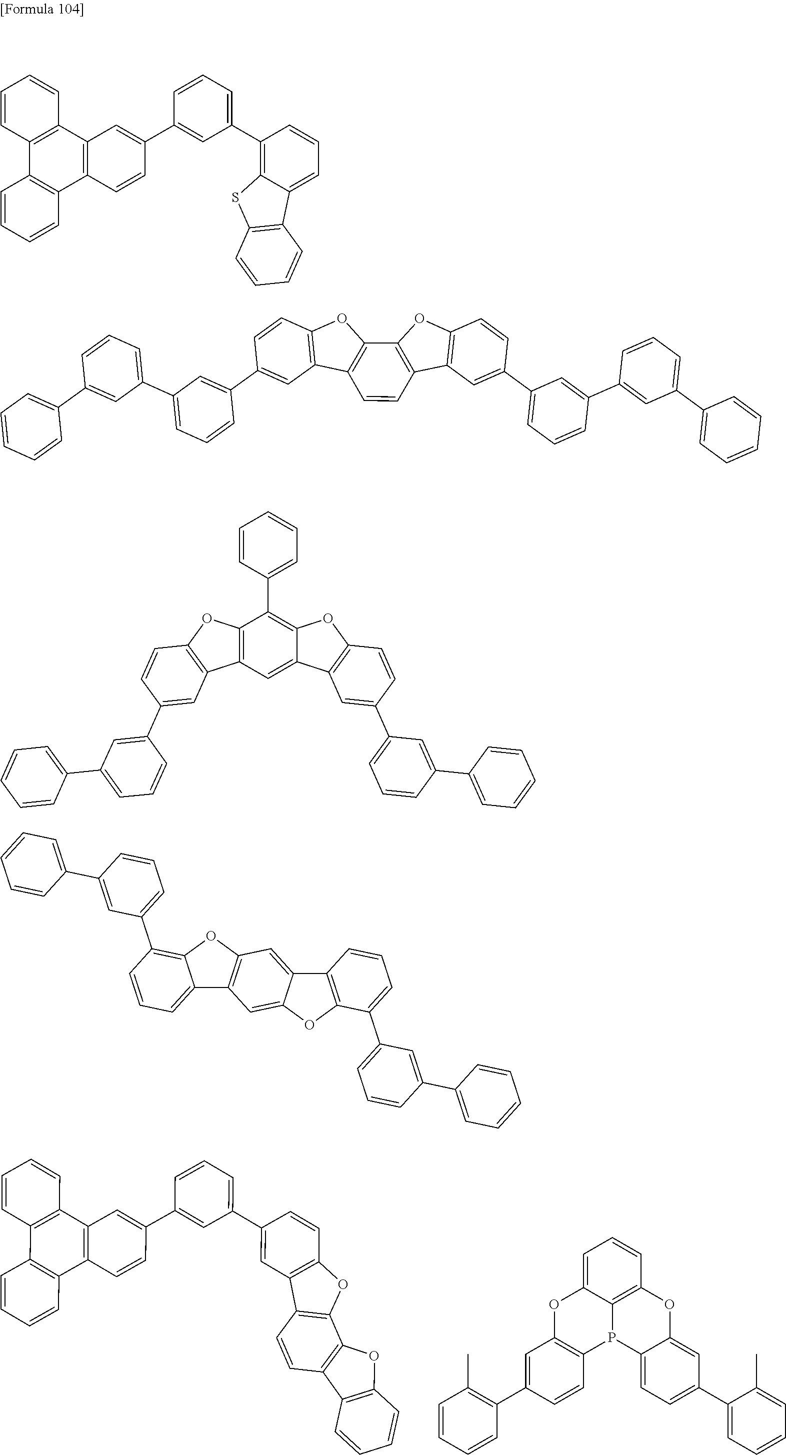

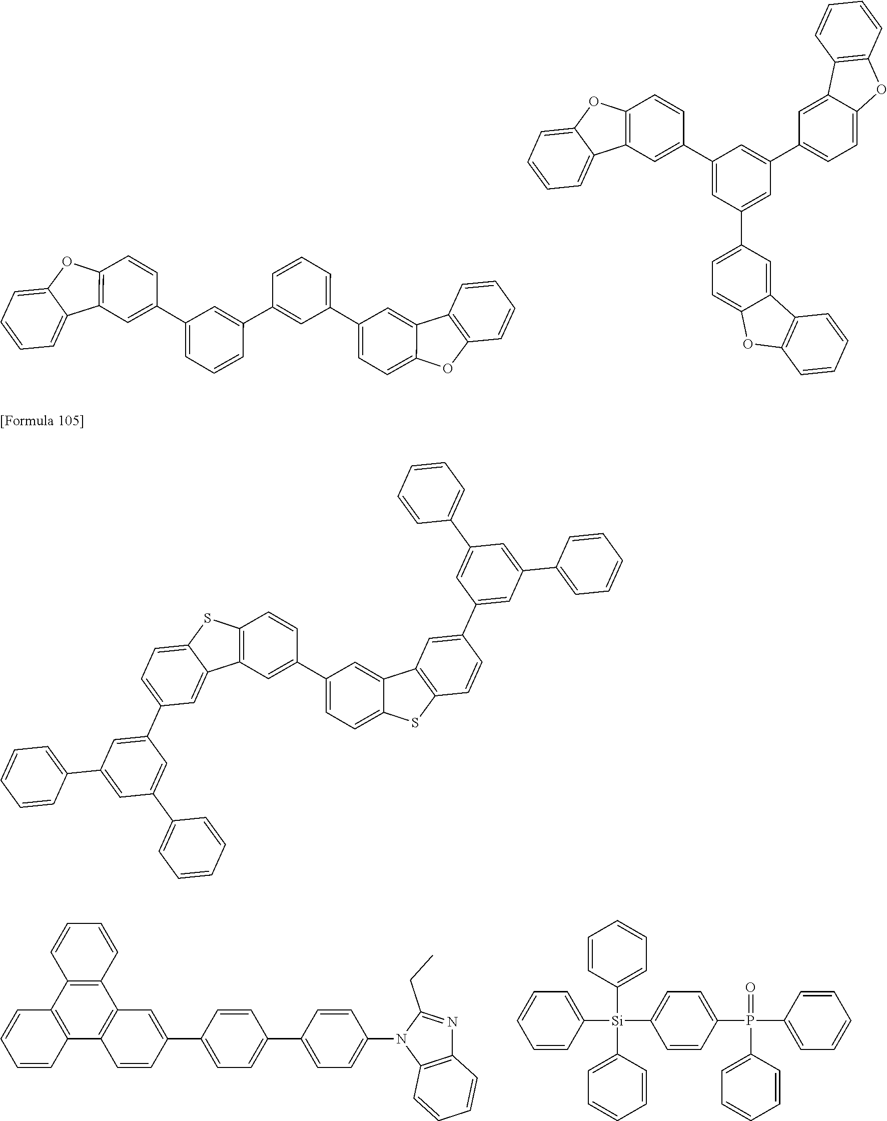 [SMILES: c1cc(-c2cccc(-c3ccc4oc5ccccc5c4c3)c2)cc(-c2ccc3oc4ccccc4c3c2)c1 ; c1ccc2c(c1)oc1ccc(-c3cc(-c4ccc5oc6ccccc6c5c4)cc(-c4ccc5oc6ccccc6c5c4)c3)cc12 ; c1ccc(-c2cc(-c3ccccc3)cc(-c3ccc4sc5ccc(-c6ccc7sc8ccc(-c9cc(-c%10ccccc%10)cc(-c%10ccccc%10)c9)cc8c7c6)cc5c4c3)c2)cc1 ; CCc1nc2ccccc2n1-c1ccc(-c2ccc(-c3ccc4c5ccccc5c5ccccc5c4c3)cc2)cc1 ; O=P(c1ccccc1)(c1ccccc1)c1ccc([Si](c2ccccc2)(c2ccccc2)c2ccccc2)cc1]

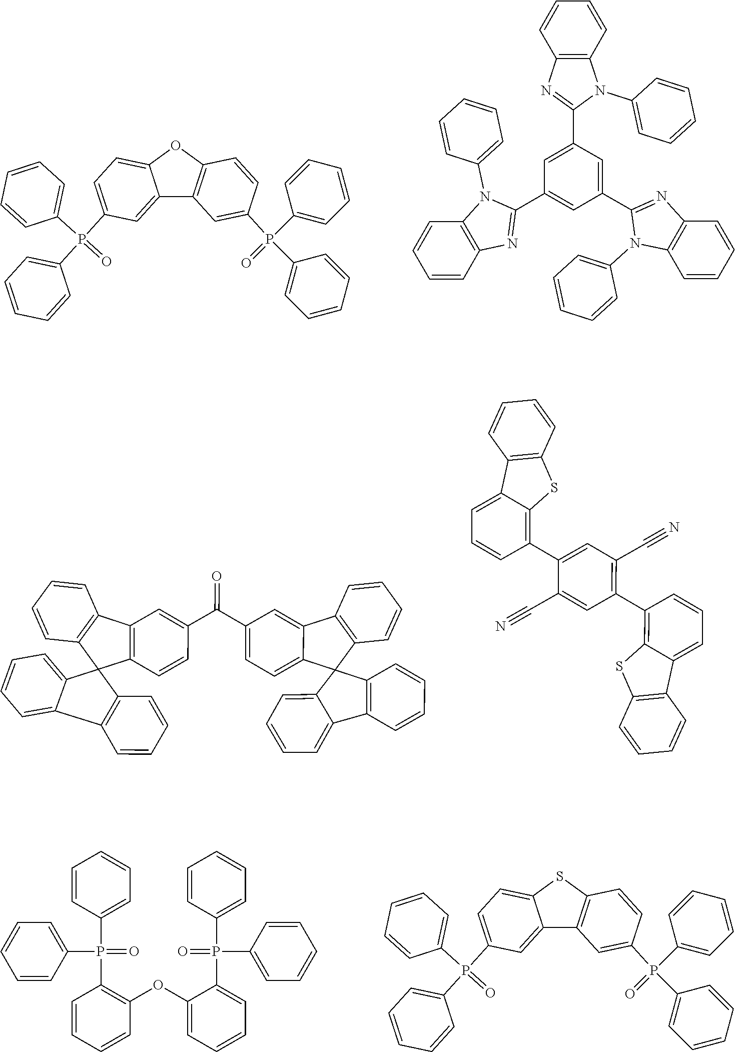 [SMILES: O=P(c1ccccc1)(c1ccccc1)c1ccc2oc3ccc(P(=O)(c4ccccc4)c4ccccc4)cc3c2c1 ; c1ccc(-n2c(-c3cc(-c4nc5ccccc5n4-c4ccccc4)cc(-c4nc5ccccc5n4-c4ccccc4)c3)nc3ccccc32)cc1 ; O=C(c1ccc2c(c1)-c1ccccc1C21c2ccccc2-c2ccccc21)c1ccc2c(c1)-c1ccccc1C21c2ccccc2-c2ccccc21 ; N#Cc1cc(-c2cccc3c2sc2ccccc23)c(C#N)cc1-c1cccc2c1sc1ccccc12 ; O=P(c1ccccc1)(c1ccccc1)c1ccccc1Oc1ccccc1P(=O)(c1ccccc1)c1ccccc1 ; O=P(c1ccccc1)(c1ccccc1)c1ccc2sc3ccc(P(=O)(c4ccccc4)c4ccccc4)cc3c2c1]

### Relationship Between Compound M1, Compound M2, and Compound M3 in Emitting Layer

In the organic EL device according to the exemplary embodiment, the singlet energy S₁(Mat2) of the compound M2 and a singlet energy S₁(Mat3) of the compound M3 preferably satisfy a relationship of a numerical formula (Numerical Formula 2A) below.

S₁(Mat3)>S₁(Mat2) (Numerical Formula 2A)

An energy gap T_{77K}(Mat3) at 77K of the compound M3 is preferably larger than the energy gap T_{77K}(Mat2) at 77K of the compound M2.

The energy gap T_{77K}(Mat3) at 77K of the compound M3 is preferably larger than the energy gap T_{77K}(Mat1) at 77K of the compound M1.

In the organic EL device according to the exemplary embodiment, the singlet energy S₁(Mat2) of the compound M2, the singlet energy S₁(Mat1) of the compound M1, and the singlet energy S₁(Mat3) of the compound M3 preferably satisfy a relationship of a numerical formula (Numerical Formula 2) below.

S₁(Mat3)>S₁(Mat2)>S₁(Mat1) (Numerical Formula 2)

In the organic EL device according to the exemplary embodiment, the energy gap T_{77K}(Mat2) at 77K of the compound M2, the energy gap T_{77K}(Mat1) at 77K of the compound M1, and the energy gap T_{77K}(Mat3) at 77K of the compound M3 preferably satisfy a relationship of a numerical formula (Numerical Formula 2B) below.

T_{77K}(Mat3)>T_{77K}(Mat2)>T_{77K}(Mat1) (Numerical Formula 2B)

It is preferable that, when the organic EL device of the exemplary embodiment emits light, the fluorescent compound M1 mainly emits light in the emitting layer.

The organic EL device of the exemplary embodiment preferably emits red light or green light.

The main peak wavelength of light emitted from the organic EL device can be measured by the same method as that for the organic EL device 1 of the first exemplary embodiment.

### Content Ratios of Compounds in Emitting Layer

For instance, content ratios of the compound M1, the compound M2, and the compound M3 in the emitting layer preferably fall within ranges shown below.

The content ratio of the compound M1 is preferably in a range from 0.01 mass % to 10 mass %, more preferably in a range from 0.01 mass % to 5 mass %, and still more preferably in a range from 0.01 mass % to 1 mass %.

The content ratio of the compound M2 is preferably in a range from 10 mass % to 80 mass %, more preferably in a range from 10 mass % to 60 mass %, and still more preferably in a range from 20 mass % to 60 mass %.

The content ratio of the compound M3 is preferably in a range from 10 mass % to 80 mass %.

The upper limit of the total of the content ratios of the compound M1, the compound M2, and the compound M3 in the emitting layer is 100 mass %. It should be noted that the emitting layer according to the exemplary embodiment may contain a material other than the compound M1, the compound M2, and the compound M3.

The emitting layer may contain a single type of the compound M1 or may contain two or more types of the compound M1. The emitting layer may contain a single type of the compound M2 or may contain two or more types of the compound M2. The emitting layer may contain a single type of the compound M3 or may contain two or more types of the compound M3.

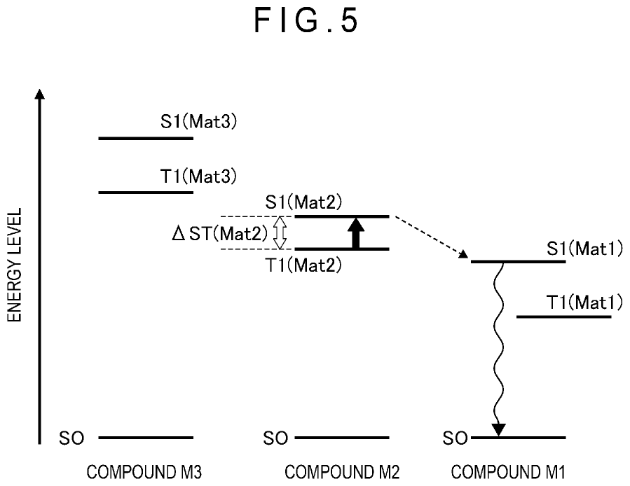

FIG. 5 shows an example of a relationship between energy levels of the compound M1, the compound M2 and the compound M3 in the emitting layer. In FIG. 5, S0 represents a ground state. S1(Mat1) represents a lowest singlet state of the compound M1, and T1(Mat1) represents a lowest triplet state of the compound M1. S1(Mat2) represents a lowest singlet state of the compound M2, and T1(Mat2) represents a lowest triplet state of the compound M2. S1(Mat3) represents a lowest singlet state of the compound M3, and T1(Mat3) represents a lowest triplet state of the compound M3. A dashed arrow directed from S1(Mat2) to S1(Mat1) in FIG. 5 represents Förster energy transfer from the lowest singlet state of the compound M2 to the lowest singlet state of the compound M1.

As shown in FIG. 5, when a compound having a small ΔST(Mat2) is used as the compound M2, inverse intersystem crossing from the lowest triplet state T1(Mat2) to the lowest singlet state S1(Mat2) can be caused by heat energy. Subsequently, Förster energy transfer from the lowest singlet state S1(Mat2) of the compound M2 to the compound M1 occurs to generate the lowest singlet state S1(Mat1). Consequently, fluorescence from the lowest singlet state S1(Mat2) of the compound M1 can be observed. It is inferred that the internal quantum efficiency can be theoretically raised up to 100% also by using delayed fluorescence by the TADF mechanism.

In the organic EL device of the second exemplary embodiment, the first layer contains the first compound (compound having at least one deuterium atom), and the emitting layer contains the delayed fluorescent compound M2, the fluorescent compound M1, and the compound M3.

In the second exemplary embodiment, an organic EL device excellent in performance (in particular, having a long lifetime) can be provided.

The organic EL device according to the second exemplary embodiment is usable in an organic EL apparatus.

The organic EL device according to the second exemplary embodiment is usable in an electronic device such as a display device and a light-emitting unit.

### Third Exemplary Embodiment

An arrangement of an organic EL device according to a third exemplary embodiment of the invention is described below. In the description of the third exemplary embodiment, the same components as those in the first and second exemplary embodiments are denoted by the same reference signs and names to simplify or omit an explanation of the components. In the third exemplary embodiment, any materials and compounds that are not specified may be the same as those in the first and second exemplary embodiments.

In the organic EL device of the third exemplary embodiment, the first layer contains the first compound having at least one deuterium atom, and the emitting layer contains the delayed fluorescent compound M2 and a compound M4.

In this arrangement, the compound M2 is preferably a dopant material, and the compound M4 is preferably a host material.

The compound M4 may be a delayed fluorescent compound or a compound exhibiting no delayed fluorescence.

The compound M4 is not particularly limited, and the compound M3 described in the second exemplary embodiment is usable as the compound M4.

The first compound and the compound M2 described in the first exemplary embodiment are each independently usable as the first compound and the compound M2.

### Relationship Between Compound M2 and Compound M4 in Emitting Layer

In the organic EL device according to the exemplary embodiment, the singlet energy S₁(Mat2) of the compound M2 and a singlet energy S₁(Mat4) of the compound M4 preferably satisfy a relationship of a numerical formula (Numerical Formula 3) below.

S₁(Mat4)>S₁(Mat2) (Numerical Formula 3)

An energy gap T_{77K}(Mat4) at 77K of the compound M4 is preferably larger than the energy gap T_{77K}(Mat2) at 77K of the compound M2.

It is preferable that, when the organic EL device of the exemplary embodiment emits light, the compound M2 mainly emits light in the emitting layer.

### Content Ratios of Compounds in Emitting Layer

For instance, content ratios of the compound M2 and the compound M4 in the emitting layer preferably fall within ranges shown below.

The content ratio of the compound M2 is preferably in a range from 10 mass % to 80 mass %, more preferably in a range from 10 mass % to 60 mass %, and still more preferably in a range from 20 mass % to 60 mass %.

The content ratio of the compound M4 is preferably in a range from 20 mass % to 90 mass %, more preferably in a range from 40 mass % to 90 mass %, and still more preferably in a range from 40 mass % to 80 mass %.

It should be noted that the emitting layer according to the exemplary embodiment may contain a material other than the compound M2 and the compound M4.

The emitting layer may contain a single type of the compound M2 or may contain two or more types of the compound M2. The emitting layer may contain a single type of the compound M4 or may contain two or more types of a fourth compound.

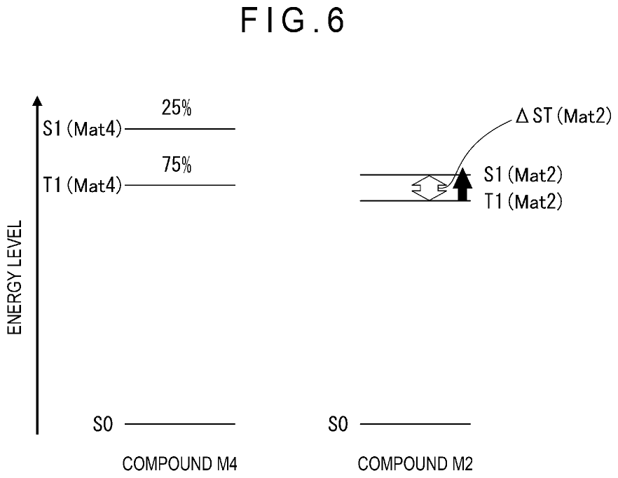

FIG. 6 shows an example of a relationship between energy levels of the compound M2 and the compound M4 in the emitting layer. In FIG. 6, S0 represents a ground state. S1(Mat2) represents a lowest singlet state of the compound M2, and T1(Mat2) represents a lowest triplet state of the compound M2. S1(Mat4) represents a lowest singlet state of the compound M4, and T1(Mat4) represents a lowest triplet state of the compound M4. As shown in FIG. 6, when a material having a small ΔST(Mat2) is used as the compound M2, inverse intersystem crossing from the lowest triplet state T1 to the lowest singlet state S1 in the compound M2 can be caused by heat energy.

The inverse intersystem crossing caused in the compound M2 enables light emission from the lowest singlet state S1(Mat2) of the compound M2 to be observed when the emitting layer does not contain a fluorescent dopant with the lowest singlet state S1 smaller than the lowest singlet state S1(Mat2) of the compound M2. It is inferred that the internal quantum efficiency can be theoretically raised up to 100% also by using delayed fluorescence by the TADF mechanism.

In the organic EL device according to the third exemplary embodiment, the first layer contains the first compound (compound having at least one deuterium atom), and the emitting layer contains the delayed fluorescent compound M2 and the compound M4.

In the third exemplary embodiment, an organic EL device excellent in performance (in particular, having a long lifetime) can be provided.

The organic EL device according to the third exemplary embodiment is usable in an organic EL apparatus.

The organic EL device according to the third exemplary embodiment is usable in an electronic device such as a display device and a light-emitting unit.

### Fourth Exemplary Embodiment

An arrangement of an organic EL device according to a fourth exemplary embodiment will be described below. In the description of the fourth exemplary embodiment, the same components as those in the first to third exemplary embodiments are denoted by the same reference signs and names to simplify or omit an explanation of the components. In the fourth exemplary embodiment, any materials and compounds that are not specified may be the same as those in the first to third exemplary embodiments.

The organic EL device according to the fourth exemplary embodiment is different from the organic EL device according to any of the exemplary embodiments in that a second layer is provided between the first layer and the cathode. The rest of the arrangement of the organic EL device according to the fourth exemplary embodiment is the same as in the above exemplary embodiments.

The organic EL device according to the fourth exemplary embodiment includes the anode, the cathode, the emitting layer between the anode and the cathode, the first layer between the emitting layer and the cathode, and the second layer between the first layer and the cathode.

The emitting layer at least contains the delayed fluorescent compound M2, and the first layer contains the first compound having at least one deuterium atom.

The second layer contains a second compound. The first compound is different from the second compound.

The emitting layer of any of the first to third exemplary embodiments is applicable as the emitting layer of the fourth exemplary embodiment.

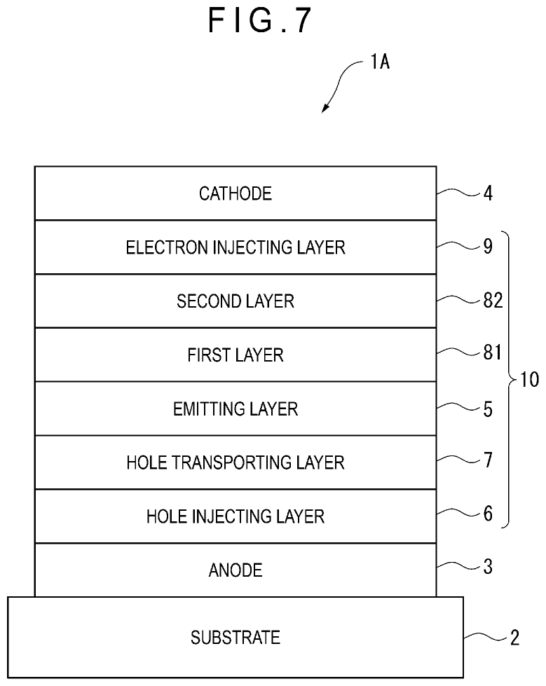

FIG. 7 schematically shows an exemplary arrangement of the organic EL device according to the fourth exemplary embodiment.

FIG. 7 shows a case where the emitting layer 5 of the first exemplary embodiment is applied as the emitting layer.

An organic EL device 1A includes the light-transmissive substrate 2, the anode 3, the cathode 4, and the organic layer 10 provided between the anode 3 and the cathode 4. The organic layer 10 includes the hole injecting layer 6, the hole transporting layer 7, the emitting layer 5, the first layer 81, a second layer 82, and the electron injecting layer 9 that are layered on the anode 3 in this order.

The first layer 81 is preferably in direct contact with the emitting layer 5.

The second layer 82 is preferably in direct contact with the first layer 81.

In the fourth exemplary embodiment, an organic EL device excellent in performance (in particular, having a long lifetime) can be provided.

The organic EL device according to the fourth exemplary embodiment is usable in an organic EL apparatus.

Further, the organic EL device according to the fourth exemplary embodiment is usable in an electronic device such as a display device and a light-emitting unit.

The second layer is explained below.

### Second Layer (Second Compound)

The second layer contains the second compound.

The second compound, which is not particularly limited, is preferably a compound represented by a formula (B) below.

That is, the second layer preferably contains the second compound represented by the formula (B).

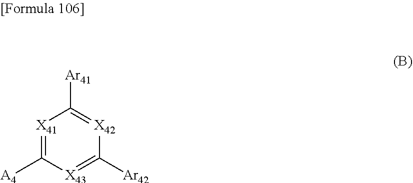

In the formula (B):
X₄₁ to X₄₃ are each independently a nitrogen atom or CR₄₁;
R₄₁ is a hydrogen atom or a substituent, or at least one combination of combinations of adjacent two or more of a plurality of R₄₁ are mutually bonded to form a ring;
at least one of X₄₁ to X₄₃ is a nitrogen atom;
R₄₁ is a hydrogen atom or a substituent;
each R₄₁ as the substituent is independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted germanium group, a substituted phosphine oxide group, a nitro group, a substituted or unsubstituted carbonyl group, or a substituted boryl group;
a plurality of R₄₁ are mutually the same or different;
Ar₄₁ and Ar₄₂ are each independently represented by a formula (1B) below, or are each independently a substituted or unsubstituted aryl group having 6 to 30 ring carbon atoms, or a substituted or unsubstituted heteroaryl group having 5 to 30 ring atoms; and
A₄ is represented by the formula (1B), or is a substituted or unsubstituted aryl group having 6 to 30 ring carbon atoms, or a substituted or unsubstituted heteroaryl group having 5 to 30 ring atoms.

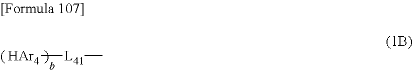

In the formula (1B):
HAr₄ is represented by a formula (2B) below;
b is 1, 2, 3, 4, or 5;
when b is 1, L₄₁ is a single bond or a divalent linking group;
when b is 2, 3, 4 or 5, L₄₁ is a trivalent to hexavalent linking group;
a plurality of HAr₄ are mutually the same or different;
L₄₁ as a linking group is a substituted or unsubstituted arylene group having 6 to 30 ring carbon atoms, or a trivalent, tetravalent, pentavalent, or hexavalent group derived from the arylene group; a substituted or unsubstituted divalent heterocyclic group having 5 to 30 ring atoms, or a trivalent, tetravalent, pentavalent, or hexavalent group derived from the heterocyclic group; or a divalent group formed by bonding two groups selected from the group consisting of a substituted or unsubstituted arylene group having 6 to 30 ring carbon atoms and a substituted or unsubstituted divalent heterocyclic group having 5 to 30 ring atoms, or a trivalent, tetravalent, pentavalent, or hexavalent group derived from the divalent group; and
the mutually bonded groups are mutually the same or different.

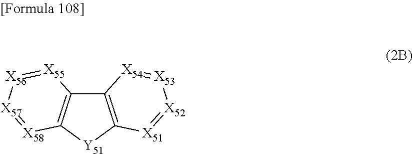

In the formula (2B):
X₅₁ to X₅₈ are each independently a nitrogen atom, CR₅₃, or a carbon atom bonded to L₄₁;
a plurality of R₅₃ are mutually the same or different;
Y₅₁ is an oxygen atom, a sulfur atom, NR₅₈, SiR₅₁R₅₂, CR₅₄R₅₅, a nitrogen atom bonded to L₄₁, a silicon atom bonded to each of R₅₆ and L₄₁, or a carbon atom bonded to each of R₅₇ and L₄₁;
among carbon atoms for X₅₁ to X₅₈, a nitrogen atom for Y₅₁, a silicon atom for Y₅₁, and a carbon atom for Y₅₁, any one atom is bonded to L₄₁;
R₅₁ to R₅₈ are each independently a hydrogen atom or a substituent, or at least one combination of a combination of adjacent ones of R₅₃, a combination of R₅₁ and R₅₂, or a combination of R₅₄ and R₅₅ are bonded to each other to form a ring; and
R₅₁ to R₅₈ as the substituents are each independently a halogen atom, a cyano group, a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted silyl group, a substituted or unsubstituted alkoxy group having 1 to 30 carbon atoms, a substituted or unsubstituted aralkyl group having 7 to 30 carbon atoms, or a substituted or unsubstituted aryloxy group having 6 to 30 ring carbon atoms.

The second compound also corresponds to a compound according to an exemplary arrangement of the first compound represented by the formula (1). Thus, a compound being the first compound and satisfying the formula (B) also corresponds to the second compound.

The second compound has or does not have at least one deuterium atom. Preferably, the second compound has no deuterium atom.

In the formula (1B), b is more preferably 1 or 2.

In the formula (2B), X₅₁ to X₅₈ are preferably each independently CR₅₃.

In the formula (2B), Y₅₁ is preferably an oxygen atom, a sulfur atom, NR₅₅, CR₅₄R₅₅, a nitrogen atom bonded to L₄₁, or a carbon atom bonded to each of R₅₇ and L₄₁.

In the formula (2B), X₅₃ or X₅₆ is preferably a carbon atom bonded to L₄₁ by a single bond.

In the formula (2B), X₅₁ or X₅₈ is also preferably a carbon atom bonded to L₄₁ by a single bond.

In the formula (2B), X₅₂ or X₅₇ is also preferably a carbon atom bonded to L₄₁ by a single bond.

In the formula (2B), X₅₄ or X₅₅ is also preferably a carbon atom bonded to L₄₁ by a single bond.

In the second compound, Ar₄₁ and Ar₄₂ are preferably each independently represented by the formula (1B), or a substituted or unsubstituted aryl group having 6 to 30 ring carbon atoms.

In the second compound, A₄ is preferably represented by the formula (1B).

In the second compound, it is preferable that each R₄₁ for CR₄₁ is independently a hydrogen atom, a substituted or unsubstituted aryl group having 6 to 30 ring carbon atoms, or a substituted or unsubstituted heteroaryl group having 5 to 30 ring atoms.

In the second compound, each R₅₃ is preferably independently a hydrogen atom, a substituted or unsubstituted alkyl group having 1 to 30 ring carbon atoms, a substituted or unsubstituted aryl group having 6 to 30 ring carbon atoms, or a substituted or unsubstituted heteroaryl group having 5 to 30 ring atoms.

In the second compound, L₄₁ is preferably a single bond; a substituted or unsubstituted arylene group having 6 to 30 ring carbon atoms, or a trivalent, tetravalent, pentavalent, or hexavalent group derived from the arylene group; or a substituted or unsubstituted divalent heterocyclic group having 5 to 30 ring atoms, or a trivalent, tetravalent, pentavalent, or hexavalent group derived from the heterocyclic group.

In the second compound, L₄₁ is more preferably a single bond; a substituted or unsubstituted arylene group having 6 to 30 ring carbon atoms, or a trivalent group derived from the arylene group; or a substituted or unsubstituted divalent heterocyclic group having 5 to 30 ring atoms, or a trivalent group derived from the heterocyclic group.

In the second compound, one or two of X₄₁, X₄₂, and X₄₃ are preferably nitrogen atoms.

In the second compound, X₄₁, X₄₂, and X₄₃ are preferably nitrogen atoms.

The compound represented by the formula (B) is also preferably a compound represented by a formula (B-1), (B-2), or (B-3) below.

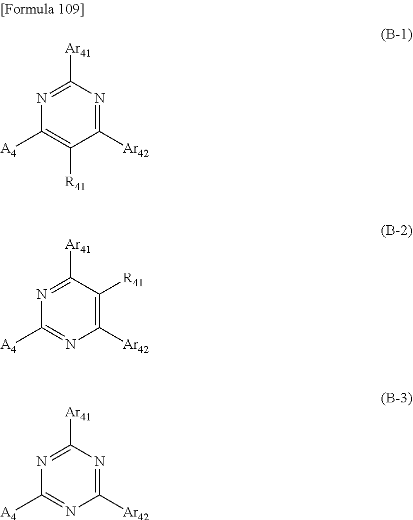

In the formulae (B-1) to (B-3), Ar₄₁, Ar₄₂, A₄ and R₄₁ each independently represent the same as Ar₄₁, Ar₄₂, A₄ and R₄₁ in the formula (B).

### Method for Producing Second Compound

The second compound can be produced by a known method.

Specific examples of the second compound include the following compounds. It should however be noted that the invention is not limited to the specific examples of the compound.

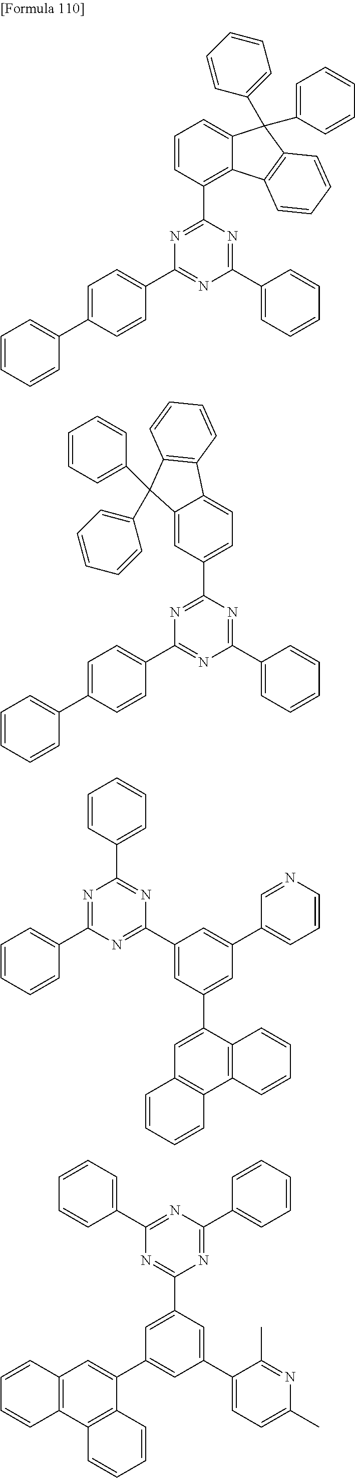 [SMILES: c1ccc(-c2ccc(-c3nc(-c4ccccc4)nc(-c4cccc5c4-c4ccccc4C5(c4ccccc4)c4ccccc4)n3)cc2)cc1 ; c1ccc(-c2ccc(-c3nc(-c4ccccc4)nc(-c4ccc5c(c4)C(c4ccccc4)(c4ccccc4)c4ccccc4-5)n3)cc2)cc1 ; c1ccc(-c2nc(-c3ccccc3)nc(-c3cc(-c4cccnc4)cc(-c4cc5ccccc5c5ccccc45)c3)n2)cc1 ; Cc1ccc(-c2cc(-c3nc(-c4ccccc4)nc(-c4ccccc4)n3)cc(-c3cc4ccccc4c4ccccc34)c2)c(C)n1]

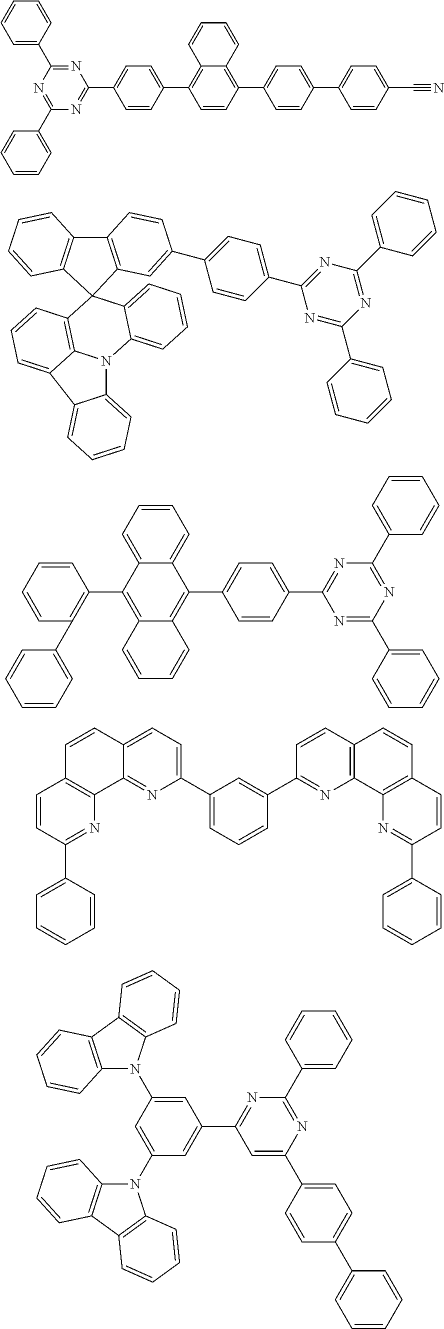

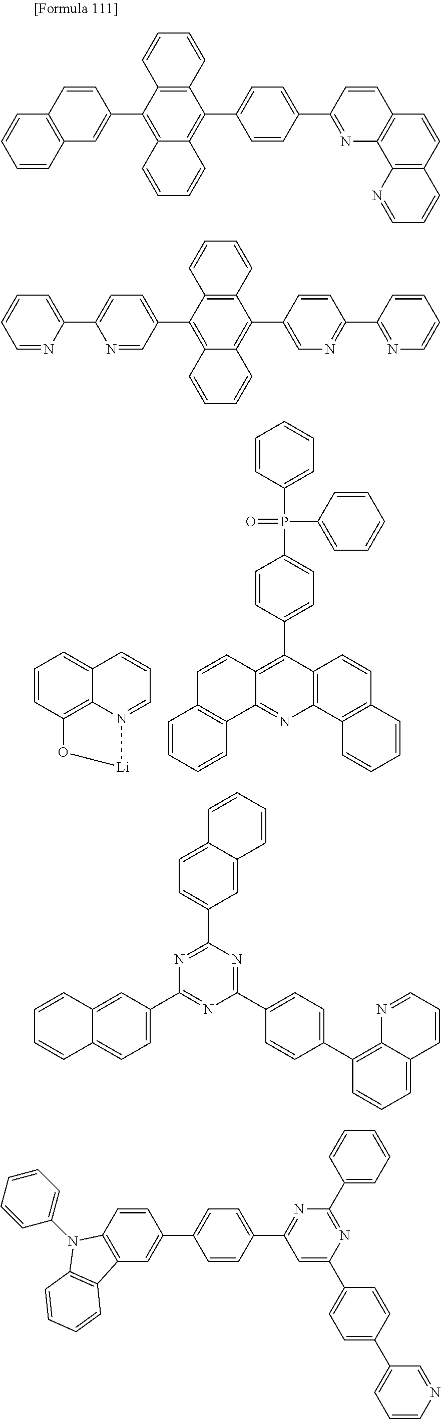

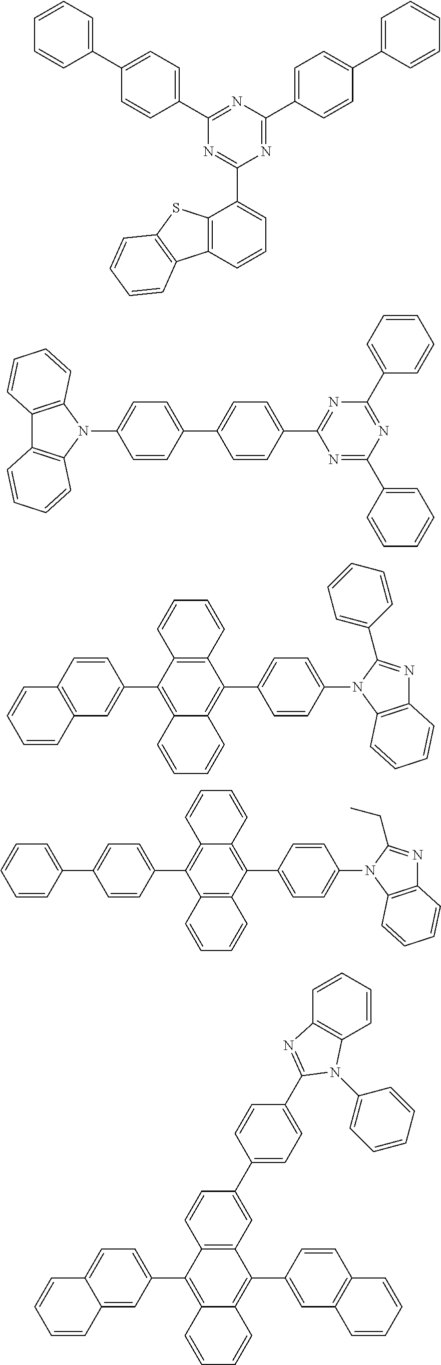

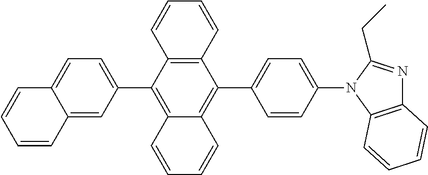 [SMILES: CCc1nc2ccccc2n1-c1ccc(-c2c3ccccc3c(-c3ccc4ccccc4c3)c3ccccc23)cc1]

### Fifth Exemplary Embodiment

### Organic Electroluminescence Apparatus

An organic EL apparatus according to a fifth exemplary embodiment includes a first device that is the organic EL device according to any of the first to fourth exemplary embodiments; a second device that is an organic EL device different from the first device; and a substrate, in which the first device and the second device are arranged in parallel on the substrate and the first layer of the first device is a common layer provided in common to the first device and the second device.

The first device is the organic EL device according to any of the above exemplary embodiments.

That is, the organic EL device according to any of the first to fourth exemplary embodiments is applicable as the first device.

The second device may be a device that fluoresces or a device that phosphoresces. The organic EL device according to any of the first to fourth exemplary embodiments is applicable as the second device. The emission color of the first and second devices is not particularly limited.

In the fifth exemplary embodiment, a case where the first device is the organic EL device 1 of the first exemplary embodiment is explained.

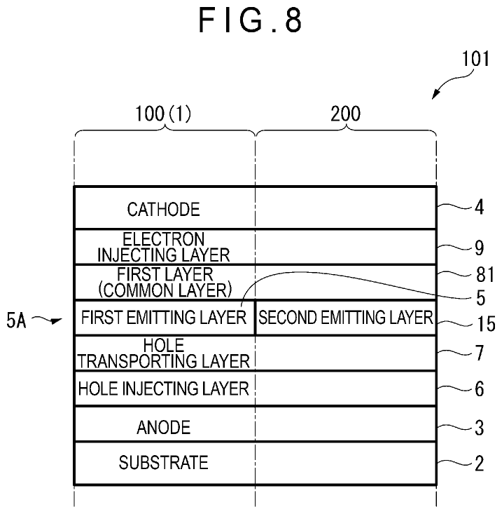

FIG. 8 schematically shows an exemplary arrangement of the organic EL apparatus according to the fifth exemplary embodiment.

FIG. 8 shows a case where the organic EL device 1 of the first exemplary embodiment is applied as the first device.

An organic EL apparatus 101 includes a first device 100 (organic EL device 1 of the first exemplary embodiment), a second device 200 different from the first device 100, and the light-transmissive substrate 2. The first device 100 and the second device 200 are arranged in parallel on the substrate 2.

The first device 100 and the second device 200 are each configured as an organic EL device.

The organic EL apparatus 101 includes the substrate 2, the anode 3, the hole injecting layer 6, the hole transporting layer 7, an emitting zone 5A, the first layer 81 as the common layer, the electron injecting layer 9, and the cathode 4. The anode 3, the hole injecting layer 6, the hole transporting layer 7, the emitting zone 5A, the first layer 81, the electron injecting layer 9, and the cathode 4 are layered in this order.

The first layer 81 of the first device 100 is a common layer provided in common to the first device 100 and the second device 200. The first layer 81 (common layer) is provided between the emitting zone 5A and the electron injecting layer 9.

An arrangement of the emitting zone 5A in the first device 100 is different from that in the second device 200. The emitting zone 5A in the first device 100 includes the first emitting layer 5 (emitting layer 5 of the first exemplary embodiment). The emitting zone 5A in the second device 200 includes a second emitting layer 15. For instance, the first emitting layer 5 is a red emitting layer that emits red light, and the second emitting layer 15 is a green emitting layer that emits green light. The emission colors of the first emitting layer 5 and the second emitting layer 15 are not limited thereto.

The first layer 81 as the common layer is preferably in direct contact with a side of the emitting zone 5A close to the cathode. That is, the first layer 81 is preferably in direct contact with the first emitting layer 5 and the second emitting layer 15.

In the organic EL apparatus according to the fifth exemplary embodiment, the first layer as the common layer contains the first compound (compound having at least one deuterium atom), and the first emitting layer at least contains the delayed fluorescent compound M2.

According to the fifth exemplary embodiment, an organic EL apparatus excellent in performance (in particular, having a long lifetime) can be provided. The organic EL apparatus according to the fifth exemplary embodiment is usable in an electronic device such as a display device and a light-emitting unit.

### Sixth Exemplary Embodiment

An arrangement of an organic EL apparatus according to a sixth exemplary embodiment is described below. In the description of the sixth exemplary embodiment, the same components as those in the fifth exemplary embodiment are denoted by the same reference signs and names to simplify or omit an explanation of the components.

The organic EL apparatus according to the sixth exemplary embodiment further includes a third device, which is a difference from the organic EL apparatus according to the fifth exemplary embodiment.

The organic EL apparatus according to the sixth exemplary embodiment further includes the third device that is an organic EL device different from the first and second devices. The first device, the second device, and the third device are arranged in parallel on the substrate. The first layer of the first device is a common layer provided in common to the first device, the second device, and the third device.

The third device may be a device that fluoresces or a device that phosphoresces. The organic EL device according to any of the first to fourth exemplary embodiments is applicable as the third device. The emission color of the third device is not particularly limited.

Similar to the fifth exemplary embodiment, in the sixth exemplary embodiment, a case where the first device is the organic EL device 1 of the first exemplary embodiment is explained.

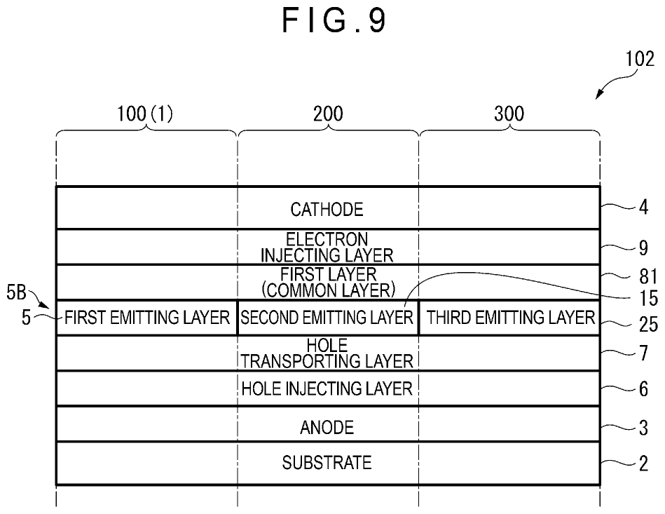

FIG. 9 schematically shows an exemplary arrangement of the organic EL apparatus according to the sixth exemplary embodiment.

An organic EL apparatus 102 includes the first device 100 (organic EL device 1 of the first exemplary embodiment), the second device 200, a third device 300, and the light-transmissive substrate 2. The first device 100, the second device 200, and the third device 300 are arranged in parallel on the substrate 2.

The first device 100, the second device 200, and the third device 300 are each configured as an organic EL device.

The organic EL apparatus 102 includes the substrate 2, the anode 3, the hole injecting layer 6, the hole transporting layer 7, an emitting zone 5B, the first layer 81 as a common layer, the electron injecting layer 9, and the cathode 4. The anode 3, the hole injecting layer 6, the hole transporting layer 7, the emitting zone 5B, the first layer 81, the electron injecting layer 9, and the cathode 4 are layered in this order.

The first layer 81 of the first device 100 is a common layer provided in common to the first device 100, the second device 200, and the third device 300. The first layer 81 (common layer) is provided between the emitting zone 5B and the electron injecting layer 9.

The first device 100, the second device 200, and the third device 300 have mutually different arrangements of the emitting zone 5B. The emitting zone 5B in the first device 100 includes the first emitting layer 5. The emitting zone 5B in the second device 200 includes the second emitting layer 15. The emitting zone 5B in the third device 300 includes a third emitting layer 25. For instance, the first emitting layer 5 is a red emitting layer that emits red light, the second emitting layer 15 is a green emitting layer that emits green light, and the third emitting layer 25 is a blue emitting layer that emits blue light. The emission colors of the first emitting layer 5, the second emitting layer 15, and the third emitting layer 25 are not limited thereto.

The first layer 81 as the common layer is preferably in direct contact with a side of the emitting zone 5B close to the cathode. That is, the first layer 81 is preferably in direct contact with the first emitting layer 5, the second emitting layer 15, and the third emitting layer 25.

In the organic EL apparatus according to the sixth exemplary embodiment, the first layer 81 as the common layer contains the first compound (compound having at least one deuterium atom), and the first emitting layer 5 at least contains the delayed fluorescent compound M2.

According to the sixth exemplary embodiment, an organic EL apparatus excellent in performance (in particular, having a long lifetime) can be provided. The organic EL apparatus according to the sixth exemplary embodiment is usable in an electronic device such as a display device and a light-emitting unit.

### Seventh Exemplary Embodiment

An arrangement of an organic EL apparatus according to a seventh exemplary embodiment is described below. In the description of the seventh exemplary embodiment, the same components as those in the fifth and sixth exemplary embodiments are denoted by the same reference signs and names to simplify or omit an explanation of the components.

The organic EL apparatus according to the seventh exemplary embodiment further includes the second layer between the first layer and the cathode, which is a difference from the organic EL apparatuses according to the fifth and sixth exemplary embodiments. The rest of the arrangement of the organic EL device according to the seventh exemplary embodiment is the same as in the fifth and sixth exemplary embodiments.

The second layer may be provided between the first layer and the cathode at least in the first device. The second layer may be a common layer provided in common to the first device and the second device. When the organic EL apparatus includes the third device 300, the second layer may be a common layer provided in common to the first device, the second device, and the third device.

The second layer of the fourth exemplary embodiment is applicable as the second layer of the seventh exemplary embodiment.

In the organic EL apparatus according to the seventh exemplary embodiment, the first layer as the common layer contains the first compound (compound having at least one deuterium atom), the second layer at least in the first device contains the second compound of the fourth exemplary embodiment, and the first emitting layer at least contains the delayed fluorescent compound M2.

According to the seventh exemplary embodiment, an organic EL apparatus excellent in performance (in particular, having a long lifetime) can be provided. The organic EL apparatus according to the seventh exemplary embodiment is usable in an electronic device such as a display device and a light-emitting unit.

### Eighth Exemplary Embodiment

### Organic EL Apparatus

An organic EL apparatus according to an eighth exemplary embodiment includes at least one of the organic EL devices according to the first to fourth exemplary embodiments.

The organic EL apparatus is exemplified by the organic EL apparatus according to each of the fifth to seventh exemplary embodiments.

### Ninth Exemplary Embodiment

### Electronic Device

An electronic device according to the ninth exemplary embodiment includes at least one of the organic EL devices according to the first to fourth exemplary embodiments or the organic EL apparatuses according to the fifth to seventh exemplary embodiments.

Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light.

### Modification of Embodiment(s)

The scope of the invention is not limited to the above-described exemplary embodiments but includes any modification and improvement as long as such modification and improvement are compatible with the invention.

For instance, the emitting layer is not limited to a single layer, but may be provided by layering a plurality of emitting layers. When the organic EL device has a plurality of emitting layers, it is only required that at least one of the emitting layers satisfies the conditions described in the above exemplary embodiment(s). For instance, in some embodiments, the rest of the emitting layers may be a fluorescent emitting layer or a phosphorescent emitting layer with use of emission caused by electron transfer from the triplet excited state directly to the ground state.

When the organic EL device includes a plurality of emitting layers, these emitting layers may be mutually adjacently provided, or may form a so-called tandem organic EL device in which a plurality of emitting units are layered via an intermediate layer.

For instance, a blocking layer may be provided adjacent to at least one of a side of the emitting layer close to the anode or a side of the emitting layer close to the cathode. The blocking layer is preferably provided in contact with the emitting layer to block at least any of holes, electrons, or excitons.

For instance, when the blocking layer is provided in contact with the side of the emitting layer close to the cathode, the blocking layer permits transport of electrons and blocks holes from reaching a layer provided closer to the cathode (e.g., the electron transporting layer) beyond the blocking layer. When the organic EL device includes the electron transporting layer, the blocking layer is preferably provided between the emitting layer and the electron transporting layer. In this arrangement, the blocking layer (an exemplary first layer) preferably contains the first compound having at least one deuterium atom. The electron transporting layer (an exemplary second layer) preferably contains the second compound of the fourth exemplary embodiment (preferably the second compound represented by the formula (B)).

Further, when the blocking layer is provided in contact with the side of the emitting layer close to the anode, the blocking layer permits transport of holes and blocks electrons from reaching a layer provided closer to the anode (e.g., the hole transporting layer) beyond the blocking layer. When the organic EL device includes the hole transporting layer, the blocking layer is preferably provided between the emitting layer and the hole transporting layer.

Alternatively, the blocking layer may be provided adjacent to the emitting layer so that the excitation energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) beyond the blocking layer.

The emitting layer is preferably joined to the blocking layer.

Specific structure, shape and the like of the components in the invention may be designed in any manner as long as an object of the invention can be achieved.

Herein, a numerical range represented by “x to y” represents a range whose lower limit is the value (x) recited before “to” and whose upper limit is the value (y) recited after “to.”

Herein, the phrase “Rx and Ry are mutually bonded to form a ring” means, for instance, that Rx and Ry contain a carbon atom, a nitrogen atom, an oxygen atom, a sulfur atom or a silicon atom, the atom(s) contained in Rx (a carbon atom, a nitrogen atom, an oxygen atom, a sulfur atom or a silicon atom) and the atom(s) contained in Ry (a carbon atom, a nitrogen atom, an oxygen atom, a sulfur atom or a silicon atom) are bonded via a single bond(s), a double bond(s), a triple bond, and/or a divalent linking group(s) to form a ring having 5 or more ring atoms (specifically, a heterocyclic ring or an aromatic hydrocarbon ring). x represents a number, a character or a combination of a number and a character. y represents a number, a character or a combination of a number and a character.

The divalent linking group is not particularly limited. Examples of the divalent linking group include —O—, —CO—, —CO₂—, —S—, —SO—, —SO₂—, —NH—, —NRa—, and a group provided by a combination of two or more of these linking groups.

Specific examples of the heterocyclic ring include a cyclic structure (heterocyclic ring) obtained by removing a bond from a “heteroaryl group Sub₂” exemplarily shown in the later-described “Description of Each Substituent in Formula.” The heterocyclic ring may have a substituent.

Specific examples of the aromatic hydrocarbon ring include a cyclic structure (aromatic hydrocarbon ring) obtained by removing a bond from a “aryl group Sub₁” exemplarily shown in the later-described “Description of Each Substituent in Formula.” The aromatic hydrocarbon ring may have a substituent.

Examples of Ra include a substituted or unsubstituted alkyl group Sub₃ having 1 to 30 carbon atoms, a substituted or unsubstituted aryl group Sub₁ having 6 to 30 ring carbon atoms, and a substituted or unsubstituted heteroaryl group Sub₂ having 5 to 30 ring atoms, which are exemplarily shown in the later-described “Description of Each Substituent in Formula.”

Rx and Ry are mutually bonded to form a ring, which means, for instance, that: an atom contained in Rx₁ and an atom contained in Ry₁ in a molecular structure represented by a formula (E1) below form a ring (cyclic structure) E represented by a formula (E2); an atom contained in Rx₁ and an atom contained in Ry₁ in a molecular structure represented by a formula (F1) below form a ring F represented by a formula (F2); an atom contained in Rx₁ and an atom contained in Ry₁ in a molecular structure represented by a formula (G1) below form a ring G represented by a formula (G2); an atom contained in Rx₁ and an atom contained in Ry₁ in a molecular structure represented by a formula (H1) below form a ring H represented by a formula (H2); and an atom contained in Rx₁ and an atom contained in Ry₁ in a molecular structure represented by a formula (I1) below form a ring I represented by a formula (I2).

In the formulae (E1) to (I1), each * independently represents a bonding position to another atom in a molecule. The two * in the formulae (E1), (F1), (G1), (H1) and (I1) correspond to two * in the formulae (E2), (F2), (G2), (H2) and (I2), respectively.

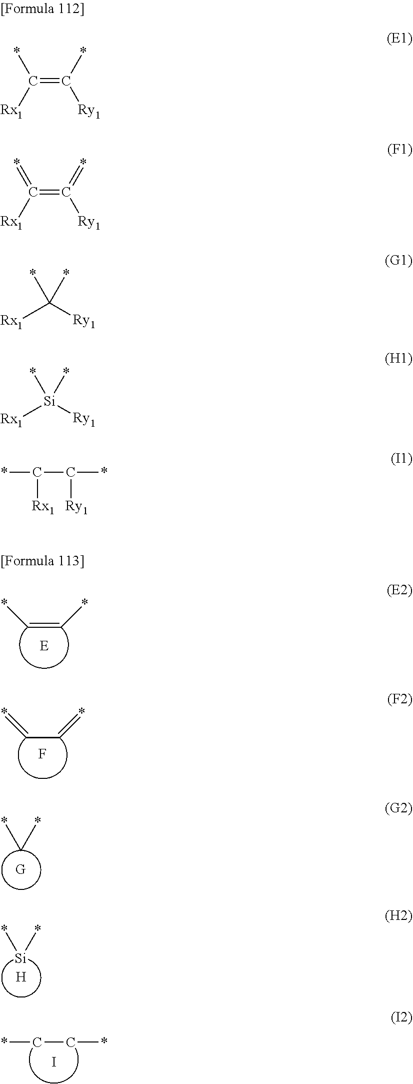

In the molecular structures represented by the formulae (E2) to (I2), E to I each represent a cyclic structure (the ring having 5 or more ring atoms). In the formulae (E2) to (I2), each * independently represents a bonding position to another atom in a molecule. The two * in the formula (E2) correspond to two * in the formula (E1). Similarly, two * in each of the formulae (F2) to (I2) correspond one-to-one to two * in in each of the formulae (F1) to (I1).

For instance, in the formula (E1), when Rx₁ and Ry₁ are mutually bonded to form the ring E in the formula (E2) and the ring E is an unsubstituted benzene ring, the molecular structure represented by the formula (E1) is a molecular structure represented by a formula (E3) below. Herein, two * in the formula (E3) each independently correspond to two * in the formula (E2) and the formula (E1).

For instance, in the formula (E1), when Rx₁ and Ry₁ are mutually bonded to form the ring E in the formula (E2) and the ring E is an unsubstituted pyrrole ring, the molecular structure represented by the formula (E1) is a molecular structure represented by a formula (E4) below. Herein, two * in the formula (E4) each independently correspond to two * in the formula (E2) and the formula (E1). In the formulae (E3) and (E4), each * independently represents a bonding position to another atom in a molecule.

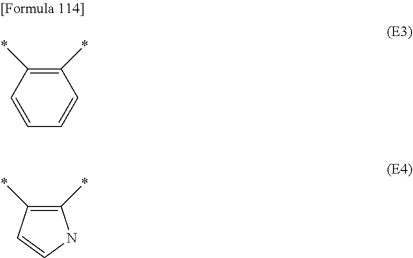 [SMILES: *c1ccccc1* ; *c1cc[nH]c1*]

Herein, the ring carbon atoms refer to the number of carbon atoms among atoms forming a ring of a compound (e.g., a monocyclic compound, fused-ring compound, crosslinking compound, carbon ring compound, and heterocyclic compound) in which the atoms are bonded to each other to form the ring. When the ring is substituted by a substituent(s), carbon atom(s) contained in the substituent(s) is not counted in the ring carbon atoms. Unless specifically described, the same applies to the “ring carbon atoms” described later. For instance, a benzene ring has 6 ring carbon atoms, a naphthalene ring has 10 ring carbon atoms, a pyridinyl group has 5 ring carbon atoms, and a furanyl group has 4 ring carbon atoms. When a benzene ring and/or a naphthalene ring is substituted by a substituent (e.g., an alkyl group), the number of carbon atoms of the alkyl group is not counted in the number of the ring carbon atoms. When a fluorene ring is substituted by a substituent (e.g., a fluorene ring) (i.e., a spirofluorene ring is included), the number of carbon atoms of the fluorene ring as the substituent is not counted in the number of the ring carbon atoms of the fluorene ring.

Herein, the ring atoms refer to the number of atoms forming a ring of a compound (e.g., a monocyclic compound, fused-ring compound, crosslinking compound, carbon ring compound, and heterocyclic compound) in which the atoms are bonded to each other to form the ring (e.g., monocyclic ring, fused ring, ring assembly). Atom(s) not forming a ring and atom(s) included in a substituent when the ring is substituted by the substituent are not counted in the number of the ring atoms. Unless specifically described, the same applies to the “ring atoms” described later. For instance, a pyridine ring has six ring atoms, a quinazoline ring has ten ring atoms, and a furan ring has five ring atoms. A hydrogen atom(s) and/or an atom(s) of a substituent which are bonded to carbon atoms of a pyridine ring and/or quinazoline ring are not counted in the ring atoms. When a fluorene ring is substituted by a substituent (e.g., a fluorene ring) (i.e., a spirofluorene ring is included), the number of atoms of the fluorene ring as the substituent is not counted in the number of the ring atoms of the fluorene ring.

### Description of Each Substituent in Formulae Herein

The aryl group (occasionally referred to as an aromatic hydrocarbon group) herein is exemplified by an aryl group Sub₁. The aryl group Sub₁ preferably has 6 to ring carbon atoms, more preferably 6 to 20 ring carbon atoms, still more preferably 6 to 14 ring carbon atoms, and still further more preferably 6 to 12 ring carbon atoms.

The aryl group Sub₁ herein is at least one group selected from the group consisting of a phenyl group, biphenyl group, terphenyl group, naphthyl group, anthryl group, phenanthryl group, fluorenyl group, pyrenyl group, chrysenyl group, fluoranthenyl group, benz[a]anthryl group, benzo[c]phenanthryl group, triphenylenyl group, benzo[k]fluoranthenyl group, benzo[g]chrysenyl group, benzo[b]triphenylenyl group, picenyl group, and perylenyl group.

Among the aryl group Sub₁, a phenyl group, biphenyl group, naphthyl group, phenanthryl group, terphenyl group and fluorenyl group are preferable. A carbon atom in a position 9 of each of 1-fluorenyl group, 2-fluorenyl group, 3-fluorenyl group and 4-fluorenyl group is preferably substituted by a substituted or unsubstituted alkyl group Sub₃ or a substituted or unsubstituted aryl group Sub₁ described later herein.

The heteroaryl group (occasionally referred to as a heterocyclic group, heteroaromatic cyclic group or aromatic heterocyclic group) herein is exemplified by a heterocyclic group Sub₂. The heterocyclic group Sub₂ is a group containing, as a hetero atom(s), at least one atom selected from the group consisting of nitrogen, sulfur, oxygen, silicon, selenium atom and germanium atom. The heterocyclic group Sub₂ preferably contains, as a hetero atom(s), at least one atom selected from the group consisting of nitrogen, sulfur and oxygen. The heterocyclic group Sub₂ preferably has 5 to 30 ring atoms, more preferably 5 to 20 ring atoms, and still more preferably 5 to 14 ring atoms.

The heterocyclic group Sub₂ herein are, for instance, at least one group selected from the group consisting of a pyridyl group, pyrimidinyl group, pyrazinyl group, pyridazinyl group, triazinyl group, quinolyl group, isoquinolinyl group, naphthyridinyl group, phthalazinyl group, quinoxalinyl group, quinazolinyl group, phenanthridinyl group, acridinyl group, phenanthrolinyl group, pyrrolyl group, imidazolyl group, pyrazolyl group, triazolyl group, tetrazolyl group, indolyl group, benzimidazolyl group, indazolyl group, imidazopyridinyl group, benzotriazolyl group, carbazolyl group, furyl group, thienyl group, oxazolyl group, thiazolyl group, isoxazolyl group, isothiazolyl group, oxadiazolyl group, thiadiazolyl group, benzofuranyl group, benzothienyl group, benzoxazolyl group, benzothiazolyl group, benzisoxazolyl group, benzisothiazolyl group, benzoxadiazolyl group, benzothiadiazolyl group, dibenzofuranyl group, dibenzothienyl group, piperidinyl group, pyrrolidinyl group, piperazinyl group, morpholyl group, phenazinyl group, phenothiazinyl group, and phenoxazinyl group.

Among the above heterocyclic group Sub₂, a 1-dibenzofuranyl group, 2-dibenzofuranyl group, 3-dibenzofuranyl group, 4-dibenzofuranyl group, 1-dibenzothienyl group, 2-dibenzothienyl group, 3-dibenzothienyl group, 4-dibenzothienyl group, 1-carbazolyl group, 2-carbazolyl group, 3-carbazolyl group, 4-carbazolyl group, and 9-carbazolyl group are further more preferable. A nitrogen atom in a position 9 of each of 1-carbazolyl group, 2-carbazolyl group, 3-carbazolyl group and 4-carbazolyl group is preferably substituted by a substituted or unsubstituted aryl group Sub₁ or a substituted or unsubstituted heterocyclic group Sub₂ described herein.

Herein, the heterocyclic group Sub₂ may be a group derived from any one of partial structures represented by formulae (XY-1) to (XY-18) below.

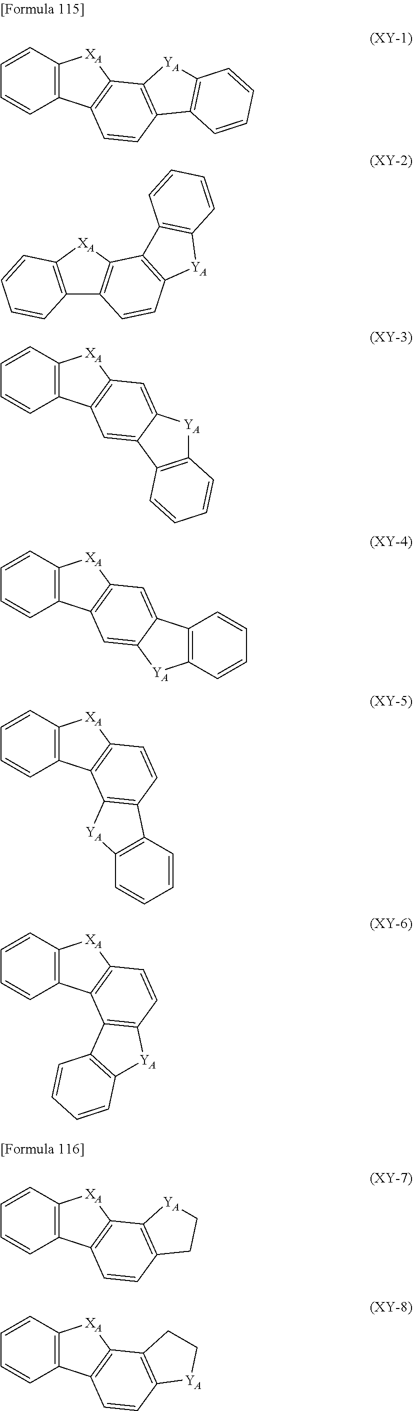

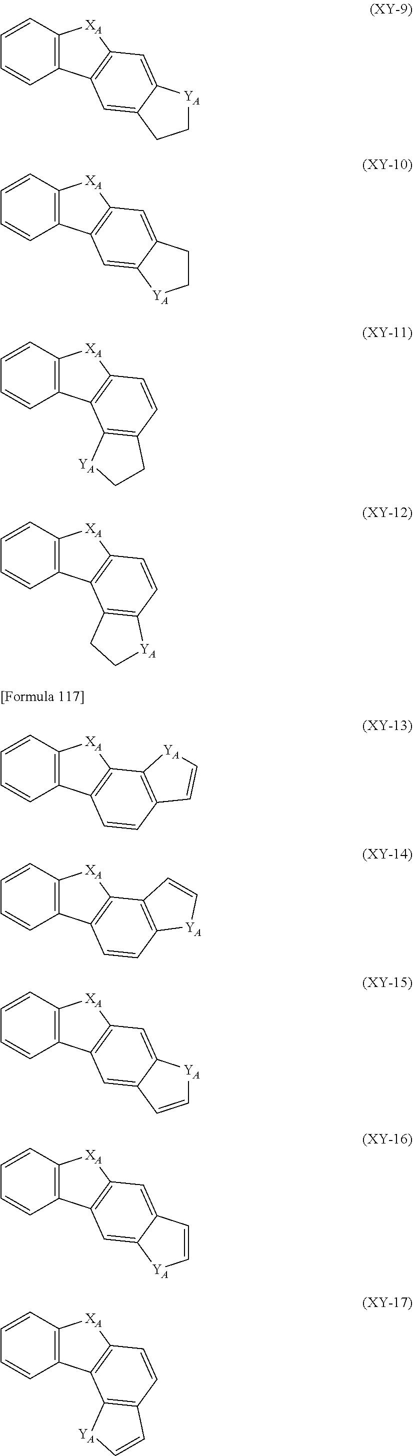

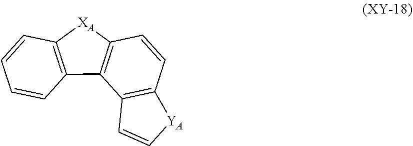

In the formulae (XY-1) to (XY-18), X_{A} and Y_{A} each independently represent a hetero atom, and preferably represent an oxygen atom, sulfur atom, selenium atom, silicon atom or germanium atom. The partial structures represented by the formulae (XY-1) to (XY-18) may each have a bond at any position to provide a heterocyclic group, in which the heterocyclic group may be substituted.

Herein, the heterocyclic group Sub₂ may be a group represented by one of formulae (XY-19) to (XY-22) below. Further, the position of the bond may be changed as needed.

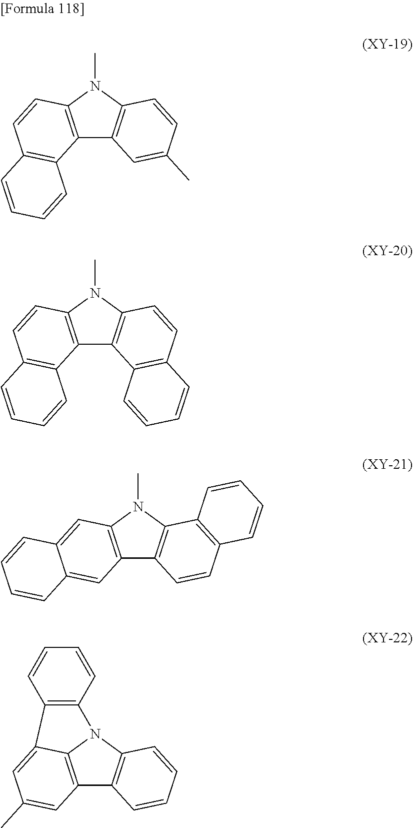

The alkyl group herein may be any one of a linear alkyl group, branched alkyl group and cyclic alkyl group.

The alkyl group herein is exemplified by an alkyl group Sub₃.

The linear alkyl group herein is exemplified by a linear alkyl group Sub₃₁.

The branched alkyl group herein is exemplified by a branched alkyl group Sub₃₂.

The cyclic alkyl group herein is exemplified by a cyclic alkyl group Sub₃₃ (also referred to as a cycloalkyl group Sub₃₃).

For instance, the alkyl group Sub₃ is at least one group selected from the group consisting of the linear alkyl group Sub₃₁, branched alkyl group Sub₃₂, and cyclic alkyl group Sub₃₃.

Herein, the linear alkyl group Sub₃₁ or branched alkyl group Sub₃₂ preferably has 1 to 30 carbon atoms, more preferably 1 to 20 carbon atoms, still more preferably 1 to 10 carbon atoms, and still further more preferably 1 to 6 carbon atoms.

Herein, the cycloalkyl group Sub₃₃ preferably has 3 to 30 ring carbon atoms, more preferably 3 to 20 ring carbon atoms, still more preferably 3 to 10 ring carbon atoms, and still further more preferably 5 to 8 ring carbon atoms.

The linear alkyl group Sub₃₁ or branched alkyl group Sub₃₂ herein is exemplified by at least one group selected from the group consisting of a methyl group, ethyl group, n-propyl group, isopropyl group, n-butyl group, s-butyl group, isobutyl group, t-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, neopentyl group, amyl group, isoamyl group, 1-methylpentyl group, 2-methylpentyl group, 1-pentylhexyl group, 1-butylpentyl group, 1-heptyloctyl group, and 3-methylpentyl group.

The linear alkyl group Sub₃₁ or branched alkyl group Sub₃₂ is still further preferably a methyl group, ethyl group, propyl group, isopropyl group, n-butyl group, s-butyl group, isobutyl group, t-butyl group, n-pentyl group, n-hexyl group, amyl group, isoamyl group and neopentyl group.

The cycloalkyl group Sub₃₃ herein is exemplified by at least one group selected from the group consisting of a cyclopropyl group, cyclobutyl group, cyclopentyl group, cyclohexyl group, 4-metylcyclohexyl group, adamantyl group and norbornyl group. Among the cycloalkyl group Sub₃₃, a cyclopentyl group and a cyclohexyl group are still further preferable.

Herein, an alkyl halide group is exemplified by an alkyl halide group Sub₄. The alkyl halide group Sub₄ is provided by substituting the alkyl group Sub₃ with at least one halogen atom, preferably at least one fluorine atom.

Herein, the alkyl halide group Sub₄ is exemplified by at least one group selected from the group consisting of a fluoromethyl group, difluoromethyl group, trifluoromethyl group, fluoroethyl group, trifluoromethylmethyl group, trifluoroethyl group, and pentafluoroethyl group.

Herein, a substituted silyl group is exemplified by a substituted silyl group Sub₅. The substituted silyl group Sub₅ is exemplified by at least one group selected from the group consisting of an alkylsilyl group Sub₅₁ and an arylsilyl group Sub₅₂.

Herein, the alkylsilyl group Sub₅₁ is exemplified by a trialkylsilyl group Sub₅₁₁ having the above-described alkyl group Sub₃.

The trialkylsilyl group Sub₅₁₁ is exemplified by at least one group selected from the group consisting of a trimethylsilyl group, triethylsilyl group, tri-n-butylsilyl group, tri-n-octylsilyl group, triisobutylsilyl group, dimethylethylsilyl group, dimethylisopropylsilyl group, dimethyl-n-propylsilyl group, dimethyl-n-butylsilyl group, dimethyl-t-butylsilyl group, diethylisopropylsilyl group, vinyl dimethylsilyl group, propyldimethylsilyl group, and triisopropylsilyl group. Three alkyl groups Sub₃ in the trialkylsilyl group Sub₅₁₁ may be mutually the same or different.

Herein, the arylsilyl group Sub₅₂ is exemplified by at least one group selected from the group consisting of a dialkylarylsilyl group Sub₅₂₁, alkyldiarylsilyl group Sub₅₂₂ and triarylsilyl group Sub₅₂₃.

The dialkylarylsilyl group Sub₅₂₁ is exemplified by a dialkylarylsilyl group including two alkyl groups Sub₃ and one aryl group Sub₁. The dialkylarylsilyl group Sub₅₂₁ preferably has 8 to 30 carbon atoms.

The alkyldiarylsilyl group Sub₅₂₂ is exemplified by an alkyldiarylsilyl group including one alkyl group Sub₃ and two aryl groups Sub₁. The alkyldiarylsilyl group Sub₅₂₂ preferably has 13 to 30 carbon atoms.

The triarylsilyl group Sub₅₂₃ is exemplified by a triarylsilyl group including three aryl groups Sub₁. The triarylsilyl group Sub₅₂₃ preferably has 18 to 30 carbon atoms.

Herein, a substituted or unsubstituted alkyl sulfonyl group is exemplified by an alkyl sulfonyl group Sub₆. The alkyl sulfonyl group Sub₆ is represented by —SO₂Rw. R_{w} in —SO₂R_{w} represents a substituted or unsubstituted alkyl group Sub₃ described above.

Herein, an aralkyl group (occasionally referred to as an arylalkyl group) is exemplified by an aralkyl group Sub₇. An aryl group in the aralkyl group Sub₇ includes, for instance, at least one of the above-described aryl group Sub₁ or the above-described heteroaryl group Sub₂.

The aralkyl group Sub₇ herein is preferably a group having the aryl group Sub₁ and is represented by —Z₃-Z₄. Z₃ is exemplified by an alkylene group corresponding to the above alkyl group Sub₃. Z₄ is exemplified by the above aryl group Sub₁. In this aralkyl group Sub₇, an aryl moiety has 6 to 30 carbon atoms (preferably 6 to 20 carbon atoms, more preferably 6 to 12 carbon atoms) and an alkyl moiety has 1 to 30 carbon atoms (preferably 1 to 20 carbon atoms, more preferably 1 to 10 carbon atoms, and still more preferably 1 to 6 carbon atoms). The aralkyl group Sub₇ is exemplified by at least one group selected from the group consisting of a benzyl group, 2-phenylpropane-2-yl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

The alkoxy group herein is exemplified by an alkoxy group Sub₈. The alkoxy group Sub₈ is represented by —OZ₁. Z₁ is exemplified by the above alkyl group Sub₃. The alkoxy group Sub₈ preferably has 1 to 30 carbon atoms, more preferably 1 to 20 carbon atoms. The alkoxy group Sub₈ is exemplified by at least one group selected from the group consisting of a methoxy group, ethoxy group, propoxy group, butoxy group, pentyloxy group and hexyloxy group.

Herein, an alkoxy halide group is exemplified by an alkoxy halide group Sub₉. The alkoxy halide group Sub₉ is provided, for instance, by substituting the alkoxy group Sub₈ with at least one halogen atom, preferably at least one fluorine atom.

Herein, an aryloxy group (occasionally referred to as an arylalkoxy group) is exemplified by an arylalkoxy group Sub₁₀. An aryl group in the arylalkoxy group Sub₁₀ includes at least one of the aryl group Sub₁ or the heteroaryl group Sub₂.

The arylalkoxy group Sub₁₀ herein is represented by —OZ₂. Z₂ is exemplified by the aryl group Sub₁ or the heteroaryl group Sub₂. The arylalkoxy group Sub₁₀ preferably has 6 to 30 ring carbon atoms, more preferably 6 to 20 ring carbon atoms. The arylalkoxy group Sub₁₀ is exemplified by a phenoxy group.

Herein, a substituted amino group is exemplified by a substituted amino group Sub₁₁. The substituted amino group Sub₁₁ is exemplified by at least one group selected from the group consisting of an arylamino group Sub₁₁₁ and an alkylamino group Sub₁₁₂.

The arylamino group Sub₁₁₁ is represented by —NHR_{V1} or —N(R_{V1})₂. R_{V1} is exemplified by the aryl group Sub₁. Two R_{V1} in —N(R_{V1})₂ are mutually the same or different.

The alkylamino group Sub₁₁₂ is represented by —NHR_{V2} or —N(R_{V2})₂. R_{V2} is exemplified by the alkyl group Sub₃. Two R_{V2} in —N(R_{V2})₂ are mutually the same or different.

Herein, the alkenyl group is exemplified by an alkenyl group Sub₁₂. The alkenyl group Sub₁₂, which is linear or branched, is exemplified by at least one group selected from the group consisting of a vinyl group, propenyl group, butenyl group, oleyl group, eicosapentaenyl group, docosahexaenyl group, styryl group, 2,2-diphenylvinyl group, 1,2,2-triphenylvinyl group, and 2-phenyl-2-propenyl group.

The alkynyl group herein is exemplified by an alkynyl group Sub₁₃. The alkynyl group Sub₁₃ may be linear or branched and is at least one group selected from the group consisting of an ethynyl group, a propynyl group and a 2-phenylethynyl group.

The alkylthio group herein is exemplified by an alkylthio group Sub₁₄.

The alkylthio group Sub₁₄ is represented by —SR_{V3}. R_{V3} is exemplified by the alkyl group Sub₃. The alkylthio group Sub₁₄ preferably has 1 to 30 carbon atoms, more preferably 1 to 20 carbon atoms.

The arylthio group herein is exemplified by an arylthio group Sub₁₅.

The arylthio group Sub₁₅ is represented by —SR_{V4}. R_{V4} is exemplified by the aryl group Sub₁. The arylthio group Sub₁₅ preferably has 6 to 30 ring carbon atoms, more preferably 6 to 20 ring carbon atoms.

Herein, examples of the halogen atom include a fluorine atom, a chlorine atom, a bromine atom and an iodine atom, among which a fluorine atom is preferable.

A substituted phosphino group herein is exemplified by a substituted phosphino group Sub₁₆. The substituted phosphino group Sub₁₆ is exemplified by a phenyl phosphanyl group.

An arylcarbonyl group herein is exemplified by an arylcarbonyl group Sub₁₇. The arylcarbonyl group Sub₁₇ is represented by —COY′. Y′ is exemplified by the aryl group Sub₁. Herein, the arylcarbonyl group Sub₁₇ is exemplified by at least one group selected from the group consisting of a phenyl carbonyl group, diphenyl carbonyl group, naphthyl carbonyl group, and triphenyl carbonyl group.

An acyl group herein is exemplified by an acyl group Sub₁₈. The acyl group Sub₁₈ is represented by —COR′. R′ is exemplified by the alkyl group Sub₃. The acyl group Sub₁₈ herein is exemplified by at least one group selected from the group consisting of an acetyl group and a propionyl group.

A substituted phosphoryl group herein is exemplified by a substituted phosphoryl group Sub₁₉. The substituted phosphoryl group Sub₁₉ is represented by a formula (P) below.

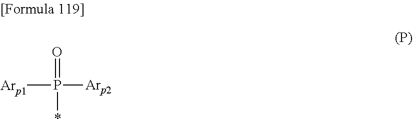

In the formula (P), Ar_{P1} and Ar_{P2} are any one substituent selected from the group consisting of the above alkyl group Sub₃ and the above aryl group Sub₁.

An ester group herein is exemplified by an ester group Sub₂₀. The ester group Sub₂₀ is exemplified by at least one group selected from the group consisting of an alkyl ester group and an aryl ester group.

An alkyl ester group herein is exemplified by an alkyl ester group Sub₂₀₁. The alkyl ester group Sub₂₀₁ is represented by —C(═O)OR^{E}. R^{E} is exemplified by a substituted or unsubstituted alkyl group Sub₃ described above.

An aryl ester group herein is exemplified by an aryl ester group Sub₂₀₂. The aryl ester group Sub₂₀₂ is represented by —C(═O)OR^{Ar}. R^{Ar} is exemplified by a substituted or unsubstituted aryl group Sub₁ described above.

A siloxanyl group herein is exemplified by a siloxanyl group Sub₂₁. The siloxanyl group Sub₂₁ is a silicon compound group through an ether bond. The siloxanyl group Sub₂₁ is exemplified by a trimethylsiloxanyl group.

A carbamoyl group herein is represented by —CONH₂.

A substituted carbamoyl group herein is exemplified by a carbamoyl group Sub₂₂. The carbamoyl group Sub₂₂ is represented by —CONH—Ar^{C} or —CONH—R^{C}. Ar^{C} is exemplified by at least one group selected from the group consisting of a substituted or unsubstituted aryl group Sub₁ described above (preferably 6 to 10 ring carbon atoms) and the above-described heteroaryl group Sub₂ (preferably 5 to 14 ring atoms). Ar^{C} may be a group formed by bonding the aryl group Sub₁ and the heteroaryl group Sub₂.

R^{C} is exemplified by a substituted or unsubstituted alkyl group Sub₃ described above (preferably having 1 to 6 carbon atoms).

Herein, “carbon atoms forming a ring (ring carbon atoms)” mean carbon atoms forming a saturated ring, unsaturated ring, or aromatic ring. “Atoms forming a ring (ring atoms)” mean carbon atoms and hetero atoms forming a hetero ring including a saturated ring, unsaturated ring, or aromatic ring.

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Hereinafter, an alkyl group Sub₃ means at least one group of a linear alkyl group Sub₃₁, a branched alkyl group Sub₃₂, or a cyclic alkyl group Sub₃₃ described in “Description of Each Substituent.”

Similarly, a substituted silyl group Sub₅ means at least one group of an alkylsilyl group Sub₅₁ or an arylsilyl group Sub₅₂.

Similarly, a substituted amino group Sub₁₁ means at least one group of an arylamino group Sub₁₁₁ or an alkylamino group Sub₁₁₂.

Herein, a substituent for a “substituted or unsubstituted” group is exemplified by a substituent R_{F1}. The substituent R_{F1} is at least one group selected from the group consisting of an aryl group Sub₁, heteroaryl group Sub₂, alkyl group Sub₃, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₅, arylcarbonyl group Sub₁₇, acyl group Sub₁₈, substituted phosphoryl group Sub₁₉, ester group Sub₂₀, siloxanyl group Sub₂₁, carbamoyl group Sub₂₂, unsubstituted amino group, unsubstituted silyl group, halogen atom, cyano group, hydroxy group, nitro group, and carboxy group.

Herein, the substituent R_{F1} for a “substituted or unsubstituted” group may be a diaryl boron group (Ar_{B1}Ar_{B2}B—). Ar_{B1} and Ar_{B2} are exemplified by the above-described aryl group Sub₁. Ar_{B1} and Ar_{B2} in Ar_{B1}Ar_{B2}B— are the same or different.

Specific examples and preferable examples of the substituent R_{F1} are the same as those of the substituents described in “Description of Each Substituent” (e.g., an aryl group Sub₁, heteroaryl group Sub₂, alkyl group Sub₃, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₆, arylcarbonyl group Sub₁₇, acyl group Sub₁₈, substituted phosphoryl group Sub₁₉, ester group Sub₂₀, siloxanyl group Sub₂₁, and carbamoyl group Sub₂₂).

The substituent R_{F1} for a “substituted or unsubstituted” group may be further substituted by at least one group (hereinafter, also referred to as a substituent R_{F2}) selected from the group consisting of an aryl group Sub₁, heteroaryl group Sub₂, alkyl group Sub₃, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₆, arylcarbonyl group Sub₁₇, acyl group Sub₁₈, substituted phosphoryl group Sub₁₉, ester group Sub₂₀, siloxanyl group Sub₂₁, carbamoyl group Sub₂₂, unsubstituted amino group, unsubstituted silyl group, halogen atom, cyano group, hydroxy group, nitro group, and carboxy group. Moreover, a plurality of substituents R_{F2} may be bonded to each other to form a ring.

“Unsubstituted” for a “substituted or unsubstituted” group means that a group is not substituted by the above-described substituent R_{F1} but bonded with a hydrogen atom.

Herein, “XX to YY carbon atoms” in the description of “substituted or unsubstituted ZZ group having XX to YY carbon atoms” represent carbon atoms of an unsubstituted ZZ group and do not include carbon atoms of the substituent R_{F1} of the substituted ZZ group.

Herein, “XX to YY atoms” in the description of “substituted or unsubstituted ZZ group having XX to YY atoms” represent atoms of an unsubstituted ZZ group and do not include atoms of the substituent R_{F1} of the substituted ZZ group.

The same description as the above applies to “substituted or unsubstituted” in compounds or partial structures thereof described herein.

Herein, when the substituents are bonded to each other to form a ring, the ring is structured to be a saturated ring, an unsaturated ring, an aromatic hydrocarbon ring or a hetero ring.

Herein, examples of the aromatic hydrocarbon group in the linking group include a divalent or multivalent group obtained by eliminating one or more atoms from the above monovalent aryl group Sub₁.

Herein, examples of the heterocyclic group in the linking group include a divalent or multivalent group obtained by eliminating one or more atoms from the above monovalent heteroaryl group Sub₂.

### EXAMPLES

### Compounds

A structure of a compound D1 as the first compound used for producing organic EL devices is shown below.

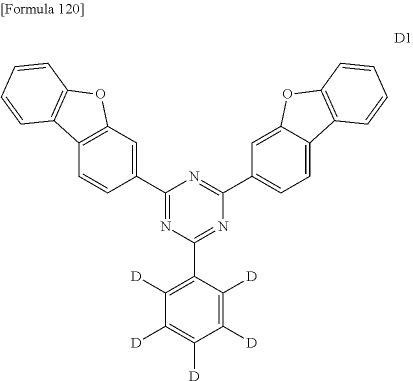 [SMILES: [2H]c1c([2H])c([2H])c(-c2nc(-c3ccc4c(c3)oc3ccccc34)nc(-c3ccc4c(c3)oc3ccccc34)n2)c([2H])c1[2H]]

A structure of a compound used for producing organic EL devices in Comparatives is shown below.

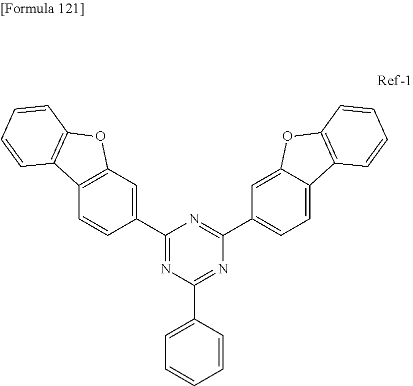 [SMILES: c1ccc(-c2nc(-c3ccc4c(c3)oc3ccccc34)nc(-c3ccc4c(c3)oc3ccccc34)n2)cc1]

Structures of other compounds used for producing organic EL devices in Examples and Comparatives are shown below.

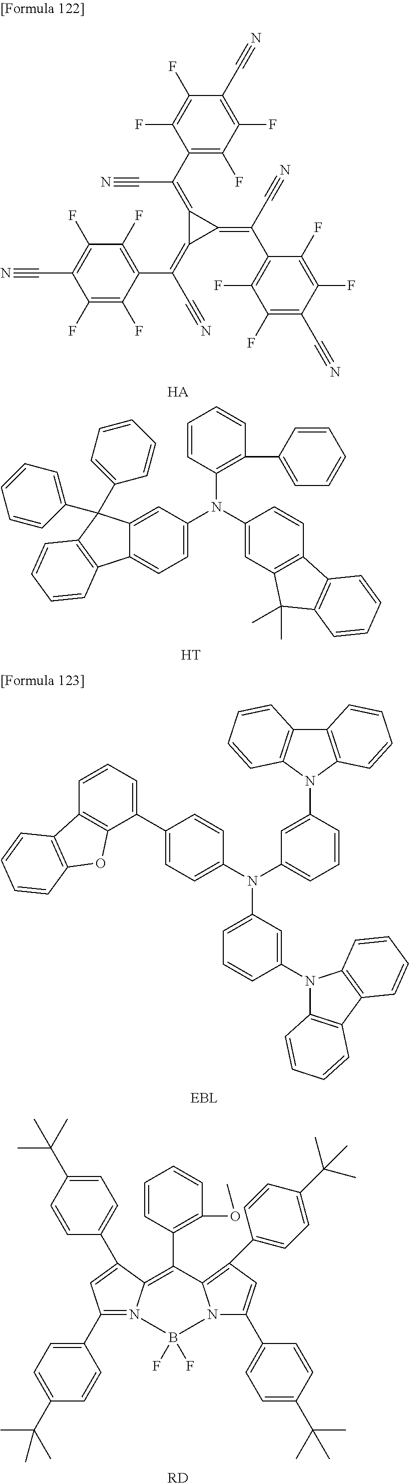

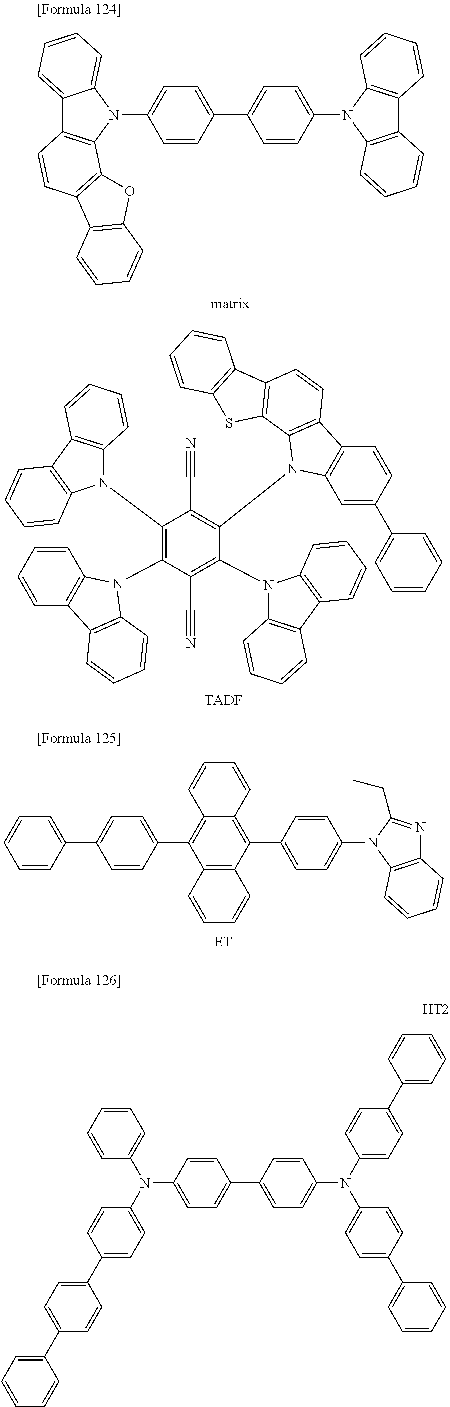

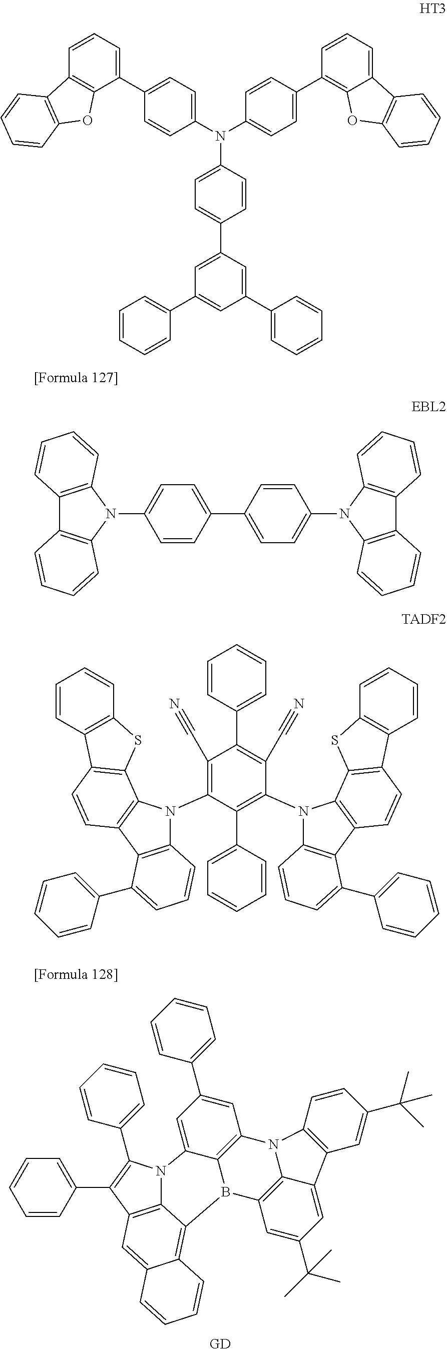 [SMILES: c1ccc(-c2cc(-c3ccccc3)cc(-c3ccc(N(c4ccc(-c5cccc6c5oc5ccccc56)cc4)c4ccc(-c5cccc6c5oc5ccccc56)cc4)cc3)c2)cc1 ; c1ccc2c(c1)c1ccccc1n2-c1ccc(-c2ccc(-n3c4ccccc4c4ccccc43)cc2)cc1 ; N#Cc1c(-c2ccccc2)c(C#N)c(-n2c3cccc(-c4ccccc4)c3c3ccc4c5ccccc5sc4c32)c(-c2ccccc2)c1-n1c2cccc(-c3ccccc3)c2c2ccc3c4ccccc4sc3c21 ; CC(C)(C)c1ccc2c(c1)c1cc(C(C)(C)C)cc3c1n2-c1cc(-c2ccccc2)cc2c1B3c1c3ccccc3cc3c(-c4ccccc4)c(-c4ccccc4)n-2c13]

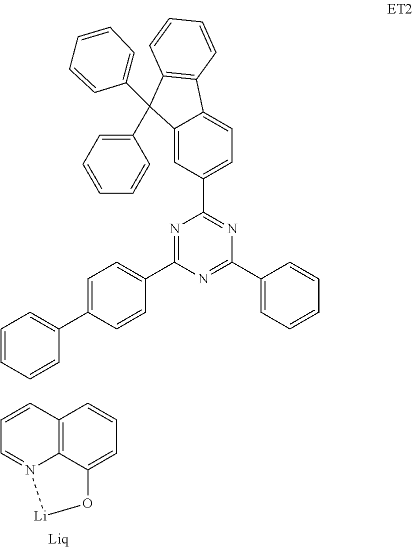

### Preparation 1 of Organic EL Device

The organic EL devices were prepared and evaluated as follows.

### Example 1

A glass substrate (size: 25 mm×75 mm×1.1 mm thick, produced by Geomatec Co., Ltd.) having an ITO transparent electrode (anode) was ultrasonic-cleaned in isopropyl alcohol for five minutes, and then UV/ozone-cleaned for one minute. The film thickness of ITO was 130 nm.

After the glass substrate having the transparent electrode line was cleaned, the glass substrate was mounted on a substrate holder of a vacuum evaporation apparatus. Firstly, a compound HT and a compound HA were co-deposited on a surface of the glass substrate where the transparent electrode line was provided in a manner to cover the transparent electrode, thereby forming a 10-nm-thick hole injecting layer. The concentrations of the compound HT and the compound HA in the hole injecting layer were 97 mass % and 3 mass %, respectively.

Next, the compound HT was vapor-deposited on the hole injecting layer to form a 200-nm-thick hole transporting layer.

Next, a compound EBL was vapor-deposited on the hole transporting layer to form a 10-nm-thick electron blocking layer.

Next, a compound matrix as the compound M3, a compound TADF as the compound M2, and a compound RD as the compound M1 were co-deposited on the electron blocking layer to form a 25-nm-thick emitting layer. The concentrations of the compound Matrix, the compound TADF, and the compound RD in the emitting layer were 74 mass %, 25 mass %, and 1 mass % respectively.

Next, the compound D1 as the first compound was vapor-deposited on the emitting layer to form a 10-nm-thick hole blocking layer (the first layer).

Next, a compound ET was deposited on the hole blocking layer (the first layer) to form a 30-nm-thick electron transporting layer.

Next, lithium fluoride (LiF) was vapor-deposited on the electron transporting layer to form a 1-nm-thick electron injecting electrode (cathode).

Subsequently, metal aluminum (Al) was vapor-deposited on the electron injectable electrode to form an 80-nm-thick metal Al cathode.

A device arrangement of the organic EL device in Example 1 is roughly shown as follows.
ITO(130)/HT: HA(10.97%:3%)/HT(200)/EBL(10)/matrix:TADF:RD(25.74%:25%:1%)/D1 (10)/ET(30)/LiF(1)/Al(80)

Numerals in parentheses represent a film thickness (unit: nm). The numerals (97%:3%) indicate a ratio (mass %) between the compound HT and the compound HA in the hole injecting layer. The numerals (74%:25%:1%) represented by percentage indicate a ratio (mass %) between the compound matrix, the compound TADF, and the compound RD in the emitting layer.

### Comparative 1

The organic EL device in Comparative 1 was produced in the same manner as in Example 1 except that the compound D1 in the hole blocking layer (the first layer) in Example 1 was replaced by a compound shown in Table 1.

### Evaluation of Organic EL Device

The organic EL devices produced in Example 1 and Comparative 1 were evaluated as follows. Table 1 shows the results. Although a compound Ref-1 used in Comparative 1 does not correspond to the first compound, the compound Ref-1 is shown in the same column as the compound D1 in Example 1 for convenience.

### Main Peak Wavelength (λp)

Voltage was applied to the organic EL devices such that a current density of the organic EL device was 10 mA/cm², where spectral radiance spectrum was measured by a spectroradiometer CS-2000 (produced by Konica Minolta, Inc.). The main peak wavelength λp (unit: nm) was calculated based on the obtained spectral-radiance spectra.

### External Quantum Efficiency EQE

Voltage was applied to the organic EL devices such that a current density was 10 mA/cm², where spectral radiance spectrum was measured by a spectroradiometer CS-2000 (produced by Konica Minolta, Inc.). The external quantum efficiency EQE (unit: %) was calculated based on the obtained spectral radiance spectra, assuming that the spectra was provided under a Lambertian radiation.

### Drive Voltage

A voltage (unit: V) was measured when current was applied between the anode and the cathode such that a current density was 10 mA/cm².

### Lifetime LT95

Voltage was applied to the organic EL devices such that a current density was 50 mA/cm², where a time (unit: h) elapsed before a luminance intensity was reduced to 95% of the initial luminance intensity was measured using a spectroradiometer CS-200 (manufactured by Konica Minolta, Inc.).

TABLE 1

| | Emitting Layer | | | Hole Blocking Layer | Device Evaluation Results | | | |
|---|---|---|---|---|---|---|---|---|
| | Compound | Compound | Compound | (First Layer) | λp | Voltage | EQE | LT95 |
| | M1 | M2 | M3 | First Compound | [nm] | [V] | [%] | [hr] |
| Ex. 1 | RD | TADF | matrix | D1 | 621 | 4.6 | 17.1 | 163 |
| Comp. 1 | RD | TADF | matrix | Ref-1 | 621 | 4.6 | 17.1 | 116 |

The lifetime of the device in Example 1 in which the compound D1 having a deuterium atom was used in the hole blocking layer (first layer) was considerably longer than that of the device in Comparative 1 in which the compound D1 in Example 1 was replaced with the “compound Ref-1 having no deuterium atom”.

### Preparation 2 of Organic EL Device

### Example 2

A glass substrate (size: 25 mm×75 mm×1.1 mm thick, produced by Geomatec Co., Ltd.) having an ITO transparent electrode (anode) was ultrasonic-cleaned in isopropyl alcohol for five minutes, and then UV/ozone-cleaned for one minute. The film thickness of ITO was 130 nm.

After the glass substrate having the transparent electrode line was cleaned, the glass substrate was mounted on a substrate holder of a vacuum evaporation apparatus. Firstly, a compound HT2 and the compound HA were co-deposited on a surface of the glass substrate where the transparent electrode line was provided in a manner to cover the transparent electrode, thereby forming a 10-nm-thick hole injecting layer. The concentrations of the compound HT2 and the compound HA in the hole injecting layer were 97 mass % and 3 mass %, respectively.

Next, the compound HT2 was vapor-deposited on the hole injecting layer to form a 110-nm-thick first hole transporting layer

Next, a compound HT3 was vapor-deposited on the first hole transporting layer to form a 5-nm-thick second hole transporting layer.

Next, a compound EBL2 was vapor-deposited on the second hole transporting layer to form a 5-nm-thick electron blocking layer.

Next, the compound matrix as the compound M3, a compound TADF2 as the compound M2, and a compound GD as the compound M1 were co-deposited on the electron blocking layer to form a 25-nm-thick emitting layer. The concentrations of the compound Matrix, the compound TADF2, and the compound GD in the emitting layer were 74 mass %, 25 mass %, and 1 mass % respectively.

Next, the compound D1 as the first compound was vapor-deposited on the emitting layer to form a 5-nm-thick hole blocking layer (the first layer).

Next, a compound ET2 and a compound Liq were co-deposited on the hole blocking layer (the first layer) to form a 50-nm-thick electron transporting layer. The concentrations of the compound ET2 and the compound Liq in the electron transporting layer were 50 mass % and 50 mass %, respectively.

Next, Yb was vapor-deposited on the electron transporting layer to form a 1-nm-thick electron injecting electrode (cathode).

Subsequently, metal aluminum (Al) was vapor-deposited on the electron injectable electrode to form an 80-nm-thick metal Al cathode.

A device arrangement of the organic EL device in Example 2 is roughly shown as follows.
ITO(130)/HT2: HA(10.97%:3%)/HT2(110)/HT3(5)/EBL2(5)/matrix:TADF2:GD(25.74%:25%:1%)/D1(5)/ET2:Liq(50.50%:50%)/Yb(1)/Al(80)

Numerals in parentheses represent a film thickness (unit: nm). The numerals (97%:3%) indicate a ratio (mass %) between the compound HT2 and the compound HA in the hole injecting layer. The numerals (74%:25%:1%) represented by percentage indicate a ratio (mass %) between the compound matrix, the compound TADF2, and the compound GD in the emitting layer. The numerals (50%:50%) indicate a ratio (mass %) between the compound ET2 and the compound Liq in the electron transporting layer.

### Comparative 2

The organic EL device in Comparative 2 was produced in the same manner as in Example 2 except that the compound D1 in the hole blocking layer (the first layer) in Example 2 was replaced by a compound shown in Table 2.

### Evaluation of Organic EL Device

The organic EL devices produced in Example 2 and Comparative 2 were measured for the main peak wavelength λp, the external quantum efficiency EQE, the drive voltage, and the lifetime LT95 in the same manner as in Example 1. Table 2 shows the results.

TABLE 2

| | Emitting Layer | | | Hole Blocking Layer | Device Evaluation Results | | | |
|---|---|---|---|---|---|---|---|---|
| | Compound | Compound | Compound | (First Layer) | λp | Voltage | EQE | LT95 |
| | M1 | M2 | M3 | First Compound | [nm] | [V] | [%] | [hr] |
| Ex. 2 | GD | TADF2 | matrix | D1 | 520 | 4.2 | 16.2 | 34 |
| Comp. 2 | GD | TADF2 | matrix | Ref-1 | 520 | 4.2 | 16.1 | 30 |

The device in Example 2 in which the compound D1 having a deuterium atom was used in the hole blocking layer (first layer) emitted light more efficiently and had a longer lifetime than the device in Comparative 2 in which the compound D1 in Example 2 was replaced with the “compound Ref-1 having no deuterium atom”.

### Evaluation of Compounds

Physical properties of compounds described in Tables 1 and 2 were measured according to the following methods. Table 3 shows the results.

### Thermally Activated Delayed Fluorescence

### Delayed Fluorescence of Compound TADF

Thermally activated delayed fluorescence was checked by measuring transient PL using a device shown in FIG. 2. The compound TADF was dissolved in toluene to prepare a dilute solution with an absorbance of 0.05 or less at the excitation wavelength to eliminate the contribution of self-absorption. In order to prevent quenching due to oxygen, the sample solution was frozen and degassed and then sealed in a cell with a lid under an argon atmosphere to obtain an oxygen-free sample solution saturated with argon.

The fluorescence spectrum of the above sample solution was measured with a spectrofluorometer FP-8600 (produced by JASCO Corporation), and the fluorescence spectrum of a 9,10-diphenylanthracene ethanol solution was measured under the same conditions. Using the fluorescence area intensities of both spectra, the total fluorescence quantum yield is calculated by an equation (1) in Morris et al. J. Phys. Chem. 80 (1976) 969.

Prompt emission was observed immediately when the excited state was achieved by exciting the compound TADF with a pulse beam (i.e., a beam emitted from a pulse laser) having a wavelength to be absorbed by the compound TADF, and Delay emission was observed not immediately when the excited state was achieved but after the excited state was achieved. The delayed fluorescence in Examples means that an amount of Delay emission is 5% or more with respect to an amount of Prompt emission. Specifically, provided that the amount of Prompt emission is denoted by XP and the amount of Delay emission is denoted by XD, the delayed fluorescence means that a value of XD/XP is 0.05 or more.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

It was confirmed that the amount of Delay emission was 5% or more with respect to the amount of Prompt emission in the compound TADF.

Specifically, the value of XD/XP was 0.05 or more in the compound TADF.

### Delayed Fluorescence of Compound TADF2

Delayed fluorescence of the compound TADF2 was checked in the same manner as the above except that the compound TADF was replaced by the compound TADF2.

In the compound TADF2, the value of XD/XP was 0.05 or more.

### Singlet Energy S₁

A singlet energy S₁ of each of the compound RD, the compound GD, the compound TADF, the compound TADF2, and the compound matrix was measured according to the above-described solution method.

### Energy Gap at 77K

An energy gap T_{77K} of each of the compound TADF, the compound TADF2, and the compound matrix at 77K was measured according to the measurement method of the energy gap T_{77K} described in the above “Relationship between Triplet Energy and Energy Gap at 77K”.

T_{77K} of the compound matrix was 2.89 eV.

### ΔST

ΔST was calculated based on the measured singlet energy S₁ and energy gap T_{77K} at 77K.

### Main Peak Wavelength λ of Compound

A main peak wavelength A of each of the compounds was measured according to the following method.

A toluene solution of a measurement target compound at a concentration of 5 μmol/L was prepared and put in a quartz cell. An emission spectrum (ordinate axis: luminous intensity, abscissa axis: wavelength) of the thus-obtained sample was measured at a normal temperature (300K). In Examples, the emission spectrum was measured using a spectrophotometer produced by Hitachi, Ltd. (device name: F-7000). It should be noted that the machine for measuring the emission spectrum is not limited to the machine used herein. A peak wavelength of the emission spectrum exhibiting the maximum luminous intensity was defined as a main peak wavelength λ.

TABLE 3

| | | S₁ | ΔST | λ |
|---|---|---|---|---|
| | Name | [eV] | [eV] | [nm] |
| Compound | RD | 2.02 | — | 609 |
| M1 | GD | 2.39 | — | 512 |
| Compound | TADF | 2.34 | <0.01 | 539 |
| M2 | TADF2 | 2.61 | <0.01 | 499 |
| Compound | matrix | 3.42 | 0.53 | — |
| M3 | | | | |

### Synthesis of Compound

### (1) Synthesis Example 1: Synthesis of Compound D1

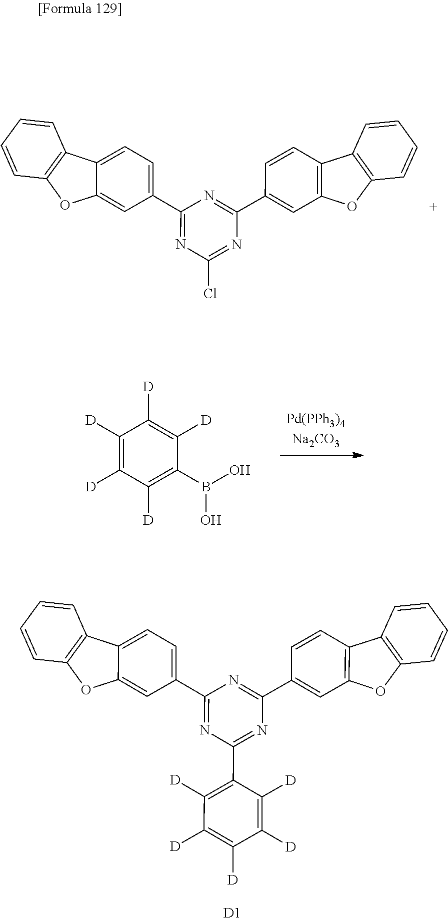

Under nitrogen atmosphere, 1,2-dimethoxyethane (70 mL) and water (35 mL) were added to a mixture of 2-chloro-4,6-bis(dibenzo[b,d]furan-3-yl)-1,3,5-triazine (4.48 g, 10.0 mmol), (phenyl-d5)boronic acid (1.65 g, 13.0 mmol), tetrakis(triphenylphosphine)palladium (577.8 mg, 0.500 mmol), and sodium carbonate (3.18 g, 30.0 mmol) and stirred at 80 degrees C. for six hours. After the reaction, a solid was filtrated and recrystallized with toluene to obtain the compound D1 (3.60 g, a yield of 73%). The obtained compound was identified as the compound D1 by analysis according to Liquid chromatography mass spectrometry (LC-MS).

### (2) Synthesis Example 2: Synthesis of Compound GD

### Production of Intermediate M21

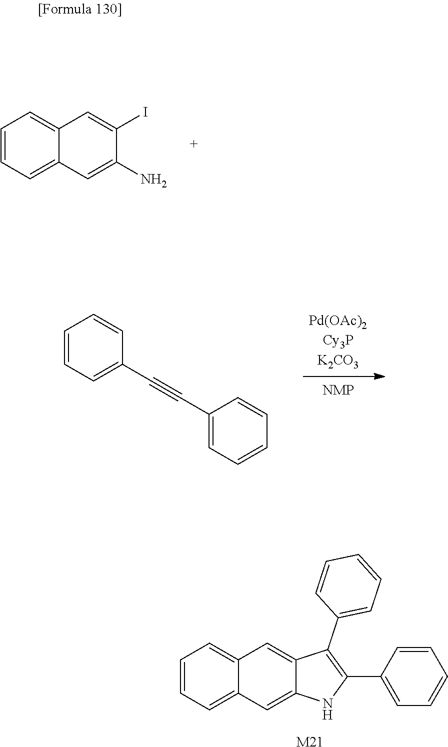

Under argon atmosphere, a mixture of 2-amino-3-iodonaphthalene (4.28 g), 1,2-diphenylacetylene (3.40 g), palladium(II) acetate (178 mg), tricyclohexylphosphine (446 mg), potassium carbonate (5.49 g) and N-methylpyrrolidone (360 mL) was stirred at 110 degrees C. for five hours. The resultant mixture was cooled to a room temperature (25 degrees C.). After the mixture was distilled under reduced pressure to remove a part of N-methylpyrrolidone, the mixture was diluted with t-butylmethylether and added to water. An aqueous layer was extracted by t-butylmethylether and an organic layer was washed with saturated saline solution. Subsequently, the organic layer was dried with magnesium sulfate and was distilled under reduced pressure to remove solvent. The obtained residue was purified by silica gel column chromatography to obtain 2.78 g of the intermediate M21 (55%). In a reaction scheme, Pd(OAc)₂ represents palladium(II) acetate, Cy₃P represents tricyclohexylphosphine, and NMP represents N-methylpyrrolidone.

### Production of Intermediate M22

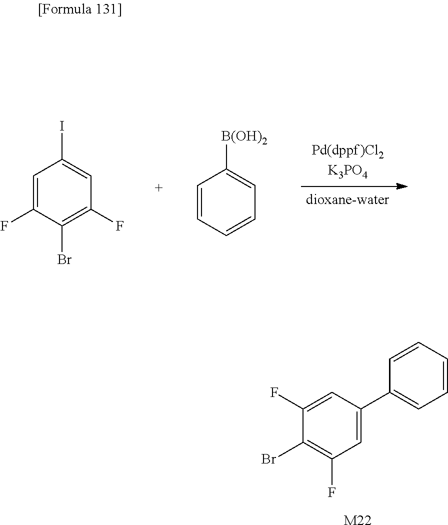

Under argon atmosphere, a mixture of 2-bromo-1,3-difluoro-5-iodobenzene (47.8 g), phenylboronic acid (18.29 g), tripotassium phosphate (39.8 g), [1,1-bis(diphenylphosphino) ferrocene]palladium(II) dichloride (1.09 g), 1,4-dioxane (250 mL), and water (125 mL) was stirred at room temperature for four hours. Toluene (250 mL) and water (200 mL) were added to the obtained mixture. An aqueous layer was extracted with toluene. After an organic layer was washed with saturated saline solution, the organic layer was dried with magnesium sulfate and was distilled under reduced pressure to remove solvent. The obtained residue was purified by silica gel column chromatography to obtain 35.1 g of the intermediate M22 (87%). In a reaction scheme, Pd(dppf)Cl₂ represents [1,1-bis(diphenylphosphino) ferrocene]palladium(II) dichloride.

### Production of Intermediate M23

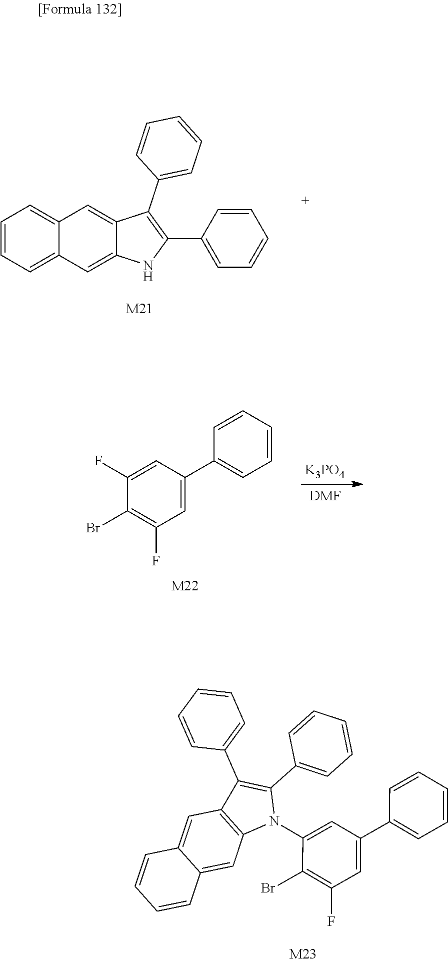

Under argon atmosphere, a mixture of the intermediate M21 (6.39 g), the intermediate M22 (10.76 g), tripotassium phosphate (21.23 g), and dimethylformamide (140 mL) was stirred at 105 degrees C. for 48 hours. After dimethylformamide was partially distilled under reduced pressure, the mixture was put into water and was subjected to extraction using t-butylmethylether. After an organic layer was washed with saturated saline solution, the organic layer was dried with magnesium sulfate and was distilled under reduced pressure to remove solvent. The obtained residue was purified by silica gel column chromatography to obtain 6.2 g of the intermediate M23 (55%). In a reaction scheme, DMF represents dimethylformamide.

### Production of Intermediate M24

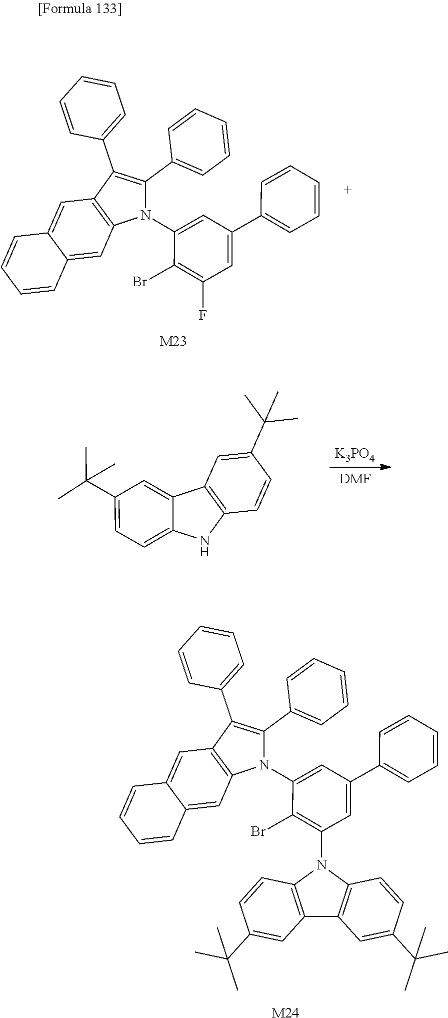

Under argon atmosphere, a mixture of the intermediate M23 (6.14 g), 3,6-di-tert-butyl-9H-carbazole (3.32 g), tripotassium phosphate (6.88 g), and dimethylformamide (96 mL) was stirred at 105 degrees C. for 20 hours. Dimethylformamide was partially distilled under reduced pressure, and the resultant mixture was put into water (150 mL) to be stirred. Deposited solid was separated by filtration and was washed with water. Subsequently, the solid was dried under reduced pressure. Further, the obtained solid was suspended in ethanol (220 mL) and heated for reflux for one hour. Then, the solid was collected by filtration to obtain an intermediate M24 (7.31 g, 82%).

### Production of Compound GD

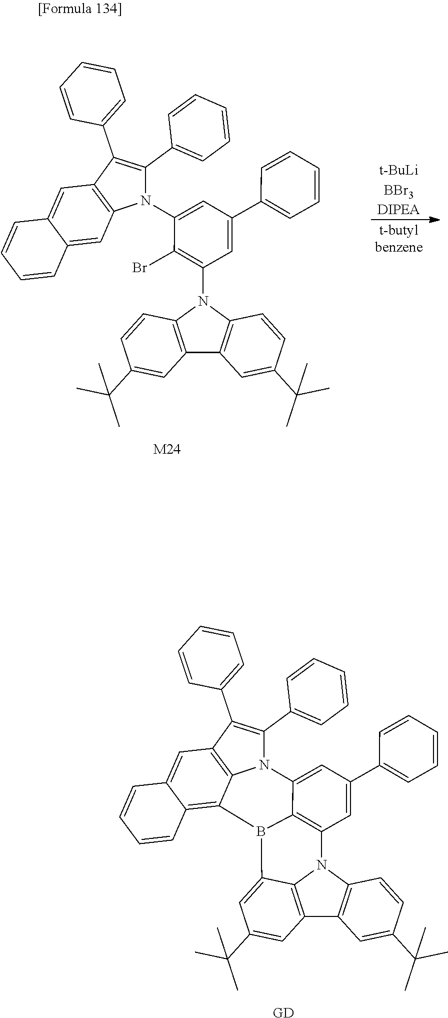

Under argon atmosphere, the intermediate M24 (2.23 g) was added to tert-butylbenzene (33 mL) and cooled to −20 degrees C., to which 1.9 M tert-butyllithium pentane solution (2.8 mL) was then added dropwise. After the dropwise addition, the obtained mixture was raised in temperature to 70 degrees C. and stirred for 30 minutes. Subsequently, a component having a boiling point lower than that of tert-butyl benzene was distilled under reduced pressure. The obtained mixture was cooled to −55 degrees C., added with boron tribromide (0.57 mL), raised to room temperature, and stirred for one hour. Subsequently, the obtained mixture was cooled to 0 degrees C., added with N,N-diisopropylethylamine (1.19 mL), stirred at room temperature until exotherm subsided, subsequently raised in temperature to 130 degrees C., and stirred overnight. After tert-butylbenzene was distilled under reduced pressure, the residue was purified by flash chromatography to obtain an orange compound (350 mg). As a result of mass spectrum analysis, this orange compound was a target compound (compound GD) and had 757.4[M+H]⁺ while a molecular weight was 756.8. In a reaction scheme, t-BuLi represents tert-butyllithium and DIPEA represents N,N-diisopropylethylamine.

### EXPLANATION OF CODES

1, 1A . . . organic EL device, 2 . . . substrate, 3 . . . anode, 4 . . . cathode, 5 . . . emitting layer, 5A, 5B . . . emitting zone, 6 . . . hole injecting layer, 7 . . . hole transporting layer, 9 . . . electron injecting layer, 81 . . . first layer, 82 . . . second layer, 100 . . . first device, 200 . . . second device, 300 . . . third device, 101, 102 . . . organic EL apparatus.

1: An organic electroluminescence device comprising:
an anode;
a cathode;
an emitting layer provided between the anode and the cathode; and
a first layer provided between the emitting layer and the cathode, wherein
the first layer comprises a first compound having at least one deuterium atom, and
the emitting layer comprises a delayed fluorescent compound.

2: The organic electroluminescence device according to claim 1, wherein
the first compound comprises at least one of partial structures represented by formulae (11) to (28) below in one molecule,
when the first compound comprises a plurality of partial structures represented by any of the formulae (11) to (14),
the partial structures represented by the formula (11) are mutually the same or different,
the partial structures represented by the formula (12) are mutually the same or different,
the partial structures represented by the formula (13) are mutually the same or different, and
the partial structures represented by the formula (14) are mutually the same or different, 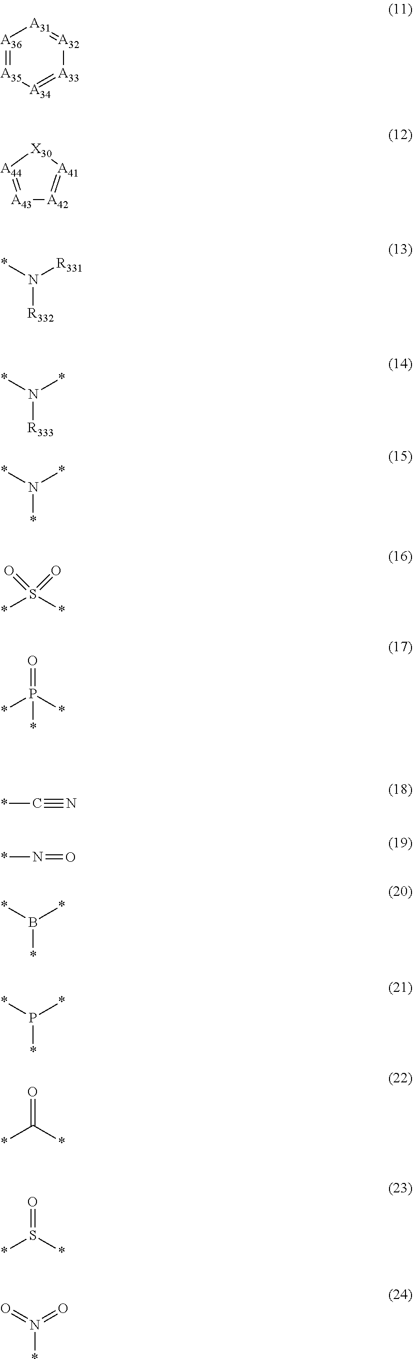 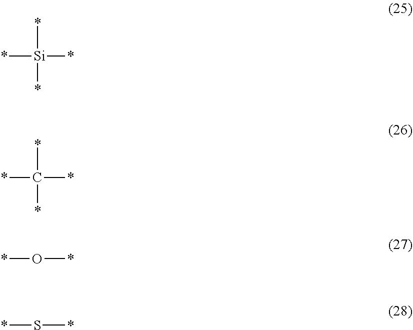
where, in the formula (11):
A₃₁ to A₃₆ are each independently a nitrogen atom, CR₃₁, or a carbon atom bonded to another atom or another structure in the molecule of the first compound;
at least one of A₃₁ to A₃₆ is a carbon atom bonded to another atom or another structure in the molecule of the first compound; and
each R₃₁ is independently a hydrogen atom or a substituent, or at least one combination of combinations of adjacent ones of R₃₁ are mutually bonded to form a ring,
in the formula (12):
A₄₁ to A₄₄ are each independently a nitrogen atom, CR₃₂, or a carbon atom bonded to another atom or another structure in the molecule of the first compound;
each R₃₂ is independently a hydrogen atom or a substituent, or at least one combination of combinations of adjacent ones of R₃₂ are mutually bonded to form a ring;
X₃₀ is NR₃₃, CR₃₄R₃₅, SiR₃₆R₃₇, an oxygen atom, a sulfur atom, a nitrogen atom bonded to another atom or another structure in the molecule of the first compound, a carbon atom bonded to R₃₈ and to another atom or another structure in the molecule of the first compound, or a silicon atom bonded to R₃₉ and to another atom or another structure in the molecule of the first compound;
at least one of carbon atoms for A₄₁ to A₄₄, a nitrogen atom for X₃₀, a carbon atom for X₃₀, or a silicon atom for X₃₀ is bonded to another atom or another structure in the molecule of the first compound, and
R₃₃ to R₃₉ are each independently a hydrogen atom or a substituent, or at least one combination of a combination of adjacent R₃₄ and R₃₅ or a combination of R₃₆ and R₃₇ are mutually bonded to form a ring,
in the formulae (13) and (14):
R₃₃₁ to R₃₃₃ are each independently a hydrogen atom or a substituent, or a combination of adjacent R₃₃₁ and R₃₃₂ are mutually bonded to form a ring;
R₃₁ to R₃₉ and R₃₃₁ to R₃₃₃ as the substituents are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms; a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted germanium group, a substituted phosphine oxide group, a nitro group, a substituted or unsubstituted carbonyl group, or a substituted boryl group;
a plurality of R₃₁ are mutually the same or different;
a plurality of R₃₂ are mutually the same or different; and
* is a bonding portion to another atom or another structure in the molecule of the first compound.

3: The organic electroluminescence device according to claim 2, wherein
in the formulae (11) and (12), at least one of R₃₁ for CR₃₁, R₃₂ for CR₃₂, or R₃₃ to R₃₉ for X₃₀ is a deuterium atom.

4: The organic electroluminescence device according to claim 2, wherein
the partial structure represented by each of the formulae (11) to (28) is a partial structure represented by any of formulae (111) to (138) below, and
when the first compound comprises a plurality of partial structures represented by any of the formulae (111) to (138), the plurality of partial structures represented by any of the formulae (111) to (138) are mutually the same or different, 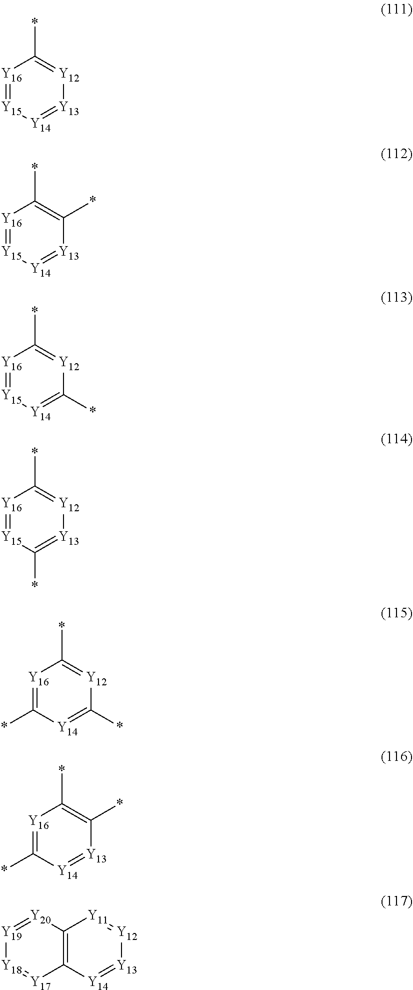 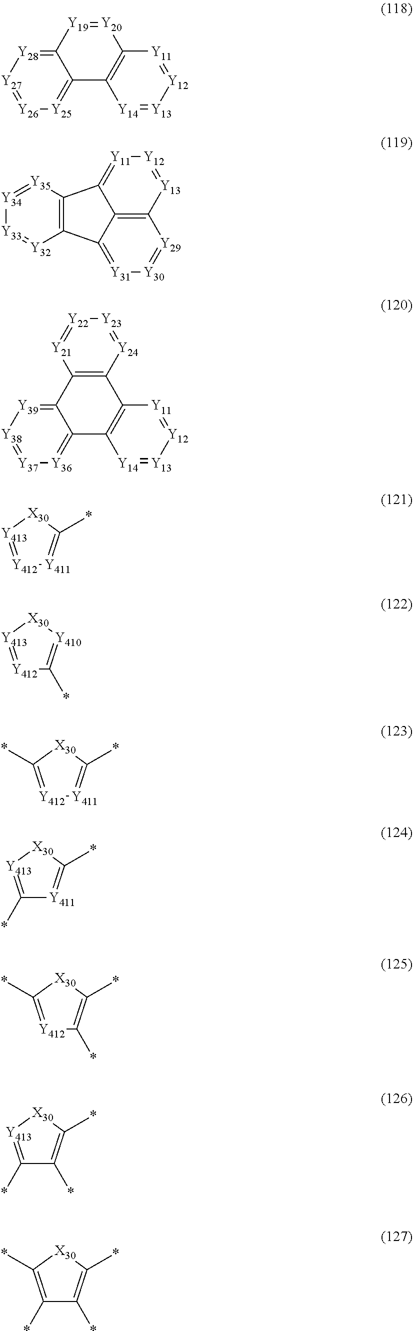 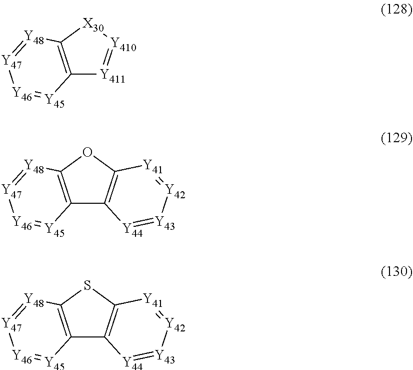 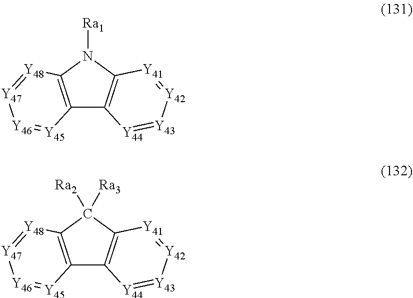 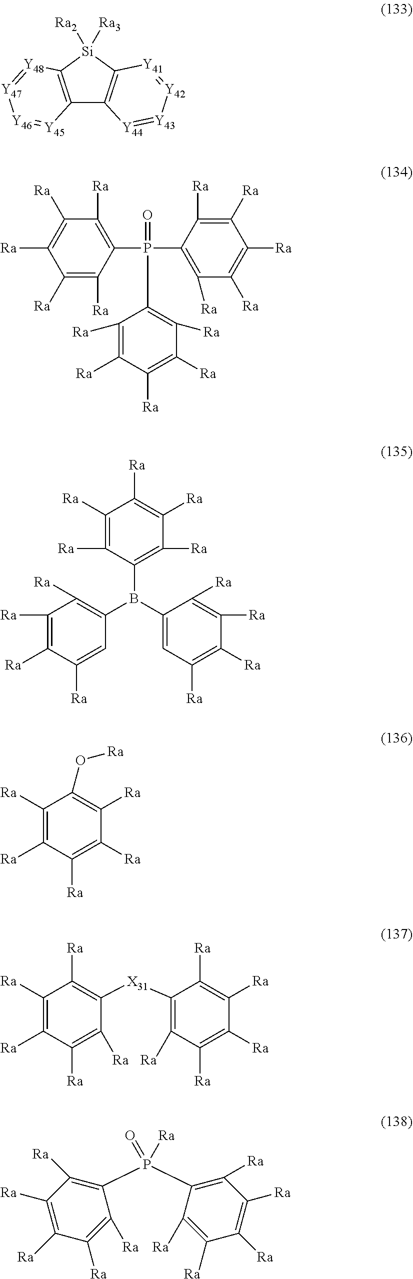
where, in the formulae (111) to (116), Y₁₂ to Y₁₆ are each independently a nitrogen atom or CR₃₁, each R₃₁ independently represents the same as R₃₁ in the formula (11), and * is a bonding portion to another atom or another structure in the molecule of the first compound;
in the formulae (117) to (120), Y₁₁ to Y₁₄ and Y₁₇ to Y₃₉ are each independently a nitrogen atom or CR₃₁, or a carbon atom bonded to another atom or another structure in the molecule of the first compound, each R₃₁ independently represents the same as R₃₁ in the formula (11), and at least one of Y₁₁ to Y₁₄ or Y₁₇ to Y₃₉ is a carbon atom bonded to another atom or another structure in the molecule of the first compound;
in the formulae (121) to (127), Y₄₁₀ to Y₄₁₃ are each independently a nitrogen atom or CR₃₂, each R₃₂ independently represents the same as R₃₂ in the formula (12), X₃₀ represents the same as X₃₀ in the formula (12), and * is a bonding portion to another atom or another structure in the molecule of the first compound;
in the formula (128), Y₄₁₀ to Y₄₁₁ and Y₄₅ to Y₄₈ are each independently a nitrogen atom or CR₃₂, or a carbon atom bonded to another atom or another structure in the molecule of the first compound, each R₃₂ independently represents the same as R₃₂ in the formula (12), X₃₀ represents the same as X₃₀ in the formula (12), at least one of carbon atoms for Y₄₁₀ to Y₄₁₁ and Y₄₅ to Y₄₈, a nitrogen atom for X₃₀, a carbon atom for X₃₀, or a silicon atom for X₃₀ is bonded to another atom or another structure in the molecule of the first compound;
in the formulae (129) to (133), Y₄₁ to Y₄₈ are each independently a nitrogen atom or CR₃₂, or a carbon atom bonded to another atom or another structure in the molecule of the first compound, Rₐ₁ to Rₐ₃ are each independently a hydrogen atom or a substituent, or a combination of Rₐ₂ and Rₐ₃ are mutually bonded to form a ring, R₃₂ represents the same as R₃₂ in the formula (12), Rₐ₁ to R₃ as the substituents each independently represent the same as R₃₂ as the substituent in the formula (12), when a plurality of Rₐ₂ are present, the plurality of Rₐ₂ are mutually the same or different, when a plurality of Rₐ₃ are present, the plurality of R₃ are mutually the same or different, and at least one of carbon atoms for Y₄₁ to Y₄₈, a nitrogen atom bonded to Rₐ₁, a carbon atom bonded to Rₐ₂, a carbon atom bonded to Rₐ₃, a silicon atom bonded to Rₐ₂, or a silicon atom bonded to Rₐ₃ is bonded to another atom or another structure in the molecule of the first compound, and
in the formulae (134) to (138), each Ra is independently a hydrogen atom or a substituent, at least one combination of combinations of adjacent ones of Ra are mutually bonded to form a ring, or Ra is a single bond bonded to another atom or another structure in the molecule of the first compound, each Ra as the substituent independently represents the same as R₃₁ as the substituent in the formula (11), a plurality of Ra are mutually the same or different, X₃₁ represents the same as X₃₀ in the formula (12), and at least one Ra is a single bond bonded to another atom or another structure in the molecule of the first compound.

5: The organic electroluminescence device according to claim 4, wherein
Rₐ₁ in the formula (131) comprises no deuterium atom.

6: The organic electroluminescence device according to claim 4, wherein
the first compound comprises no partial structure represented by the formula (131).

7: The organic electroluminescence device according to claim 4, wherein
the first compound comprises no partial structure represented by the formula (132) in which a combination of Rₐ₂ and Rₐ₃ are mutually bonded.

8: The organic electroluminescence device according to claim 4, wherein
the first compound comprises no spirofluorene structure.

9: The organic electroluminescence device according to claim 1, wherein
the first compound is not a compound represented by a formula (133A) below, 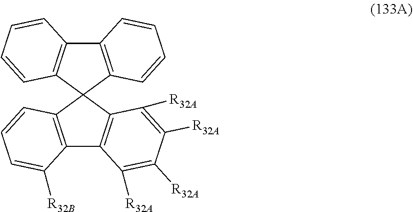
where, in the formula (133A):
each R_{32A} is independently a hydrogen atom or a substituent, or at least one combination of combinations of adjacent ones of R_{32A} are mutually bonded to form a ring, each R_{32A} as the substituent is independently a substituted or unsubstituted aryl group having 6 to 30 ring carbon atoms or a substituted or unsubstituted heteroaryl group having 5 to 30 ring atoms, and a plurality of R_{32A} are mutually the same or different; and
R_{32B} is a substituted or unsubstituted aryl group having 6 to 30 ring carbon atoms or a substituted or unsubstituted heteroaryl group having 5 to 30 ring atoms.

10: The organic electroluminescence device according to claim 2, wherein
the first compound comprises a substituted or unsubstituted electron-withdrawing group.

11: The organic electroluminescence device according to claim 10, wherein
the electron-withdrawing group comprises at least one deuterium atom.

12: The organic electroluminescence device according to claim 10, wherein
when the electron-withdrawing group comprises a substituent E1, the substituent E1 comprises at least one deuterium atom; or
when the substituent E1 further comprises a substituent E2, the substituent E2 comprises at least one deuterium atom.

13: The organic electroluminescence device according to claim 10, wherein
when the electron-withdrawing group comprises a substituent E1, the substituent E1 comprises at least one deuterium atom.

14: The organic electroluminescence device according to claim 10, wherein
when the electron-withdrawing group comprises a substituent E1 and the substituent E1 further comprises a substituent E2, the substituent E2 comprises at least one deuterium atom.

15: The organic electroluminescence device according to claim 12, wherein
the substituent E1 and the substituent E2 are each independently a substituted or unsubstituted aryl group having 6 to 22 ring carbon atoms, or a substituted or unsubstituted heterocyclic group having 5 to 20 ring atoms.

16: The organic electroluminescence device according to claim 10, wherein
a deuterium atom is bonded to at least one of a first to an eleventh atoms counting from the electron-withdrawing group.

17: The organic electroluminescence device according to claim 10, wherein
a deuterium atom is bonded to at least one of a first to an eighth atoms counting from the electron-withdrawing group.

18: The organic electroluminescence device according to claim 10, wherein
a deuterium atom is bonded to at least one of a first to a fourth atoms counting from the electron-withdrawing group.

19: The organic electroluminescence device according to claim 10, wherein
the electron-withdrawing group is a halogen atom, a cyano group, a carbonyl group, a nitro group, or a substituted or unsubstituted alkyl halide group; a monovalent or higher-valent group obtained by removing at least one hydrogen atom from a compound selected from the group consisting of a substituted or unsubstituted phosphine oxide, a substituted or unsubstituted sulfone, a substituted or unsubstituted sulfoxide, a substituted or unsubstituted nitroso, a substituted or unsubstituted pyridine, a substituted or unsubstituted pyrimidine, a substituted or unsubstituted pyridazine, a substituted or unsubstituted pyrazine, a substituted or unsubstituted triazine, a substituted or unsubstituted imidazole, a substituted or unsubstituted oxazole, a substituted or unsubstituted thiazole, a substituted or unsubstituted triazole, a substituted or unsubstituted benzimidazole, a substituted or unsubstituted benzoxazole, a substituted or unsubstituted benzothiazole, a substituted or unsubstituted benzotriazole, a substituted or unsubstituted boryl, a substituted or unsubstituted dibenzofuran, a substituted or unsubstituted dibenzothiophene, a substituted or unsubstituted fluoranthene, a substituted or unsubstituted phenanthrene, a substituted or unsubstituted chrysene, a substituted or unsubstituted triphenylene and, a substituted or unsubstituted naphthalene; a monovalent or higher-valent group formed by further fusing the monovalent or higher-valent group itself; or a monovalent or higher-valent group obtained by removing at least one hydrogen atom from a compound selected from the group consisting of a substituted or unsubstituted azadibenzofuran and a substituted or unsubstituted azadibenzothiophene.

20: The organic electroluminescence device according to claim 10, wherein
the electron-withdrawing group is a halogen atom, a cyano group, a carbonyl group, a nitro group, or a substituted or unsubstituted alkyl halide group; a monovalent or higher-valent group obtained by removing at least one hydrogen atom from a compound selected from the group consisting of a substituted or unsubstituted phosphine oxide, a substituted or unsubstituted sulfone, a substituted or unsubstituted sulfoxide, a substituted or unsubstituted nitroso, a substituted or unsubstituted pyridine, a substituted or unsubstituted pyrimidine, a substituted or unsubstituted pyridazine, a substituted or unsubstituted pyrazine, a substituted or unsubstituted triazine, a substituted or unsubstituted imidazole, a substituted or unsubstituted oxazole, a substituted or unsubstituted thiazole, a substituted or unsubstituted triazole, a substituted or unsubstituted benzimidazole, a substituted or unsubstituted benzoxazole, a substituted or unsubstituted benzothiazole, a substituted or unsubstituted benzotriazole, a substituted or unsubstituted boryl, a substituted or unsubstituted dibenzofuran, a substituted or unsubstituted dibenzothiophene, and a substituted or unsubstituted fluoranthene; a monovalent or higher-valent group formed by further fusing the monovalent or higher-valent group itself; or a monovalent or higher-valent group obtained by removing at least one hydrogen atom from a compound selected from the group consisting of a substituted or unsubstituted azadibenzofuran and a substituted or unsubstituted azadibenzothiophene.

21: The organic electroluminescence device according to claim 1, wherein
the first compound is a compound represented by a formula (1) below, 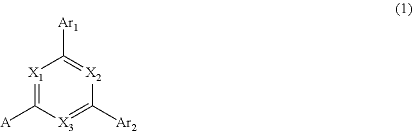
where, in the formula (1):
X₁ to X₃ are each independently a nitrogen atom or CR₁;
R₁ is a hydrogen atom or a substituent, or at least one combination of combinations of adjacent two or more of a plurality of R₁ are bonded to each other to form a ring;
at least one of X₁ to X₃ is a nitrogen atom;
each R₁ as the substituent is independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms; a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted germanium group, a substituted phosphine oxide group, a nitro group, a substituted or unsubstituted carbonyl group, or a substituted boryl group;
a plurality of R₁ are mutually the same or different;
Ar₁ and Ar₂ are each independently represented by a formula (11) below, or are each independently a substituted or unsubstituted aryl group having 6 to 30 ring carbon atoms, or a substituted or unsubstituted heteroaryl group having 5 to 30 ring atoms; and
A is represented by the formula (11), or is a substituted or unsubstituted aryl group having 6 to 30 ring carbon atoms, or a substituted or unsubstituted heteroaryl group having 5 to 30 ring atoms, 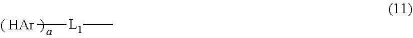
where, in the formula (11):
HAr is represented by a formula (12) below;
a is 1, 2, 3, 4 or 5;
when a is 1, L₁ is a single bond or a divalent linking group;
when a is 2, 3, 4 or 5, L₁ is a trivalent to hexavalent linking group;
a plurality of HAr are mutually the same or different;
L₁ as the linking group is a substituted or unsubstituted arylene group having 6 to 30 ring carbon atoms, or a trivalent, tetravalent, pentavalent, or hexavalent group derived from the arylene group; a substituted or unsubstituted divalent heterocyclic group having 5 to 30 ring atoms, or a trivalent, tetravalent, pentavalent, or hexavalent group derived from the heterocyclic group; or a divalent group formed by bonding two groups selected from the group consisting of a substituted or unsubstituted arylene group having 6 to 30 ring carbon atoms and a substituted or unsubstituted divalent heterocyclic group having 5 to 30 ring atoms, or a trivalent, tetravalent, pentavalent, or hexavalent group derived from the divalent group; and
the mutually bonded groups are mutually the same or different, 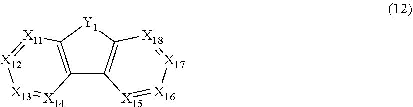
where, in the formula (12):
X₁₁ to X₁₈ are each independently a nitrogen atom, CR₁₃, or a carbon atom bonded to L₁;
a plurality of R₁₃ are mutually the same or different;
Y₁ is an oxygen atom, a sulfur atom, NR₁₈, SiR₁₁R₁₂, CR₁₄R₁₅, a nitrogen atom bonded to L₁, a silicon atom bonded to each of R₁₆ and L₁, or a carbon atom bonded to each of R₁₇ and L₁;
among carbon atoms for X₁₁ to X18, a nitrogen atom for Y₁, a silicon atom for Y₁, and a carbon atom for Y₁, any one atom is bonded to L₁;
R₁₁ to R₁₈ are each independently a hydrogen atom or a substituent, or at least one combination of a combination of adjacent ones of R₁₃, a combination of R₁₁ and R₁₂, or a combination of R₁₄ and R₁₅ are bonded to each other to form a ring; and
R₁₁ to R₁₈ as the substituents are each independently a halogen atom, a cyano group, a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkenyl group having 2 to carbon atoms, a substituted or unsubstituted alkynyl group having 2 to 30 carbon atoms; a substituted or unsubstituted silyl group, a substituted or unsubstituted alkoxy group having 1 to 30 carbon atoms, a substituted or unsubstituted aralkyl group having 7 to 30 carbon atoms, or a substituted or unsubstituted aryloxy group having 6 to 30 ring carbon atoms.

22: The organic electroluminescence device according to claim 21, wherein
the first compound is a compound represented by a formula (1A) below, 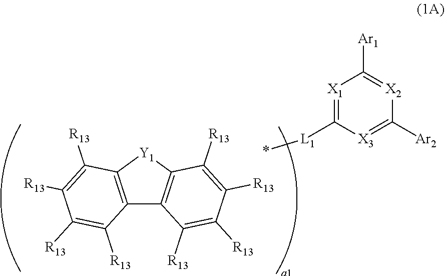
where, in the formula (1A):
X1 to X₃, Ar₁ and Ar₂ each independently represent the same as X₁ to X₃, Ar₁ and Ar₂ in the formula (1);
L₁ represents the same as L₁ in the formula (11);
a1 is 1, 2, or 3;
Y₁ represents the same as Y₁ in the formula (12);
each R₁₃ independently represents the same as R₁₃ in the formula (12);
when a1 is 1, L₁ is a single bond or a divalent linking group;
when a1 is 2, L₁ is a trivalent linking group;
when a1 is 3, L₁ is a tetravalent linking group; and
among a carbon atom bonded to R₁₃, a nitrogen atom for Y₁, a silicon atom for Y₁, and a carbon atom for Y₁, any one atom is a bonding position to L₁ (*).

23: The organic electroluminescence device according to claim 21, wherein
at least one R₁₃ for CR₁₃ is a deuterium atom, or
at least one of Ar₁ or Ar₂ comprises at least one deuterium atom.

24: The organic electroluminescence device according to claim 21, wherein
all of R₁₃ for CR₁₃ are deuterium atoms.

25: The organic electroluminescence device according to claim 21, wherein
when Ar₁ comprises one or more hydrogen atoms, all the one or more hydrogen atoms are deuterium atoms.

26: The organic electroluminescence device according to claim 21, wherein
when Ar₂ comprises one or more hydrogen atoms, all the one or more hydrogen atoms are deuterium atoms.

27: The organic electroluminescence device according to claim 21, wherein
one or two of X₁, X₂, and X₃ are each a nitrogen atom.

28: The organic electroluminescence device according to claim 21, wherein
X₁, X₂, and X₃ are each a nitrogen atom.

29: The organic electroluminescence device according to claim 21, wherein L₁ is a single bond; a substituted or unsubstituted arylene group having 6 to 30 ring carbon atoms, or a trivalent, tetravalent, pentavalent, or hexavalent group derived from the arylene group; or a substituted or unsubstituted divalent heterocyclic group having 5 to 30 ring atoms, or a trivalent, tetravalent, pentavalent, or hexavalent group derived from the heterocyclic group.

30: The organic electroluminescence device according to claim 21, wherein L₁ is a single bond; a substituted or unsubstituted arylene group having 6 to 30 ring carbon atoms, or a trivalent group derived from the arylene group; or a substituted or unsubstituted divalent heterocyclic group having 5 to 30 ring atoms, or a trivalent group derived from the heterocyclic group.

31: The organic electroluminescence device according to claim 21, wherein
Ar₁ and Ar₂ are each independently represented by the formula (11), or are each independently a substituted or unsubstituted aryl group having 6 to 30 ring carbon atoms.

32: The organic electroluminescence device according to claim 1, wherein
the delayed fluorescent compound is a compound represented by a formula (2) or a formula (22) below, 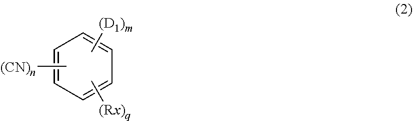
where, in the formula (2):
n is 1, 2, 3 or 4;
m is 1, 2, 3 or 4;
q is 0, 1, 2, 3 or 4;
m+n+q=6 is satisfied;
CN is a cyano group;
D₁ is a group represented by a formula (2a), (2b) or (2c) below, and when a plurality of D₁ are present, the plurality of D₁ are mutually the same or different;
Rx is a hydrogen atom or a substituent, or at least one combination of combinations of adjacent ones of Rx are mutually bonded to form a ring, and when a plurality of Rx are present, the plurality of Rx are mutually the same or different;
each Rx as the substituent is independently a halogen atom, a substituted or unsubstituted aryl group having 6 to 30 ring carbon atoms, a substituted or unsubstituted heterocyclic group having 5 to 30 ring atoms, a substituted or unsubstituted amino group, a substituted or unsubstituted carbonyl group, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkylsilyl group having 3 to 30 carbon atoms, or a substituted or unsubstituted arylsilyl group having 6 to 60 ring carbon atoms; and
CN, D₁ and Rx are bonded to respective carbon atoms of a six-membered ring, 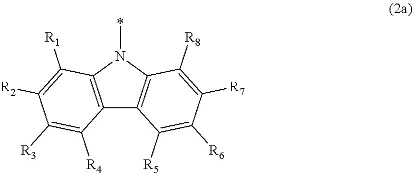
where, in the formula (2a):
R₁ to R₈ are each independently a hydrogen atom or a substituent, or at least one combination of a combination of R₁ and R₂, a combination of R₂ and R₃, a combination of R₃ and R₄, a combination of R₅ and R₆, a combination of R₆ and R₇, or a combination of R₇ and R₈ are mutually bonded to form a ring;
R₁ to R₈ as the substituents are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkylsilyl group having 3 to 30 carbon atoms, a substituted or unsubstituted arylsilyl group having 6 to 60 ring carbon atoms, a hydroxy group, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 ring carbon atoms, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and
* represents a bonding position to a carbon atom in a six-membered ring in the formula (2), 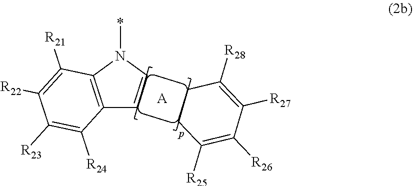
where, in the formula (2b):
R₂₁ to R₂₈ are each independently a hydrogen atom or a substituent, or at least one combination of a combination of R₂₁ and R₂₂, a combination of R₂₂ and R₂₃, a combination of R₂₃ and R₂₄, a combination of R₂₅ and R₂₆, a combination of R₂₆ and R₂₇, or a combination of R₂₇ and R₂₈ are mutually bonded to form a ring;
R₂₁ to R₂₈ as the substituents each independently represent the same as R₁ to R₈ in the formula (2a);
A represents a cyclic structure represented by a formula (211) or (212) below, and the cyclic structure A is fused with at least one adjacent cyclic structure at any position;
p is 1, 2, 3 or 4;
when p is 2, 3 or 4, a plurality of cyclic structures A are mutually the same or different; and
* represents a bonding position to a carbon atom in a six-membered ring in the formula (2), 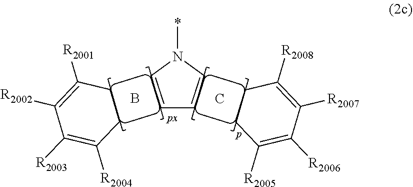
where, in the formula (2c):
R₂₀₀₁ to R₂₀₀₈ are each independently a hydrogen atom or a substituent, or at least one combination of a combination of R₂₀₀₁ and R₂₀₀₂, a combination of R₂₀₀₂ and R₂₀₀₃, a combination of R₂₀₀₃ and R₂₀₀₄, a combination of R₂₀₀₅ and R₂₀₀₆, a combination of R₂₀₀₆ and R₂₀₀₇, or a combination of R₂₀₀₇ and R₂₀₀₈ are mutually bonded to form a ring;
R₂₀₀₁ to R₂₀₀₈ as the substituents each independently represent the same as R₁ to R₈ as the substituents in the formula (2a);
B represents a cyclic structure represented by the formula (211) or (212), and the cyclic structure B is fused with at least one adjacent cyclic structure at any position;
px is 1, 2, 3 or 4;
when px is 2, 3 or 4, a plurality of cyclic structures B are mutually the same or different;
C represents a cyclic structure represented by the formula (211) or (212), and the cyclic structure C is fused with at least one adjacent cyclic structure at any position;
py is 1, 2, 3 or 4;
when py is 2, 3 or 4, a plurality of cyclic structures C are mutually the same or different; and
* represents a bonding position to a carbon atom in a six-membered ring in the formula (2), 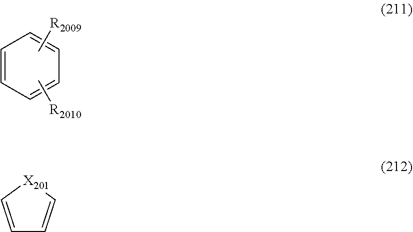
where, in the formula (211), R₂₀₀₉ and R₂₀₁₀ are each independently a hydrogen atom or a substituent, or bonded to a part of an adjacent cyclic structure to form a ring, or a combination of R₂₀₀₉ and R₂₀₁₀ are mutually bonded to form a ring;
in the formula (212), X₂₀₁ is CR₂₀₁₁R₂₀₁₂, NR₂₀₁₃, a sulfur atom, or an oxygen atom, and R₂₀₁₁, R₂₀₁₂ and R₂₀₁₃ are each independently a hydrogen atom or a substituent, or R₂₀₁₁ and R₂₀₁₂ are mutually bonded to form a ring; and
R₂₀₀₉, R₂₀₁₀, R₂₀₁₁, R₂₀₁₂ and R₂₀₁₃ as the substituents each independently represent the same as R₁ to R₈ as the substituents in the formula (2a), 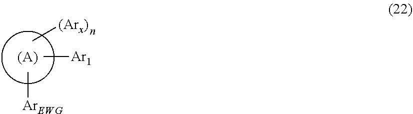
where, in the formula (22):
Ar₁ is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by formulae (1a) to (1j) below;
Ar_{EWG} is a substituted or unsubstituted heteroaryl group having 5 to 30 ring atoms that comprises at least one nitrogen atom in a ring, or an aryl group having 6 to 30 ring carbon atoms that is substituted by at least one cyano group;
each Ar_{X} is independently a hydrogen atom or a substituent, and Ar_{X} as the substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by the formulae (1a) to (1j);
n is 0, 1, 2, 3, 4 or 5, and when n is 2, 3, 4 or 5, a plurality of Ar_{X} are mutually the same or different;
a ring (A) is a substituted or unsubstituted aromatic hydrocarbon ring or a substituted or unsubstituted heterocycle, the ring (A) is a five-membered ring, a six-membered ring, or a seven-membered ring, and Ar_{EWG}, Ar₁ and Ar_{X} are bonded to respective ones of elements forming the ring (A); and
at least one of Ar₁ or Ar_{X} is a group selected from the group consisting of groups represented by the formulae (1a) to (1j), 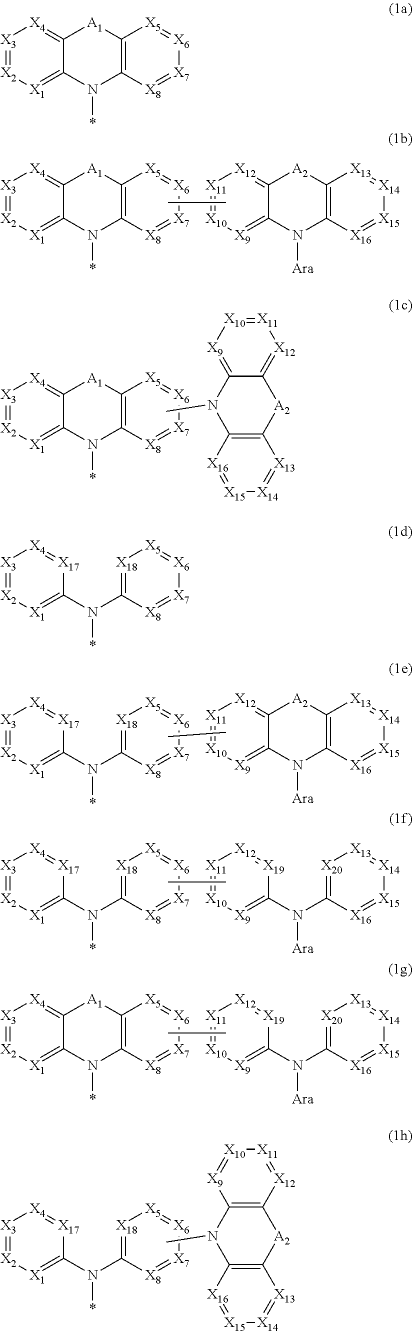 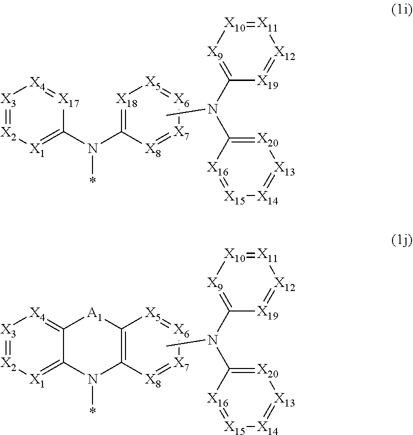
where, in the formulae (1a) to (1j), X₁ to X₂₀ are each independently a nitrogen atom (N) or a carbon atom bonded with R_{A1} (C—R_{A1});
in the formula (1b), one of X₅ to X8 is a carbon atom bonded to one of X₉ to X₁₂, and one of X₉ to X₁₂ is a carbon atom bonded to one of X₅ to X₈;
in the formula (1c), one of X₅ to X8 is a carbon atom bonded to a nitrogen atom in a ring comprising A₂;
in the formula (1e), one of X₅ to X8 and X18 is a carbon atom bonded to one of X₉ to X₁₂, and one of X₉ to X₁₂ is a carbon atom bonded to one of X₅ to X₈ and X₁₈;
in the formula (1f), one of X₅ to X8 and X18 is a carbon atom bonded to one of X₉ to X₁₂ and X₁₉, and one of X₉ to X₁₂ and X₁₉ is a carbon atom bonded to one of X₅ to X8 and X18;
in the formula (1g), one of X₅ to X8 is a carbon atom bonded to one of X₉ to X₁₂ and X₁₉, and one of X₉ to X₁₂ and X₁₉ is a carbon atom bonded to one of X₅ to X₈;
in the formula (1h), one of X₅ to X₅ and X₁₈ is a carbon atom bonded to a nitrogen atom in a ring comprising A₂;
in the formula (1i), one of X₅ to X₈ and X₁₈ is a carbon atom bonded to a nitrogen atom that links a ring comprising X₉ to X₁₂ and X₁₉ with a ring comprising X₁₃ to X₁₆ and X₂₀;
in the formula (1j), one of X₅ to X₈ is a carbon atom bonded to a nitrogen atom that links a ring comprising X₉ to X₁₂ and X₁₉ with a ring comprising X₁₃ to X₁₆ and X₂₀;
each R_{A1} is independently a hydrogen atom or a substituent, or at least one combination of combinations of a plurality of R_{A1} are mutually directly bonded to form a ring or bonded via a hetero atom to form a ring; and
R_{A1} as the substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;
when a plurality of R_{A1} as substituents are present, the plurality of R_{A1} are mutually the same or different;
in the formulae (1a) to (1j), * represents a bonding position to the ring (A);
in the formulae (1a) to (1j), A₁ and A₂ are each independently a single bond, an oxygen atom (O), a sulfur atom (S), C(R₂₀₂₁)(R₂₀₂₂), Si(R₂₀₂₃)(R₂₀₂₄), C(═O), S(═O), SO₂ or N(R₂₀₂₅);
R₂₀₂₁ to R₂₀₂₅ are each independently a hydrogen atom or a substituent, and R₂₀₂₁ to R₂₀₂₅ as the substituents are each independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group; and
in the formulae (1a) to (1j), Ara is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, and a substituted silyl group.

33: The organic electroluminescence device according to claim 32, wherein
D₁ is a group represented by one of formulae (D-21) to (D-37) below, 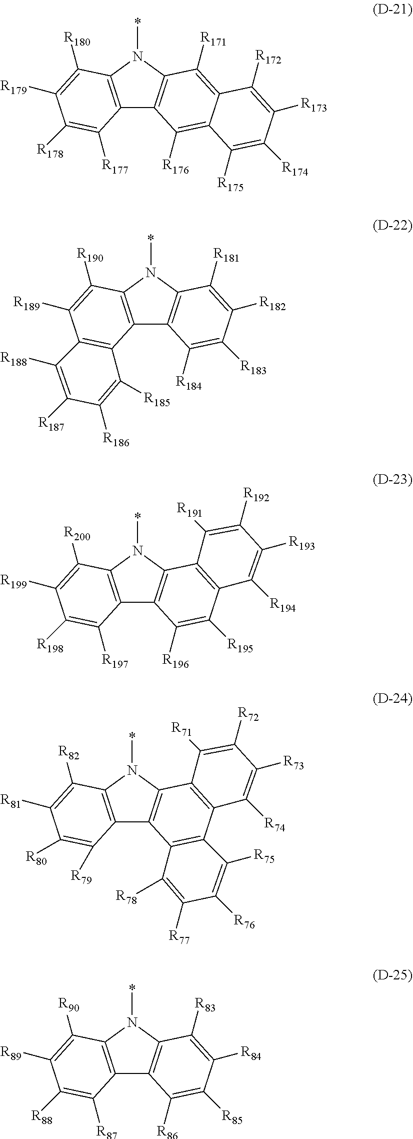
where, in the formulae (D-21) to (D-25), R₁₇₁ to R₂₀₀ and R₇₁ to R₉₀ are each independently a hydrogen atom or a substituent, or at least one combination of a combination of R₁₇₁ and R₁₇₂, a combination of R₁₇₂ and R₁₇₃, a combination of R₁₇₃ and R₁₇₄, a combination of R₁₇₄ and R₁₇₅, a combination of R₁₇₅ and R₁₇₆, a combination of R₁₇₇ and R₁₇₈, a combination of R₁₇₈ and R₁₇₉, a combination of R₁₇₉ and R₁₈₀, a combination of R₁₈₁ and R₁₈₂, a combination of R₁₈₂ and R₁₈₃, a combination of R₁₈₃ and R₁₈₄, a combination of R₁₈₅ and R₁₈₆, a combination of R₁₈₆ and R₁₈₇, a combination of R₁₈₇ and R₁₈₈, a combination of R₁₈₈ and R₁₈₉, a combination of R₁₈₉ and R₁₉₀, a combination of R₁₉₁ and R₁₉₂, a combination of R₁₉₂ and R₁₉₃, a combination of R₁₉₃ and R₁₉₄, a combination of R₁₉₄ and R₁₉₅, a combination of R₁₉₅ and R₁₉₆, a combination of R₁₉₇ and R₁₉₈, a combination of R₁₉₈ and R₁₉₉, a combination of R₁₉₉ and R₂₀₀, a combination of R₇₁ and R₇₂, a combination of R₇₂ and R₇₃, a combination of R₇₃ and R₇₄, a combination of R₇₅ and R₇₆, a combination of R₇₆ and R₇₇, a combination of R₇₇ and R₇₈, a combination of R₇₉ and R₈₀, a combination of R₈₀ and R₈₁, or a combination of R₈₁ and R₈₂ are bonded to each other to form a ring;
R₁₇₁ to R₂₀₀ and R₇₁ to R₉₀ as the substituents are each independently a halogen atom, a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkylsilyl group having 3 to 6 carbon atoms, a hydroxy group, a substituted or unsubstituted alkoxy group having 1 to 6 carbon atoms, a substituted or unsubstituted alkoxy halide group having 1 to 6 carbon atoms, a substituted or unsubstituted aryloxy group having 6 to 14 ring carbon atoms, a substituted or unsubstituted alkylamino group having 2 to 12 carbon atoms, a thiol group, a substituted or unsubstituted alkylthio group having 1 to 6 carbon atoms, or a substituted or unsubstituted arylthio group having 6 to 14 ring carbon atoms; and
* represents a bonding position to a carbon atom in a six-membered ring in the formula (2), 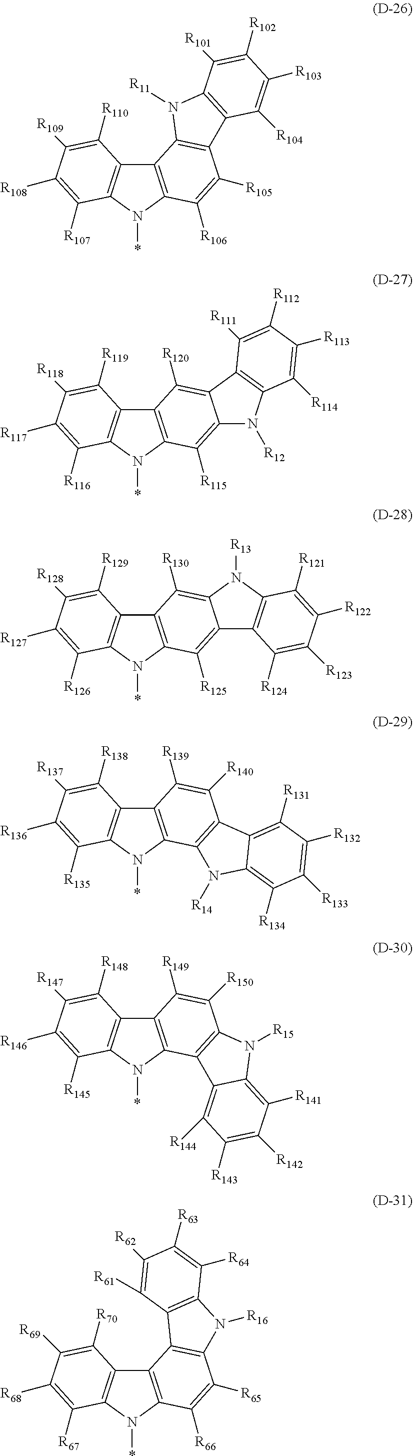
where, in the formulae (D-26) to (D-31), R₁₁ to R₁₆ are each a substituent, and R₁₀₁ to R₁₅₀ and R₆₁ to R₇₀ are each independently a hydrogen atom or a substituent, or at least one combination of a combination of R₁₀₁ and R₁₀₂, a combination of R₁₀₂ and R₁₀₃, a combination of R₁₀₃ and R₁₀₄, a combination of R₁₀₅ and R₁₀₆, a combination of R₁₀₇ and R₁₀₈, a combination of R₁₀₈ and R₁₀₉, a combination of R₁₀₉ and R₁₁₀, a combination of R₁₁₁ and R₁₁₂, a combination of R₁₁₂ and R₁₁₃, a combination of R₁₁₃ and R₁₁₄, a combination of R₁₁₆ and R₁₁₇, a combination of R₁₁₇ and R₁₁₈, a combination of R₁₁₈ and R₁₁₉, a combination of R₁₂₁ and R₁₂₂, a combination of R₁₂₂ and R₁₂₃, a combination of R₁₂₃ and R₁₂₄, a combination of R₁₂₆ and R₁₂₇, a combination of R₁₂₇ and R₁₂₈, a combination of R₁₂₈ and R₁₂₉, a combination of R₁₃₁ and R₁₃₂, a combination of R₁₃₂ and R₁₃₃, a combination of R₁₃₃ and R₁₃₄, a combination of R₁₃₅ and R₁₃₆, a combination of R₁₃₆ and R₁₃₇, a combination of R₁₃₇ and R₁₃₈, a combination of R₁₃₉ and R₁₄₀, a combination of R₁₄₁ and R₁₄₂, a combination of R₁₄₂ and R₁₄₃, a combination of R₁₄₃ and R₁₄₄, a combination of R₁₄₅ and R₁₄₆, a combination of R₁₄₆ and R₁₄₇, a combination of R₁₄₇ and R₁₄₈, a combination of R₁₄₉ and R₁₅₀, a combination of R₆₁ and R₆₂, a combination of R₆₂ and R₆₃, a combination of R₆₃ and R₆₄, a combination of R₆₅ and R₆₆, a combination of R₆₇ and R₆₈, a combination of R₆₈ and R₆₉, or a combination of R₆₉ and R₇₀ are bonded to each other to form a ring;
R₁₀₁ to R₁₅₀ and R₆₁ to R₇₀ as the substituents are each independently a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkylsilyl group having 3 to 6 carbon atoms, a hydroxy group, a substituted or unsubstituted alkoxy group having 1 to 6 carbon atoms, a substituted or unsubstituted aryloxy group having 6 to 14 ring carbon atoms, a substituted or unsubstituted arylamino group having 6 to 28 ring carbon atoms, a substituted or unsubstituted alkylamino group having 2 to 12 carbon atoms, a thiol group, a substituted or unsubstituted alkylthio group having 1 to 6 carbon atoms, or a substituted or unsubstituted arylthio group having 6 to 14 ring carbon atoms;
R₁₁ to R₁₆ as the substituents are each independently a substituted or unsubstituted alkyl group having 1 to 6 carbon atoms, a substituted or unsubstituted aryl group having 6 to 14 ring carbon atoms, a substituted or unsubstituted heterocyclic group having 5 to 14 ring atoms, a substituted or unsubstituted alkylsilyl group having 3 to 6 carbon atoms, a substituted or unsubstituted aryloxy group having 6 to 14 ring carbon atoms, a substituted or unsubstituted alkylamino group having 2 to 12 carbon atoms, a substituted or unsubstituted alkylthio group having 1 to 6 carbon atoms, or a substituted or unsubstituted arylthio group having 6 to 14 ring carbon atoms; and
* represents a bonding position to a carbon atom in a six-membered ring in the formula (2), 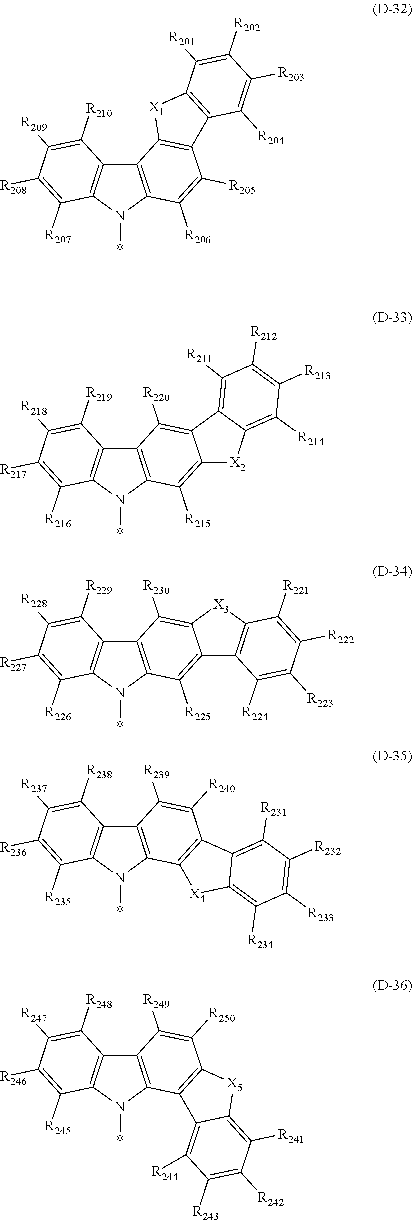 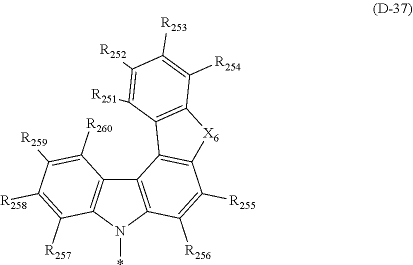
where, in the formulae (D-32) to (D-37):
X₁ to X₆ are each independently an oxygen atom, a sulfur atom, or CR₁₅₁R₁₅₂;
R₁₅₁ and R₁₅₂ are each independently a hydrogen atom or a substituent, or R₁₅₁ and R₁₅₂ are bonded to each other to form a ring;
R₂₀₁ to R₂₆₀ are each independently a hydrogen atom or a substituent, or at least one combination of a combination of R₂₀₁ and R₂₀₂, a combination of R₂₀₂ and R₂₀₃, a combination of R₂₀₃ and R₂₀₄, a combination of R₂₀₅ and R₂₀₆, a combination of R₂₀₇ and R₂₀₈, a combination of R₂₀₈ and R₂₀₉, a combination of R₂₀₉ and R₂₁₀, a combination of R₂₁₁ and R₂₁₂, a combination of R₂₁₂ and R₂₁₃, a combination of R₂₁₃ and R₂₁₄, a combination of R₂₁₆ and R₂₁₇, a combination of R₂₁₇ and R₂₁₈, a combination of R₂₁₈ and R₂₁₉, a combination of R₂₂₁ and R₂₂₂, a combination of R₂₂₂ and R₂₂₃, a combination of R₂₂₃ and R₂₂₄, a combination of R₂₂₆ and R₂₂₇, a combination of R₂₂₇ and R₂₂₈, a combination of R₂₂₈ and R₂₂₉, a combination of R₂₃₁ and R₂₃₂, a combination of R₂₃₂ and R₂₃₃, a combination of R₂₃₃ and R₂₃₄, a combination of R₂₃₅ and R₂₃₆, a combination of R₂₃₆ and R₂₃₇, a combination of R₂₃₇ and R₂₃₈, a combination of R₂₃₉ and R₂₄₀, a combination of R₂₄₁ and R₂₄₂, a combination of R₂₄₂ and R₂₄₃, a combination of R₂₄₃ and R₂₄₄, a combination of R₂₄₅ and R₂₄₆, a combination of R₂₄₆ and R₂₄₇, a combination of R₂₄₇ and R₂₄₈, a combination of R₂₄₉ and R₂₅₀, a combination of R₂₅₁ and R₂₅₂, a combination of R₂₅₂ and R₂₅₃, a combination of R₂₅₃ and R₂₅₄, a combination of R₂₅₅ and R₂₅₆, a combination of R₂₅₇ and R₂₅₈, a combination of R₂₅₈ and R₂₅₉, or a combination of R₂₅₉ and R₂₆₀ are bonded to each other to form a ring;
R₁₅₁, R₁₅₂ and R₂₀₁ to R₂₆₀ as the substituents are each independently a halogen atom, a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkyl halide group having 1 to 6 carbon atoms, a substituted or unsubstituted cycloalkyl group having 3 to 30 ring carbon atoms, a substituted or unsubstituted alkylsilyl group having 3 to 6 carbon atoms, a hydroxy group, a substituted or unsubstituted alkoxy group having 1 to 6 carbon atoms, a substituted or unsubstituted alkoxy halide group having 1 to 6 carbon atoms, a substituted or unsubstituted aryloxy group having 6 to 14 ring carbon atoms, a substituted or unsubstituted arylamino group having 6 to 28 ring carbon atoms, a substituted or unsubstituted alkylamino group having 2 to 12 carbon atoms, a thiol group, a substituted or unsubstituted alkylthio group having 1 to 6 carbon atoms, or a substituted or unsubstituted arylthio group having 6 to 14 ring carbon atoms; and
* represents a bonding position to a carbon atom in a six-membered ring in the formula (2).

34: The organic electroluminescence device according to claim 1, wherein
the emitting layer comprises a compound M2 as the delayed fluorescent compound and a fluorescent compound M1, and
a singlet energy S₁(Mat2) of the compound M2 and a singlet energy S₁(Mat1) of the compound M1 satisfy a relationship of a numerical formula (Numerical Formula 1) below,
S₁(Mat2)>S₁(Mat1) (Numerical Formula 1).

35: The organic electroluminescence device according to claim 34, wherein
the emitting layer further comprises a compound M3, and
the singlet energy S₁(Mat2) of the compound M2, the singlet energy S₁(Mat1) of the compound M1, and a singlet energy S₁(Mat3) of the compound M3 satisfy a relationship of a numerical formula (Numerical Formula 2) below,
S₁(Mat3)>S₁(Mat2)>S₁(Mat1) (Numerical Formula 2).

36: The organic electroluminescence device according to claim 35, wherein
the compound M3 comprises at least one of partial structures represented by formulae (311) to (331) below in one molecule, and
when the compound M3 comprises a plurality of partial structures represented by any of the formulae (311) to (331), the plurality of partial structures represented by any of the formulae (311) to (331) are the same or different, 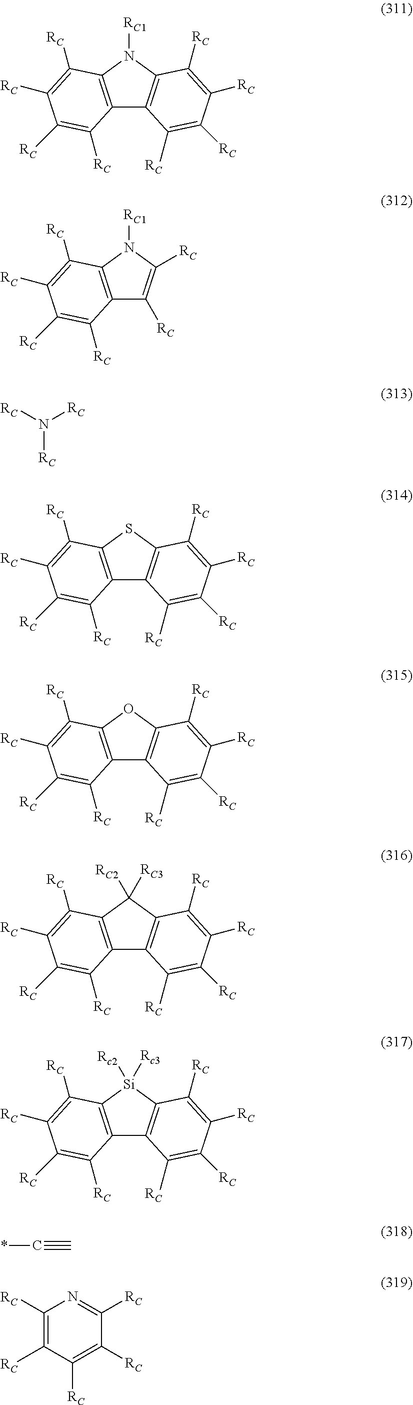 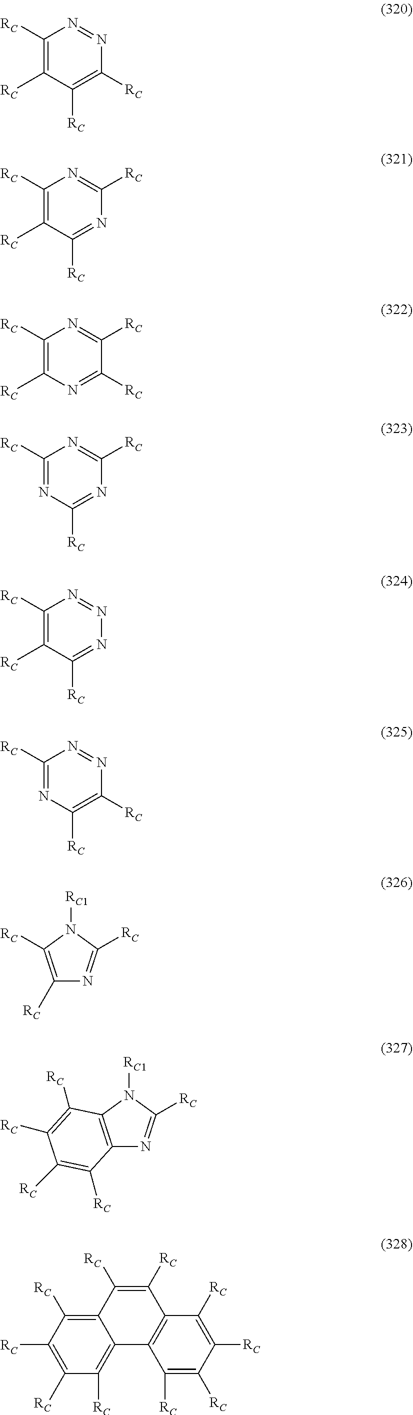 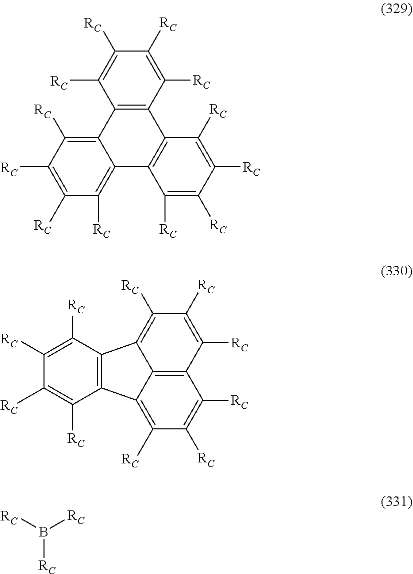
where, in the formulae (311) to (317) and (319) to (331):
R_{C} and R_{C1} to R_{C3} are each independently a hydrogen atom or a substituent; at least one combination of combinations of adjacent ones of R_{C} or a combination of R_{C2} and R_{C3} are mutually bonded to form a ring; or R_{C} and R_{C1} to R_{C3} are each independently a single bond bonded to another atom or another structure in the molecule of the compound M3;
at least one of R_{C} or R_{C1} to R_{C3} is a single bond bonded to another atom or another structure in the molecule of the compound M3;
* in the formula (318) represents a bonding position to another atom or another structure in the molecule of the compound M3;
R_{C} and R_{C1} to R_{C3} as the substituents are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms; a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted germanium group, a substituted phosphine oxide group, a nitro group, a substituted or unsubstituted carbonyl group, or a substituted boryl group;
a plurality of R_{C} are mutually the same or different;
when a plurality of R_{C1} are present, the plurality of R_{C1} are mutually the same or different;
when a plurality of R_{C2} are present, the plurality of R_{C2} are mutually the same or different; and
when a plurality of R_{C3} are present, the plurality of R_{C3} are mutually the same or different.

37: The organic electroluminescence device according to claim 36, wherein
the compound M3 comprises at least one partial structure represented by the formula (311), the formulae (314) to (319), the formula (321), the formula (323), or the formula (330) in one molecule.

38: The organic electroluminescence device according to claim 36, wherein
the compound M3 comprises at least one partial structure represented by the formula (311), the formulae (314) to (315), the formula (321), or the formula (323) in one molecule.

39: The organic electroluminescence device according to claim 36, wherein
the compound M3 comprises a partial structure represented by the formula (311) in one molecule.

40: The organic electroluminescence device according to claim 1, wherein
the emitting layer comprises the compound M2 as the delayed fluorescent compound and a compound M4, and
the singlet energy S₁(Mat2) of the compound M2 and a singlet energy S₁(Mat4) of the compound M4 satisfy a relationship of a numerical formula (Numerical Formula 3) below,
S₁(Mat4)>S₁(Mat2) (Numerical Formula 3).

41: The organic electroluminescence device according to claim 1, wherein
the first layer is in direct contact with the emitting layer.

42: The organic electroluminescence device according to claim 1, wherein
a second layer is provided between the first layer and the cathode,
the second layer comprises a second compound represented by a formula (B) below, and
the first compound is different from the second compound, 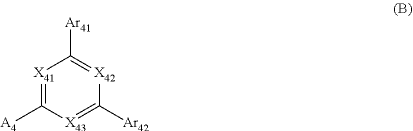
where, in the formula (B):
X₄₁ to X₄₃ are each independently a nitrogen atom or CR₄₁;
R₄₁ is a hydrogen atom or a substituent, or at least one combination of combinations of adjacent two or more of a plurality of R₄₁ are mutually bonded to form a ring;
at least one of X₄₁ to X₄₃ is a nitrogen atom;
R₄₁ is a hydrogen atom or a substituent;
each R₄₁ as the substituent is independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted alkenyl group having 2 to 30 carbon atoms, a substituted or unsubstituted alkynyl group having 2 to 30 carbon atoms; a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, a substituted or unsubstituted arylthio group having 6 to 30 ring carbon atoms, a substituted or unsubstituted aralkyl group having 7 to 30 carbon atoms, a substituted germanium group, a substituted phosphine oxide group, a nitro group, a substituted or unsubstituted carbonyl group, or a substituted boryl group;
a plurality of R₄₁ are mutually the same or different;
Ar₄₁ and Ar₄₂ are each independently represented by a formula (1B) below, or are each independently a substituted or unsubstituted aryl group having 6 to 30 ring carbon atoms, or a substituted or unsubstituted heteroaryl group having 5 to 30 ring atoms; and
A₄ is represented by the formula (1B), or is a substituted or unsubstituted aryl group having 6 to 30 ring carbon atoms, or a substituted or unsubstituted heteroaryl group having 5 to 30 ring atoms, 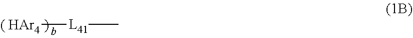
where, in the formula (1B):
HAr₄ is represented by a formula (2B) below;
b is 1, 2, 3, 4, or 5;
when b is 1, L₄₁ is a single bond or a divalent linking group;
when b is 2, 3, 4 or 5, L₄₁ is a trivalent to hexavalent linking group;
a plurality of HAr₄ are mutually the same or different;
L₄₁ as the linking group is a substituted or unsubstituted arylene group having 6 to 30 ring carbon atoms, or a trivalent, tetravalent, pentavalent, or hexavalent group derived from the arylene group; a substituted or unsubstituted divalent heterocyclic group having 5 to 30 ring atoms, or a trivalent, tetravalent, pentavalent, or hexavalent group derived from the heterocyclic group; or a divalent group formed by bonding two groups selected from the group consisting of a substituted or unsubstituted arylene group having 6 to 30 ring carbon atoms and a substituted or unsubstituted divalent heterocyclic group having 5 to 30 ring atoms, or a trivalent, tetravalent, pentavalent, or hexavalent group derived from the divalent group; and
the mutually bonded groups are mutually the same or different, 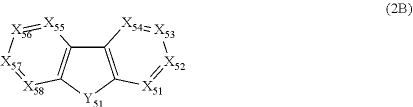
where, in the formula (2B):
X₅₁ to X₅₈ are each independently a nitrogen atom, CR₅₃, or a carbon atom bonded to L₄₁;
a plurality of R₅₃ are mutually the same or different;
Y₅₁ is an oxygen atom, a sulfur atom, NR₅₈, SiR₅₁R₅₂, CR₅₄R₅₅, a nitrogen atom bonded to L₄₁, a silicon atom bonded to each of R₅₆ and L₄₁, or a carbon atom bonded to each of R₅₇ and L₄₁;
among carbon atoms for X₅₁ to X₅₈, a nitrogen atom for Y₅₁, a silicon atom for Y₅₁, and a carbon atom for Y₅₁, any one atom is bonded to L₄₁;
R₅₁ to R₅₈ are each independently a hydrogen atom or a substituent, or at least one combination of a combination of adjacent ones of R₅₃, a combination of R₅₁ and R₅₂, or a combination of R₅₄ and R₅₅ are bonded to each other to form a ring; and
R₅₁ to R₅₈ as the substituents are each independently a halogen atom, a cyano group, a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted alkenyl group having 2 to carbon atoms, a substituted or unsubstituted alkynyl group having 2 to 30 carbon atoms, a substituted or unsubstituted silyl group, a substituted or unsubstituted alkoxy group having 1 to 30 carbon atoms, a substituted or unsubstituted aralkyl group having 7 to 30 carbon atoms, or a substituted or unsubstituted aryloxy group having 6 to 30 ring carbon atoms.

43: An organic electroluminescence apparatus, comprising:
a first device that is the organic electroluminescence device according to claim 1;
a second device that is an organic electroluminescence device different from the first device; and
a substrate, wherein
the first device and the second device are arranged in parallel on the substrate, and
the first layer of the first device is a common layer provided in common to the first device and the second device.

44: The organic electroluminescence apparatus according to claim 43, further comprising a third device that is an organic electroluminescence device different from the first device and the second device, wherein
the first device, the second device, and the third device are arranged in parallel on the substrate, and
the first layer of the first device is a common layer provided in common to the first device, the second device, and the third device.

45: An electronic device comprising the organic electroluminescence device according to claim 1.

46: An electronic device comprising the organic electroluminescence apparatus according to claim 43.
